(19) United States
(12) Patent Application Publication
(10) Pub. No.: US 2025/0152824 A1
(43) Pub. Date: May 15, 2025

(54) MEDICAMENT DELIVERY DEVICES FOR ADMINISTRATION OF A MEDICAMENT WITHIN A PREFILLED SYRINGE

(71) Applicant: kaleo, Inc., Richmond, VA (US)

(72) Inventors: Eric S. EDWARDS, Moseley, VA (US); Evan T. EDWARDS, Charlottesville, VA (US); Mark J. LICATA, Doswell, VA (US); Paul F. MEYERS, Fishers, IN (US)

(73) Assignee: kaleo, Inc., Richmond, VA (US)

(21) Appl. No.: 19/022,755

(22) Filed: Jan. 15, 2025

Related U.S. Application Data
(63) Continuation of application No. 17/869,570, filed on Jul. 20, 2022.
Application No. 17/869,570 is a continuation of application No. 16/504,725, filed on Jul. 8, 2019, now Pat. No. 11,426,520.
Application No. 16/504,725 is a continuation of application No. 16/280,488, filed on Feb. 20, 2019, now Pat. No. 10,342,924.
Application No. 16/280,488 is a continuation of application No. 14/803,821, filed on Jul. 20, 2015, now Pat. No. 10,238,806.
Application No. 14/803,821 is a continuation of application No. 13/357,935, filed on Jan. 25, 2012, now Pat. No. 9,084,849.
(60) Provisional application No. 61/436,301, filed on Jan. 26, 2011.

(51) Int. Cl. A61M 5/20; A61M 5/315; A61M 5/32

(52) U.S. Cl. CPC A61M 5/2033; A61M 5/20; A61M 5/315; A61M 5/3202; A61M 2005/2013; A61M 2005/206; A61M 5/2066; A61M 2005/2073; A61M 2005/208; A61M 2005/2086; A61M 5/3204

(57) ABSTRACT

An apparatus includes a housing, a medicament container and a movable member. The medicament container is movable within the housing between a first position and a second position in response to a force produced by an energy storage member. A proximal end portion of the medicament container includes a flange and has a plunger disposed therein. A first shoulder of the movable member exerts the force on the flange to move the medicament container from the first position to the second position. A portion of the first shoulder deforms when the medicament container is in the second position such that at least a portion of the force is exerted upon the plunger. A second shoulder of the movable member exerts a retraction force on the flange to move the medicament container from the second position towards the first position.

### CROSS-REFERENCE TO RELATED APPLICATIONS

This application is a continuation of U.S. patent application Ser. No. 17/869,570, entitled “Medicament Delivery Devices for Administration of a Medicament within a Prefilled Syringe,” filed on Jul. 20, 2022, which is a continuation of U.S. patent application Ser. No. 16/504,725, now U.S. Pat. No. 11,426,520, entitled “Medicament Delivery Devices for Administration of a Medicament within a Prefilled Syringe,” filed on Jul. 8, 2019, which is a continuation of U.S. patent application Ser. No. 16/280,488, now U.S. Pat. No. 10,342,924, entitled “Medicament Delivery Devices for Administration of a Medicament within a Prefilled Syringe,” filed on Feb. 20, 2019, which is a continuation of U.S. patent application Ser. No. 14/803,821, now U.S. Pat. No. 10,238,806, entitled “Medicament Delivery Devices for Administration of a Medicament within a Prefilled Syringe,” filed on Jul. 20, 2015, which is a continuation of U.S. patent application Ser. No. 13/357,935, now U.S. Pat. No. 9,084,849, entitled “Medicament Delivery Devices for Administration of a Medicament within a Prefilled Syringe,” filed on Jan. 25, 2012, which claims priority to U.S. Provisional Application Ser. No. 61/436,301, entitled “Devices and Methods for Delivering Lyophilized Medicaments,” filed Jan. 26, 2011, the disclosures of each of which are incorporated herein by reference in their entireties.

### BACKGROUND

The embodiments described herein relate to medicament delivery devices. More particularly, the embodiments described herein relate to medicament delivery devices for delivery of medicaments contained within a prefilled syringe.

Known prefilled syringes are commonly used to contain and inject medicaments. Known prefilled syringes include a syringe body, often constructed from glass, within which a medicament is contained. The distal end portion of some known prefilled syringes includes a staked needle (i.e., a needle that is permanently coupled to the syringe body during manufacture), the end of which is disposed within a needle cover to maintain the sterility of the needle prior to use. Other known prefilled syringes include a Luer fitting or adapted such that the distal end portion of the syringe body can be coupled to a needle. The proximal end portion of the syringe body of known prefilled syringes includes a plunger (usually constructed from an elastomer) that defines a portion of the container closure, and that can be moved within the syringe body to inject the medicament. The proximal end portion also includes a flange to allow the user to grasp the syringe body and manually apply a force to a piston to move the plunger, thereby causing injection of the medicament.

Although prefilled syringes can be cost effective devices for storing and delivering medicaments, known methods for using prefilled syringes include manually inserting the needle into the body followed by manually applying the injection force. Moreover, upon completion of the injection, known methods include covering the needle to avoid needle sticks. Thus, known prefilled syringes are often used by healthcare professionals that are trained in such procedures. To facilitate the self-administration of medicaments contained in prefilled syringes, some known autoinjectors have been adapted to contain prefilled syringes. In this manner, the autoinjector provides a source of stored energy for inserting the needle and/or injecting the medicament.

Known autoinjectors, however, are often designed for a medicament container having a specific size and/or shape, and are therefore often not configured to receive known prefilled syringes. For example, using a prefilled syringe within a known autoinjector can often result in high forces being applied to the flange of the syringe body during the insertion operation, which can lead to breakage of the syringe flange or body. Moreover, because many known prefilled syringes include a staked needle that is in fluid communication with the medicament, applying a force to the plunger during storage and/or during an insertion operation is undesirable. For example, the application of a force against the plunger during storage, which can result, for example, when a spring-loaded member is placed in contact with the plunger, can cause in leakage of the medicament. As another example, the application of a force against the plunger during a needle insertion event can result in the injection of the medicament before the needle is inserted to the desired location. Similarly stated, some known autoinjectors are not configured to control the force applied to the plunger within the syringe body during storage and/or needle insertion.

Thus, a need exists for improved methods and devices for delivering medicaments contained within a prefilled syringe.

### SUMMARY

Medicament delivery devices for administration of medicaments contained within a prefilled syringe are described herein. In some embodiments, an apparatus includes a housing, a medicament container and a movable member. The medicament container is configured to move within the housing between a first position and a second position in response to a force produced by an energy storage member. A proximal end portion of the medicament container includes a flange and has a plunger disposed therein. The movable member is configured to move within the housing. A first shoulder of the movable member is configured to exert the force on the flange to move the medicament container from the first position to the second position. A portion of the first shoulder is configured to deform when the medicament container is in the second position such that at least a portion of the force is exerted upon the plunger. A second shoulder of the movable member is configured to exert a retraction force on the flange to move the medicament container from the second position towards the first position.

### BRIEF DESCRIPTION OF THE DRAWINGS

FIGS. 1-4 are schematic illustrations of a medicament delivery device according to an embodiment, in a first, second, third and fourth configuration, respectively.

FIGS. 5-8 are schematic illustrations of a medicament delivery device according to an embodiment, in a first, second, third and fourth configuration, respectively.

FIGS. 9 and 10 are perspective views of a medical injector according to an embodiment, in a first configuration.

FIG. 11 is a front view of the medical injector illustrated in FIG. 9 with a cover removed.

FIG. 12 is a back view of the medical injector illustrated in FIG. 9 with the cover removed.

FIG. 13 is a front view of a portion of the medical injector illustrated in FIG. 9.

FIG. 14 is a perspective view of a portion of the medical injector illustrated in FIG. 9.

FIG. 15 is a bottom perspective view of a housing of the medical injector illustrated in FIG. 9.

FIG. 16 is a top perspective view of a housing of the medical injector illustrated in FIG. 9.

FIG. 17 is a perspective view of a proximal cap of the medical injector illustrated in FIG. 9.

FIGS. 18 and 19 are front views of a medicament delivery mechanism of the medical injector illustrated in FIG. 9.

FIG. 20 is a perspective view of a portion of the medical injector illustrated in FIG. 9.

FIG. 21 is an enlarged cross-sectional view of a portion of the medical injector illustrated in FIG. 9.

FIG. 22 is an exploded view of a medicament container of the medical injector illustrated in FIG. 9.

FIGS. 23 and 24 are perspective views of a carrier included in the medical injector illustrated in FIG. 9 in a first configuration.

FIG. 25 is a perspective view of the carrier included in the medical injector illustrated in FIG. 9 in a second configuration.

FIG. 26 is a perspective view of a portion of the medical injector illustrated in FIG. 9.

FIG. 27 is an enlarged front cross-sectional view of the portion of the medical injector illustrated in FIG. 26.

FIG. 28 is an enlarged side cross-sectional view of the portion of the medical injector illustrated in FIG. 26.

FIG. 29 is a back view of an electronic circuit system of the medical injector illustrated in FIG. 9.

FIG. 30 is a front view of a portion of the electronic circuit system of the medical injector illustrated in FIG. 29.

FIG. 31 is a side view of the electronic circuit system of the medical injector illustrated in FIG. 29.

FIG. 32 is a front view of an electronic circuit system housing of the electronic circuit system illustrated in FIG. 29.

FIG. 33 is a perspective view of the electronic circuit system housing of the electronic circuit system illustrated in FIG. 32.

FIG. 34 is a perspective view of a battery clip of the electronic circuit system illustrated in FIG. 29.

FIG. 35 is a perspective view of a portion of an electronic circuit system of the medical injector illustrated in FIG. 9, in a first configuration.

FIG. 36 is a front view of the medical injector illustrated in FIG. 9 in a first configuration showing the electronic circuit system.

FIGS. 37-39 are front views of a portion of the electronic circuit system of the medical injector labeled as Region Z in FIG. 36 in a first configuration, a second configuration and a third configuration, respectively.

FIGS. 40 and 41 are perspective views of a cover of the medical injector illustrated in FIG. 9.

FIG. 42 is a perspective view of a safety lock of the medical injector illustrated in FIG. 9.

FIG. 43 is a front view of the safety lock of the medical injector illustrated in FIG. 42.

FIG. 44 is a bottom view of the safety lock of the medical injector illustrated in FIG. 42.

FIG. 45 is a cross-sectional view of the safety lock of the medical injector illustrated in FIG. 42.

FIG. 46 is a perspective view of a needle sheath of the safety lock of the medical injector illustrated in FIG. 42.

FIG. 47 is a perspective view of a base of the medical injector illustrated in FIG. 9.

FIG. 48 is a front view of the base of the medical injector illustrated in FIG. 47.

FIG. 49 is a back view of the medical injector illustrated in FIG. 9 in a second configuration.

FIG. 50 is a back view of the medical injector illustrated in FIG. 9 in a third configuration.

FIG. 51 is a back view of the medical injector illustrated in FIG. 9 in a fourth configuration (i.e., the needle insertion configuration).

FIG. 52 is a front view of a portion of the medical injector illustrated in FIG. 9 in the fourth configuration (i.e., the needle insertion configuration).

FIG. 53 is a front view of a portion of the medical injector illustrated in FIG. 9 in a fifth configuration (i.e., the injection configuration).

FIG. 54 is a front view of the medical injector illustrated in FIG. 9 in a sixth configuration (i.e., the retraction configuration).

FIG. 55 is an enlarged front cross-sectional view of a portion the medical injector illustrated in FIG. 9 in the sixth configuration (i.e., the retraction configuration).

FIG. 56 is a cross-sectional front view of a medical injector according to an embodiment, in a first configuration.

FIG. 57 is a cross-sectional front view of the medical injector illustrated in FIG. 56, in a second configuration.

FIG. 58 is a perspective view of a portion of the medical injector illustrated in FIG. 56, in a first configuration.

FIG. 59 is a perspective view of a portion of the medical injector illustrated in FIG. 56, in a second configuration.

FIGS. 60 and 61 are perspective views of a medical injector according to an embodiment, in a first configuration.

FIG. 62 is a front view of the medical injector illustrated in FIG. 60 with a cover removed.

FIG. 63 is a back view of the medical injector illustrated in FIG. 60 with the cover removed.

FIG. 64 is a back view of a portion of the medical injector illustrated in FIG. 60.

FIG. 65 is a bottom perspective view of a housing of the medical injector illustrated in FIG. 64.

FIG. 66 is a front perspective views of a first portion of the housing of the medical injector illustrated in FIGS. 62 and 63.

FIG. 67 is a rear perspective views of the first portion of the housing of the medical injector illustrated in FIG. 66.

FIG. 68 is a front perspective views of a second portion of the housing of the medical injector illustrated in FIGS. 62 and 63.

FIG. 69 is a rear perspective views of the second portion of the housing of the medical injector illustrated in FIG. 68.

FIG. 70 is an enlarged view of a portion of the second portion of housing of the medical injector illustrated in FIG. 69.

FIG. 71 is a front view of a medicament delivery mechanism of the medical injector illustrated in FIG. 60.

FIG. 72 is an enlarged view of a portion of the medicament delivery mechanism on the medical injector illustrated in FIG. 71.

FIG. 73 is an enlarged view of a portion of the medicament delivery mechanism on the medical injector illustrated in FIG. 71.

FIG. 74 is an exploded view of a medicament container of the medical injector illustrated in FIG. 60.

FIG. 75 is a front view of a first movable member of the medical injector illustrated in FIG. 60, in a first configuration.

FIG. 76 is a front perspective view of the first movable member of the medical injector illustrated in FIG. 75, in a first configuration.

FIG. 77 is a rear perspective view of the first movable member of the medical injector illustrated in FIG. 75, in a first configuration.

FIG. 78 is a front view of a portion of the medical injector illustrated in FIG. 60.

FIG. 79 is a front perspective view of a second movable member of the medical injector illustrated in FIG. 60, in a first configuration.

FIG. 80 is a rear perspective view of the second movable member of the medical injector illustrated in FIG. 79 in a first configuration.

FIGS. 81 and 82 are perspective views of a cover of the medical injector illustrated in FIG. 60.

FIG. 83 is a perspective view of a safety lock of the medical injector illustrated in FIG. 60.

FIG. 84 is a front view of the safety lock of the medical injector illustrated in FIG. 83.

FIG. 85 is a bottom view of the safety lock of the medical injector illustrated in FIG. 83.

FIG. 86 is a cross-section view of the safety lock of the medical injector illustrated in FIG. 83.

FIG. 87 is a perspective view of a needle sheath of the safety lock of the medical injector illustrated in FIG. 83.

FIG. 88 is a perspective view of a base of the medical injector illustrated in FIG. 60.

FIG. 89 is a front view of the base of the medical injector illustrated in FIG. 88.

FIG. 90 is a front view of the medical injector illustrated in FIG. 60 in a third configuration.

FIG. 91 is a front view of a portion of the medical injector illustrated in FIG. 60 in the third configuration.

FIG. 92 is a front view of the medical injector illustrated in FIG. 60 in a fourth configuration (i.e., the needle insertion configuration).

FIG. 93 is a front view of a portion of the medical injector illustrated in FIG. 60 in the fourth configuration (i.e., the needle insertion configuration).

FIG. 94 is an enlarged perspective view of a portion of the medical injector illustrated in FIG. 60 in the fourth configuration (i.e., the needle insertion configuration).

FIG. 95 is a front view of the medical injector illustrated in FIG. 60 in a fifth configuration (i.e., the injection configuration).

FIG. 96 is a perspective view of a first movable member of the medical injector illustrated in FIG. 60 in a second configuration.

FIG. 97 is a front view of the medical injector illustrated in FIG. 60 in a sixth configuration (i.e., the retraction configuration).

FIG. 98 is a front perspective view of a second movable member of the medical injector illustrated in FIG. 60 in a second configuration.

FIGS. 99-102 are schematic illustrations of a medicament delivery device according to an embodiment, in a first, second, third, and fourth configuration, respectively.

FIGS. 103 and 104 are perspective views of a medical injector according to an embodiment, in a first configuration.

FIG. 105 is a rear view of the medical injector illustrated in FIG. 103 with a cover removed.

FIG. 106 is a front view of the medical injector illustrated in FIG. 103 with the cover removed.

FIG. 107 is a rear view of a portion of the medical injector illustrated in FIG. 103.

FIG. 108 is a bottom perspective view of a housing of the medical injector illustrated in FIG. 103.

FIG. 109 is a front perspective view of a first portion of the housing of the medical injector illustrated in FIG. 103.

FIG. 110 is a rear perspective view of the first portion of the housing of the medical injector illustrated in FIG. 109.

FIG. 111 is a front perspective view of a second portion of the housing of the medical injector illustrated in FIG. 103.

FIG. 112 is a rear perspective view of the second portion of the housing of the medical injector illustrated in FIG. 111.

FIG. 113 is a front view of a medicament delivery mechanism of the medical injector illustrated in FIG. 103.

FIG. 114 is a rear view of a medicament delivery mechanism of the medical injector illustrated in FIG. 103.

FIG. 115 is an enlarged front view of a portion of the medicament delivery mechanism of the medical injector illustrated in FIG. 114.

FIG. 116 is an enlarged rear view of a portion of the medicament delivery mechanism of the medical injector illustrated in FIG. 114.

FIG. 117 is a top view of a portion of the medical injector illustrated in FIG. 103.

FIG. 118 is an exploded view of a medicament container of the medical injector illustrated in FIG. 103.

FIG. 119 is a front view of the medicament container shown in FIG. 118.

FIGS. 120-123 illustrate an elastomeric member included in the medicament container of FIG. 118.

FIGS. 124-127 illustrate an elastomeric member included in the medicament container of FIG. 118.

FIGS. 128 and 129 are perspective views of a carrier included in the medical injector illustrated in FIG. 103.

FIG. 130 is a perspective view of a movable assembly of the medical injector illustrated in FIG. 103.

FIGS. 131-133 illustrate a first movable member included in the movable assembly illustrated in FIG. 130.

FIG. 134 illustrates a second movable member included in the movable assembly illustrated in FIG. 130.

FIG. 135 is a perspective view of a transfer member included in the medical injector illustrated in FIG. 103.

FIG. 136 is a rear view of an electronic circuit system of the medical injector illustrated in FIG. 103.

FIG. 137 is a front view of a portion of the electronic circuit system of the medical injector illustrated in FIG. 136.

FIG. 138 is a perspective view of a portion of an electronic circuit system of the medical injector illustrated in FIG. 103, in a first configuration.

FIGS. 139-141 are front views of a portion of the electronic circuit system of the medical injector labeled as Region Z in FIG. 138 in a first configuration, a second configuration and a third configuration, respectively.

FIGS. 142 and 143 are perspective views of a cover of the medical injector illustrated in FIG. 103.

FIG. 144 is a perspective view of a safety lock of the medical injector illustrated in FIG. 103.

FIG. 145 is a front view of the safety lock of the medical injector illustrated in FIG. 144.

FIG. 146 is a bottom view of the safety lock of the medical injector illustrated in FIG. 144.

FIG. 147 is a cross-sectional view of the safety lock of the medical injector illustrated in FIG. 144.

FIG. 148 is a perspective view of a needle sheath of the safety lock of the medical injector illustrated in FIG. 144.

FIG. 149 is a perspective view of a mixing actuator included in the system actuator assembly of the medical injector illustrated in FIG. 103.

FIG. 150 is a perspective view of a base included in a system actuator assembly of the medical injector illustrated in FIG. 103.

FIG. 151 is a front view of the base included in the medical injector illustrated in FIG. 150.

FIG. 152 is a back view of the medical injector illustrated in FIG. 103 in a second configuration.

FIG. 153 is a front cross-sectional view of the medical injector illustrated in FIG. 103 in the second configuration.

FIG. 154 is a front view of a portion of the medical injector of FIG. 103 just prior to transitioning to a third configuration (i.e., the mixing configuration).

FIG. 155 is a top perspective view of the medical injector illustrated in FIG. 103 in the third configuration.

FIG. 156 is a front cross-sectional view of the medical injector illustrated in FIG. 103 in the third configuration.

FIG. 157 is an enlarged view of a portion of the front cross-section illustrated in FIG. 156.

FIG. 158 is a front cross-sectional view of the medical injector illustrated in FIG. 103 in a fourth configuration (i.e., the needle insertion configuration).

FIG. 159 is an enlarged view of a portion of the cross-section illustrated in FIG. 158.

FIG. 160 is a front cross-sectional view of the medical injector illustrated in FIG. 103 in a fifth configuration (i.e., the injection configuration).

FIG. 161 is a front cross-sectional view of the medical injector illustrated in FIG. 103 in a sixth configuration (i.e., the retraction configuration).

FIG. 162 is a front perspective view of a medical injector according to an embodiment, in a first configuration.

FIG. 163 is a side view of the medical injector illustrated in FIG. 162.

FIG. 164 is a cross-sectional view taken along line X1-X1 of the medical injector illustrated in FIG. 163.

FIG. 165 is a front perspective view of the medical injector illustrated in FIG. 162 in a second configuration.

FIG. 166 is a rear view of the medical injector illustrated in FIG. 162 in the second configuration.

FIG. 167 is a front view of a portion of the medical injector illustrated in FIG. 162 in the second configuration.

FIG. 168 is a front view of a medicament container assembly of the medical injector illustrated in FIG. 162.

FIG. 169 is a side view of a portion of the medicament container assembly illustrated in FIG. 168.

FIG. 170 is a perspective view of a portion of the medicament container assembly illustrated in FIG. 168.

FIG. 171 is a front perspective view of a portion of the medical injector illustrated in FIG. 162.

FIG. 172 is a perspective exploded view of a portion of the movable assembly and the system actuator assembly illustrated in FIG. 171.

FIG. 173 is a cross-sectional view of a portion of the movable assembly illustrated in FIG. 171.

FIG. 174 is a cross-sectional view of a portion of the movable assembly illustrated in FIG. 171.

FIG. 175 is a front view of medicament delivery mechanism included in the medical injector illustrated in FIG. 162.

FIG. 176 is front perspective view of a portion of the medical delivery mechanism illustrated in FIG. 175.

FIG. 177 is rear perspective view of the portion of the medical delivery mechanism illustrated in FIG. 175.

FIG. 178 is a side view of the portion of the medicament delivery mechanism illustrated in FIG. 175.

FIG. 179 is a perspective view of the transfer member illustrated in FIG. 175.

FIG. 180 is a perspective view of a portion of the medical injector of FIG. 162 illustrating a retraction member.

FIG. 181 is a perspective view of a portion of an electronic assembly of the medical injector illustrated in FIG. 162.

FIG. 182 is a rear perspective view of a portion of the electronic assembly of the injector shown in FIG. 162.

FIG. 183 is a rear perspective view of a portion of the electronic assembly and the base included in the medical injector illustrated in FIG. 162.

FIG. 184 is a rear exploded view of a portion of the electronic assembly, the base, and the safety lock included in the medical injector illustrated in FIG. 162.

FIGS. 185-190 are cross-sectional views illustrating the operation of a medical injector according to an embodiment.

FIGS. 191-196 are cross-sectional views illustrating the operation of a medical injector according to an embodiment.

FIGS. 197-202 are cross-sectional views illustrating the operation of a medical injector according to an embodiment.

FIG. 203 is a front view of a portion of a medical injector in a first configuration, according to an embodiment.

FIG. 204 is a front cross-sectional view of the portion of the medical injector illustrated in FIG. 203 in the first configuration.

FIG. 205 is a front cross-sectional view of a portion of the medical injector illustrated in FIG. 203 in a second configuration.

FIG. 206 is an exploded perspective view of a portion of the medical injector illustrated in FIG. 203.

FIG. 207 is a top view of the medical injector illustrated in FIG. 203.

FIG. 208 is a bottom view of the medical injector illustrated in FIG. 203.

FIG. 209 is a cross-sectional view of the medical injector illustrated in FIG. 203.

FIG. 210 is a cross-sectional view of the medical injector illustrated in FIG. 124 taken along the line W1-W1 in FIG. 209.

FIGS. 211-215 are cross-sectional views illustrating the operation of the medical injector illustrated in FIG. 203.

FIG. 216 is a top view of the medical injector illustrated in FIG. 203.

FIG. 217 is a cross-sectional view of the medical injector illustrated in FIG. 216 taken along the line W2-W2.

FIG. 218 is a perspective view of the medical injector illustrated in FIG. 203 in the first configuration.

### DETAILED DESCRIPTION

Medicament delivery devices for administration of medicaments contained within a prefilled syringe are described herein. In some embodiments, an apparatus includes a housing, a medicament container and a movable member. The medicament container, which can be, for example, a prefilled syringe, is configured to move within the housing between a first position and a second position in response to a force produced by an energy storage member. The energy storage member can be, for example, a spring, a compressed gas container, an electrical energy storage member or the like. A proximal end portion of the medicament container includes a flange and has a plunger disposed therein. The movable member is configured to move within the housing. A first shoulder of the movable member is configured to exert the force on the flange to move the medicament container from the first position to the second position. A portion of the first shoulder is configured to deform when the medicament container is in the second position such that at least a portion of the force is exerted upon the plunger. A second shoulder of the movable member is configured to exert a retraction force on the flange to move the medicament container from the second position towards the first position.

In some embodiments, a medicament delivery device includes a housing, a medicament container, a movable member and an energy storage member. The medicament container is configured to move within the housing between a first position and a second position in response to a force produced by the energy storage member. A proximal end portion of the medicament container includes a flange and has a plunger disposed therein. The movable member is configured to exert the force on the medicament container to move the medicament container from the first position to the second position. An engagement portion of the movable member is configured to limit movement of a piston surface relative to the plunger when the medicament container moves from the first position to the second position such that the piston surface is spaced apart from the plunger. The engagement portion is configured to deform when the medicament container is in the second position such that the piston surface is in contact with the plunger.

In some embodiments, a medicament delivery device includes a housing, a medicament container, a first movable member and a second movable member. The medicament container is configured to move within the housing between a first position and a second position in response to a force produced by an energy storage member. A proximal end portion of the medicament container includes a flange and has a plunger disposed therein. The first movable member is configured to move within the housing, and is operably coupled to the energy storage member such that a first portion of the first movable member is configured to exert at least a portion of the force on the flange to move the medicament container from the first position to the second position. A second portion of the first movable member is configured to deform when the medicament container is in the second position such that at least a portion of the force is exerted upon the plunger. The second movable member is configured to move with the medicament container when the medicament container moves from the first position to the second position. The second movable member is configured to move relative to the medicament container to move the plunger within the medicament container after the second portion of the first movable member is deformed.

In some embodiments, a medical device includes a carrier configured to be disposed within a housing of the medical device. The carrier is configured to contain at least a proximal portion of a medicament container, such as, for example a prefilled syringe having a flange. A first shoulder of the carrier is in contact with a proximal surface of the flange and a second shoulder of the carrier is in contact with a distal surface of the flange. The carrier has a first engagement portion configured to engage a movable member such that when a first force is exerted by the movable member on the first engagement portion, the first shoulder transfers at least a portion of the first force to the proximal surface of the flange. The carrier has a second engagement portion configured to engage a retraction spring such that when a second force is exerted by the retraction spring on the second engagement portion, the second shoulder transfers at least a portion of the second force to the distal surface of the flange.

In some embodiments, the medical device further includes a damping member disposed between the first shoulder of the carrier and the proximal surface of the flange of the medicament container, or between the second shoulder of the carrier and the proximal surface of the flange of the medicament container. The damping member can be disposed such that a portion of the first force or a portion of the second force is received and/or absorbed by the damping member to reduce the possibility of damage to the medicament container and/or flange.

In some embodiments, a medical device includes a housing, a movable member and a medicament container. The movable member is disposed within the housing and has a first engagement portion, a second engagement portion and a retraction portion. The first engagement portion is configured to be coupled to an energy storage member. The second engagement portion is configured to be coupled to the medicament container such that a shoulder of the second engagement portion exerts a first force produced by the energy storage member on the medicament container to move the medicament container within the housing in a first direction. The retraction portion is configured to produce a second force to move the medicament container within the housing in a second direction. In some embodiments, the retraction portion includes a spring that is monolithically constructed with at least the second engagement portion.

As used in this specification and the appended claims, the words “proximal” and “distal” refer to direction closer to and away from, respectively, an operator of the medical device. Thus, for example, the end of the medicament delivery device contacting the patient's body would be the distal end of the medicament delivery device, while the end opposite the distal end would be the proximal end of the medicament delivery device.

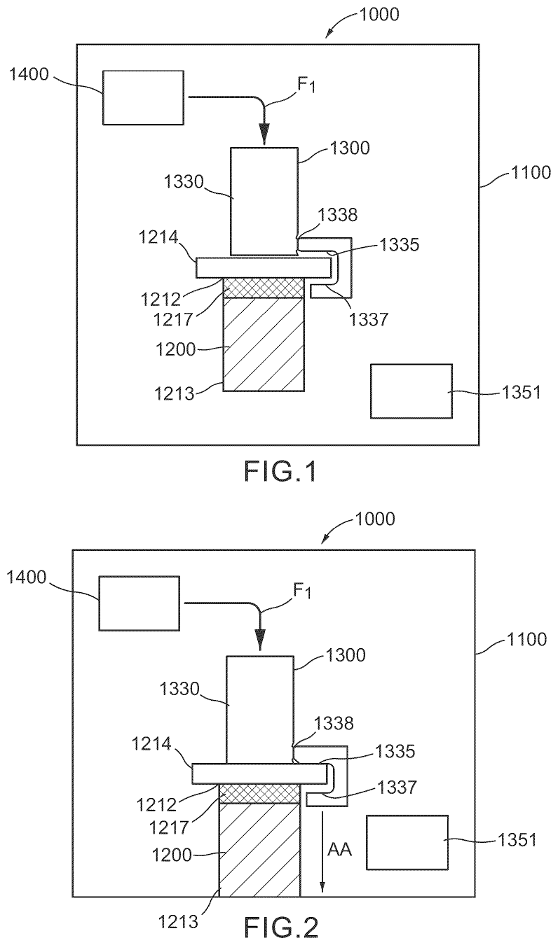

FIGS. 1-4 are schematic illustrations of a medicament delivery device 1000 according to an embodiment in a first, second, third and fourth configuration, respectively. The medicament delivery device 1000 includes a housing 1100, a medicament container 1200, a movable member 1300, an energy storage member 1400 and a retraction member 1351. The housing 1100 can be any suitable size, shape, or configuration and can be made of any suitable material. For example, in some embodiments, the housing 1100 is an assembly of multiple parts formed from a plastic material and defines a substantially rectangular shape when assembled.

The medicament container 1200 is disposed within the housing 1100, and contains (i.e., is filled or partially filled with) a medicament. The medicament container 1200 includes a proximal end portion 1212 that has a flange 1214 and a distal end portion 1213 that is coupled to a needle (not shown in FIGS. 1-4). The medicament container 1200 includes an elastomeric member 1217 (also referred to herein as a “plunger”). The elastomeric member 1217 is formulated to be compatible with the medicament housed within the medicament container 1200. Similarly stated, the elastomeric member 1217 is formulated to minimize any reduction in the efficacy of the medicament that may result from contact (either direct or indirect) between the elastomeric member 1217 and the medicament. For example, in some embodiments, the elastomeric member 1217 can be formulated to minimize any leaching or out-gassing of compositions that may have an undesired effect on the medicament. The elastomeric member 1217 is disposed within the medicament container 1200 to seal the proximal end portion 1212 of the medicament container 1200. In some embodiments, the elastomeric member 1217 can be formulated to maintain its chemical stability, flexibility and/or sealing properties when in contact (either direct or indirect) with a medicament over a long period of time (e.g., for up to six months, one year, two years, five years or longer). The medicament container 1200 can be any container suitable for storing the medicament. In some embodiments, the medicament container 1200 can be, for example, a prefilled syringe having a staked needle at the distal end thereof. In those embodiments in which the medicament container 1200 is a prefilled syringe, the elastomeric member 1217 can be disposed within the medicament container 1200 during the fill process (e.g., before being placed in the housing 1100).

The energy storage member 1400 can be any suitable device or mechanism that, when actuated, produces a force F₁ to deliver the medicament contained within the medicament container 1200. Similarly stated, the energy storage member 1400 can be any suitable device or mechanism that produces the force F₁ such that the medicament is conveyed from the medicament container 1200 into a body of a patient. More specifically, the energy storage member 1400 produces the force F₁ that moves the medicament container 1200 from a first position to a second position in a first direction indicated by the arrow AA in FIG. 2 and/or that moves the plunger 1217 from a first plunger position to a second plunger position as shown by the arrow BB in FIG. 3. The medicament can be conveyed into a body via any suitable mechanism, such as, for example, by injection. By employing the energy storage member 1400 to produce the force F₁ rather than relying on a user to manually produce the delivery force, the medicament can be delivered into the body at the desired pressure and/or flow rate, and with the desired delivery characteristics. Moreover, this arrangement reduces the likelihood of partial delivery (e.g., that may result if the user is interrupted or otherwise rendered unable to manually produce the force to complete the delivery).

In some embodiments, the energy storage member 1400 can be a mechanical energy storage member, such as a spring, a device containing compressed gas, a device containing a vapor pressure-based propellant or the like. In other embodiments, the energy storage member 1400 can be an electrical energy storage member, such as a battery, a capacitor, a magnetic energy storage member or the like. In yet other embodiments, the energy storage member 1400 can be a chemical energy storage member, such as a container containing two substances that, when mixed, react to produce energy.

The energy storage member 1400 can be disposed within the housing in any position and/or orientation relative to the medicament container 1200. In some embodiments, for example, the energy storage member 1400 can be positioned within the housing 1100 spaced apart from the medicament container 1200. Moreover, in some embodiments, the energy storage member 1400 can be positioned such that a longitudinal axis of the energy storage member 1400 is offset from the medicament container 1200. In other embodiments, the energy storage member 1400 can substantially surround the medicament container 1200.

As shown in FIG. 1, the energy storage member 1400 is operably coupled to the movable member 1300, the medicament container 1200 and/or the medicament therein such that the force F₁ delivers the medicament. In some embodiments, for example, the force F₁ can be transmitted to the medicament container 1200 and/or the medicament therein via the movable member 1300. The movable member 1300 can be any suitable member, device, assembly or mechanism configured to move within the housing 1100. As shown in FIGS. 1-4, the movable member 1300 includes a piston portion 1330 configured to transmit the force F₁ to the plunger 1217 disposed within the medicament container 1200.

The movable member 1300 includes a first shoulder 1335 and a second shoulder 1337. The first shoulder 1335 of the movable member 1300 is configured to exert the force F₁, produced by the energy storage member 1400, on the flange 1214 of the medicament container 1200. In this manner, when the medicament delivery device 1000 is actuated to produce the force F₁, movable member 1300 moves the medicament container 1200 from the first position (see FIG. 1, which corresponds to the first configuration of the medicament delivery device 1000) to the second position (see FIG. 2, which corresponds to the second configuration of the medicament delivery device 1000). In some embodiments, the movement of the medicament container 1200 within the housing 1100 results in a needle insertion operation. Although the first shoulder 1335 is shown as directly contacting the flange 1214 when the medicament delivery device 1000 is in the second configuration (FIG. 2), in other embodiments, there can be intervening structure (e.g., an o-ring, a damping member, or the like) disposed between the first shoulder 1335 and the flange 1214.

In some embodiments, the first shoulder 1335 of the movable member 1300 can be configured to maintain a distance between the piston portion 1330 of the movable member 1300 and the plunger 1217 when the medicament delivery device 1000 is in the first configuration (FIG. 1). Similarly stated, in some embodiments, the movable member 1300 and the medicament container 1200 are collectively configured such that the piston portion 1330 is spaced apart from the plunger 1217 when the medicament delivery device 1000 is in its storage configuration and/or when the medicament container 1200 is moving between its first position and its second position. In this manner, any preload or residual force produced by the energy storage member 1400 on the movable member 1300 is not transferred to the plunger 1217. Said another way, the plunger 1217 is isolated from the energy storage member 1400 during the storage configuration. Accordingly, this arrangement reduces and/or eliminates medicament leakage from the medicament container 1200.

As shown in FIG. 3, the first shoulder 1335 includes a deformable portion 1338 configured to deform when the medicament container 1200 is in the second position such that at least a portion of the force F₁ is exerted upon the plunger 1217. In some embodiments, the deformable portion 1338 can be separated from the piston portion 1330 of the movable member 1300. In other embodiments, the deformable portion 1338 is configured to bend, deform, rotate and/or otherwise move relative to the piston portion 1300 such that the piston portion 1330 is placed into contact (directly or indirectly via intervening structure) with the plunger 1217. Similarly stated, in some embodiments, the deformable portion 1338 is configured to bend, deform, rotate and/or otherwise move relative to the piston portion 1300 such that the first shoulder 1335 no longer maintains the distance between the piston portion 1300 and the plunger 1217. In this manner, the piston portion 1330 transmits at least a portion of the force F₁ to the plunger 1217, thereby placing the medicament container 1200 into the third configuration (FIG. 3). More specifically, when the deformable portion 1338 deforms, the piston portion 1330 moves within the medicament container 1200 in the direction of the arrow BB (FIG. 3) and moves the plunger 1217 from the proximal end portion 1212 of the medicament container 1200 towards the distal end portion 1213 of the medicament container 1200. This arrangement allows for the delivery of the medicament contained within the medicament container 1200 into a body of a patient.

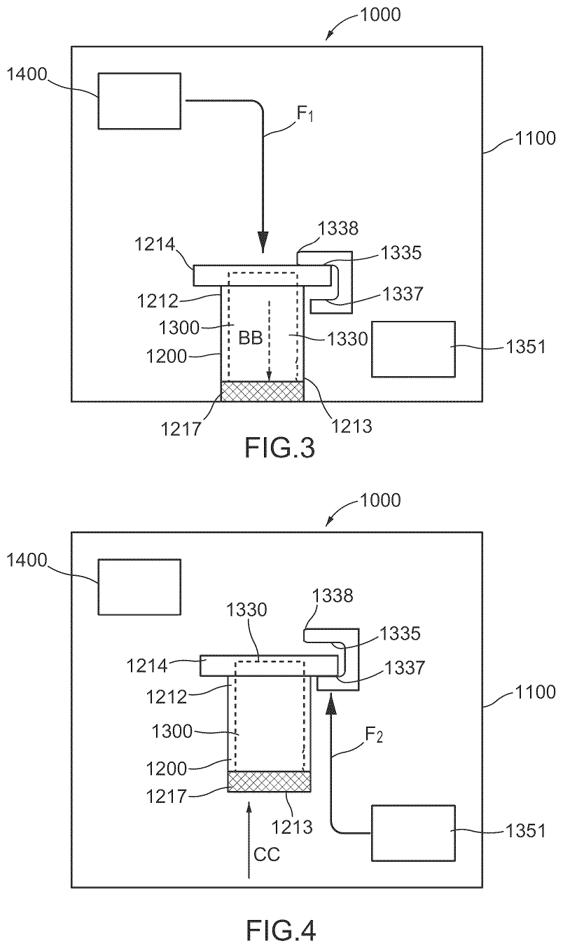

When the medicament is delivered, the retraction member 1351 exerts a retraction force F₂ on at least the second shoulder 1337 of the movable member 1300 in a second direction, opposite the first direction. When the retraction force F₂ is exerted, the second shoulder 1337 engages a distal surface of the flange 1214 of the medicament container 1200, thereby exerting at least a portion of the retraction force F₂ on the flange 1214. Although the second shoulder 1337 is shown as directly contacting the flange 1214 when the medicament delivery device 1000 is in the fourth configuration (FIG. 4), in other embodiments, there can be intervening structure (e.g., an o-ring, a damping member, or the like) disposed between the second shoulder 1337 and the flange 1214. The exertion of the retraction force F₂ on the flange 1214 moves the medicament container 1200 from the second position (e.g., the second and third configuration, as shown in FIGS. 2 and 3) in the direction of the arrow CC toward the first position. In this manner, the retraction member 1351 produces the retraction force F₂ and moves the distal end portion 1213 of the medicament container 1200 (which can include, for example, a needle) away from the body of the patient and into the housing 1100 of the medicament delivery device 1000.

The retraction member 1351 can be any suitable device or mechanism that, when actuated, produces a force F₂ to move the medicament container 1200 in the second direction as indicated by the arrow CC in FIG. 4. In some embodiments, the retraction member 1351 can be a mechanical energy storage member, such as a spring, a device containing compressed gas, a device containing a vapor pressure-based propellant or the like. In other embodiments, the retraction member 1351 can be an electrical energy storage member, such as a battery, a capacitor, a magnetic energy storage member or the like. In yet other embodiments, the retraction member 1351 can be a chemical energy storage member, such as a container containing two substances that, when mixed, react to produce energy. Although the retraction member 1351 is shown as being separate and distinct from the energy storage member 1400, in some embodiments, the energy storage member 1400 can be configured to produce the retraction force F₂.

The retraction member 1351 can be in any position and/or orientation relative to the medicament container 1200. In some embodiments, for example, the retraction member 1351 can be positioned within the housing 1100 spaced apart from the medicament container 1200. Moreover, in some embodiments, the retraction member 1351 can be positioned such that a longitudinal axis of the retraction member 1351 is offset from the medicament container 1200. In other embodiments, the retraction member 1351 can substantially surround the medicament container 1200. In some embodiments, the retraction member 1351 is coupled to the second shoulder 1337 of the movable member 1300. In other embodiments, the retraction member 1351 is monolithically formed with the movable member 1300.

FIGS. 5-8 are schematic illustrations of a medicament delivery device 2000 according to an embodiment in a first, second, third and fourth configuration, respectively. The medicament delivery device 2000 includes a housing 2100, a medicament container 2200, a first movable member 2300, a second movable member 2345 and an energy storage member 2400. The housing 2100 can be any suitable size, shape, or configuration and can be made of any suitable material. For example, in some embodiments, the housing 2100 is an assembly of multiple parts formed from a plastic material and defines a substantially rectangular shape when assembled.

The medicament container 2200 is disposed within the housing 2100, and contains (i.e., is filled or partially filled with) a medicament. The medicament container 2200 includes a proximal end portion 2212 that has a flange 2214 and a distal end portion 2213 that is coupled to a delivery member, such as a needle, nozzle or the like (not shown in FIGS. 5-8). The medicament container 2200 includes an elastomeric member 2217. The elastomeric member 2217 is formulated to be compatible with the medicament housed within the medicament container 2200. Similarly stated, the elastomeric member 2217 is formulated to minimize any reduction in the efficacy of the medicament that may result from contact (either direct or indirect) between the elastomeric member 2217 and the medicament. For example, in some embodiments, the elastomeric member 2217 can be formulated to minimize any leaching or out-gassing of compositions that may have an undesired effect on the medicament. The elastomeric member 2217 is disposed within the medicament container 2200 to seal the proximal end portion 2212 of the medicament container 2200. In some embodiments, the elastomeric member 2217 can be formulated to maintain its chemical stability, flexibility and/or sealing properties when in contact (either direct or indirect) with a medicament over a long period of time (e.g., for up to six months, one year, two years, five years or longer). The medicament container 2200 can be any container suitable for storing the medicament. In some embodiments, the medicament container 2200 can be, for example, a prefilled syringe having a staked needle at the distal end thereof. In those embodiments in which the medicament container 1200 is a prefilled syringe, the elastomeric member 2217 is disposed within the medicament container 2200 during the fill process (e.g., before the prefilled syringe is placed in the housing 2100).

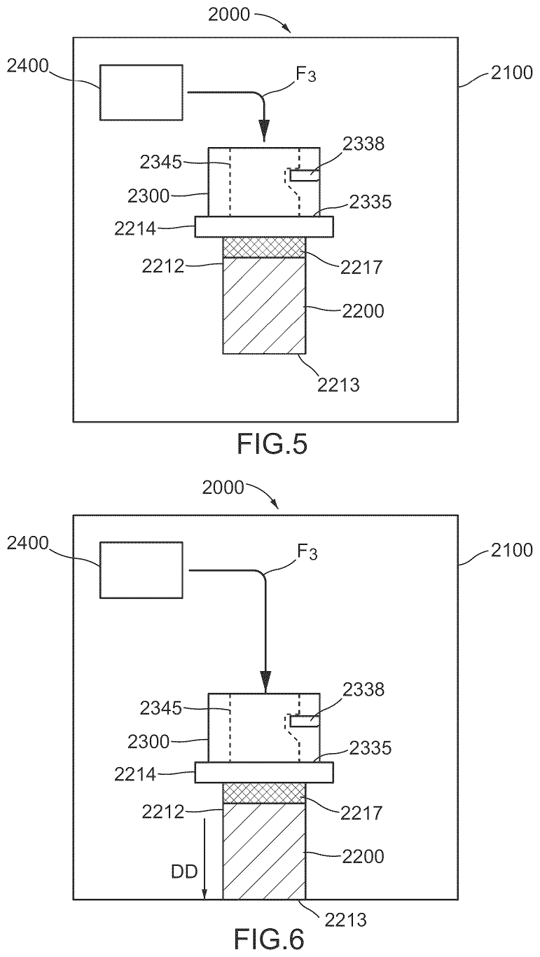

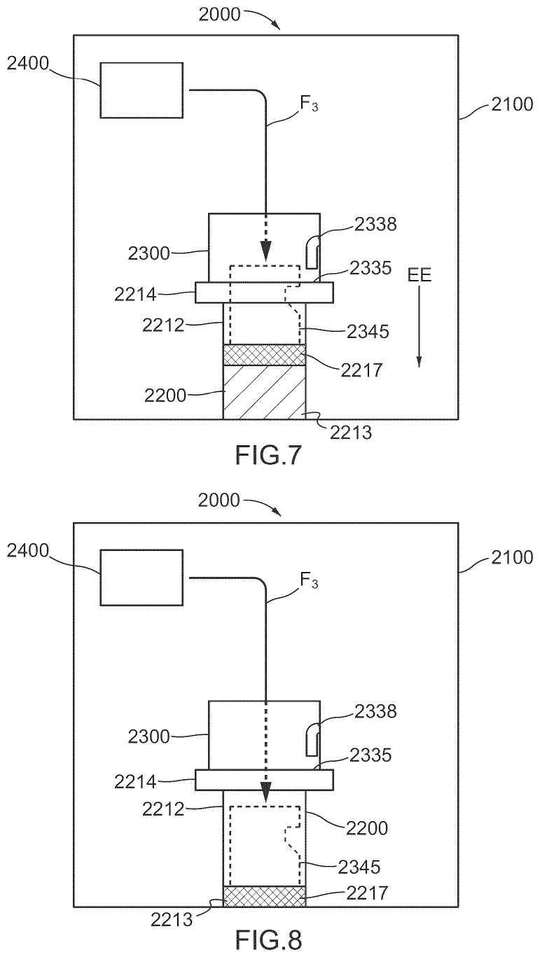

The energy storage member 2400 can be any suitable device or mechanism that, when actuated, produces a force F₃ to deliver the medicament contained within the medicament container 2200. Similarly stated, the energy storage member 2400 can be any suitable device or mechanism that produces the force F₃ such that the medicament is conveyed from the medicament container 2200 into a body of a patient. More specifically, the energy storage member 2400 produces the force F₃ that moves the medicament container 2200 from a first position to a second position in a first direction indicated by the arrow DD in FIG. 6 and/or that moves the plunger 2217 from a first plunger position to a second plunger position, as shown by the arrow EE in FIG. 7. The medicament can be conveyed into a body via any suitable mechanism, such as, for example, by injection via a needle, nozzle or the like.

In some embodiments, the energy storage member 2400 can be a mechanical energy storage member, such as a spring, a device containing compressed gas, a device containing a vapor pressure-based propellant or the like. In other embodiments, the energy storage member 2400 can be an electrical energy storage member, such as a battery, a capacitor, a magnetic energy storage member or the like. In yet other embodiments, the energy storage member 2400 can be a chemical energy storage member, such as a container containing two substances that, when mixed, react to produce energy.

The energy storage member 2400 can be in any position and/or orientation relative to the medicament container 2200. In some embodiments, for example, the energy storage member 2400 can be positioned within the housing 2100 spaced apart from the medicament container 2200. Moreover, in some embodiments, the energy storage member 2400 can be positioned such that a longitudinal axis of the energy storage member 2400 is offset from the medicament container 2200. In other embodiments, the energy storage member 2400 can substantially surround the medicament container 2200.

As shown in FIG. 5, the energy storage member 2400 is operably coupled to the first movable member 2300, the second movable member 2345, the medicament container 2200 and/or the medicament therein such that the force F₃ delivers the medicament. In some embodiments, for example, the force F₃ can be transmitted to the medicament and/or the medicament container 2200 via the first movable member 2300 and/or the second movable member 2345. As described in more detail herein, the first movable member 2300 and the second movable member 2345 are collectively configured to transmit the force F₃ to the plunger 2217 disposed within the medicament container 2200.

The first movable member 2300 includes a first portion 2335 and a second portion 2338. The first portion 2335 of the movable member 2300 is configured to transmit and/or exert at least a portion of the force F₃ produced by the energy storage member 2400 on the flange 2214 of the medicament container 2200 to move the medicament container 2200 from the first position (see FIG. 5, which corresponds to the first configuration of the medicament delivery device 2000) to the second position (see FIG. 6, which corresponds to the second configuration of the medicament delivery device 2000). Although the medicament container 2200 is shown as being within the housing 2100 when the medicament container 2200 is in the second position, in some embodiments, the movement of the medicament container 2200 can result in a needle insertion operation in which a needle (not shown in FIGS. 5-8) is extended outside of the housing 2100. The first portion 2335 of the movable member 2300 can be, for example, a first shoulder, protrusion, sleeve or the like. Although the first portion 2335 is shown as directly contacting the flange 2214 when the medicament delivery device 2000 is in the second configuration (FIG. 6), in other embodiments, there can be intervening structure (e.g., an o-ring, a damping member, or the like) disposed between the first portion 2335 and the flange 2214.

The second portion 2338 of the first movable member 2300 maintains the second movable member 2345 in a first position (FIGS. 5 and 6), relative to the medicament container 2200 and/or the first movable member 2300 when the medicament delivery device 2000 is in the first (i.e., storage) configuration (FIG. 5). In this manner, as shown in FIG. 6, at least a portion of the force F₃ can be transferred from the energy storage member 2400 to the first movable member 2300 (and to the flange 2214) via the second movable member 2345. Thus, when the medicament container 2200 is moved from its first position to its second position, the second movable member 2345 moves with the medicament container 2200 and/or the first movable member 2300.

In some embodiments, the second portion 2338 can engage the second movable member 2345 to maintain a distance (e.g., an air gap, space, or void) between the second movable member 2345 and the plunger 2217, when the medicament container 2200 is in the first configuration (FIG. 1) and/or when the medicament container 2200 is moving between its first position and its second position. In this manner, any preload or residual force produced by the energy storage member 1400 on the second movable member 2345 is not transferred to the plunger 2217. Said another way, the plunger 2217 is substantially isolated from the energy storage member 2400 during the storage configuration and/or when the medicament container 2200 is moving. Accordingly, this arrangement reduces and/or eliminates medicament leakage from the medicament container 2200.

When the medicament container 2200 in the second position (FIGS. 6 and 7), the second portion 2338 of the first movable member 2300 is configured to deform (e.g., by a portion of the force F₃), thereby allowing movement of the second movable member 2345 relative to the first movable member 2300 and/or the medicament container 2200. Thus, when the second portion 2338 of the first movable member 2300 deforms, at least a portion of the force F₃ is exerted upon the plunger 2217. Similarly stated, when the medicament delivery device 2000 is in the second configuration (FIG. 6), a portion of the force F₃ can deform the second portion 2338 of the movable member 2300 (FIG. 7). After the second portion 2338 is deformed, at least a portion of the force F₃ is transmitted from the second movable member 2345 to the plunger 2217 to place the medicament container 2200 in the third configuration (FIG. 7). More specifically, when the second portion 2338 deforms, the second movable member 2345 moves in the direction of the arrow EE (FIG. 7) and moves the plunger 2217 from the proximal end portion 2212 of the medicament container 2200 toward the distal end portion 2213 of the medicament container 2200. Similarly stated, when the second portion 2338 deforms, the second movable member 2345 moves relative to the medicament container 2200 to move the plunger 2217 within the medicament container 2200. This arrangement allows for the delivery of the medicament contained within the medicament container 2200 into a body of a patient, as shown in FIG. 8.

In some embodiments, the medicament delivery device 2000 can include a retraction member (not shown in FIGS. 5-8). The retraction member can be any suitable device and/or mechanism configured to move the medicament container 2200 from the second position (e.g., the fourth configuration shown in FIG. 8) toward the first position (e.g. the first configuration shown in FIG. 5). In some embodiments, the retraction member can be substantially similar to the retraction member 1351 described with respect to FIGS. 1-4. In such embodiments, the retraction member can be configured to transmit a force to the flange 2214 of the medicament container 2200 and move the medicament container 2200 in a second direction opposite the first direction indicated by the arrow DD in FIG. 6.

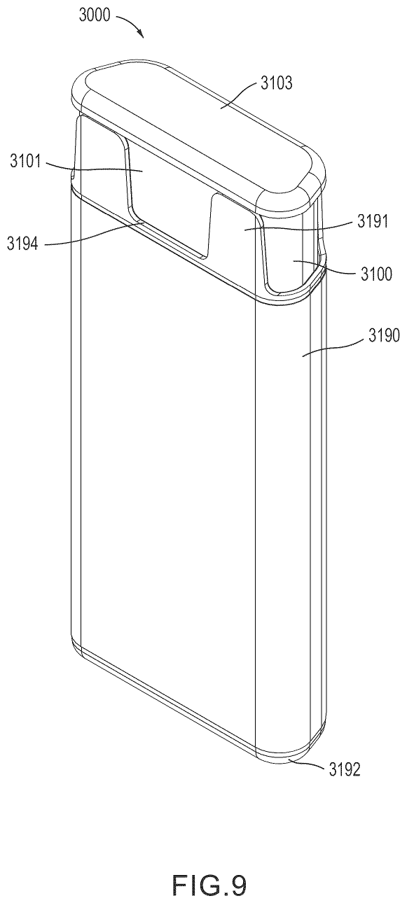

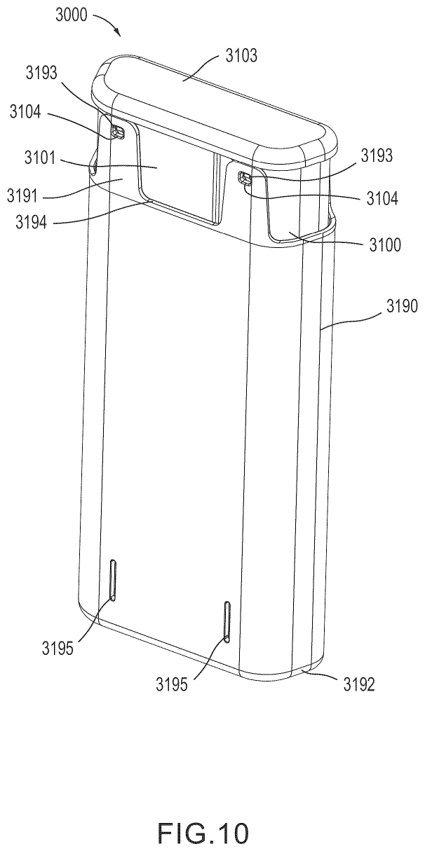

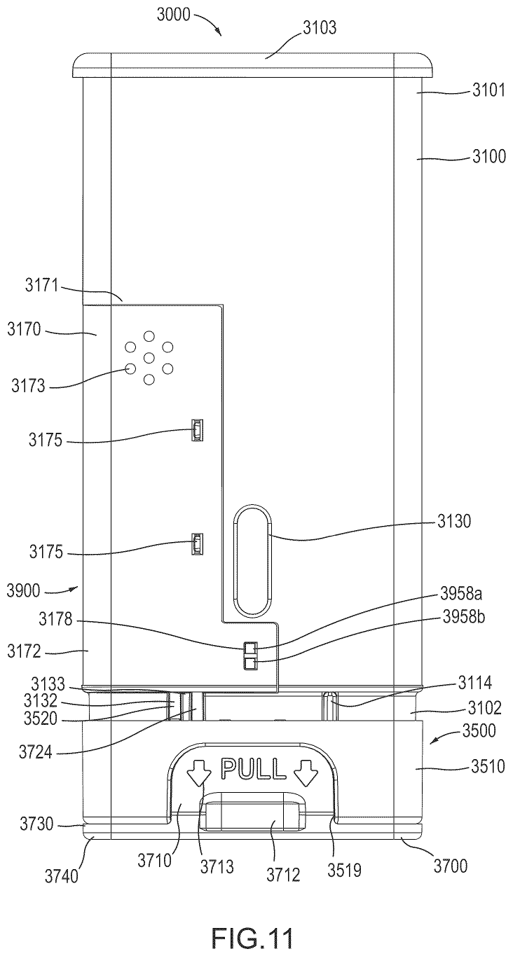

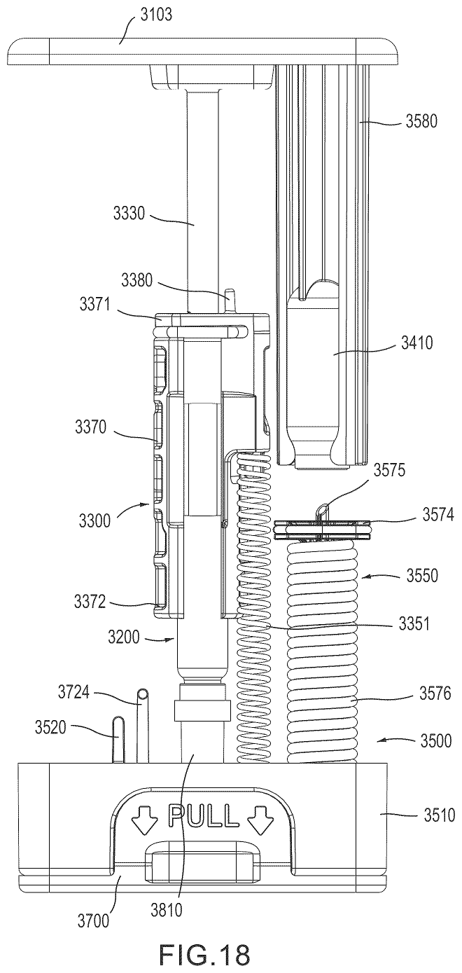

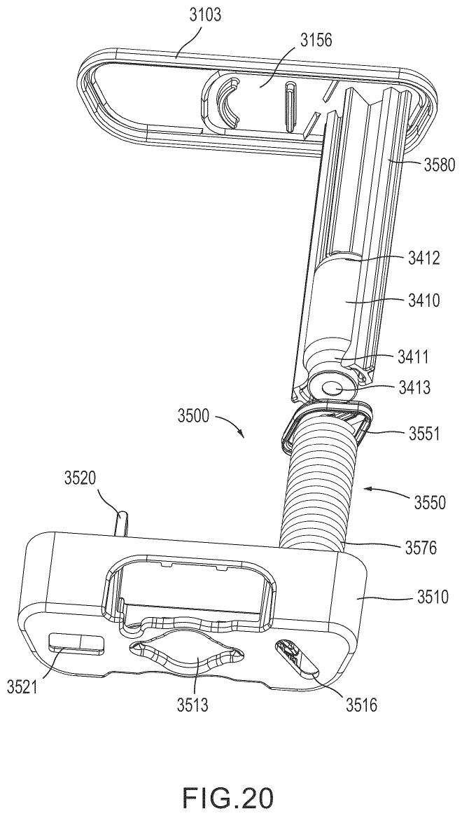

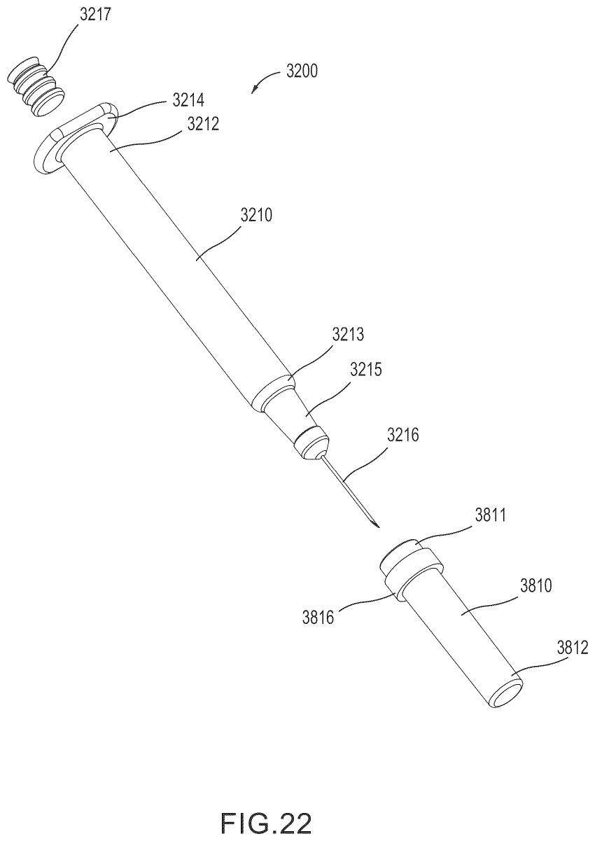

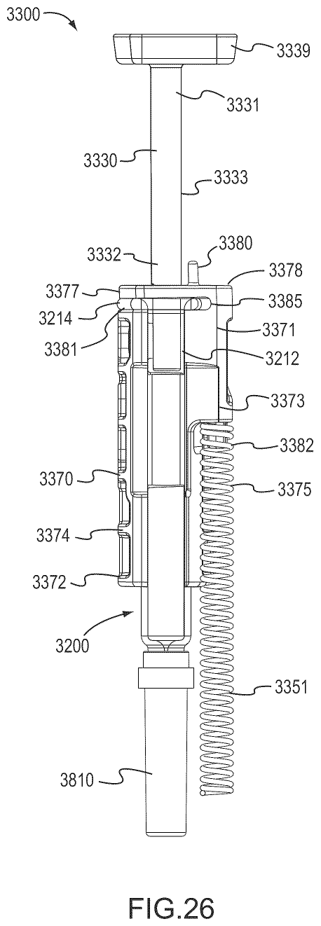

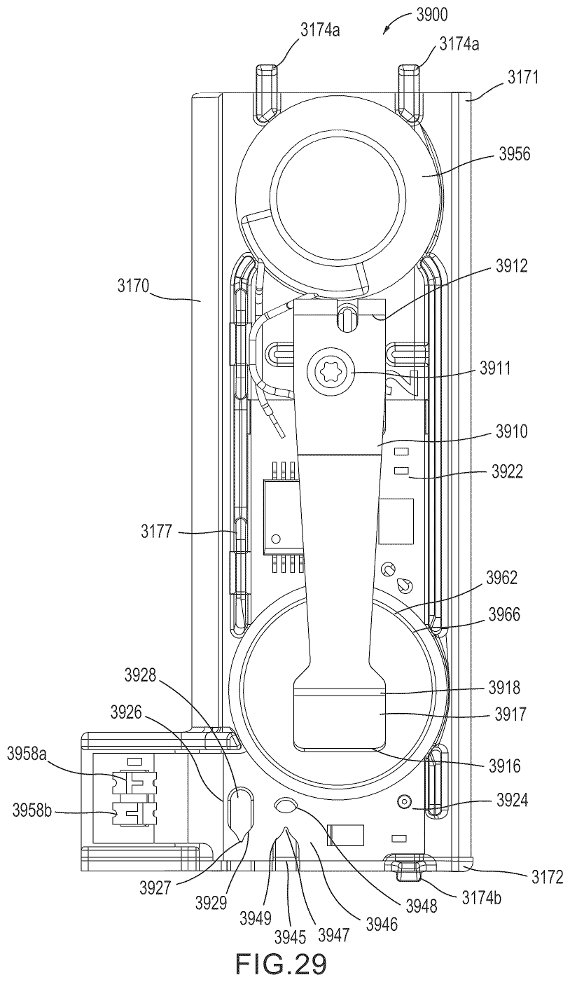

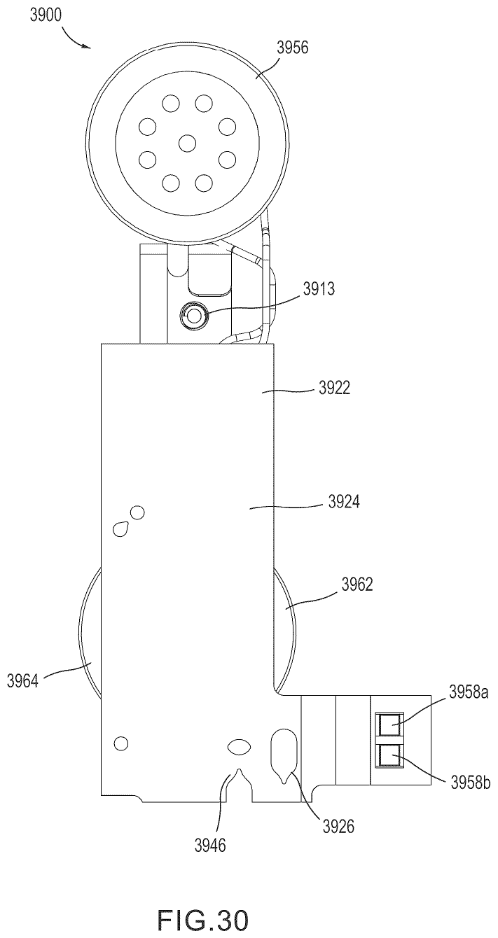

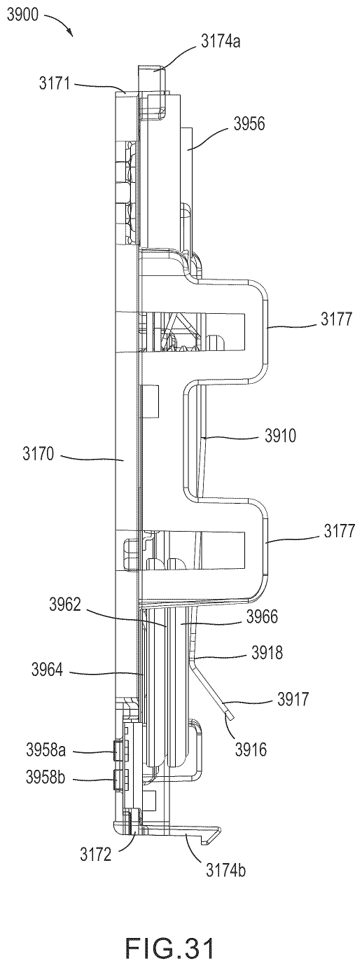

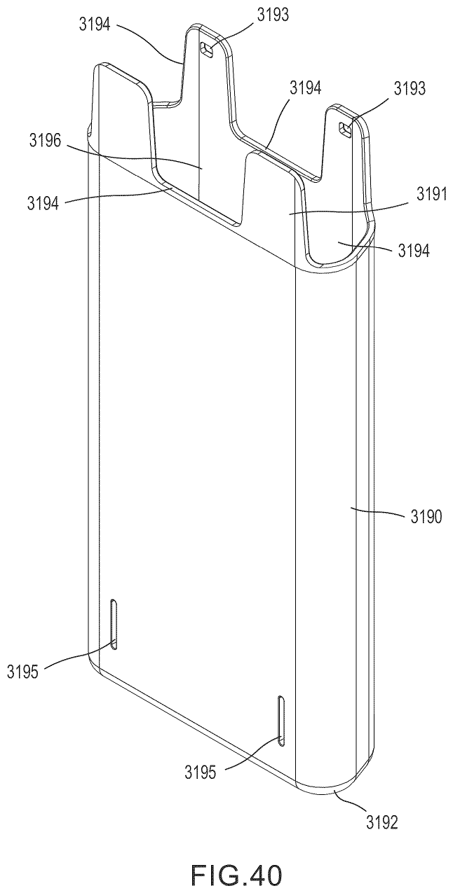

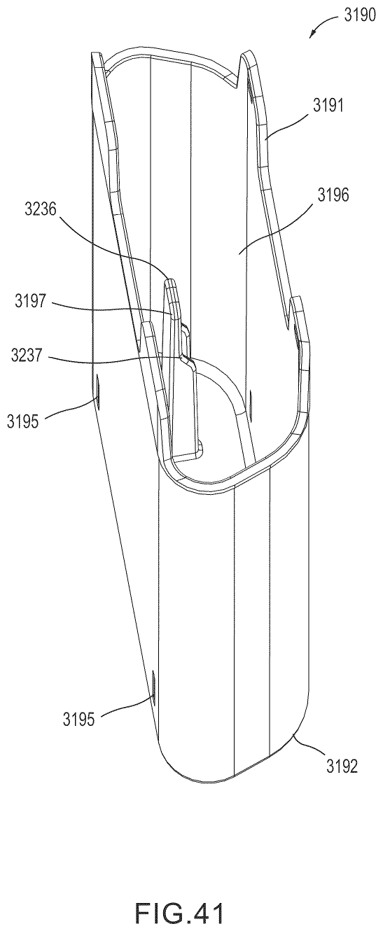

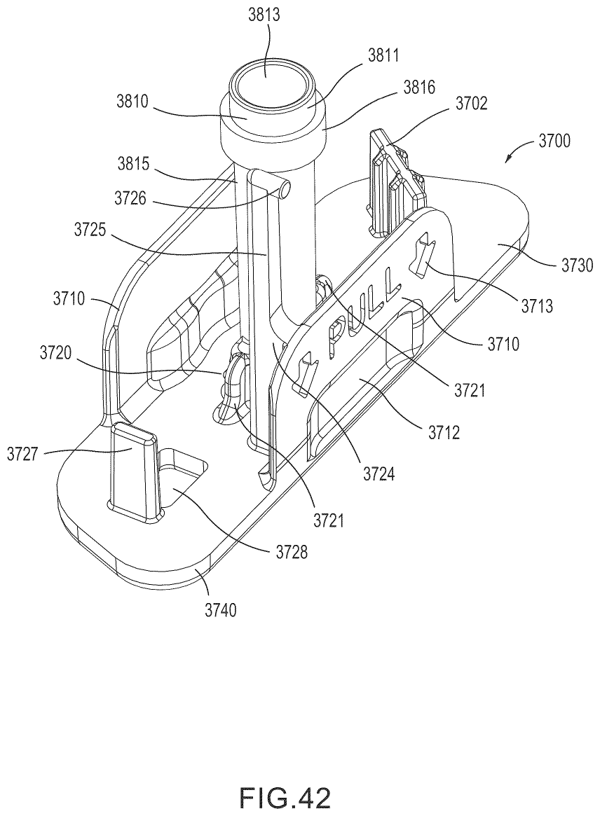

In some embodiments, the medicament delivery device can be a medical injector configured to automatically deliver a medicament contained within a medicament container, such as, for example a prefilled syringe. For example, FIGS. 9-55 show a medical injector 3000, according to an embodiment. FIGS. 9-10 are perspective views of the medical injector 3000 in a first configuration (i.e., prior to use). The medical injector 3000 includes a housing 3100 (see e.g., FIGS. 11-17), a system actuation assembly 3500 (see e.g., FIGS. 18-21), a medicament container 3200 containing a medicament 3220 (see e.g., FIG. 22), a medicament delivery mechanism 3300 (see e.g., FIG. 26-28), an electronic circuit system 3900 (see e.g., FIGS. 29-39), a cover 3190 (see e.g., FIGS. 40-41), and a safety lock 3700 (see e.g., FIGS. 42-46). A discussion of the components of the medical injector 3000 will be followed by a discussion of the operation of the medical injector 3000.

As shown in FIGS. 11-17, the housing 3100 has a proximal end portion 3101 and a distal end portion 3102. The housing 3100 defines a first status indicator aperture 3130 and a second status indicator aperture 3160. The first status indicator aperture 3130 defined by the housing 3100 is located on a first side of the housing 3100, and the second status indicator aperture 3160 of the housing 3100 is located on a second side of the housing 3100. The status indicator apertures 3130, 3160 can allow a patient to monitor the status and/or contents of the medicament container 3200 contained within the housing 3100. For example, by visually inspecting the status indicator apertures 3130, 3160, a patient can determine whether the medicament container 3200 contains a medicament 3220 and/or whether the medicament 3220 has been dispensed.

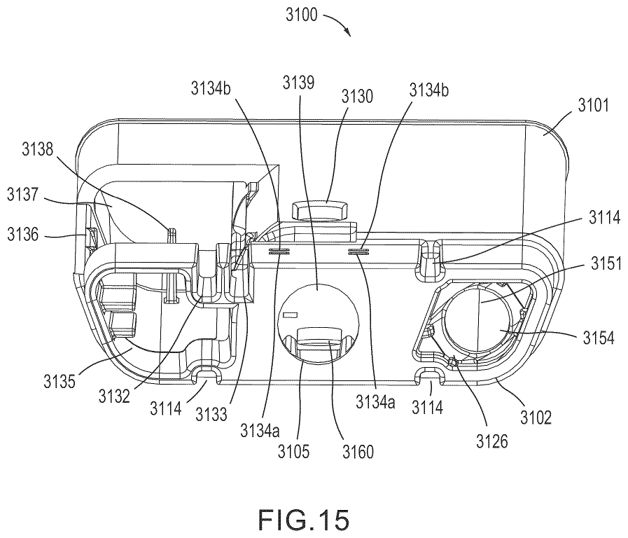

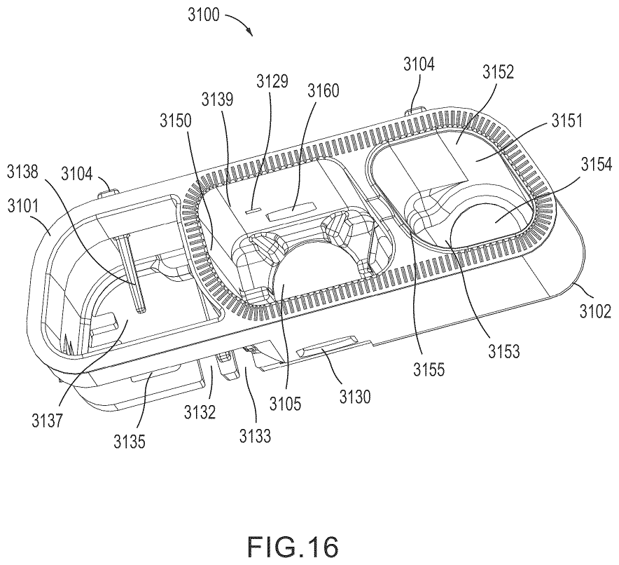

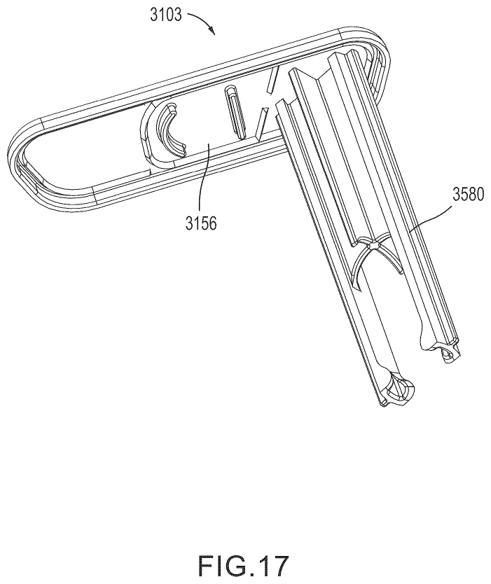

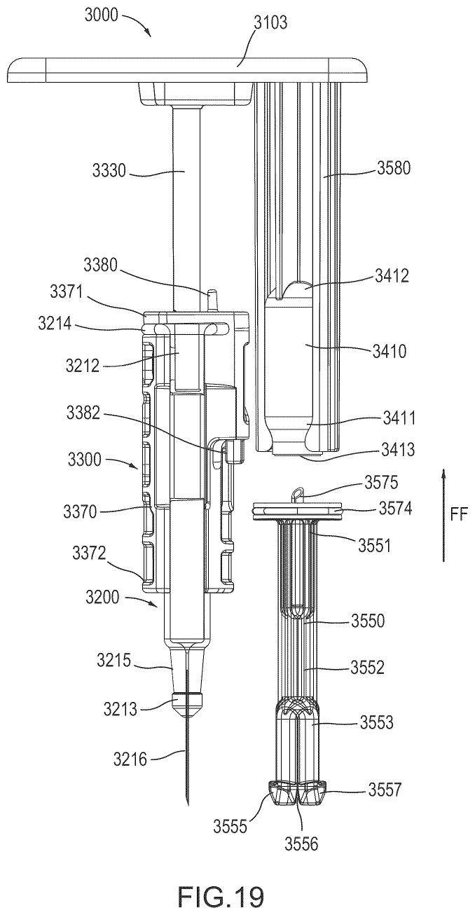

As shown in FIGS. 15 and 16, the housing 3100 defines a gas cavity 3151, a medicament cavity 3139 and an electronic circuit system cavity 3137. The gas cavity 3151 has a proximal end portion 3152 and a distal end portion 3153. The gas cavity 3151 is configured to receive the gas container 3410 and a portion of the system actuator assembly 3500 (e.g., a release member 3550 and the spring 3576, as shown in FIGS. 18 and 19) as described in further detail herein. The proximal end portion 3152 of the gas cavity 3151 is configured to receive the gas container retention member 3580 of a proximal cap 3103 of the housing 3100, as described in further detail herein. The gas cavity 3151 is in fluid communication with the medicament cavity 3139 via a gas passageway 3156 (see e.g., FIG. 17), as described in further detail herein, and the gas cavity 3151 is in fluid communication with a region outside the housing 3100 via a release member aperture 3154 (see e.g., FIGS. 15 and 16).

The medicament cavity 3139 is configured to receive the medicament container 3200 and at least a portion of the medicament delivery mechanism 3300. In particular, as described below, the medicament delivery mechanism 3300 includes a carrier 3370 and piston member 3330 movably disposed in the medicament cavity 3139. The medicament cavity 3139 is in fluid communication with a region outside the housing 3100 via a needle aperture 3105 (see e.g., FIGS. 15 and 16).

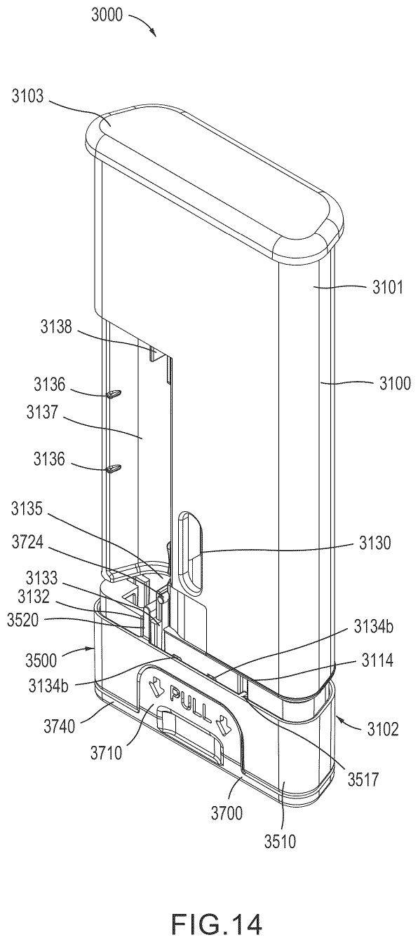

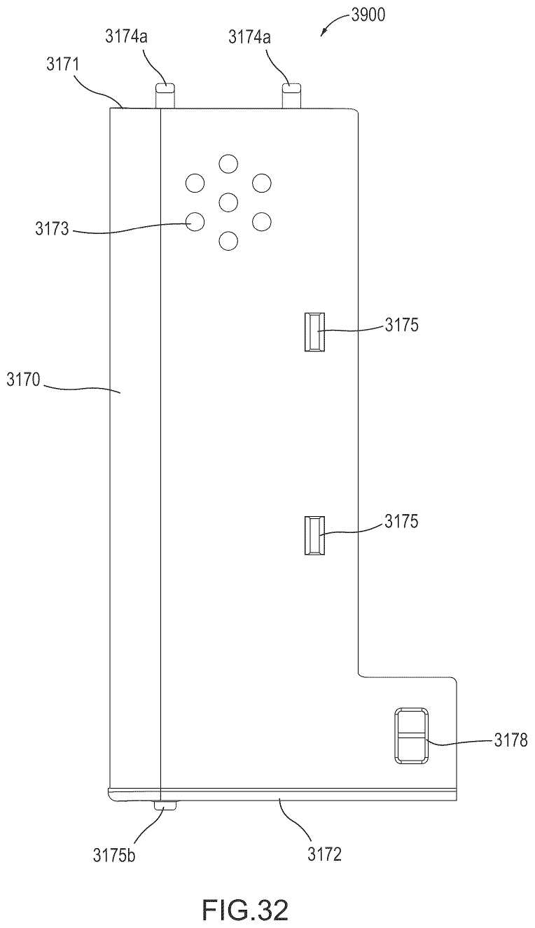

The electronic circuit system cavity 3137 is configured to receive the electronic circuit system 3900. The housing 3100 has protrusions 3136 (see e.g., FIG. 14) configured to stabilize the electronic circuit system 3900 when the electronic circuit system 3900 is disposed within the electronic circuit system cavity 3137. The outer surface of the housing 3100 is configured to receive a set of connection protrusions 3174A and connection protrusion 3177B of the electronic circuit system 3900 (see e.g., FIG. 32). In this manner, the electronic circuit system 3900 can be coupled to the housing 3100 within the electronic circuit system cavity 3137. In other embodiments, the electronic circuit system 3900 can be coupled within the electronic circuit system cavity 3137 by other suitable means such as an adhesive, a clip, a label and/or the like.

The electronic circuit system cavity 3137 is fluidically and/or physically isolated from the gas cavity 3151 and/or the medicament cavity 3139 by a sidewall 3150. The sidewall 3150 can be any suitable structure to isolate the electronic circuit system cavity 3137 within the housing 3100 from the gas cavity 3151 and/or the medicament cavity 3139 within the housing 3100. Similarly, the gas cavity 3151 and the medicament cavity 3139 are separated by a sidewall 3155 (see FIG. 16). In some embodiments, sidewall 3155 can be similar to the sidewall 3150, which isolates the gas cavity 3151 and the medicament cavity 3139 from the electronic circuit system cavity 3137. In other embodiments, the gas cavity 3151 can be fluidically and/or physically isolated from the medicament cavity 3139 by any suitable means. In yet other embodiments, the medicament cavity 3139 need not be fluidically and/or physically isolated from the electronic circuit system cavity 3137 and/or the gas cavity 3151.

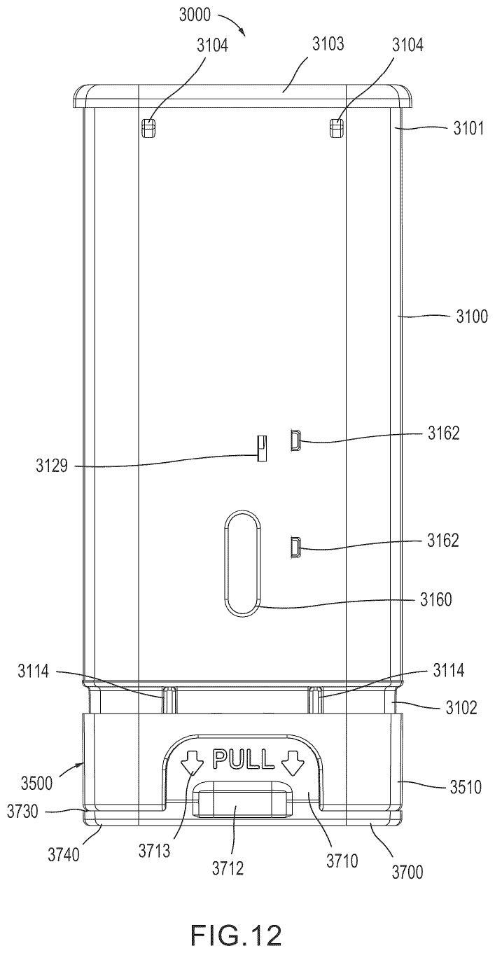

The proximal end portion 3101 of the housing 3100 includes a proximal cap 3103 (see e.g., FIG. 17), a speaker protrusion 3138 (see e.g., FIGS. 14-16), and cover retention protrusions 3104 (see e.g., FIGS. 10 and 12). The speaker protrusion 3138 is configured to maintain a position of an audio output device 3956 of the electronic circuit system 3900 relative to the housing 3100 when the electronic circuit system 3900 is attached to the housing 3100, as described herein. The cover retention protrusions 3104 are configured to be received within corresponding openings 3193 defined by the cover 3190 (see e.g., FIG. 10) to retain the cover 3190 about the housing 3100. In this manner, as described in more detail herein, the cover 3190 is removably coupled to and disposed about at least a portion of the housing 3100.

As shown in FIG. 17, the proximal cap 3103 includes a gas container retention member 3580 and defines a gas passageway 3156. The gas container retention member 3580 is configured to receive and/or retain a gas container 3410 that contains a pressurized gas, as shown in FIG. 18. When the medical injector 3000 is actuated, pressurized gas from the gas container 3140 is conveyed from the gas cavity 3151 to the medicament cavity 3139 via the gas passageway 3156, as further described herein. Said another way, the gas passageway 3156 places the gas cavity 3151 in fluid communication with the medicament cavity 3139.

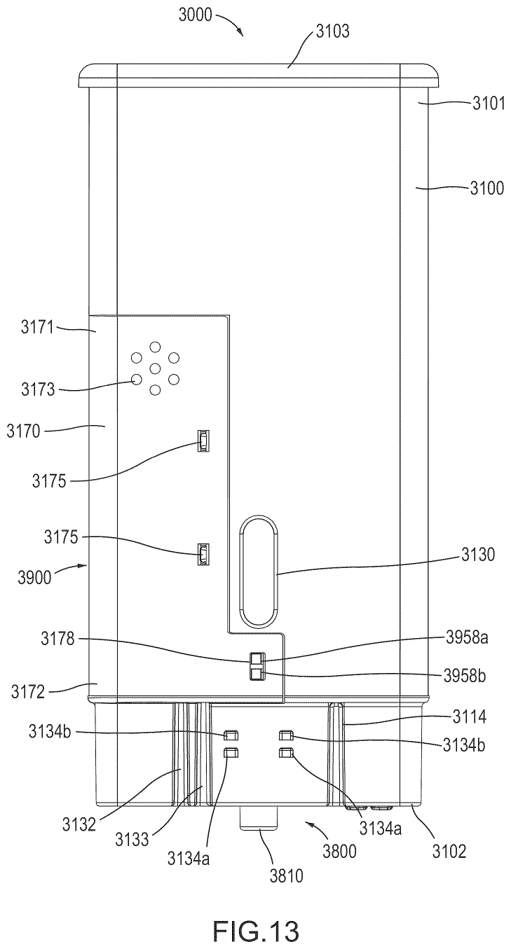

As shown in FIGS. 13 and 15, the distal end portion 3102 of the housing 3100 defines a battery isolation protrusion aperture 3135, a needle aperture 3105, a safety lock actuator groove 3133, a release member contact surface 3126, a release member aperture 3154, a base protrusion groove 3132, base retention recesses 3134A, 3134B, and base rail grooves 3114. The battery isolation protrusion aperture 3135 receives the battery isolation protrusion 3197 of the cover 3190 (see e.g., FIG. 41) when the cover 3190 is disposed about at least a portion of the housing 3100. The needle aperture 3105 is the opening through which the needle 3216 is disposed (see e.g., FIGS. 19, 51 and 52) when the medical injector 3000 is actuated, as described in further detail herein.

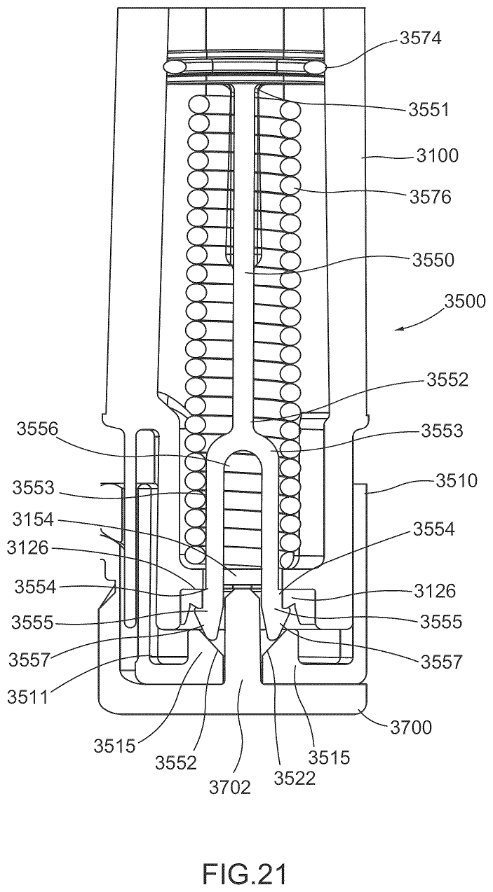

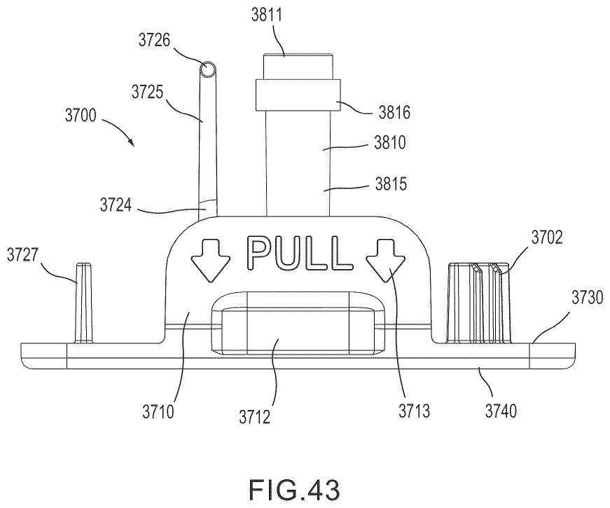

The safety lock actuator groove 3133 receives an actuator 3724 of the safety lock 3700 (see e.g., FIG. 43). As described in more detail herein, the actuator 3724 is configured to engage and/or activate the electronic circuit system 3900 when the safety lock 3700 is moved with respect to the housing 3100. The release member contact surface 3126 defines the release member aperture 3154. As shown in FIG. 21 and described in more detail below, the release member aperture 3154 receives a distal end portion 3552 of a release member 3550. As described in more detail below, a safety lock protrusion 3702 (see e.g., FIG. 42) is disposed within an opening 3556 between extensions 3553 of the release member 3550 (see e.g., FIGS. 19 and 21) such that an engagement surface 3554 of the extensions 3553 is engaged with the release member contact surface 3126 to prevent activation of the medical injector 3000. The safety lock 3700, its components and functions are described in more detail below.

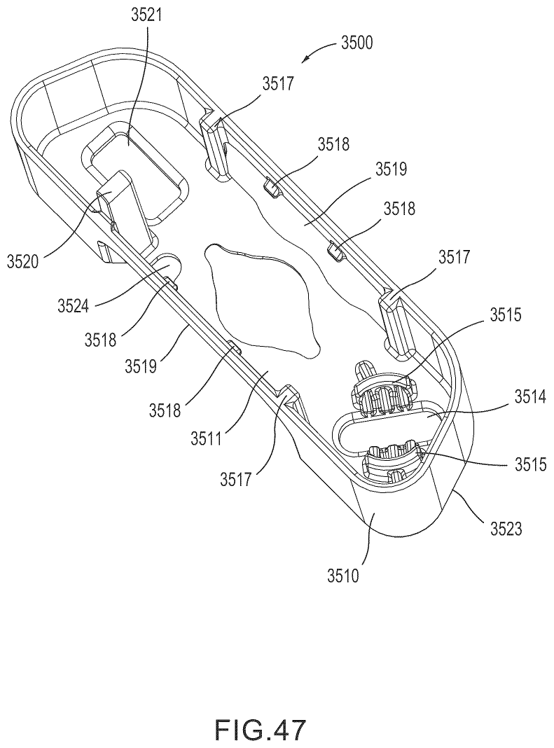

The distal base retention recesses 3134A are configured to receive the base connection knobs 3518 of the actuator 3510 (also referred to herein as “base 3510,” see e.g., FIG. 47) when the base 3510 is in a first position relative to the housing 3100. The proximal base retention recesses 3134B are configured to receive the base connection knobs 3518 of the base 3510 when the base 3510 is in a second position relative to the housing 3100. The base retention recesses 3134A, 3134B have a tapered proximal sidewall and a non-tapered distal sidewall. This allows the base retention recesses 3134A, 3134B to receive the base connection knobs 3518 such that the base 3510 can move proximally relative to the housing 3100, but cannot move distally relative to the housing 3100. Said another way, the distal base retention recesses 3134A are configured to prevent the base 3510 from moving distally when the base 3510 is in a first position and the proximal base retention recesses 3134B are configured to prevent the base 3510 from moving distally when the base 3510 is in a second position. Similarly stated, the proximal base retention recesses 3134B and the base connection knobs 3518 cooperatively to limit movement of the base to prevent undesirable movement of the base 3510 after the medical injector 3000 is actuated. The proximal base retention recesses 3134B and the base connection knobs 3518 also provide a visual cue to the user that the medical injector 3000 has been used.

The base actuator groove 3132 receives a protrusion 3520 of the base 3510. As described in more detail herein, the protrusion 3520 of the base 3510 is configured to engage the electronic circuit system 3900 when the base 3510 is moved with respect to the housing 3100. The base rail grooves 3114 receive the guide members 3517 of the base 3510 (see FIG. 47). The guide members 3517 of the base 3510 and the base rail grooves 3114 of the housing 3100 engage each other in a way that allows the guide members 3517 of the base 3510 to slide in a proximal and/or distal direction within the base rail grooves 3114 while limiting lateral movement of the guide members 3517. This arrangement allows the base 3510 to move in a proximal and/or distal direction with respect to the housing 3100 but prevents the base 3510 from moving in a lateral direction with respect to the housing 3100.

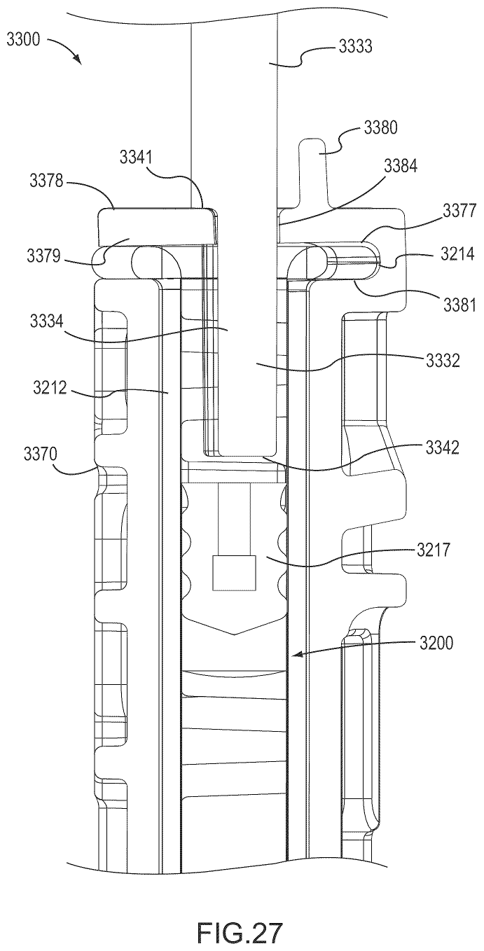

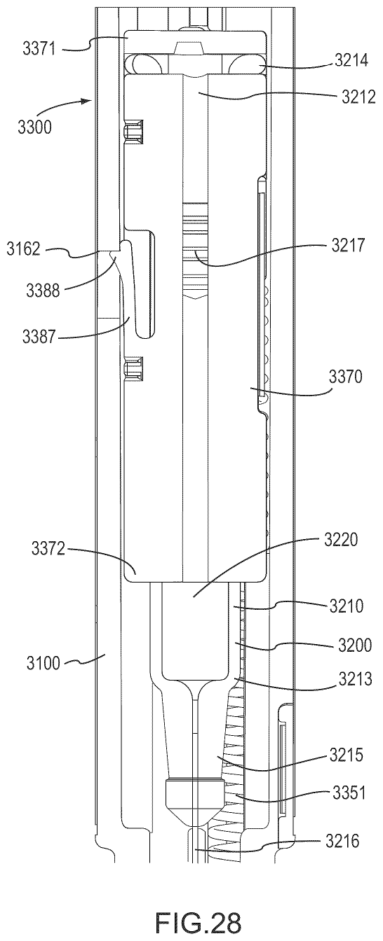

FIGS. 18-28 show the medicament container 3200, the system actuator assembly 3500 and the medicament delivery mechanism 3300 of the medical injector 3000. The medicament container 3200 has a body 3210 with a distal end portion 3213 and a proximal end portion 3212. The body 3210 defines a volume that contains (i.e., is filled with or partially filled with) a medicament 3220 (see, e.g., FIGS. 22 and 28). The distal end portion 3213 of the medicament container 3200 includes a neck 3215 that is coupled to the needle 3216, as described below. The proximal end portion 3212 of the medicament container 3200 includes an elastomeric member 3217 (i.e., a plunger) that seals the medicament 3220 within the body 3210. The elastomeric member 3217 is configured to move within the body to inject the medicament 3220 from the medicament container 3200. More particularly, as shown in FIG. 27, the elastomeric member 3217 is configured to receive and/or contact a piston rod 3333 of a piston member 3330 (also referred to herein as “second movable member 3330”) of the medicament delivery mechanism 3300.

The elastomeric member 3217 can be of any design or formulation suitable for contact with the medicament 3220. For example, the elastomeric member 3217 can be formulated to minimize any reduction in the efficacy of the medicament 3220 that may result from contact (either direct or indirect) between the elastomeric member 3217 and the medicament 3220. For example, in some embodiments, the elastomeric member 3217 can be formulated to minimize any leaching or out-gassing of compositions that may have an undesired effect on the medicament 3220. In other embodiments, the elastomeric member 3217 can be formulated to maintain its chemical stability, flexibility and/or sealing properties when in contact (either direct or indirect) with the medicament 3220 over a long period of time (e.g., for up to six months, one year, two years, five years or longer).

In some embodiments, the elastomeric member 3217 can be constructed from multiple different materials. For example, in some embodiments, at least a portion of the elastomeric member 3217 can be coated. Such coatings can include, for example, polydimethylsiloxane. In some embodiments, at least a portion of the elastomeric member 3217 can be coated with polydimethylsiloxane in an amount of between approximately 0.02 mg/cm² and approximately 0.80 mg/cm².

The proximal end portion 3212 of the body 3210 includes a flange 3214 configured to be disposed within a portion of the carrier 3370 (also referred to as a first movable member 3370), as described in further detail herein. The flange 3214 can be of any suitable size and/or shape. Although shown as substantially circumscribing the body 3210, in other embodiments, the flange 3214 can only partially circumscribe the body 3210.

The medicament container 3200 can have any suitable size (e.g., length and/or diameter) and can contain any suitable volume of the medicament 3220. Moreover, the medicament container 3200 and the second movable member 3330 can be collectively configured such that the second movable member 3330 travels a desired distance within the medicament container 3200 (i.e., the “stroke”) during an injection event. In this manner, the medicament container 3200, the volume of the medicament 3220 within the medicament container 3200 and the second movable member 3330 can be collectively configured to provide a desired fill volume and delivery volume. For example, the medicament container 3200, as shown in FIG. 22, is a prefilled syringe having a predetermined fill volume. Based on the predetermined fill volume, the second movable member 3330 can be configured to provide a desired delivery volume.

Moreover, the length of the medicament container 3200 and the length of the second movable member 3330 can be configured such that the medicament delivery mechanism 3300 can fit within the same housing 3100 regardless of the fill volume, the delivery volume and/or the ratio of the fill volume to the delivery volume. In this manner, the same housing and production tooling can be used to produce devices having various dosages of the medicament 3220. For example, in a first embodiment (e.g., having a fill volume to delivery volume ratio of 0.4), the medicament container has a first length and the second movable member has a first length. In a second embodiment (e.g., having a fill volume to delivery volume ratio of 0.6), the medicament container has a second length shorter than the first length, and the second movable member has a second length longer than the first length. In this manner, the stroke of the device of the second embodiment is longer than that of the device of the first embodiment, thereby allowing a greater dosage. The medicament container of the device of the second embodiment, however, is shorter than the medicament container of the device of the first embodiment, thereby allowing the components of both embodiments to be disposed within the same housing and/or a housing having the same length.

As shown in FIGS. 18-21, the system actuator assembly 3500 includes the base 3510, a release member 3550 and a spring 3576. FIG. 19 shows certain internal components of the medical injector 3000 without the base 3510 and the spring 3576 so that the release member 3550 can be more clearly shown.

The release member 3550 has a proximal end portion 3551 and a distal end portion 3552, and is movably disposed within the distal end portion 3153 of the gas cavity 3151. The proximal end portion 3551 of the release member 3550 includes a sealing member 3574 and a puncturer 3575. The sealing member 3574 is configured to engage the sidewall of the housing 3100 defining the gas cavity 3151 such that the proximal end portion 3152 of the gas cavity 3151 is fluidically isolated from the distal end portion 3153 of the gas cavity 3151. In this manner, when gas is released from the gas container 3410, the gas contained in the proximal end portion 3152 of the gas cavity 3151 is unable to enter the distal end portion 3153 of the gas cavity 3151. The puncturer 3575 of the proximal end portion 3551 of the release member 3550 is configured to contact and puncture a frangible seal 3413 on the gas container 3410 when the release member 3550 moves proximally within the gas cavity 3151, as shown by the arrow FF in FIG. 19.

The distal end portion 3552 of the release member 3550 includes extensions 3553. The extensions 3553 have projections 3555 that include tapered surfaces 3557 and engagement surfaces 3554. Further, the extensions 3553 define an opening 3556 between the extensions 3553. The engagement surfaces 3554 of the projections 3555 are configured to extend through the release member aperture 3154 of the housing 3100 and contact the release member contact surface 3126 of the housing 3100, as shown in FIG. 21. In this manner, the engagement surfaces 3554 of the projections 3555 limit proximal movement of the release member 3550 when the engagement surfaces 3554 are in contact with the release member contact surface 3126 of the housing 3100.

The opening 3556 defined by the extensions 3553 is configured to receive the safety lock protrusion 3702 of the safety lock 3700 (see e.g., FIGS. 21 and 42) when the safety lock 3700 is coupled to the housing 3100 and/or the base 3510. The safety lock protrusion 3702 is configured to prevent the extensions 3553 from moving closer to each other. Said another way, the safety lock protrusion 3702 is configured to ensure that the extensions 3553 remain spaced apart and the engagement surfaces 3554 of the projections 3555 remain in contact with the release member contact surface 3126 of the housing 3100. In some embodiments, for example, the release member 3550 and/or the extensions 3553 can be constructed from any suitable material configured to withstand deformation that may occur when exposed to a load over an extended period of time. In some embodiments, for example, the release member 3550 and/or the extensions 3553 can be constructed from brass.

The tapered surfaces 3557 of the projections 3555 are configured to contact tapered surfaces 3522 of contact protrusions 3515 on a proximal surface 3511 of the base 3510 (see e.g., FIGS. 21 and 47) when the base 3510 is moved proximally relative to the housing 3100. Accordingly, when the base 3510 is moved proximally relative to the housing 3100, the extensions 3553 are moved together by the tapered surfaces 3522 of the contact protrusions 3515. The inward movement of the extensions 3553 causes the release member 3550 to disengage the release member contact surface 3126 of the housing 3100, thereby allowing the release member 3550 to be moved proximally along its longitudinal axis as the spring 3576 expands.

The medicament delivery mechanism 3300 includes a gas container 3410, the carrier 3370 (also referred to herein as the first movable member 3370), the piston member 3330 (also referred to herein as the second movable member 3330), and a retraction spring 3351. As described above, the carrier 3370 and the piston member 3330 are each movably disposed within the medicament cavity 3139 of the housing 3100. The gas container 3410 is disposed within the gas cavity 3151 of the housing 3100.

The gas container 3410 includes a distal end portion 3411 and a proximal end portion 3412, and is configured to contain a pressurized gas. The distal end portion 3411 of the gas container 3410 contains a frangible seal 3413 configured to break when the puncturer 3575 of the proximal end portion 3551 of the release member 3550 contacts the frangible seal 3413. The gas container retention member 3580 of the proximal cap 3103 of the housing 3100 is configured to receive and/or retain the proximal end portion 3412 of the gas container 3410. Said another way, the position of the gas container 3410 within the gas cavity 3151 is maintained by the gas container retention member 3580. As shown in FIGS. 18 and 19, the length of the gas container retention member 3580 and the length of the release member 3550 collectively determine the distance between the puncturer 3575 and the frangible seal 3413 when the medical injector 3000 is in the storage configuration. Accordingly, this distance, which is the distance through which the puncturer 3575 travels when the medical injector 3000 is actuated, can be adjusted by changing the length of the gas container retention member 3580 and/or the length of the release member 3550. In some embodiments, the actuation time and/or the force exerted by the puncturer 3575 on the frangible seal 3413 can be adjusted by changing the distance between the puncturer 3575 and the frangible seal 3413.

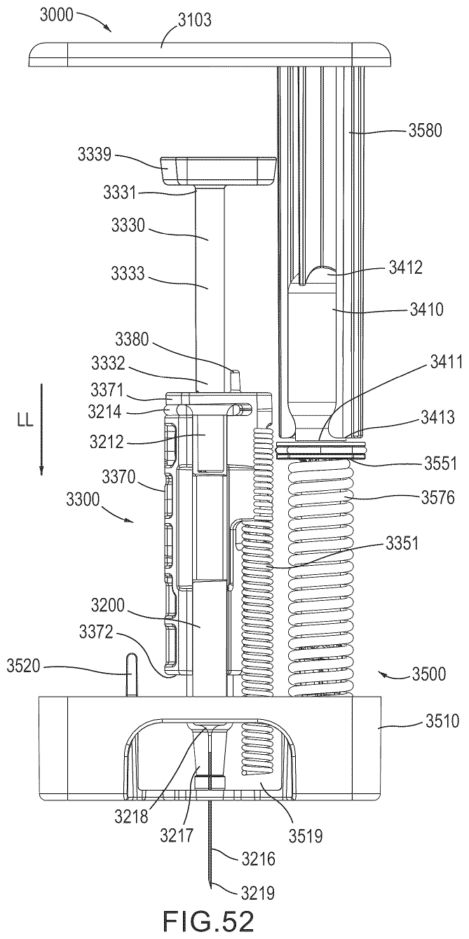

As shown in FIGS. 26 and 52, the piston member 3330 includes a piston rod 3333, and has a proximal end portion 3331 and a distal end portion 3332. The proximal end portion 3331 includes a sealing member 3339. The sealing member 3339 engages the sidewall of the housing 3100 to define a gas chamber (i.e., a volume within the medicament cavity 3139 between the proximal end of the housing 3100 and the proximal end of the piston member 3330) that receives the pressurized gas from the gas container 3410. The sealing member 3339 can be any suitable structure and or component to produce a substantially fluid-tight seal between the sidewall of the housing 3100 and the piston member 3330. The proximal end portion 3331 also includes a gas relief valve 3340 (see e.g., FIGS. 26 and 53-55) configured to be selectively actuated to allow fluid communication between the gas chamber and a volume outside of the gas chamber (e.g., the distal end portion of the medicament cavity 3139). As described in more detail below, the gas relief valve 3340 allows the gas pressure within the gas chamber to be reduced upon completion of the injection event.

Referring to FIG. 27, the distal end portion 3332 includes a first surface 3341 and a second surface 3342. The second surface 3342 is disposed through a piston rod opening 3384 of the carrier 3370 and within the proximal end portion 3212 of the medicament container 3200. The first surface 3341 is configured to contact a proximal surface 3378 of an engagement portion 3379 of the carrier 3370 when the medicament injector 3000 is in a first configuration (i.e., when the medicament container 3200 is in its first position). The distance between the first surface 3341 and the second surface 3342 is such that when the first surface 3341 is in contact with the engagement portion 3379 of the carrier 3370, the second surface 3342 is spaced apart from the elastomeric member 3217 within the medicament container 3200 (see e.g., FIG. 27). This arrangement limits any preload and/or residual force applied to the piston member 3330 (e.g., via the retraction spring 3351 and/or the pressurized gas) from being transferred to the plunger 3217. Said another way, the plunger 3217 is isolated from the piston member 3330 during the storage configuration and/or when the medicament container 3200 is moving distally within the housing 3100. Accordingly, this arrangement reduces and/or eliminates leakage of the medicament 3220 from the medicament container 3200.

As described in more detail herein, the piston member 3330 is configured to move within the medicament container 3200. Because the first surface 3341 is configured to contact the engagement portion 3379, the piston member 3330 applies a force to the proximal surface 3378 of the first shoulder 3377 such that the carrier 3370 and the piston member 3330 move together within the medicament cavity 3139. Moreover, when the medicament container 3200 is in its second position, the piston member 3330 can move relative to the carrier 3370 and/or the medicament container 3200 such that the second surface 3342 engages and/or contacts the elastomeric member 3217 to convey the medicament 3220 contained in the medicament container 3200. The piston member 3330 can be constructed of a resilient, durable and/or sealing material or combination of materials, such as a rubber.

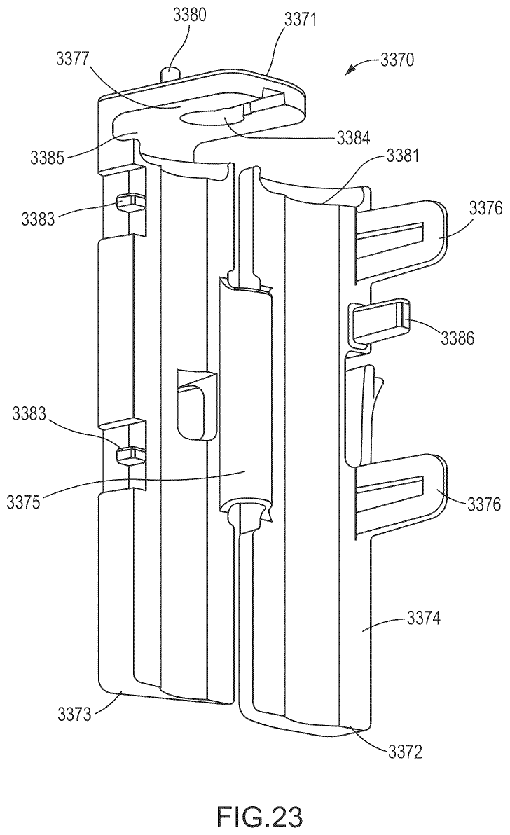

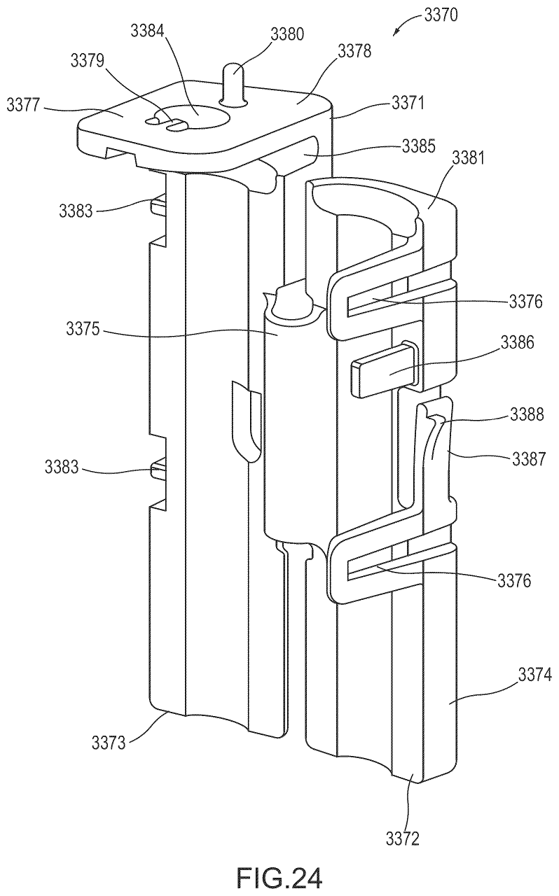

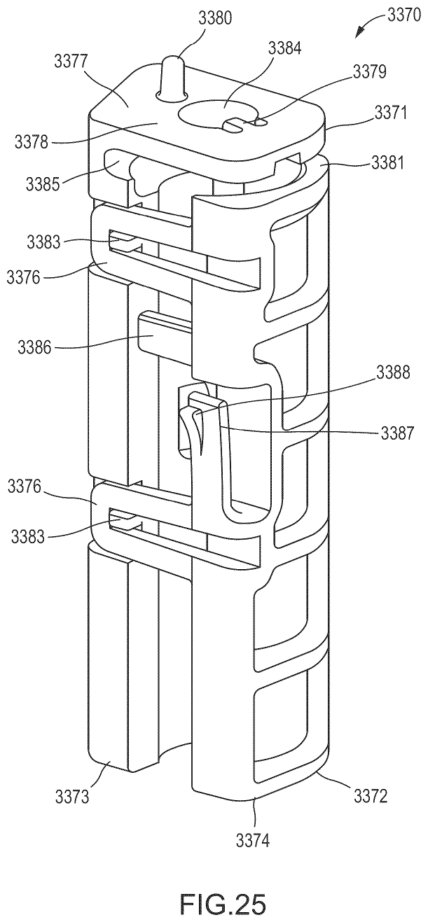

The carrier 3370 of the medicament delivery mechanism 3300 includes a distal end portion 3372, a proximal end portion 3371, a first side portion 3373, a second side portion 3374 and a hinge portion 3375 (see e.g., FIGS. 23-28). The first side portion 3373 includes latch protrusions 3383 configured to be coupled to the corresponding latches 3376 of the second side portion 3374. The second side portion 3374 is configured to move relative to the first side portion 3373 via the hinge portion 3375 between an opened configuration (FIGS. 23 and 24) and a closed configuration (FIG. 25). This arrangement allows at least the proximal end portion 3212 of the medicament container 3200 to be disposed within (and/or removed from) the carrier 3370 when the carrier 3370 is in the opened configuration (see e.g., FIGS. 23 and 24). When the carrier 3370 is in the closed configuration (see e.g., FIGS. 25-28), the latches 3376 of the second side portion 3374 engage the latch protrusions 3383 of the first side portion 3373 to maintain the medicament container 3200 within the carrier 3370.

The proximal end portion 3371 of the carrier 3370 includes a first shoulder 3377 and a second shoulder 3381 that collectively define a flange groove 3385. The flange groove 3385 is configured to receive the flange 3214 of the proximal end portion 3212 of the medicament container 3200 (see e.g., FIG. 26). More particularly, the first shoulder 3377 is defined by the first side portion 3373, and the second shoulder 3381 is defined by portions of both the first side portion 3373 and the second side portion 3374. In this manner, the first shoulder 3377 is configured to contact a proximal surface of the flange 3214, either directly or via intervening structure (e.g., an o-ring, a damping member, or the like). Similarly, the second shoulder 3381 is configured to contact a distal surface of the flange 3214, either directly or via intervening structure (e.g., an o-ring, a damping member, or the like). In this manner, as described in more detail below, the first shoulder 3377 can transfer at least a portion of a distal force (i.e., an insertion force) to the flange 3214 to produce distal movement of the carrier 3370 and/or the medicament container 3200 within the housing 3100. The second shoulder 3381 can transfer at least a portion of a proximal force (i.e., a retraction force) to the flange 3214 to produce proximal movement of the carrier 3370 and/or the medicament container 3200 within the housing 3100.

The second side portion 3374 includes a protrusion 3386 configured to contact a surface of the first side portion 3373 when the carrier 3370 is in the closed configuration (FIG. 25). In this manner, the protrusion 3386 and the corresponding portion of the first side portion 3373 limits the movement of the second side portion 3374 relative to the first side portion 3373 when the carrier 3370 is in the closed configuration. Similarly stated, the protrusion 3386 of the second side portion 3374 contacts the first side portion 3373 to prevent the carrier 3370 from squeezing the medicament container 3200, when the carrier 3370 is in the closed configuration.

The second side portion 3374 includes a latch 3387 having a protrusion 3388. The protrusion 3388 of the latch 3387 is configured to engage a retraction lock protrusion 3162 defined by the sidewall of the housing 3100 defining the medicament cavity 3139 (see e.g., FIG. 28) when the carrier 3370 and the medicament container 3200 are in the first (i.e., storage) position. This arrangement allows the medicament delivery mechanism 3300 (e.g., the carrier 3370, the piston member 3330) and the medicament container 3200 to move in the distal direction within the housing 3100 but limits the movement of the carrier 3370 and the medicament container 3200 in the proximal direction. In this manner, the preload of the retraction spring 3351 is not transferred to the piston member 3330 and/or the engagement portion 3379 of the carrier 3370. Similarly stated, this arrangement prevents the medicament delivery mechanism 3300 from moving in the proximal direction when the medical injector 3000 is in the first configuration. This arrangement also limits proximal motion of the medicament delivery mechanism 3300 during assembly (e.g., when the needle sheath is being pressed about the needle).

As described above, the carrier 3370 includes the engagement portion 3379 configured to engage the first surface 3341 of the piston member 3330. The first shoulder 3377 is in contact with the proximal surface of the flange 3214 and therefore transmits a force from the piston member 3330 to move the medicament container 3200 from a first position to a second position when the medicament injector 3000 is actuated.

As shown in FIG. 26, the carrier 3370 also includes an engagement portion 3382 configured to engage the retraction spring 3351. Although the engagement portion 3382 is shown as including a protrusion about which a portion of the retraction spring 3351 is disposed, in other embodiments, the engagement portion 3382 can include any suitable features for engaging and/or retaining the retraction spring 3351 (e.g., a recess). The second shoulder 3381 is configured to engage the distal end of the flange 3214 and therefore transmits a retraction force produced by the retraction spring 3351 to move the medicament container 3200 from the second position toward the first position.

A proximal surface 3378 of the first shoulder 3377 of the carrier 3370 includes a gas valve actuator 3380. The gas valve actuator 3380 is configured to engage the gas relief valve 3340 (see e.g., FIG. 26) of the piston member 3330 to allow the pressurized gas contained within the gas chamber (i.e., the volume within the medicament cavity 3139 between the proximal end of the housing 3100 and the proximal end of the piston member 3330) to escape when the injection event is complete. Thus, after the gas pressure within the medicament cavity 3139 decreases below a certain level, the force exerted by the retraction spring 3351 on the carrier 3370 is sufficient to cause the carrier 3370 to move proximally within the housing 3100 (i.e., to retract). In addition, this arrangement results in there being substantially no residual force (from the pressurized gas) within the housing, which decreases stress on the components after the injection event.

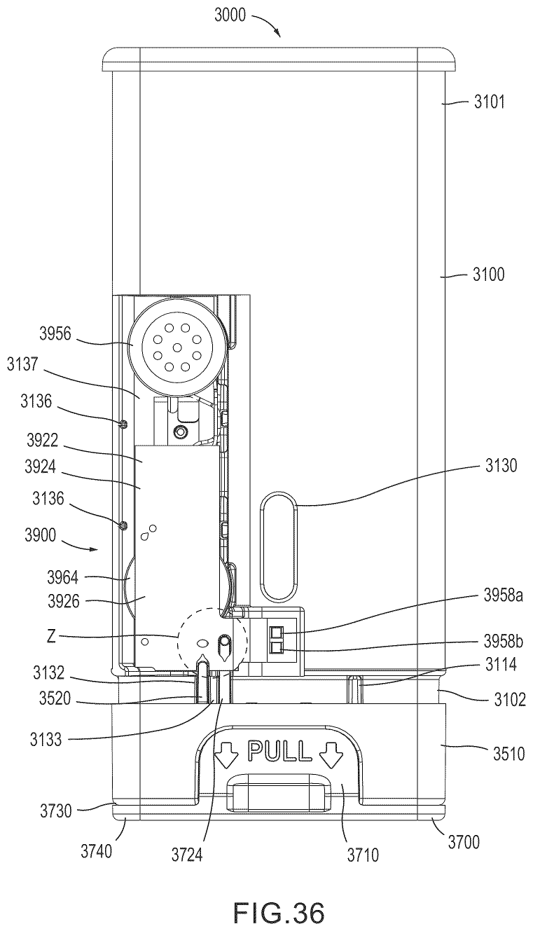

FIGS. 29-39 show the electronic circuit system 3900. The electronic circuit system 3900 of the medical injector 3000 includes an electronic circuit system housing 3170, a printed circuit board 3922, a battery assembly 3962, an audio output device 3956, two light emitting diodes (LEDs) 3958A, 3958B and a battery clip 3910. As shown in FIG. 36, the electronic circuit system 3900 is disposed within the electronic circuit system cavity 3137 of the housing 3100. As described herein, the electronic circuit system 3900 is configured to output an electronic output associated with the use of the medical injector 3000.

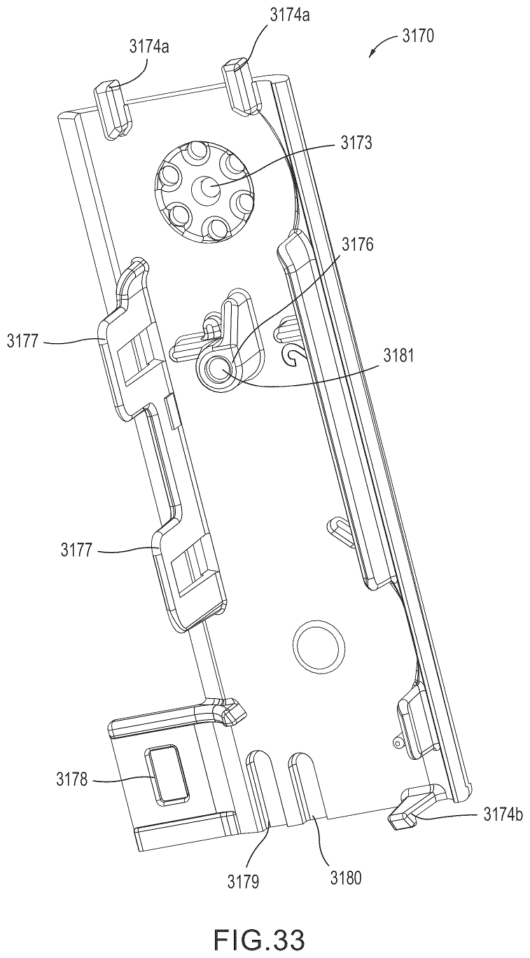

The electronic circuit system housing 3170 of the electronic circuit system 3900 includes a distal end portion 3172 and a proximal end portion 3171. The proximal end portion 3171 includes connection protrusions 3174A and a battery clip protrusion 3176 (see e.g., FIG. 33). The connection protrusions 3174A are configured to matingly engage a surface of the sidewalls of the housing 3100 that define the electronic cavity 3137, as described above. In this manner, the electronic circuit system 3900 can be coupled to the housing 3100 within the electronic circuit system cavity 3137. In other embodiments, the electronic circuit system 3900 can be coupled to the housing 3100 by other suitable means such as an adhesive, a clip, a label and/or the like. As described in more detail herein, the battery clip protrusion 3176 is configured to hold the battery clip 3910 in place.

The proximal end portion 3171 of the electronic circuit system housing 3170 defines multiple sound apertures 3173. The audible output device 3956 is disposed against the proximal end portion 3171 of the electronic circuit system housing 3170 such that the front face of the audible output device 3956 is disposed adjacent the sound apertures 3173. In this manner, the sound apertures 3173 are configured to allow sound produced by the audio output device 3956 to pass from the audio output device 3956 to a region outside of the housing 3100.

As shown in FIGS. 32 and 33, the distal end portion 3172 of the electronic circuit system housing 3170 includes the connection protrusion 3174B, a stiffening protrusion 3177 and defines an LED aperture 3178, apertures 3175, a safety lock actuator groove 3179 and a base actuator groove 3180. The LED aperture 3178 is configured to receive the LEDs 3958A, 3958B such that a user can view the LEDs 3958A, 3958B, which are described in more detail herein.

The connection protrusion 3174B extends from the distal end portion 3172 of the electronic circuit system housing 3170, and is configured to attach the electronic circuit system 3900 to the housing 3100, as described above. The stiffening protrusion 3177 is configured to have at least a portion received within and/or accessible via the apertures 3175 defined by the housing 3100 (see e.g., FIG. 11). The stiffening protrusion 3177 is configured to limit the bending (e.g., buckling) of the electronic circuit system housing 3170 when the electronic circuit system housing 3170 is coupled to the housing 3100. Moreover, a user can access the stiffening protrusion 3177 via the apertures 3175. In this manner, for example, the user can disengage the stiffening protrusion 3177 from the apertures 3175.

The safety lock actuator groove 3179 of the electronic circuit system housing 3170 is configured to be disposed adjacent the safety lock actuator groove 3133 of the distal end portion 3102 of the housing 3100. In this manner, the safety lock actuator groove 3179 of the electronic circuit system housing 3170 and the safety lock actuator groove 3133 of the distal end portion 3102 of the housing 3100 collectively receive the actuator 3724 of the safety lock 3700, which is described in more detail herein. Similarly, the base actuator groove 3180 of the electronic circuit system housing 3170 is configured to be disposed adjacent the base actuator groove 3132 of the distal end portion 3102 of the housing 3100. The base actuator groove 3180 of the electronic circuit system housing 3170 and the base actuator groove 3132 of the distal end portion 3102 of the housing 3100 collectively receive the protrusion 3520 of the base 3510, which is described in more detail herein.

The printed circuit board 3922 of the electronic circuit system 3900 includes a substrate 3924, a first actuation portion 3926 and a second actuation portion 3946. The substrate 3924 of the printed circuit board 3922 includes the electrical components for the electronic circuit system 3900 to operate as desired. For example, the electrical components can be resistors, capacitors, inductors, switches, microcontrollers, microprocessors and/or the like. The printed circuit board may also be constructed of materials other than a flexible substrate such as a FR4 standard board (rigid circuit board).

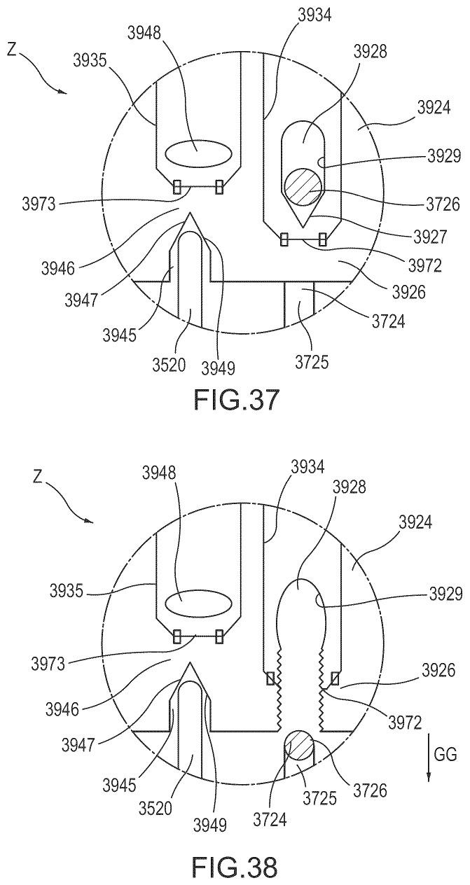

As shown in FIGS. 37-39, the first actuation portion 3926 includes a first electrical conductor 3934 and defines an opening 3928 having a boundary 3929. The opening 3928 of the first actuation portion 3926 is configured to receive a protrusion 3726 of the actuator 3724 of the safety lock 3700. The boundary 3929 of the first opening 3928 has a discontinuous shape, such as, for example, a teardrop shape, that includes a stress concentration riser 3927. The discontinuity and/or the stress concentration riser 3927 of the boundary 3929 can be of any suitable shape to cause the substrate 3924 to deform in a predetermined direction when the protrusion 3726 of the actuator 3724 of the safety lock 3700 is moved relative to the opening 3928, as shown by the arrow GG in FIG. 38.

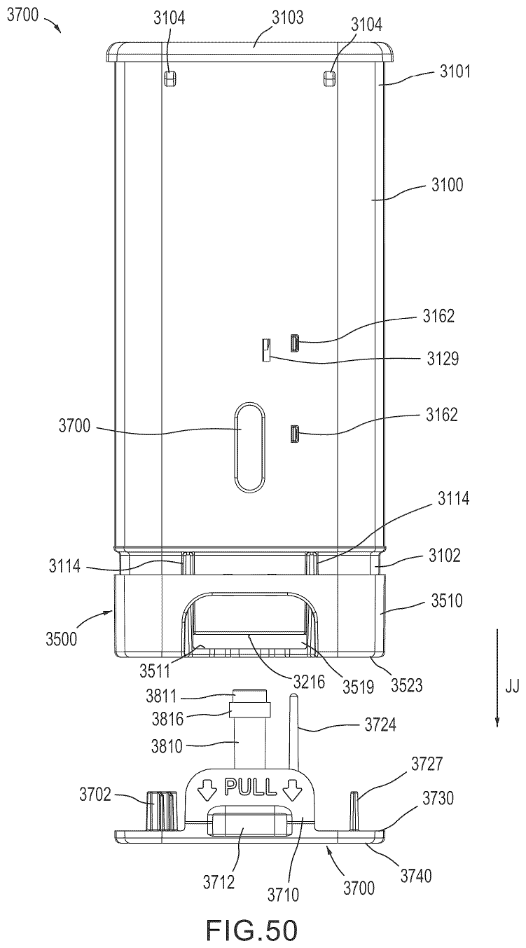

The opening 3928 is defined adjacent the first electrical conductor 3934 that electronically couples the components included in the electronic circuit system 3900. The first electrical conductor 3934 includes a first switch 3972, which can be, for example a frangible portion of the first electrical conductor 3934. In use, when the safety lock 3700 is moved from a first position (see e.g., FIG. 37) to a second position (see e.g., FIG. 38), the actuator 3724 moves in a direction substantially parallel to a plane defined by a surface of the first actuation portion 3926 of the substrate 3924. The movement of the actuator 3724 causes the protrusion 3726 to move within the first opening 3928, as indicated by the arrow GG in FIG. 38. The movement of the protrusion 3726 tears the first actuation portion 3926 of the substrate 3924, thereby separating the portion of the first electrical conductor 3934 including the first switch 3972. Said another way, when the safety lock 3700 is moved from its first position to its second position (see e.g., FIG. 50), the actuator 3724 moves irreversibly the first switch 3972 from a first state (e.g., a state of electrical continuity) to a second state (e.g., a state of electrical discontinuity). Said yet another way, when the safety lock 3700 is moved from its first position to its second position, the actuator 3724 disrupts the first electrical conductor 3934.

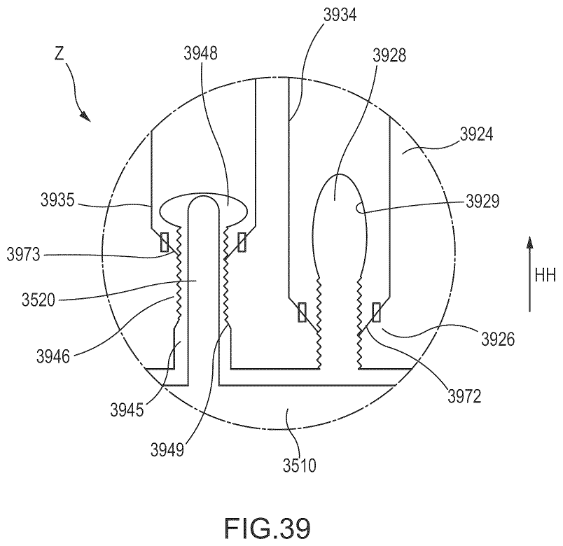

The second actuation portion 3946 includes a second electrical conductor 3935 and defines an opening 3945, having a boundary 3949 and a tear propagation limit aperture 3948. As shown in FIGS. 36-39, the opening 3945 of the second actuation portion 3946 is configured to receive a portion of an actuator 3520 of the base 3510. The boundary 3949 of the opening 3945 has a discontinuous shape that includes a stress concentration riser 3947. The discontinuity and/or the stress concentration riser 3947 of the boundary 3949 can be of any suitable shape to cause the substrate 3924 to deform in a predetermined direction when the actuator 3520 of the base 3510 is moved in a proximal direction relative to the opening 3945, as shown by the arrow HH in FIG. 39.

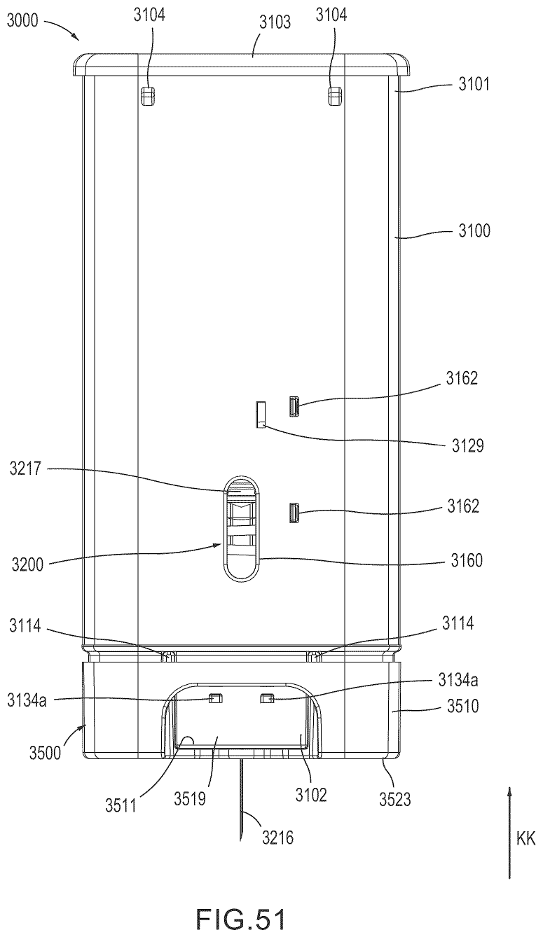

The second electrical conductor 3935 includes a second switch 3973 disposed between the opening 3945 and the tear propagation limit aperture 3948, which can be, for example, a frangible portion of the second electrical conductor 3935. In use, when the base 3510 is moved from its first position to its second position (see e.g., FIG. 51), the actuator 3520 moves in a proximal direction, substantially parallel to a plane defined by a surface of the second actuation portion 3946 of the substrate 3924. The proximal movement of the actuator 3520 tears the second actuation portion 3946 of the substrate 3924, thereby separating the portion of the second electrical conductor 3935 including the second switch 3973. Said another way, when the base 3510 is moved from its first position to its second position, the actuator 3520 moves irreversibly the second switch 3973 from a first state (e.g., a state of electrical continuity) to a second state (e.g., a state of electrical discontinuity). The tear propagation limit aperture 3948 is configured to limit the propagation of the tear in the substrate 3924 in the proximal direction. Said another way, the tear propagation limit aperture 3948 is configured to ensure that the tear in the substrate 3924 does not extend beyond the tear propagation limit aperture 3948. The tear propagation limit aperture 3948 can be any shape configured to stop the propagation of a tear and/or disruption of the substrate 3924. For example, the tear propagation limit aperture 3948 can be oval shaped. In other embodiments, the proximal boundary of the tear propagation limit aperture 3948 can be reinforced to ensure that the tear in the substrate 3924 does not extend beyond the tear propagation limit aperture 3948.

In some embodiments, the safety lock 3700 and base 3510 can be configured to interact with mechanical and/or optical switches to produce an electronic output in a reversible manner.

The battery assembly 3962 of the electronic circuit system 3900 includes two batteries stacked on top of one another. In other embodiments, the electronic circuit system can include any number of batteries and/or any suitable type of power source. In some embodiments, for example, the battery assembly can include Lithium batteries such as, for example, CR1616, CR2016s, type AAA or the like. The battery assembly 3962 has a first surface 3964 and a second surface 3966. The first surface 3964 of the battery assembly 3962 can contact an electrical contact (not shown) disposed on the substrate 3924. The second surface 3966 of the battery assembly 3962 is configured to contact a contact portion 3918 of a distal end portion 3916 of a battery clip 3910. When both the electrical contact of the substrate 3924 and the contact portion 3918 of the distal end portion 3916 of the battery clip 3910 contact the battery assembly 3962, the batteries of the battery assembly 3962 are placed in electrical communication with the electronic circuit system 3900. Said another way, when the electrical contact of the substrate 3924 and the contact portion 3918 of the distal end portion 3916 of the battery clip 3910 contact the battery assembly 3962, the battery assembly 3962 is configured to supply power to the electronic circuit system 3900.

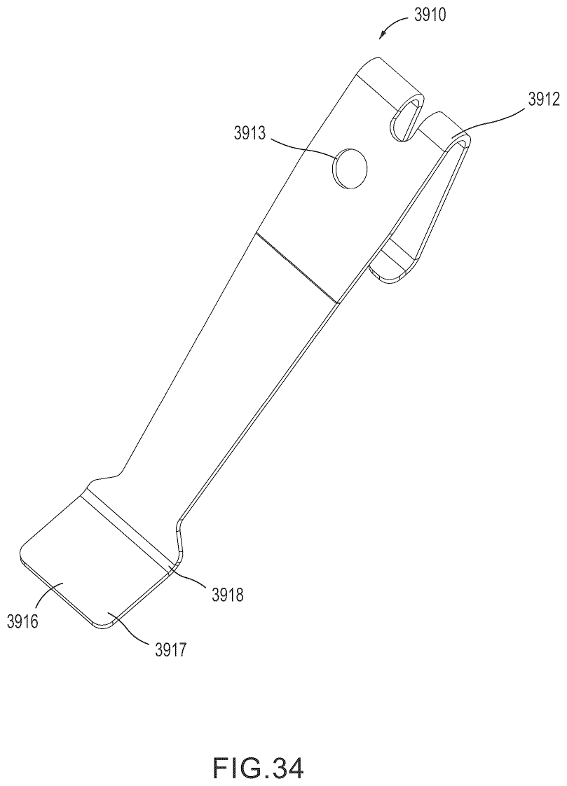

The battery clip 3910 (shown in FIG. 34) includes a proximal end portion 3912 and a distal end portion 3916. The proximal end portion 3912 defines a retention aperture 3913. The retention aperture 3913 is configured to receive a screw 3911 to couple the battery clip 3910 to the battery clip protrusion 3176 of the electronic circuit system housing 3170. In this manner, the battery clip protrusion 3176 maintains the position of the battery clip 3910 with respect to the electronic circuit system housing 3170 and/or the battery assembly 3962.

The distal end portion 3916 of the battery clip 3910 includes a contact portion 3918 and an angled portion 3917. As described above, the contact portion 3918 is configured to contact the second surface 3966 of the battery assembly 3962 to place the battery assembly 3962 in electrical communication with the electronic circuit system 3900. The angled portion 3917 of the distal end portion 3916 of the battery clip 3910 is configured to allow a proximal end portion 3236 of a battery isolation protrusion 3197 (see e.g., FIG. 41) to be disposed between the second surface 3966 of the battery assembly 3962 and the contact portion 3918 of the distal end portion 3916 of the battery clip 3910. When the battery isolation protrusion 3197 is disposed between the second surface 3966 of the battery assembly 3962 and the contact portion 3918 of the distal end portion 3916 of the battery clip 3910, the electrical path between the battery assembly 3962 and the remainder of the electrical circuit system 3900 is disrupted, thereby removing power from the electronic circuit system 3900. The contact portion 3918 of the distal end portion 3916 of the battery clip 3910 is biased such that when the battery isolation protrusion 3197 is removed, the contact portion 3918 will move into contact the second surface 3966 of the battery assembly 3962, thereby restoring electrical communication between the battery assembly 3962 and the electronic circuit system 3900. In some embodiments, the battery isolation protrusion 3197 can be repeatedly removed from between the second surface 3966 of the battery assembly 3962 and the contact portion 3918 of the distal end portion 3916 of the battery clip 3910 and reinserted. Said another way, the battery isolation protrusion 3197 and the battery clip 3910 collectively form a reversible on/off switch.

The audio output device 3956 of the electronic circuit system 3900 is configured to output audible sound to a user in response to use of the medical injector 3000. In some embodiments, the audible output device 3956 can be a speaker. In some embodiments, the audible sound can be, for example, associated with a recorded message and/or a recorded speech. In other embodiments, the audible instructions can be an audible beep, a series of tones and/or or the like.

In other embodiments, the medical injector 3000 can have a network interface device (not shown) configured to operatively connect the electronic circuit system 3900 to a remote device (not shown) and/or a communications network (not shown). In this manner, the electronic circuit system 3900 can send information to and/or receive information from the remote device. The remote device can be, for example, a remote communications network, a computer, a compliance monitoring device, a cell phone, a personal digital assistant (PDA) or the like. Such an arrangement can be used, for example, to download replacement processor-readable code from a central network to the electronic circuit system 3900. In some embodiments, for example, the electronic circuit system 3900 can download information associated with a medical injector 3000, such as an expiration date, a recall notice, updated use instructions or the like. Similarly, in some embodiments, the electronic circuit system 3900 can upload information associated with the use of the medical injector 3000 via the network interface device (e.g., compliance information or the like).

FIGS. 40 and 41 show the cover 3190 of the medical injector 3000. The cover 3190 includes a proximal end portion 3191 and a distal end portion 3192, and defines a cavity 3196. The cavity 3196 of the cover 3190 is configured to receive at least a portion of the housing 3100. Thus, when the portion of the housing 3100 is disposed within the cover 3190, the cover 3190 blocks an optical pathway between the medicament container 3200 and a region outside of the housing 3100. Similarly stated, when the portion of the housing 3100 is disposed within the cover 3190, the cover 3190 obstructs the first status indicator aperture 3130 and/or the second status indicator aperture 3160 of the housing 3100 to reduce the amount of light transmitted to the medicament 3220 within the medicament container 3200. In this manner, the life of the medicament 3220 can be extended by the prevention and/or reduction of degradation to the medicament 3220 that may be caused by ultra-violet radiation. In other embodiments, however, such those containing a medicament that is not sensitive to ultraviolet (UV) radiation, the cover 3190 can include viewing windows and/or openings that substantially correspond to the aperture 3130 and/or the aperture 3160.

The proximal end portion 3191 of the cover 3190 defines apertures 3193 configured to receive the cover retention protrusions 3104 of the housing 3100 (shown in FIGS. 10 and 12). In this manner, the apertures 3193 and the cover retention protrusions 3104 of the housing 3100 removably retain the cover 3190 about at least a portion of the housing 3100. Said another way, the apertures 3193 and the cover retention protrusions 3104 of the housing 3100 are configured such that the cover 3190 can be removed from a portion of the housing 3100 and then replaced about the portion of the housing 3100.

As described above, the electronic circuit system 3900 can be actuated when the housing 3100 is at least partially removed from the cover 3190. More particularly, the distal end portion 3192 of the cover 3190 includes the battery isolation protrusion 3197. The battery isolation protrusion 3197 includes a proximal end portion 3236 and a tapered portion 3237. The proximal end portion 3236 of the battery isolation protrusion 3197 is configured to be removably disposed between the second surface 3966 of the battery assembly 3962 and the contact portion 3918 of the distal end portion 3916 of the battery clip 3910, as described above.

The cover 3190 can be any suitable configuration and can include any suitable feature. For example, the cover 3190 includes openings 3195 and notches 3194. In some embodiments, the openings 3195 can receive inserts (not shown). The inserts can be flexible inserts and can increase friction between the cover 3190 and a surface. For example, the inserts can increase the friction between the cover 3190 and a surface on which the medical injector 3000 is placed, to prevent sliding. The notches 3194 are disposed at the proximal end of the cover 3190. In some embodiments, the notches 3194 can be used to reduce the material needed to manufacture the cover 3190.

FIGS. 42-46 show the safety lock 3700 of the medical injector 3000. The safety lock 3700 of the medical injector 3000 includes a proximal surface 3730, a distal surface 3740 opposite the proximal surface 3730 and a needle sheath 3810. The safety lock 3700 defines a needle sheath aperture 3703 and a battery isolation protrusion aperture 3728. The battery isolation protrusion aperture 3728 is configured to receive the battery isolation protrusion 3197 of the cover 3190 such that the battery isolation protrusion 3197 can be disposed within the electronic circuit system cavity 3137 and/or in engagement with the electronic circuit system 3900, as described above. Similarly stated, the battery isolation protrusion aperture 3728 of the safety lock 3700 is aligned with the battery isolation protrusion aperture 3135 of the housing 3100, such that the battery isolation protrusion 3197 can be disposed within the electronic circuit system cavity 3137 when the cover 3190 is disposed about a portion of the housing 3100.

The proximal surface 3730 of the safety lock 3700 includes a safety lock protrusion 3702, a stopper 3727, an actuator 3724, two opposing pull-tabs 3710 and an engagement portion 3720. As described above, when the safety lock 3700 is in a first (locked) position, the safety lock protrusion 3702 is configured to be disposed in the opening 3556 defined by the extensions 3553 of the distal end portion 3552 of the release member 3550 (see e.g., FIG. 21). Accordingly, the safety lock protrusion 3702 is configured to prevent the extensions 3553 from moving closer to each other, thereby preventing proximal movement of the release member 3550 and/or delivery of the medicament 3220. The stopper 3727 of the safety lock 3700 is a protrusion extending from the proximal surface 3730 of the safety lock 3700. The stopper 3727 is configured to contact a portion of the housing 3100 to limit the proximal movement of the safety lock 3700 relative to the housing 3100. In other embodiments, the stopper 3727 can be any structure configured to limit the proximal movement of the safety lock 3700.

The actuator 3724 of the safety lock 3700 has an elongated portion 3725 and a protrusion 3726. The elongated portion 3725 extends in a proximal direction from the proximal surface 3730. In this manner, the elongated portion 3725 can extend through a safety lock actuator opening 3524 of the base 3510 (see e.g., FIG. 47) and within the safety lock actuator groove 3133 of the housing 3100 and the safety lock actuator groove 3179 of the electronic circuit system housing 3170. The protrusion 3726 extends in a direction substantially transverse to the elongated portion 3725 and/or substantially parallel to the proximal surface 3730 of the safety lock 3700. As described above, the opening 3928 of the first actuation portion 3926 of the printed circuit board 3922 is configured to receive the protrusion 3726 of the actuator 3724 of the safety lock 3700.

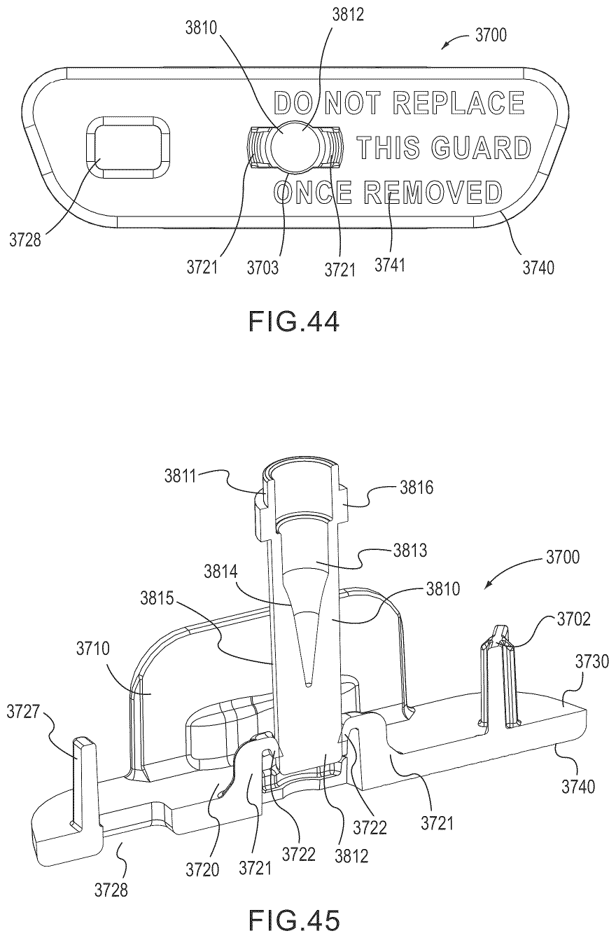

The pull-tabs 3710 of the safety lock 3700 include a grip portion 3712 and indicia 3713. The grip portion 3712 of the pull-tabs 3710 provides an area for the user to grip and/or remove the safety lock 3700 from the rest of the medicament delivery system 3700. The indicia 3713 provide instruction on how to remove the safety lock 3700. The distal end surface 3740 also includes indicia 3741 (see e.g., FIG. 44). In some embodiments, for example, indicia can indicate the direction the user should pull the safety lock 3700 to remove the safety lock 3700.

The engagement portion 3720 of the safety lock 3700 includes engagement members 3721. The engagement members 3721 extend in a proximal direction from the proximal surface 3730. The engagement members 3721 have tabs 3722 that extend from a surface of the engagement members 3721. The tabs 3722 are configured to engage an outer surface 3815 of a distal end portion 3812 of the needle sheath 3810.

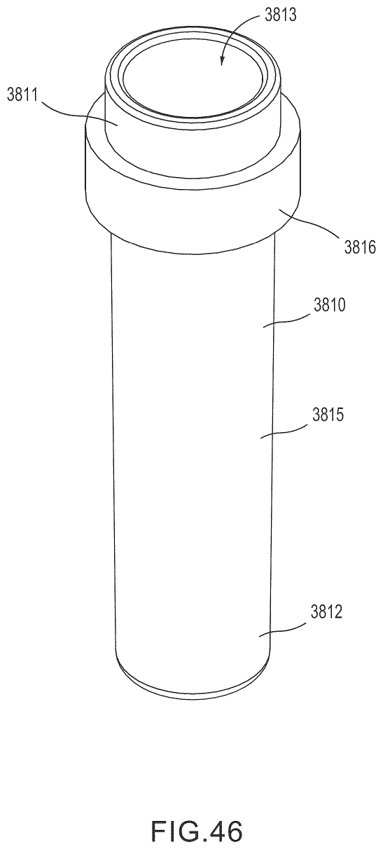

As shown in FIGS. 45 and 46, the needle sheath 3810 includes the distal end portion 3812, a proximal end portion 3811 and a rib 3816. The needle sheath 3810 also defines a bore 3813. The bore 3813 is defined by a contoured portion 3814 of the needle sheath 3810, and is configured to receive the needle 3216 and/or a distal end portion of the 3213 of the medicament container 3200. The inner portion of the needle sheath 3810 defines a friction fit with the distal end portion 3213 of the medicament container 3200. In this manner, the needle sheath 3810 can protect the user from the needle 3216 and/or can keep the needle 3216 sterile before the user actuates the medical injector 3000. The proximal end portion 3811 of the needle sheath is configured to contact the body 3210 of the medicament container 3200.

The distal end portion 3812 of the needle sheath 3810 is configured to be inserted into a space defined between the tabs 3722 of the engagement members 3721 of the safety lock 3700. The tabs 3722 are angled and/or bent towards the distal direction to allow the distal end portion 3812 of the needle sheath 3810 to move between the engagement members 3721 in a distal direction, but not in a proximal direction. Similarly stated, the tabs 3722 include an edge that contacts the outer surface 3815 of the needle sheath 3810 to prevent the safety lock 3700 from moving in a distal direction relative to the needle sheath 3810. In this manner, the needle sheath 3810 is removed from the needle 3216 when the safety lock 3700 is moved in a distal direction with respect to the housing 3100 (see e.g., FIG. 50).

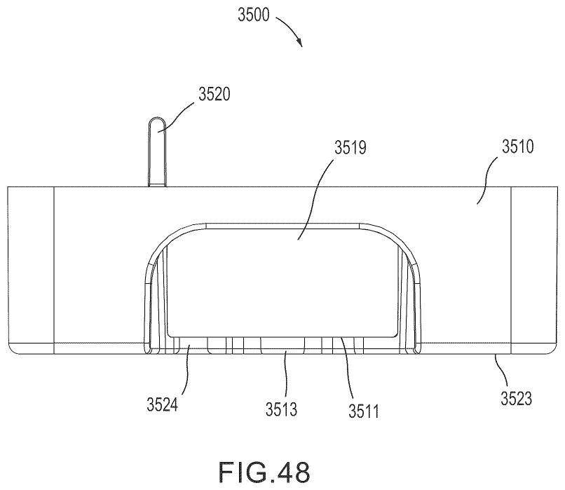

FIGS. 47 and 48 show the base (or actuator) 3510 of the medical injector 3000. The base 3510 includes a proximal surface 3511, a distal surface 3523 and base connection knobs 3518. The base 3510 defines a needle aperture 3513, a safety lock protrusion aperture 3514, a battery isolation protrusion aperture 3521, a safety lock actuator opening 3524 and pull-tab openings 3519. The needle aperture 3513 is configured to receive the needle 3216 when the medical injector 3000 is actuated. The safety lock protrusion aperture 3514 of the base 3510 receives the safety lock protrusion 3702 of the safety lock 3700 when the safety lock 3700 is coupled to the housing 3100 and/or the base 3510. The battery isolation protrusion aperture 3521 of the base 3510 receives the battery isolation protrusion 3197 of the cover 3190 and the stopper 3727 of the safety lock 3700. The safety lock actuator opening 3524 receives the safety lock actuator 3724 of the safety lock 3700. The pull-tab openings 3519 are configured to receive the pull-tabs 3710 of the safety lock 3700.

The proximal surface 3511 of the base 3510 includes a protrusion 3520, guide members 3517 and protrusions 3515. The protrusion 3520 is configured to engage the substrate 3924 of the electronic circuit system 3900. As described above, the opening 3945 of the second actuation portion 3946 of the printed circuit board 3922 is configured to receive the actuator 3520 of the base 3510. The guide members 3517 of the base 3510 engage and/or slide within the base rail grooves 3114 of the housing 3100, as described above. The protrusions 3515 of the base 3510 engage the tapered surfaces 3557 of the extensions 3553 of the release member 3550. As described in further detail herein, when the safety lock 3700 is removed and the base 3510 is moved in a proximal direction with respect to the housing 3100, the protrusions 3515 of the base 3510 are configured to move the extensions 3553 of the release member 3550 closer to each other, actuating the medicament delivery mechanism 3300. As described above, the base connection knobs 3518 engage the base retention recesses 3134A, 3134B in a way that allows proximal movement of the base 3510 but limits distal movement of the base 3510.

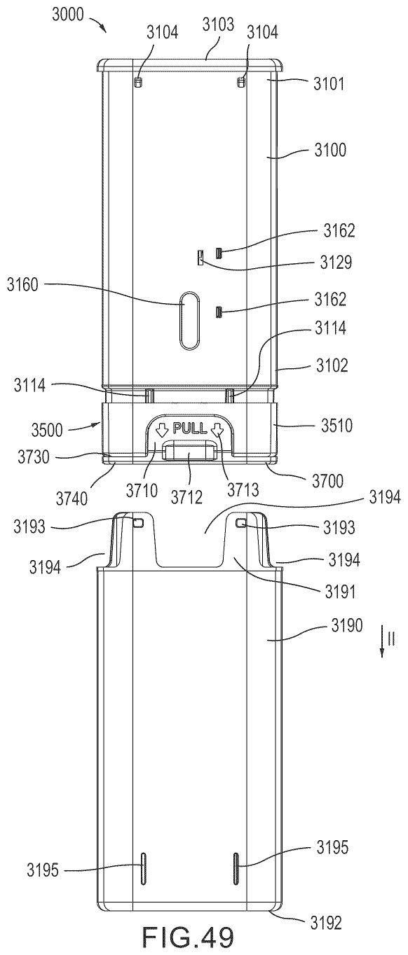

As shown in FIG. 49, the medical injector 3000 is first enabled by moving the medicament delivery device 3000 from a first configuration to a second configuration by moving the cover 3190 from a first position to a second position. The cover 3190 is moved from the first position to the second position by moving it with respect to the housing 3100 in the direction shown by the arrow II in FIG. 49. When the cover 3190 is moved with respect to the housing 3100 in the direction II, the battery isolation protrusion 3197 is removed from the area between the battery clip 3910 and the second surface 3966 of the battery assembly 3962. In this manner, the battery assembly 3962 is operatively coupled to the electronic circuit system 3900 when the cover 3190 is removed, thereby providing power to the electronic circuit system 3900. Similarly stated, this arrangement allows the electronic circuit system 3900 to be actuated when the cover 3190 is removed.

When power is provided, as described above, the electronic circuit system 3900 can output one or more predetermined electronic outputs. For example, in some embodiments, the electronic circuit system 3900 can output an electronic signal associated with recorded speech to the audible output device 3956. Such an electronic signal can be, for example, associated with a. WAV file that contains a recorded instruction, instructing the user in the operation of the medical injector 3000. Such an instruction can state, for example, “Remove the safety tab near the base of the auto-injector.” The electronic circuit system 3900 can simultaneously output an electronic signal to one and/or both of the LEDs 3958A, 3958B thereby causing one and/or both of the LEDs 3958A, 3958B to flash a particular color. In this manner, the electronic circuit system 3900 can provide both audible and visual instructions to assist the user in the initial operation of the medical injector 3000.

In other embodiments, the electronic circuit system 3900 can output an electronic output associated with a description and/or status of the medical injector 3000 and/or the medicament 3220 contained therein. For example, in some embodiments, the electronic circuit system 3900 can output an audible message indicating the symptoms for which the medicament 3220 should be administered, the expiration date of the medicament 3220, the dosage of the medicament 3220 or the like.

As described above, the medical injector 3000 can be repeatedly moved between the first configuration and the second configuration when the cover 3190 is moved repeatedly between the first position and the second position respectively. Said another way, the cover 3190 can be removed and replaced about the housing 3100 any number of times. When the cover 3190 is moved from the second position to the first position, the battery isolation protrusion 3197 is inserted between the battery clip 3910 and the second surface 3966 of the battery assembly 3962, deactivating the electronic circuit system 3900. When the cover is moved from the first position to the second position a second time, the electronic circuit system 3900 is once again activated. In this manner, the cover 3190 can be removed and the electronic circuit system 3900 can output an electronic output without compromising the sterility of the needle 3216.

After the cover 3190 is removed from the housing 3100, the medical injector 3000 can be moved from the second configuration (FIG. 49) to a third configuration (FIG. 50) by moving the safety lock 3700 from a first position to a second position. The safety lock 3700 is moved from a first position to a second position by moving the safety lock 3700 with respect to the housing 3100 in the direction shown by the arrow JJ in FIG. 50. When the safety lock 3700 is moved from the first position to the second position, the safety lock protrusion 3702 is removed from between the extensions 3553 of the release member 3550, thereby enabling the medicament delivery mechanism 3300. Moreover, as shown in FIGS. 37 and 38, when the safety lock 3700 is moved from the housing 3100, the actuator 3724 of the safety lock 3700 moves in the direction GG as shown in FIG. 38, irreversibly moving the first switch 3972 from a first state (e.g., a state of electrical continuity) to a second state (e.g., a state of electrical discontinuity). When the actuator 3724 of the safety lock 3700 moves irreversibly the first switch 3972 of the electronic circuit system 3900 to the second state, the electronic circuit system 3900 can output one or more predetermined electronic outputs. For example, in some embodiments, a processor (not shown) can output an electronic signal associated with recorded speech to the audible output device 3956. Such an electronic signal can be, for example, associated with a recorded message notifying the user of the status of the medical injector 3000. Such a status message can state, for example, “If ready to use the medical injector, pull off the red safety guard.” The electronic circuit system 3900 can also simultaneously output an electronic signal to one and/or both of the LEDs 3958A, 3958B, thereby causing one and/or both of the LEDs 3958A, 3958B to stop flashing, change color or the like.

In some embodiments, the first actuation portion 3926 and the actuator 3724 can be configured such that the actuator 3724 must move a predetermined distance before the actuator 3724 engages the boundary 3929 of the opening 3928. For example, in some embodiments, the actuator 3724 must move approximately 0.200 inches before the actuator 3724 engages the boundary 3929 of the opening 3928. In this manner, the safety lock 3700 can be moved slightly without irreversibly moving the first switch 3972 of the electronic circuit system 3900 to the second state. Accordingly, this arrangement will permit the user to inadvertently and/or accidentally move the safety lock 3700 without actuating the electronic circuit system 3900.

In some embodiments, the electronic circuit system 3900 can be configured to output the status message for a predetermined time period, such as, for example, five seconds. After the predetermined time period has elapsed, the electronic circuit system 3900 can output an audible message further instructing the user in the operation of the medical injector 3000. Such an instruction can state, for example, “Place the base of the auto-injector against the patient's thigh. To complete the injection, press the base firmly against the patient's thigh.” In some embodiments, the electronic circuit system 3900 can simultaneously output an electronic signal to one and/or both of the LEDs 3958A, 3958B, thereby causing one and/or both of the LEDs 3958A, 3958B to flash a particular color. In this manner, the electronic circuit system 3900 can provide both audible and/or visual instructions to assist the user in the placement and actuation of the medical injector 3000. In some embodiments, the electronic circuit system 3900 can be configured to repeat the instructions after a predetermined time period has elapsed.

As described above, in other embodiments, the medical injector 3000 can have a network interface device (not shown) configured to operatively connect the electronic circuit system 3900 to a remote device (not shown) and/or a communications network (not shown). In this manner, the electronic circuit system 3900 can send a wireless signal notifying a remote device that the safety lock 3700 of the medical injector 3000 has been removed and that the medical injector 3000 has been armed. In other embodiments, the electronic circuit system 3900 can send a wireless signal (e.g., a wireless 911 call) notifying an emergency responder that the medical injector 3000 has been armed, for example, via removal of the safety lock 3700.

After the safety lock 3700 is moved from the first position to the second position, the medical injector 3000 can be moved from the third configuration (FIG. 50) to a fourth configuration (FIG. 51) by moving the base 3510 from a first position to a second position. Similarly stated, the medical injector 3000 can be actuated by the system actuator assembly 3500 by moving the base 3510 proximally relative to the housing 3100. The base 3510 is moved from its first position to its second position by placing the medical injector 3000 against the body of the patient and moving the base 3510 with respect to the housing 3100 in the direction shown by the arrow KK in FIG. 51. Moving the base 3510 from the first position to the second position causes the protrusions 3515 on the proximal surface 3511 of the base 3510 to engage the tapered surfaces 3557 of the extensions 3553 of the release member 3550, thereby moving the extensions 3313 together. The inward movement of the extensions 3553 causes engagement surface 3554 of the release member 3550 to become disengaged from the base release surface 3126 of the housing 3100, thereby allowing the release member 3550 to be moved proximally along its longitudinal axis as the spring 3576 expands.

When the base 3510 is moved from the first position to the second position, the system actuator assembly 3500 actuates the medicament delivery mechanism 3300, thereby placing the medical injector 3000 in its fourth configuration (i.e., the needle insertion configuration), as shown in FIGS. 51 and 52. More particularly, when the medical injector 3000 is in its fourth configuration, the puncturer 3575 of the release member 3550 is in contact with and/or disposed through the frangible seal 3413 of the gas container 3410.

After the frangible seal 3413 has been punctured, an actuating portion of a compressed gas flows from the gas container 3410, via the gas passageway 3156 and into the medicament cavity 3139. The gas applies gas pressure to the piston member 3330 causing the piston member 3330 and the carrier 3370 to move in a distal direction within the medicament cavity 3139, as shown by the arrow LL in FIG. 52. When the carrier 3370 moves distally within the medicament cavity 3139, the carrier 3370 and the medicament container 3200 are in a first configuration and collectively move toward a second position. In this manner, the medicament container 3200 and the needle 3216 contemporaneously move with piston member 3330 and/or the carrier 3370 in a distal direction. The movement of the needle 3216 in a distal direction causes the distal end portion of the needle 3216 to exit the housing 3100 and enter the body of a patient prior to administering the medicament 3220.

As described above, at least a portion of the force exerted by the compressed gas within the gas chamber upon the piston member 3330 is transferred to the first shoulder 3377 of the carrier 3370 by the contact between the first surface 3341 of the piston member 3330 and the engagement portion 3379 of the carrier 3370. This arrangement further allows at least a portion of the force to be transferred to the flange 3214 of the medicament container 3200. In this manner, the application of the force on the piston member 3330 results in the distal movement of the carrier 3370 and the medicament container 3200. Moreover, because the distal end portion 3332 of the piston member 3330 is configured such that the second surface 3342 is spaced apart from the elastomeric member 3217 within the medicament container 3200 (see e.g., FIG. 27), the force is not transferred to the elastomeric member 3217. In this manner, the elastomeric member 3217 is isolated from the piston member 3330 when the medicament container 3200 is moving distally within the housing 3100, which reduces and/or eliminates injection or leakage of the medicament 3220 from the medicament container 3200 during the needle insertion operation.

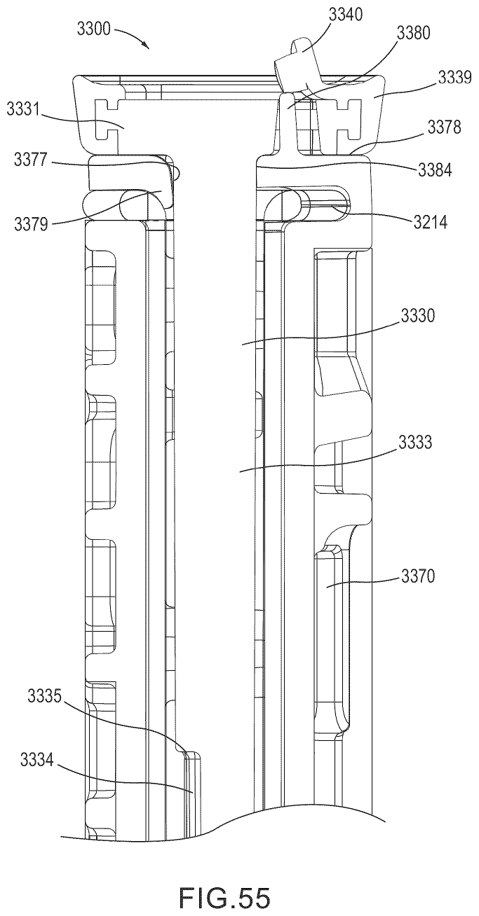

After the carrier 3370 and/or the needle 3216 have moved within the medicament cavity 3139 a predetermined distance, the carrier 3370 and the medicament container 3200 are moved from the first configuration to a second configuration. For example, in some embodiments, the retraction spring 3351 can be fully compressed and prevent the carrier 3370 from moving further in the distal direction. In other embodiments, a portion of the medicament container 3200 and/or a portion of the carrier 3370 can contact the housing 3100 when the needle insertion operation is completed, thereby limiting further distal movement of the carrier 3370, medicament container 3200 and/or the needle 3216. When the distal movement of the carrier 3370 is prevented, the gas within the gas chamber continues to apply gas pressure to the piston member 3330 causing the first surface 3341 of the piston member 3330 to deform a portion of the engagement portion 3379. Similarly stated, when the distal movement of the carrier 3370 is complete, the force applied by the pressurized gas exceeds a threshold value, thereby causing the piston member 3330 to deform the engagement portion 3379. In this manner, the engagement portion 3379 deforms (see e.g., FIG. 55) to place the carrier 3370 in its second configuration, in which the first surface 3341 of the piston member 3330 is no longer in contact with the engagement portion 3379 and/or the first shoulder 3377.

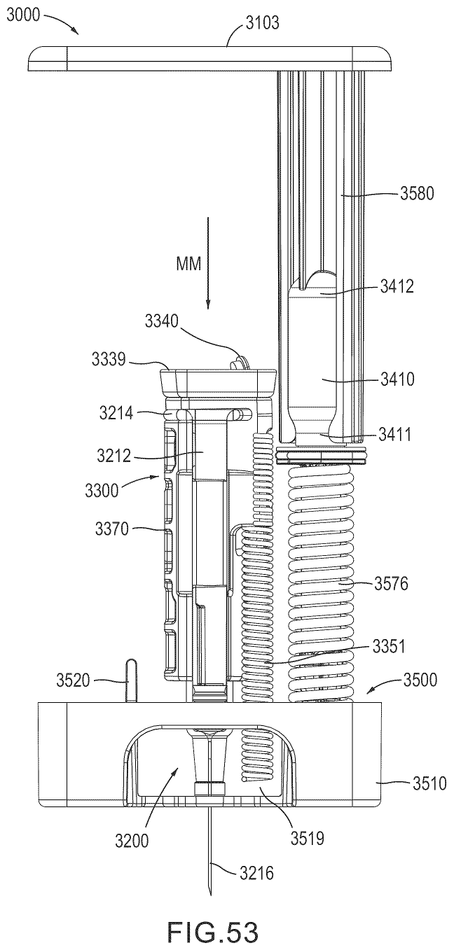

When the carrier 3370 is in the second configuration, the piston member 3330 continues to move in the distal direction relative to the carrier 3370 and/or the medicament container 3200. Similarly stated, the piston member 3330 moves with the carrier 3370 during the insertion operation (i.e., when the carrier 3370 is in its first configuration) and the piston member 3330 moves relative to the carrier 3370 (and the medicament container 3200) during the injection operation (i.e., when the carrier 3370 is in its second configuration). More particularly, after the engagement portion 3379 deforms, the piston rod 3333 of the piston member 3330 moves within the piston rod opening 3384 of the carrier 3370 and within the medicament container 3200, as shown by the arrow MM in FIG. 53. As the piston rod 3333 of the piston member 3330 moves within the carrier 3370 and medicament container 3200, the second surface 3342 of the piston rod 3333 contacts the elastomeric member 3217 and generates a pressure upon the medicament 3220 contained within the medicament container 3200, thereby allowing at least a portion of the medicament 3220 to flow out of the medicament container 3200 via the needle 3216. The medicament 3220 is delivered to a body of a user via the medicament delivery path defined by the medicament container 3200 and the needle 3216.

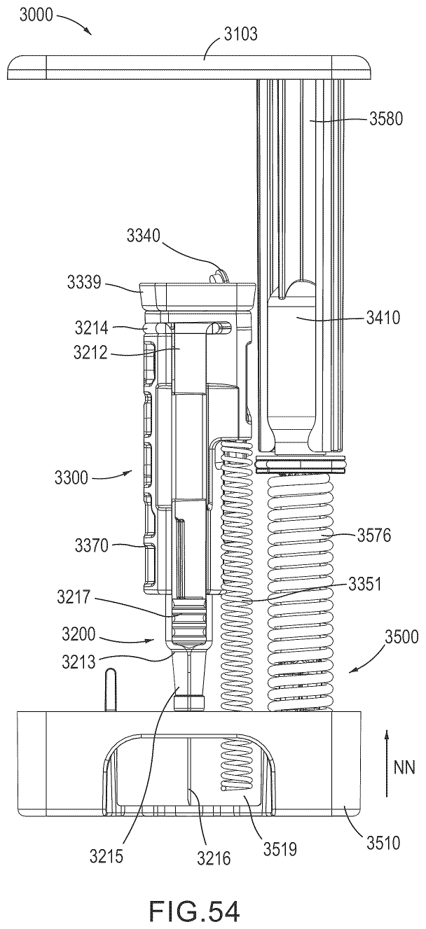

As shown in FIGS. 54 and 55, after the piston member 3330 moves a predetermined distance within the medicament container 3200, the gas valve actuator 3380 of the carrier 3370 engages the gas relief valve 3340 (see e.g., FIG. 55) of the piston member 3330 thereby allowing the pressurized gas contained within the gas chamber (i.e., the volume within the medicament cavity 3139 between the proximal end of the housing 3100 and the proximal end of the piston member 3330) to escape. Similarly stated, as the gas valve actuator 3380 of the carrier 3370 engages the gas relief valve 3340 of the piston member 3330, the pressure within the housing 3100 is reduced, thereby ending the injection event. In this manner, the pre-injection distance between the proximal end portion 3331 of the piston member 3330 and the gas valve actuator 3380 of the carrier 3370 can be adjusted to control the amount of the medicament 3220 to be injected. After the gas pressure within the medicament cavity 3139 decreases below a certain level, the force exerted by the retraction spring 3351 on the engagement portion 3382 of the carrier 3370 is sufficient to cause the carrier 3370 to move proximally within the housing 3100 (i.e., to retract). Additionally, the second shoulder 3381 engages the distal surface of the flange 3214 of the medicament container 3200 to move the medicament container 3200 proximally within the housing 3100, as shown by the arrow NN in FIG. 54.

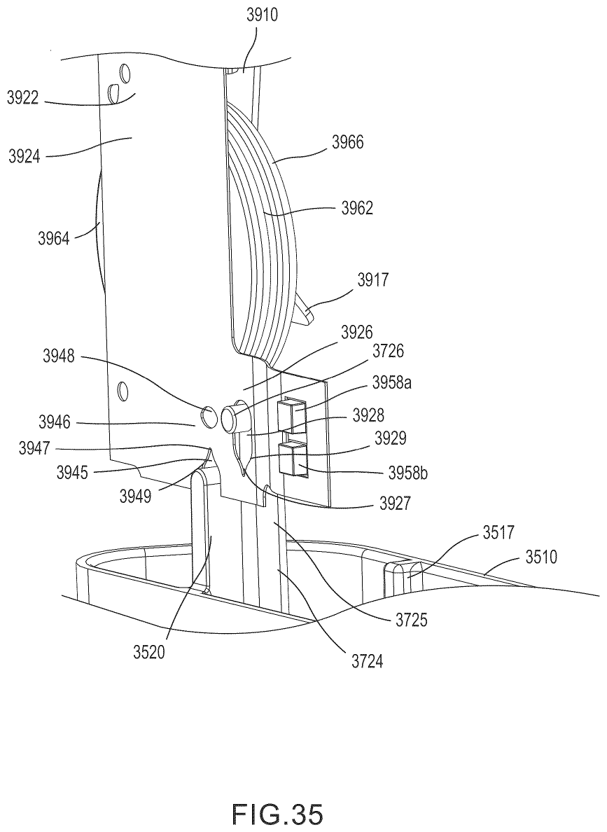

As described above, the protrusion 3520 of the base 3510 actuates the electronic circuit 3900 to trigger a predetermined output or sequence of outputs when the base 3510 is moved from its first position to its second position (see, e.g., FIGS. 35-39). When the protrusion 3520 is moved in a proximal direction relative to the opening 3945, as shown by the arrow HH in FIG. 39, the electronic circuit system 3900 is actuated to output one or more predetermined electronic outputs. For example, in some embodiments, the electronic circuit system 3900 can output an electronic signal associated with recorded speech to the audible output device 3956. Such an electronic signal can be, for example, associated with an audible countdown timer, instructing the user on the duration of the injection procedure. Said another way, if it takes, for example, ten seconds to complete an injection, an audible countdown timer can count from ten to zero ensuring that the user maintains the medical injector 3000 in place for the full ten seconds. In other embodiments, the electronic signal can be, for example, associated with a recorded message notifying the user that the injection is complete, instructing the user on post-injection disposal and safety procedures, instructing the user on post-injection medical treatment or the like. Such a status message can state, for example, “The injection is now complete. Please seek further medical attention from a doctor.” The electronic circuit system 3900 can also simultaneously output an electronic signal to one and/or both LEDs 3958A, 3958B, thereby causing one and/or both LEDs 3958A, 3958B to stop flashing, change color or the like, to provide a visual indication that the injection is complete. In other embodiments, the electronic circuit system 3900 can send a wireless signal notifying a remote device that the injection is complete. In this manner, a patient's compliance and/or adherence with the use of the system can be monitored.

In some embodiments, the second actuation portion 3946 and the protrusion 3520 of the base 3510 can be configured such that the base 3510 and/or the actuator 3520 must move a predetermined distance before the protrusion 3520 engages the boundary 3949 of the opening 3945. For example, in some embodiments, the protrusion 3520 must move approximately 0.200 inches before the actuator 3520 engages the boundary 3949 of the opening 3945. In this manner, the base 3510 can be moved slightly without irreversibly moving the second switch 3973 of the electronic circuit system 3900 to the second state. Accordingly, this arrangement will permit the user to inadvertently and/or accidentally move the base 3510 without actuating the electronic circuit system 3900.

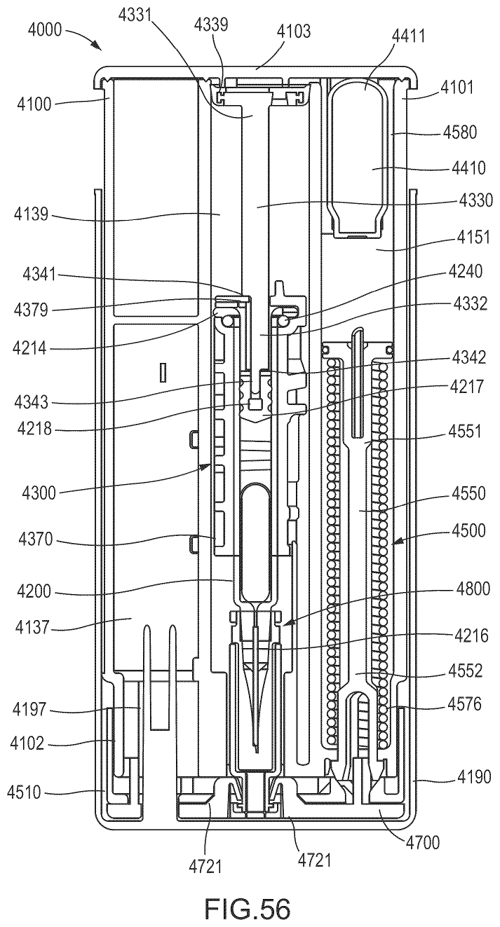

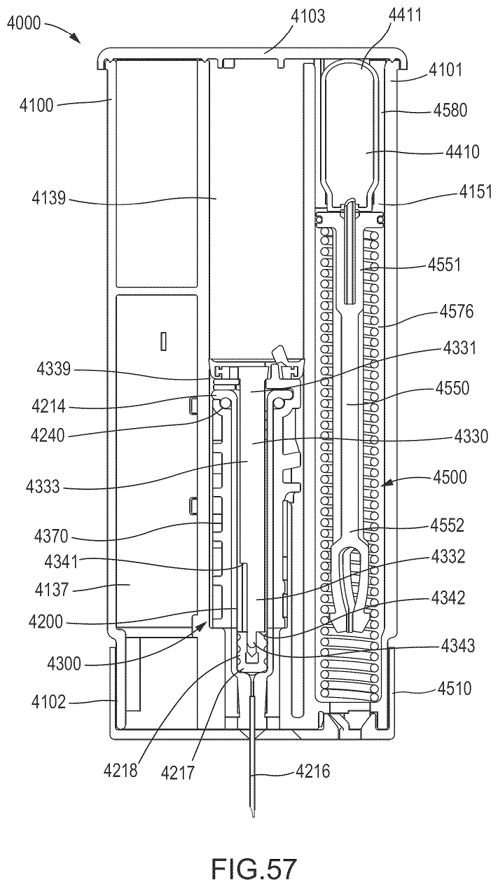

While specific components are discussed with respect to the medical injector 3000, in other embodiments, some components can be modified and/or removed without substantially changing the medicament injection event. For example, FIGS. 56-59 show a portion of a medical injector 4000. That does not include an electronic circuit system (e.g., an electronic circuit system substantially similar to the electronic circuit system 3900 included in the medical injector 3000). In some embodiments, the electronic circuit system can be removed to limit the cost of the medical injector 4000. In those embodiments devoid of an electronic circuit system, for example the medical injector 4000 shown in FIGS. 56 and 57, the medical injector 4000 can still include components and/or portions configured to engage and/or interact with an electronic circuit system. For example, the medical injector 4000 includes a battery isolation protrusion 4197 of a cover 4190. In this manner, the cost of production and tooling can be reduced by reducing the number of component variations. Additionally, an electronic circuit system (e.g., similar to the electronic circuit system 3900 included in the medical injector 3000) can be easily added to the medical injector 4000 and disposed within an electronic circuit system cavity 4137 defined by the housing 4100.

The medical injector 4000 is similar to the medical injector 3000 described above. As shown in FIGS. 56 and 57, the medical injector 4000 includes a housing 4100, the cover 4190 (FIG. 56), a safety lock 4700 (FIG. 56), a base 4510, a system actuator assembly 4500, a delivery mechanism 4300, a medicament container 4200 and a needle guard assembly 4800. The structure and operation of the cover 4190, the safety lock 4700 and the base 4510 are similar to the structure and operation of the cover 3190, the safety lock 3700 and the base 3510, respectively. Accordingly, only the delivery mechanism 4300, the system actuator assembly 4500 and the needle guard assembly 4800 are described in detail below.

As shown in FIG. 56, the housing 4100 has a proximal end portion 4101 and a distal end portion 4102. The housing 4100 defines a gas cavity 4151, a medicament cavity 4139 and the electronic circuit system cavity 4137. The gas cavity 4151, medicament cavity 4139 and the electronic circuit system cavity 4137 of the housing 4100 of the medical injector 4000 are similar to the gas cavity 3151, the medicament cavity 3139 and the electronic circuit system cavity 3137, shown and described above with reference to FIGS. 15 and 16.

The distal end portion 4102 of the housing 4100 is similar to the distal end portion 3102 of the housing 3100, described above in reference to FIG. 15. The proximal end portion 4101 includes a proximal cap 4103. The proximal cap 4103 includes a gas container retention member 4580 and defines a gas passageway (not shown in FIGS. 56 and 57). The gas container retention member 4580 is configured to receive a gas container 4410. The gas container retention member 4580 extends from a distal surface of the proximal cap 4103 and is configured to place a proximal end 4411 of the gas container adjacent to the proximal cap 4103. Similarly stated, the gas container retention member 4580 extends a given distance from the proximal cap 4103 such that the gas container 4410 is disposed adjacent to the proximal cap 4103 within a proximal end of the gas cavity 4151. In this manner, the gas container retention member 4580 differs from the gas container retention member 3580, which positions the gas container 3410 apart from the proximal cap 3103.

The system actuator assembly 4500 includes the base 4510, a release member 4550 and a spring 4576. The release member 4550 has a proximal end portion 4551 and a distal end portion 4552, and is movably disposed within the gas cavity 4151. The proximal end portion 4551 and the distal end portion 4552 of the release member 4550 are similar to the corresponding structure of the release member 3550 of the medical injector 3000, described above with reference to FIGS. 18-21. The release member 4550 differs from the release member 3550, however, in that the release member 4550 is substantially longer than the length of the release member 3550 of the medical injector 3000. In this manner, the release member 4550 is able to engage the gas container 4410 disposed at the proximal end of the gas cavity 4151. Similarly stated, with the gas container 4410 disposed at the proximal end of the gas cavity 4151, the length of the release member 4550 is increased, compared to the release member 3550 of the medical injector 3000, so that the release member 4550 can engage the gas container 4410. Consequently, the length of the spring 4576 (in the compressed state) is longer than the length of the spring 3576 included in the medical injector 3000, described above with reference to FIGS. 18-21.

The arrangement of the system actuator assembly 4500, the gas container 4410 and the gas container retention member 4580 function similar to the system actuator assembly 3500, the gas container 3410 and the gas container retention member 3580, respectively, to activate the delivery mechanism 4300. In some embodiments, the gas container retention member 4580 can be configured to place the gas container 4410 at any suitable position within the gas cavity 4151. In this manner, the length of the release member 4550 and the spring 4576 can be any given length such that the proximal end portion 4551 of the release member can engage the gas container 4410, as shown in FIG. 57.

The medicament delivery mechanism 4300 includes a carrier 4370 (also referred to herein as the “first movable member” 4370) and a piston member 4330 (also referred to herein as the “second movable member” 4330). The carrier 4370 is similar to the carrier 3370 included in the medical injector 3000 and is movably disposed within the medicament cavity 4139. Therefore, the carrier 4370 is not described in detail herein.

The piston member 4330 includes a proximal end portion 4331, a distal end portion 4332 and a piston rod 4333. The piston portion 4330 is movably disposed within the medicament cavity 4139. The proximal end portion 4331 includes a sealing member 4339 and is similar in form and function to the proximal end portion 3331 of piston member 3330 of the medical injector 3000 described above. The distal end portion 4332 includes a first surface 4341, a second surface 4342 and an elongate protrusion 4343. The second surface 4342 and the elongate protrusion 4343 are disposed within a portion of the carrier 4370 and within the medicament container 4200. The first surface 4341 is configured to contact an engagement portion 4379 of the carrier 4370 when the medicament container 4200 is in a first configuration to maintain a given distance between the second surface 4342 and an elastomeric member 4217 of the medicament container 4200 (see e.g., FIG. 56), in a similar manner as described above. The elongate protrusion 4343 is configured to be disposed within a channel 4218 defined by the elastomeric member 4217. Similarly stated, the piston portion 4330 includes a portion and/or surface in contact with the elastomeric member 4217 and a portion and/or surface not in contact with the elastomeric member 4217, when the carrier 4370 is in the first configuration. In some embodiments, the elongate protrusion 4343 can be used to align the piston rod 4333 with the elastomeric member 4217 disposed within the medicament container 4200.

The piston member 4330 is configured to move within the housing 4100 (e.g., in response to the release of a pressurized gas). When the piston member 4330 moves, the first surface 4341 of the piston portion 4330 can apply a force to a portion of the carrier 4370 such that the carrier 4370 and the piston portion 4330 move together within the medicament cavity 4139. As described above, after the carrier 4370 is placed in its second (or deformed) configuration, the piston rod 4333 can move relative to the carrier 4370 and the elongate 4343 and the second surface 4342 can engage the elastomeric member 4217 to convey the medicament 4220 contained in the medicament container 4200 (see e.g., FIG. 57).

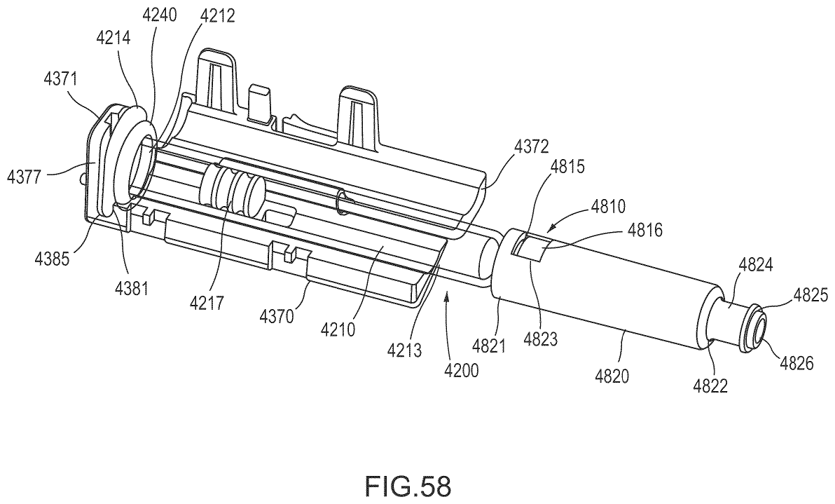

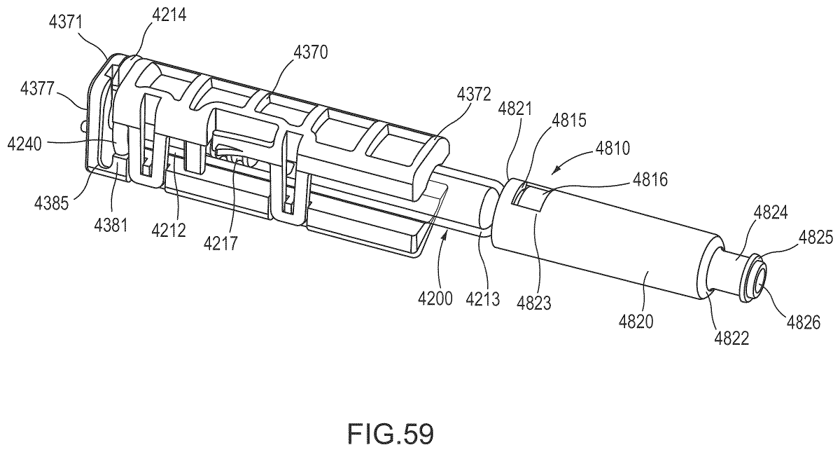

As shown in FIGS. 58 and 59, the medicament container 4200 is configured to be disposed within the carrier 4370. The medicament container 4200 includes a proximal end portion 4212 and a distal end portion 4213. The proximal end portion 4212 includes a flange 4214. The distal end portion 4213 is in fluid communication with a needle 4216 (see e.g., FIG. 59). The form and function of the medicament container 4200 is similar to the form and function of the medicament container 3200 of the medical injector 3000. The medicament container 4200 also includes a damping member 4240 disposed at a distal surface of the flange 4214.

The flange 4214 of the medicament container 4200 is disposed with in a flange groove 4385 defined by a first shoulder 4377 and a second shoulder 4381 of the carrier 4370. The flange groove 4385 includes a portion configured to receive the damping member 4240. In this manner, the damping member 4240 is configured to dampen a portion of a retraction force applied to the flange 4214 of the medicament container 4200 by the second shoulder 4381. The arrangement of the damping member 4240 within the flange groove 4381 reduces the likelihood of the flange 4214 breaking under the force applied by the second shoulder 4381, which can prevent the retraction of the medicament container 4200.

The needle guard assembly 4800 includes an inner needle sheath 4810 and an outer needle sheath 4820. The inner needle sheath 4810 includes an outer surface 4815 that has a ring 4816. The inner needle sheath 4810 is disposed within the outer needle sheath 4820 (see e.g., FIGS. 58 and 59). The inner needle sheath 4810 is similar to the needle sheath 3810 of the medical injector 3000, described above with reference to FIG. 46. Therefore, details of the inner needle sheath 4810 are not described in detail herein.

The outer needle sheath 4820 includes a proximal end portion 4821 and a distal end portion 4822, and defines a lumen 4826 therebetween. The lumen 4826 is configured to receive the inner needle sheath 4810. The proximal end portion 4821 includes an inner sheath aperture 4823 configured to receive the ring 4816 of the inner needle sheath 4810. The ring 4816 extends from the outer surface 4815 of the inner needle sheath 4810 and a portion of the ring is disposed within the inner sheath aperture 4823. The arrangement of the ring 4816 of the inner needle sheath 4810 and the inner sheath aperture 4823 prevent the movement of the inner needle sheath 4810 within the outer needle sheath 4810.

The distal end portion 4822 includes a neck 4824 that has a rib 4825. The neck 4824 of the distal end portion 4822 is configured to contact engagement members 4721 of the safety lock 4700. Similarly stated, the neck 4824 of the distal end portion 4822 is disposed within a space defined between the engagement members 4721 of the safety lock 4700. The engagement members 4721 allow the distal end portion 4822 of the outer needle sheath 4820 to move between the engagement members 4721 in a distal direction, but not in a proximal direction. Similarly stated, the engagement members 4721 include an edge that contacts the rib 4825 of the outer needle sheath 4820 such as to prevent the safety lock 4700 from moving in a distal direction relative to the outer needle sheath 4820. Said another way, the needle guard assembly 4800 is removed from the needle 4216 when the safety lock 4700 is moved in a distal direction with respect to the housing 4100 (similar to the result as shown for the medical injector 3000 in FIG. 50).

The function of the medical injector 4000 is substantially similar to the function of the medical injector 3000, described with reference to FIGS. 9-55. In this manner, the user of the medical injector 4000 can actuate the medical injector 4000 to inject a medicament, disposed within the medicament container 4200, into an injection site of a patient.

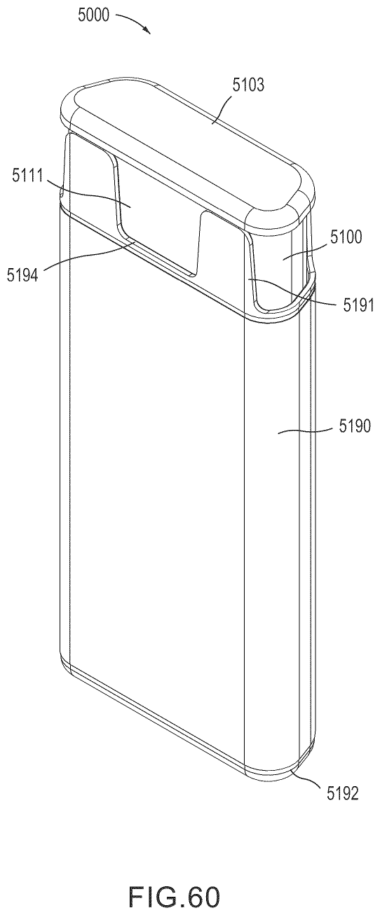

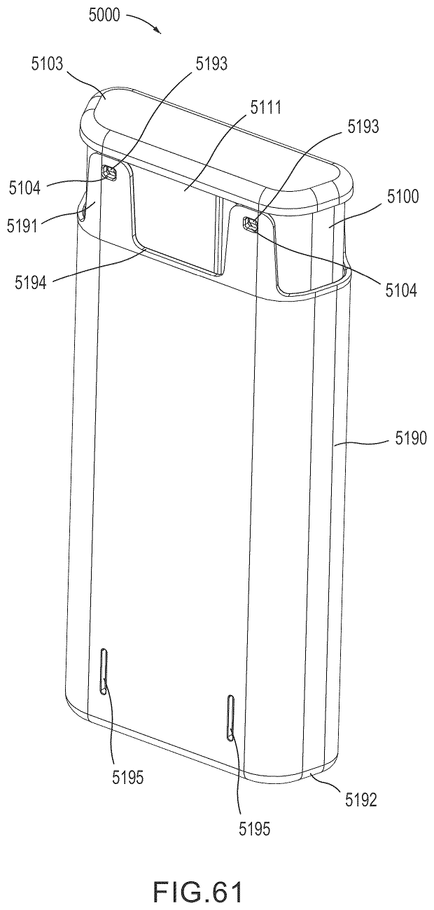

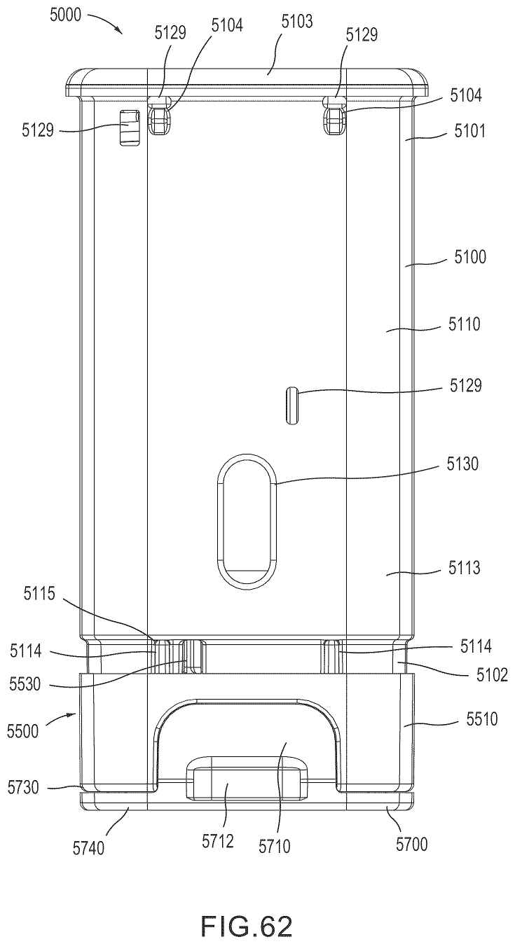

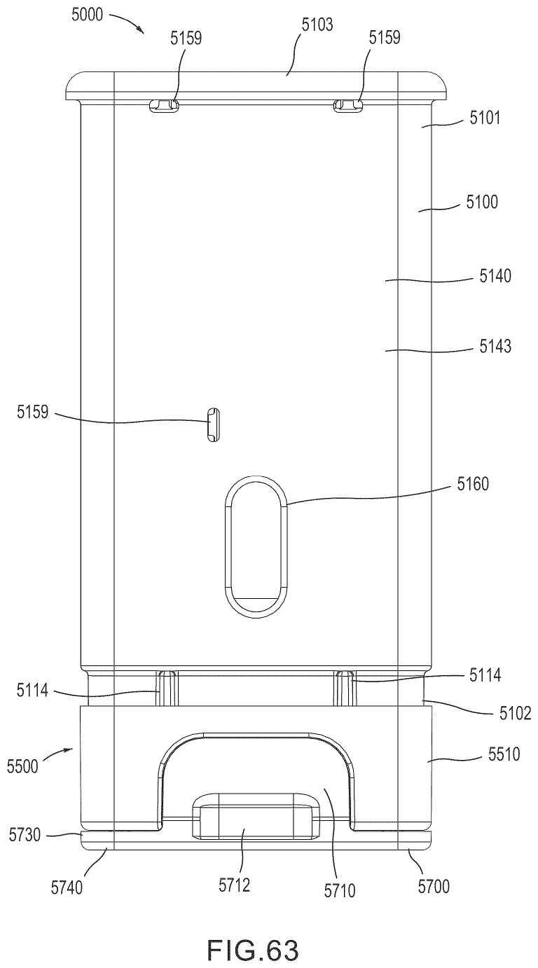

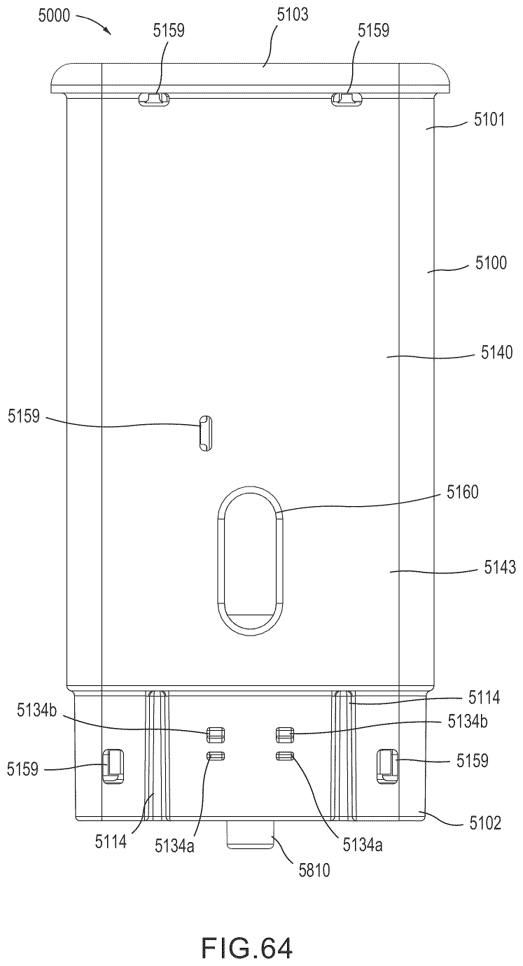

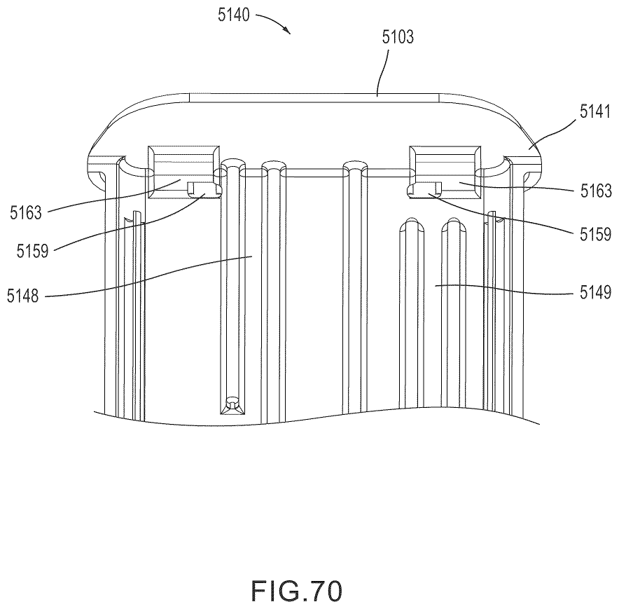

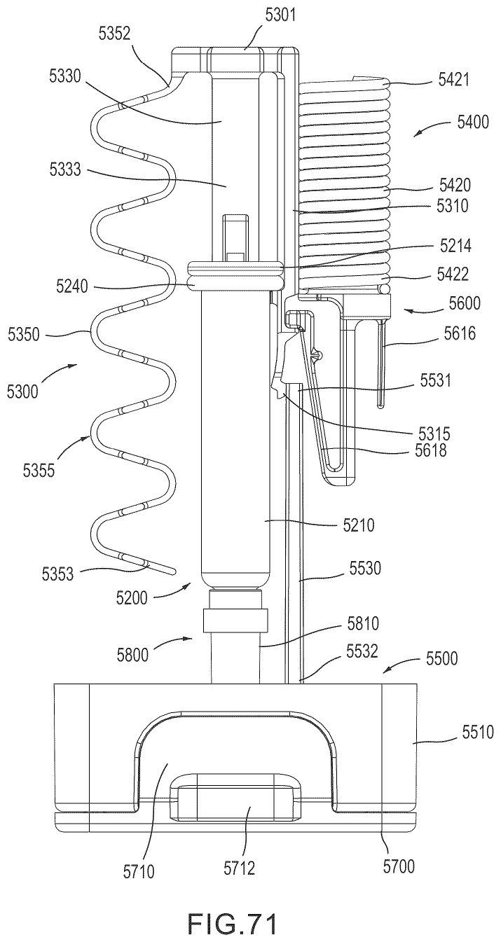

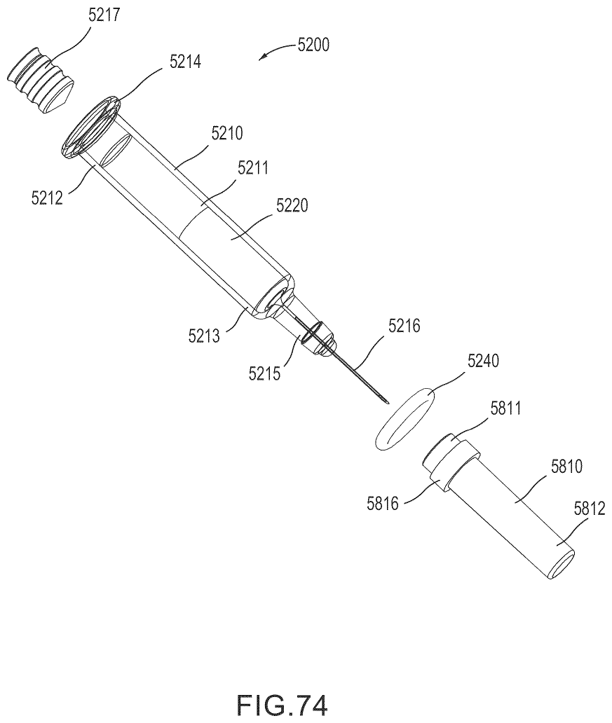

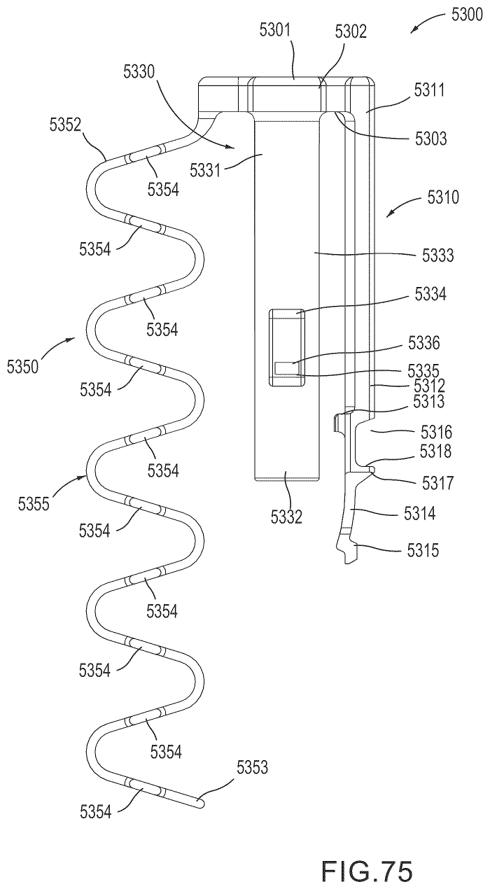

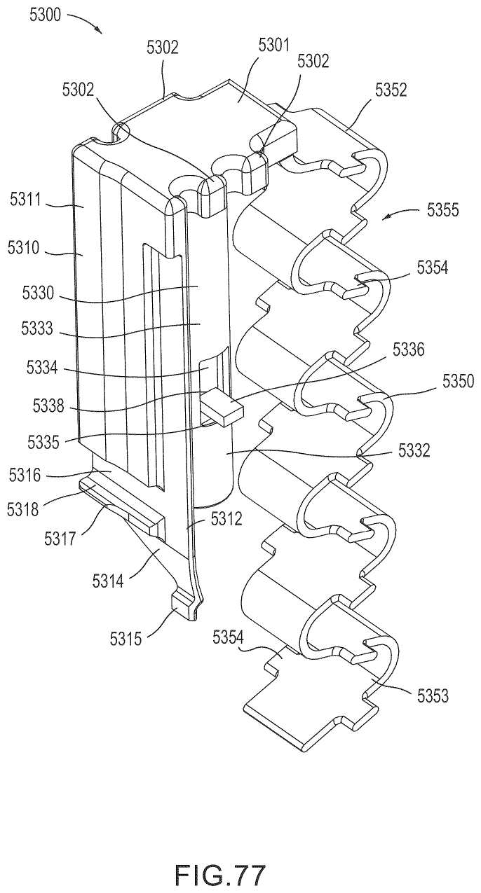

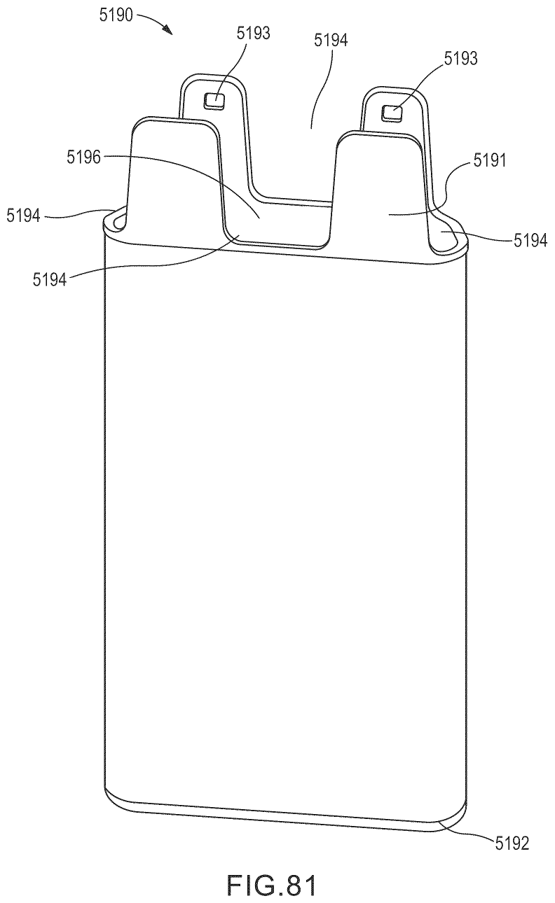

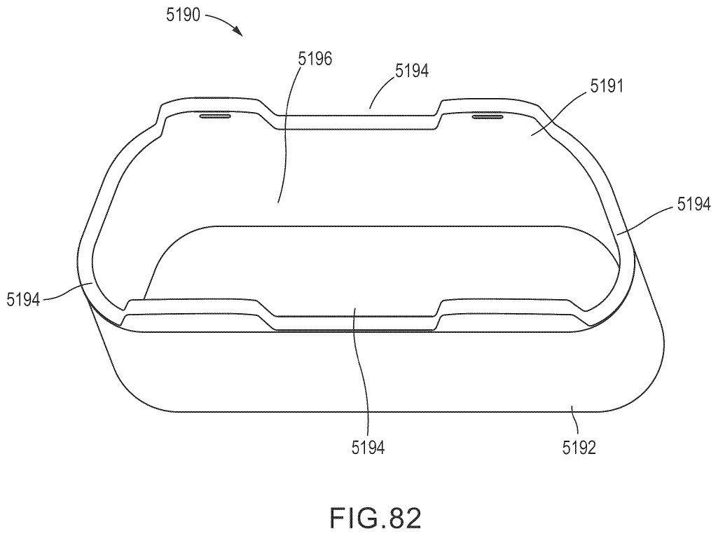

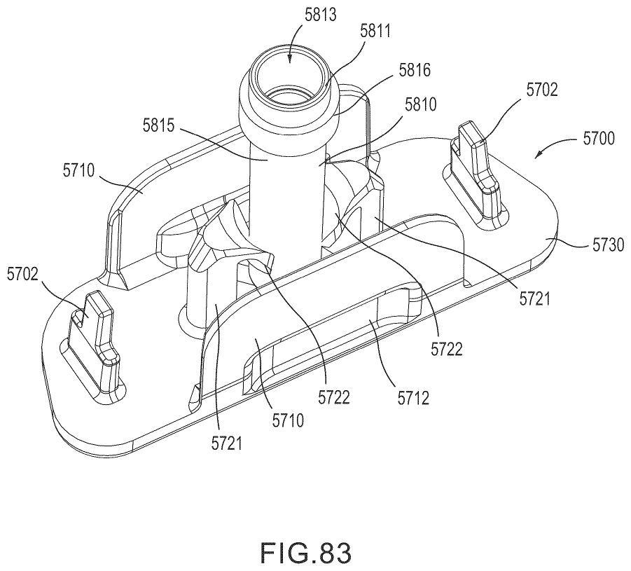

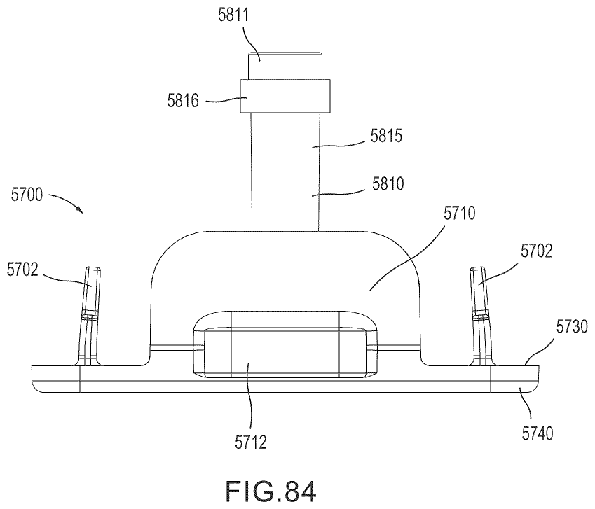

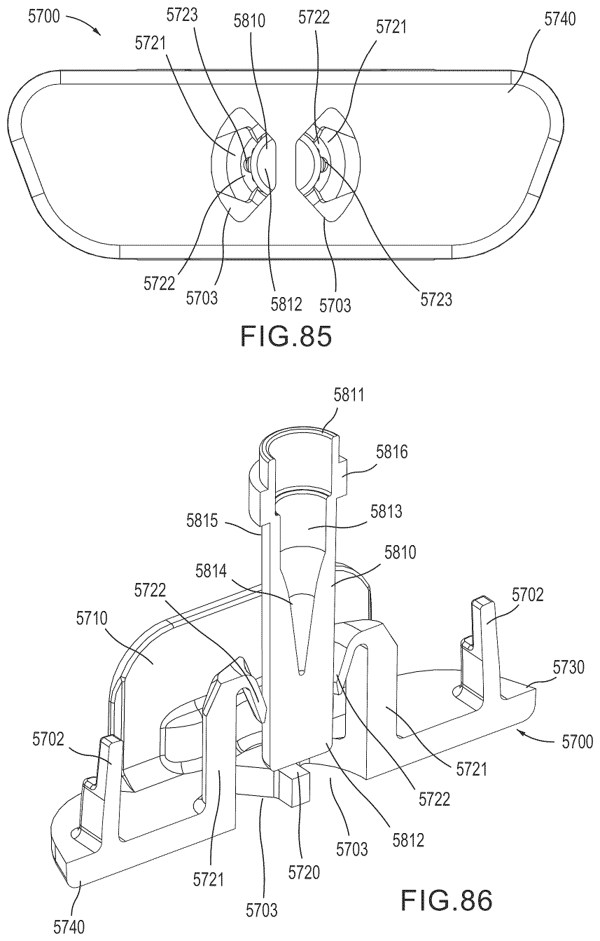

Although the medicament injector 3000 and the medical injector 4000 are shown and described above as including a system actuation including the release of a pressurized gas, in other embodiments, a medicament delivery device can include any suitable method of delivery of a medicament disposed within. For example, FIGS. 60-98 show a medical injector 5000, according to an embodiment that includes a mechanical energy storage member, rather than a compressed gas container. FIGS. 60-61 are perspective views of the medical injector 5000 in a first configuration (i.e., prior to use). The medical injector 5000 includes a housing 5100 (see e.g., FIGS. 62-70), a system actuator 5500 (see e.g., FIGS. 71-73), a medicament container 5200 containing a medicament 5220 (see e.g., FIG. 74), a medicament delivery mechanism 5300, a transfer member 5600 (see e.g., FIG. 75-80), a cover 5190 (see e.g., FIGS. 81-82), and a safety lock 5700 (see e.g., FIGS. 83-87). A discussion of the components of the medical injector 5000 will be followed by a discussion of the operation of the medical injector 5000.

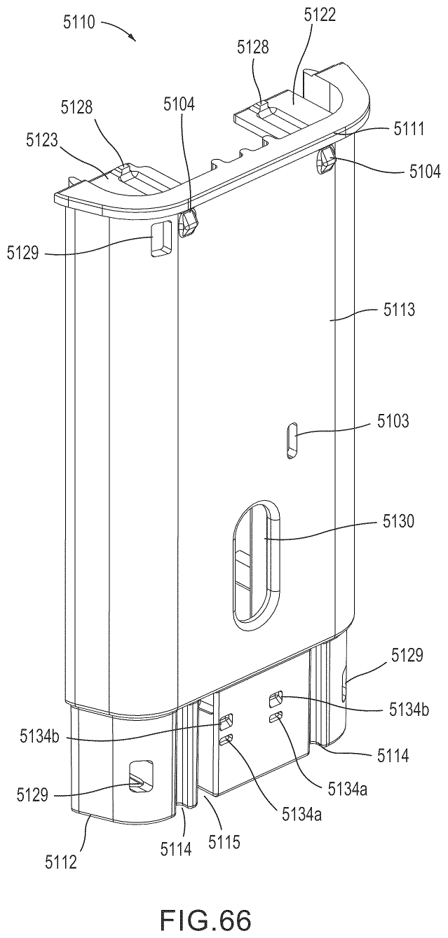

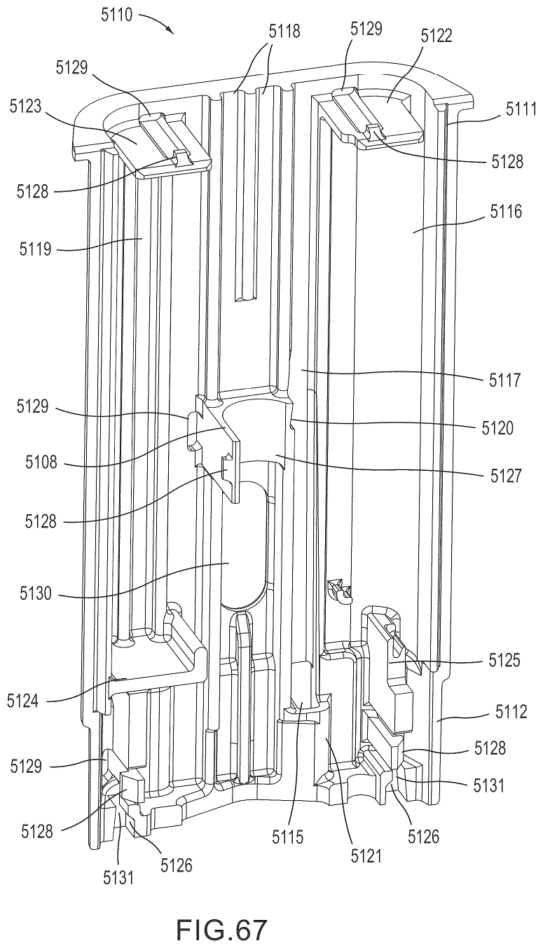

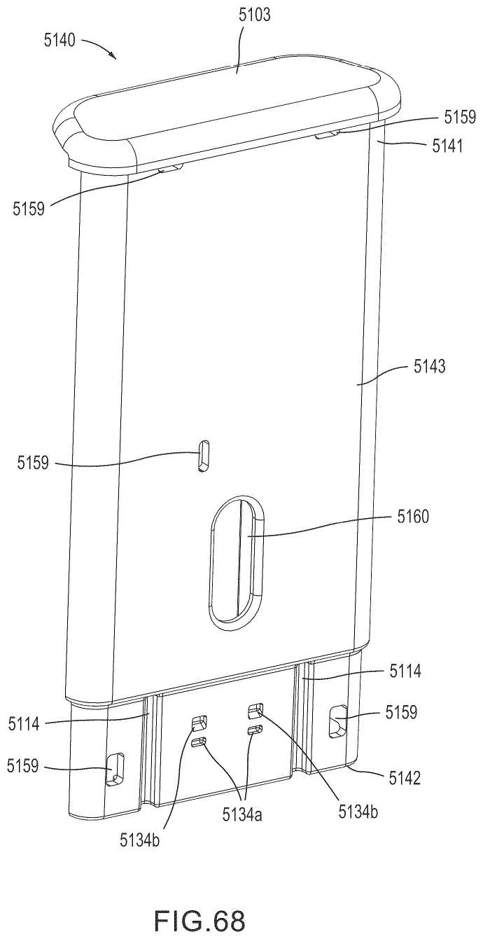

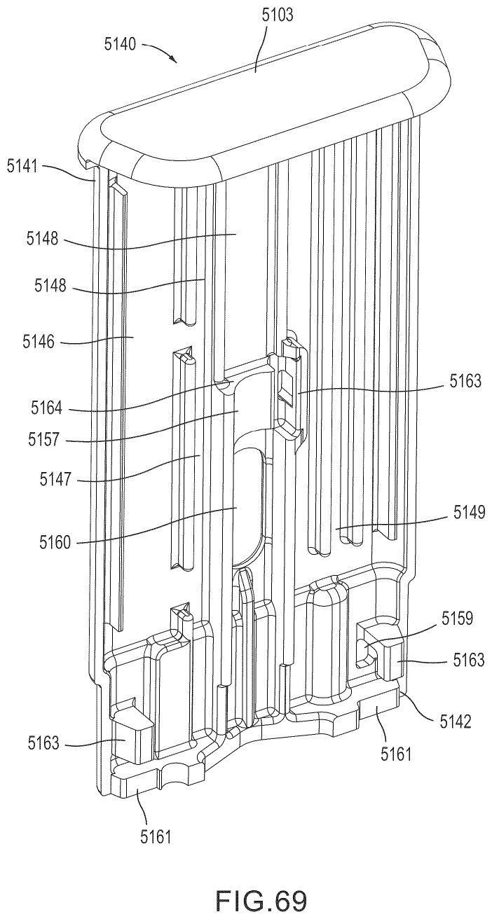

As shown in FIGS. 62-70, the housing 5100 includes a first housing member 5110 (FIGS. 66 and 67) and a second housing member 5140 (FIGS. 68 and 69) that can couple to form the housing 5100. The housing 5100 has a proximal end portion 5101 and a distal end portion 5102. The housing 5100 defines a first status indicator aperture 5130 (defined by the first housing member 5110) and a second status indicator aperture 5160 (defined by the second housing member 5140). The status indicator apertures 5130, 5160 can allow a patient to monitor the status and/or contents of the medicament container 5200 contained within the housing 5100. For example, by visually inspecting the status indicator aperture 5130 and/or 5160, a patient can determine whether the medicament container 5200 contains a medicament 5220 and/or whether the medicament 5220 has been dispensed.

As shown in FIGS. 66-67, the first housing member 5110 includes an outer surface 5113 and an inner surface 5116, and a proximal end portion 5111 and a distal end portion 5112. The outer surface 5113 includes cover retention protrusions 5104 at the proximal end portion 5111 of the first housing member 5110 (see e.g., FIGS. 61, 62 and 66). The cover retention protrusions 5104 are configured to be received within corresponding openings 5193 defined by the cover 5190 to retain the cover 5190 about the housing 5100. In this manner, as described in more detail herein, the cover 5190 is removably coupled to and disposed about at least a portion of the housing 5100.

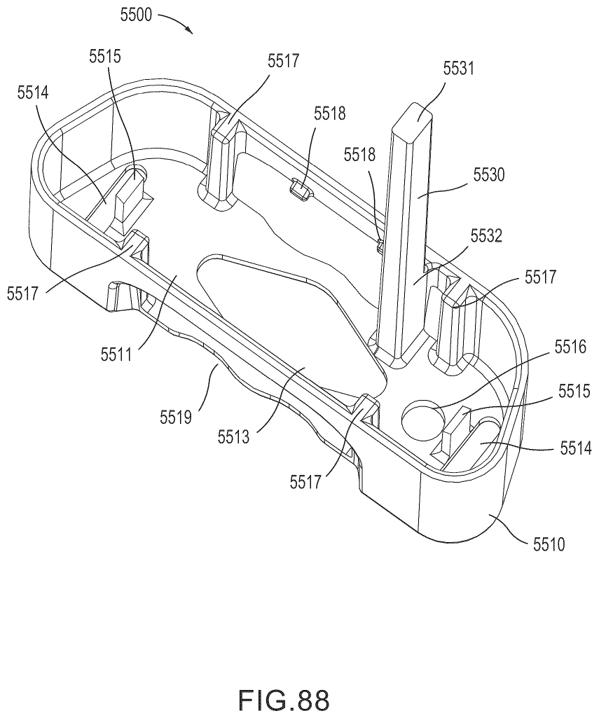

The outer surface 5113 defines base retention recesses 5134A and 5134B, an activation rod groove 5115, and base rail grooves 5114, at the distal end portion 5112 of the first housing member 5110. The distal base retention recesses 5134A are configured to receive base connection knobs 5518 of an actuator 5510 (also referred to herein as “base 5510,” see e.g., FIG. 88) when the base 5510 is in a first position relative to the housing 5100. The proximal base retention recesses 5134B are configured to receive the base connection knobs 5518 of the base 5510 when the base 5510 is in a second position relative to the housing 5100. The base retention recesses 5134A, 5134B have a tapered proximal sidewall and a non-tapered distal sidewall. This allows the base retention recesses 5134A, 5134B to receive the base connection knobs 5518 such that the base 5510 can move proximally relative to the housing 5100, but cannot move distally relative to the housing 5100. Said another way, the distal base retention recesses 5134A are configured to prevent the base 5510 from moving distally when the base 5510 is in a first position and the proximal base retention recesses 5134B are configured to prevent the base 5510 from moving distally when the base 5510 is in a second position. Similarly stated, the proximal base retention recesses 5134B and the base connection knobs 5518 cooperatively to limit movement of the base 5510 to prevent undesirable movement of the base 5510 after the medical injector 5000 is actuated. The proximal base retention recesses 5134B and the base connection knobs 5518 also provide a visual cue to the user that the medical injector 5000 has been used.

The activation rod groove 5115 is configured to receive an activator 5530 (also referred to herein as “release member 5530,” see e.g., FIG. 88) of the base 5510. As described in more detail herein, the release member 5530 of the base 5510 is configured to engage a portion of the medicament delivery mechanism 5300 when the base 5510 is moved with respect to the housing 5100. The base rail grooves 5114 are configured to receive guide members 5517 of the base 5510. The guide members 5517 of the base 5510 and the base rail grooves 5114 of the housing 5100 engage each other in a way that allows the guide members 5517 of the base 5510 to slide in a proximal and/or distal direction within the base rail grooves 5114 while limiting lateral movement of the guide members 5517. This arrangement allows the base 5510 to move in a proximal and/or distal direction with respect to the housing 5100 but prevents the base 5510 from moving in a lateral direction with respect to the housing 5100.

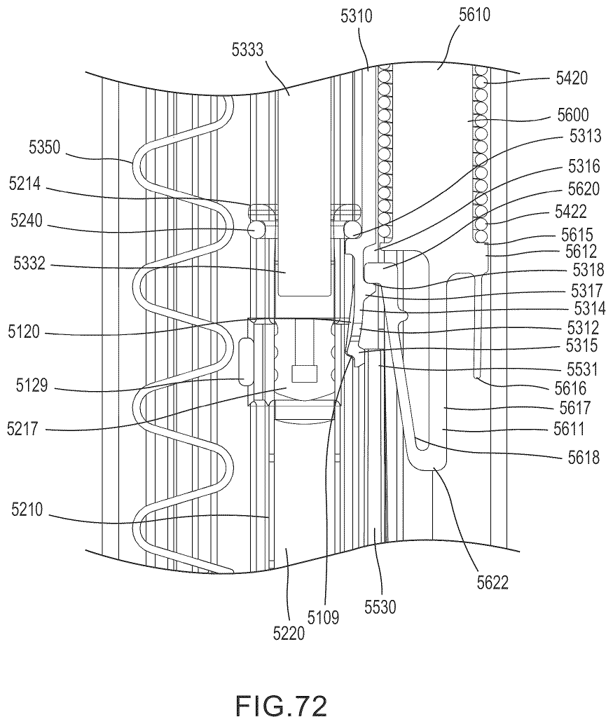

The inner surface 5116 of the first housing member 5110 includes a medicament container holder 5127, an upper spring plate 5122 and an upper bias member plate 5123. The inner surface 5166 also includes a series of protrusions that define a transfer member groove 5117, piston portion grooves 5118 and a bias portion groove 5119 (see e.g., FIG. 67). The medicament container holder 5127 is configured to receive a body 5210 of the medicament container 5200 (e.g., a prefilled syringe). The medicament container holder 5127 defines a latch member notch 5120 that includes an engagement surface 5109 (see e.g. FIG. 72) configured to engage a latch protrusion 5315 of a latch portion 5310 of the medicament delivery mechanism 5300. The medicament container holder 5127 includes a proximal end surface 5108. The proximal end surface 5108 is configured to contact a portion of the medicament container 5200 (either directly or via intervening structure, such as an o-ring or damping member) when the medicament container 5200 is in a second position, as described in further detail herein.

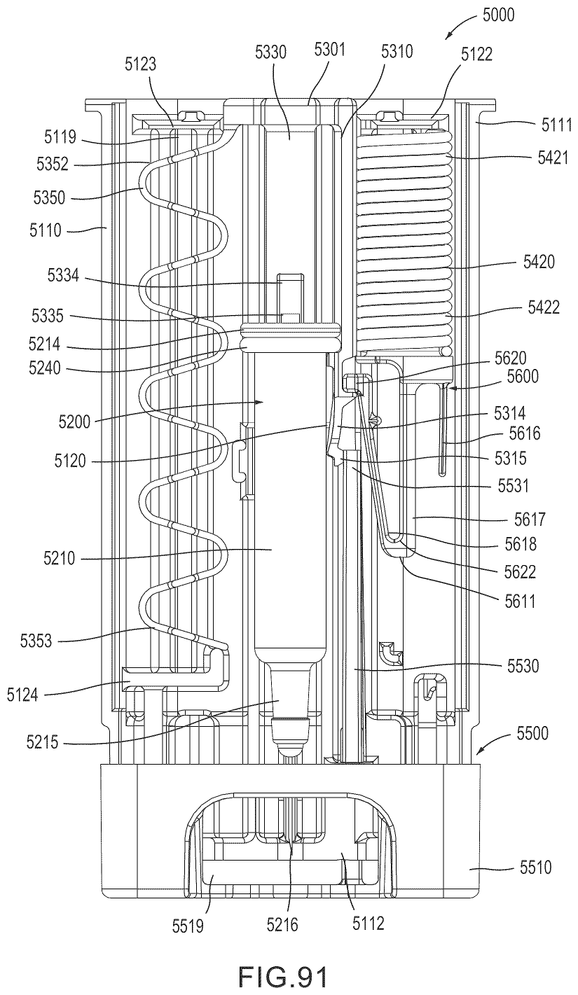

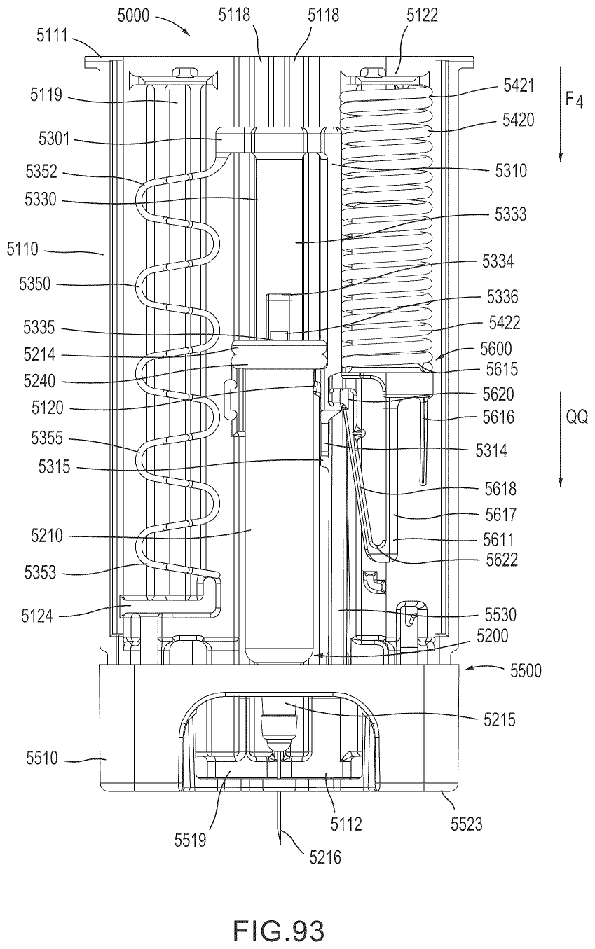

The upper spring plate 5122 is disposed at the proximal end portion 5111 of the first housing member 5110. The upper spring plate 5122 extends from the inner surface 5116 and is configured to contact a proximal end portion 5421 of a spring 5420 (see FIG. 91). In this manner, when activated, the upper spring plate 5122 limits proximal movement of the spring 5420 such that the spring expands distally to move the medicament delivery mechanism 5300 in a distal direction (see e.g., FIG. 93). Similarly stated, the upper spring plate 5122 receives a force from the spring 5420 and applies an equal and opposite reaction force to the proximal end portion 5421 of the spring 5420 such that a distal end portion 5422 of the spring 5420 expands in a distal direction, as described in further detail herein.

The upper bias plate 5123 is disposed at the proximal end portion 5111 of the first housing member 5110 and extends from the inner surface 5116. The upper bias plate 5123 is configured to selectively engage a bias portion 5350 of the medicament delivery mechanism 5300 (see FIG. 91). In this manner, the upper bias plate 5123 is configured to limit the proximal movement of the bias portion 5350 of the medicament delivery mechanism 5300, as described in further detail herein.

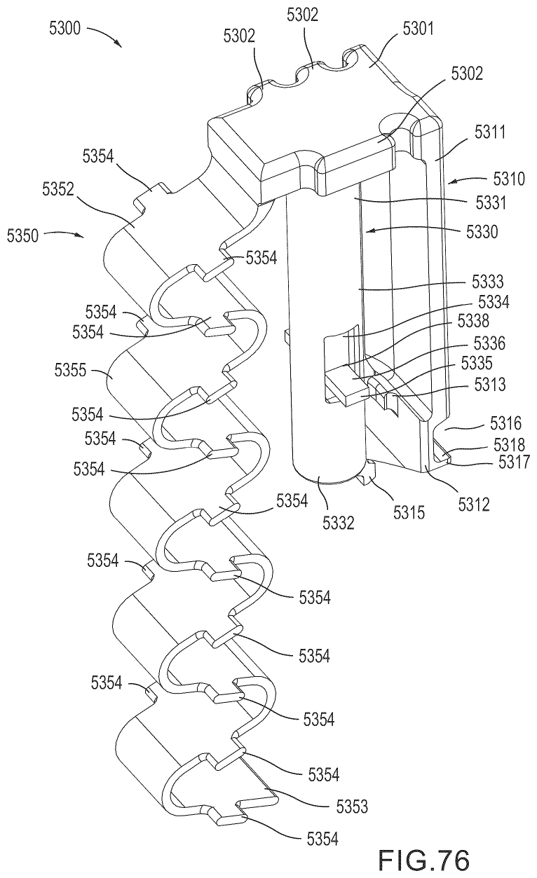

As described above, the inner surface 5116 includes protrusions that define the transfer member groove 5117, the piston portion grooves 5118 and the bias portion groove 5119. The transfer member groove 5117 is configured to receive a guide protrusion 5619 of the transfer member 5600 (see FIG. 80). The guide protrusion 5619 of the transfer member 5600 and the transfer member groove 5117 defined by the inner surface 5116 of the first housing member 5110 engage each other in a way that allows the guide protrusion 5619 of the transfer member 5600 to slide in a proximal and/or distal direction within the transfer member groove 5117 while limiting lateral movement of the guide protrusion 5619. This arrangement allows the transfer member 5600 to move in a proximal and/or distal direction with respect to the housing 5100 but prevents the transfer member 5600 from moving in a lateral direction with respect to the housing 5100. Similarly, the piston portion grooves 5118 are configured to receive the guide protrusions 5302 of the piston portion 5330 of the medicament delivery mechanism 5300 (see FIG. 76). The bias portion groove 5119 is configured to receive the guide protrusion 5354 of the bias portion 5350 of the medicament delivery mechanism 5300 (see FIG. 76). In this manner, the piston portion grooves 5118 and the bias member groove 5119 engage the guide protrusions 5302 of the piston portion 5330 and the guide protrusion 5354 of the bias portion 5350, respectively, to prevent the medicament delivery mechanism 5300 from moving in a lateral direction with respect to the housing 5100 and/or rotating within the housing 5100.

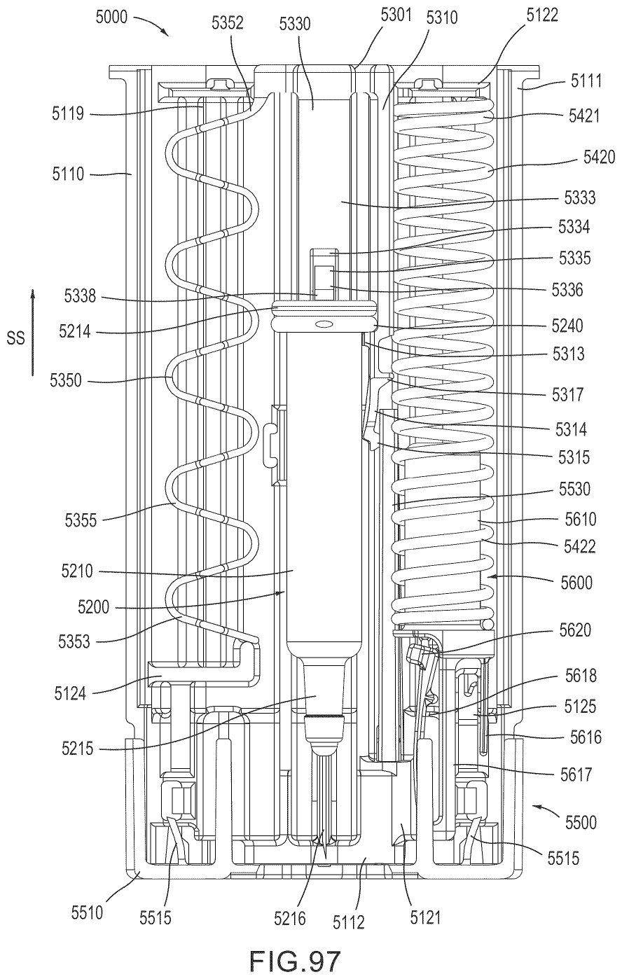

The inner surface 5116 of the first housing member 5110 further includes a transfer member release protrusion 5121, a transfer member release support protrusion 5125, a lower bias plate 5124, and base lock protrusions 5126. The transfer member release protrusion 5121 is configured to engage a latch arm 5618 of the transfer member 5600 to place the transfer member 5600 in a second configuration when the transfer member 5600 moves to a second position (see e.g., FIG. 97). Contemporaneously, the transfer member release support protrusion 5125 supports the latch arm 5618 of the transfer member 5600 as the transfer member is placed in the second configuration, as described in further detail herein.

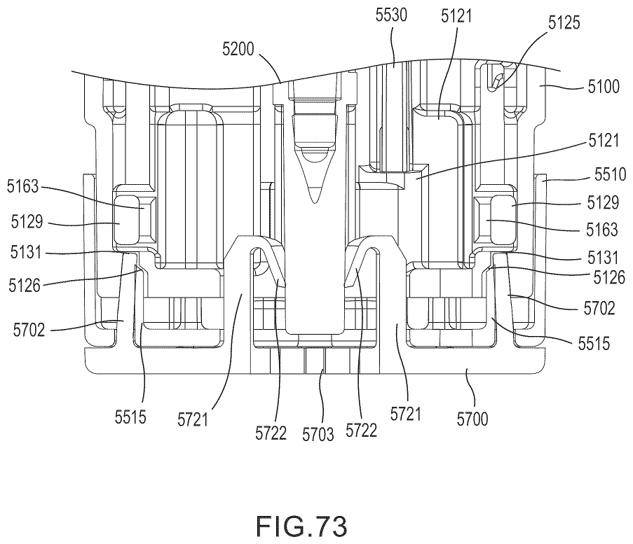

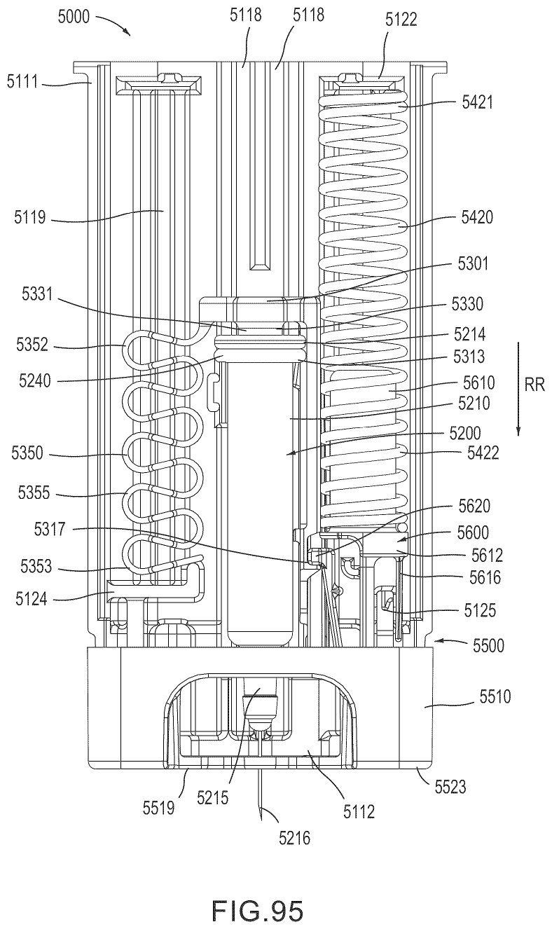

The lower bias plate 5124 engages a distal end portion 5353 of the bias portion 5350 of the delivery mechanism 5300 (see e.g., FIG. 95), as described in further detail herein. The base lock protrusions 5126 are configured to engage base locks 5515 of the base 5510 when the safety lock 5700 is in contact with the medical injector 5000 (see FIG. 73). Similarly stated, the safety lock 5700, the base lock protrusions 5126, and the base locks 5515 collectively prevent the base 5510 from moving in a proximal direction relative to the housing 5100 when the base locks 5515 of the base 5510 are in contact with the base lock protrusions 5126 of the first housing portion 5110, as described in further detail herein.

The first housing member 5110 further includes a set of tabs 5128 and a set of openings 5129. The tabs 5128 extend from portions of the inner surface 5116 of the first housing member 5110. The first housing member 5110 can include any number of tabs 5128 that can have any suitable shape or size. For example, in some embodiments, the tabs 5128 vary in size. The tabs 5128 are configured to engage portions of the second housing member 5140 to couple the first housing member 5110 to the second housing member 5140, as described in further detail herein.

As shown in FIGS. 68-70, the second housing member 5140 includes an outer surface 5143 and an inner surface 5146. The second housing member 5140 also includes a proximal end portion 5141, a proximal cap 5103, and a distal end portion 5142. The outer surface 5143 defines base retention recesses 5134A and 5134B and base rail grooves 5114, at the distal end portion 5142 of the second housing member 5140. The distal base retention recesses 5134A are configured to receive base connection knobs 5518 of the base 5510 when the base 5510 is in a first position relative to the housing 5100. The proximal base retention recesses 5134B are configured to receive the base connection knobs 5518 of the base 5510 when the base 5510 is in a second position relative to the housing 5100. The base retention recesses 5134A, 5134B have a tapered proximal sidewall and a non-tapered distal sidewall. This allows the base retention recesses 5134A, 5134B to receive the base connection knobs 5518 such that the base 5510 can move proximally relative to the housing 5100, but cannot move distally relative to the housing 5100. Said another way, the distal base retention recesses 5134A are configured to prevent the base 5510 from moving distally when the base 5510 is in a first position and the proximal base retention recesses 5134B are configured to prevent the base 5510 from moving distally when the base 5510 is in a second position. Similarly stated, the proximal base retention recesses 5134B and the base connection knobs 5518 cooperatively limit movement of the base 5510 to prevent undesirable movement of the base 5510 after the medical injector 5000 is actuated. The proximal base retention recesses 5134B and the base connection knobs 5518 also provide a visual cue to the user that the medical injector 5000 has been used.

The base rail grooves 5114 are configured to receive guide members 5517 of the base 5510. The guide members 5517 of the base 5510 and the base rail grooves 5114 of the second housing member 5140 engage each other in a way that allows the guide members 5517 of the base 5510 to slide in a proximal and/or distal direction within the base rail grooves 5114 while limiting lateral movement of the guide members 5517. This arrangement allows the base 5510 to move in a proximal and/or distal direction with respect to the housing 5100 but prevents the base 5510 from moving in a lateral direction with respect to the housing 5100.

The proximal cap 5103 extends from the proximal end portion 5141 of the second housing member 5140 and encloses the proximal end portion 5101 of the housing 5100 when the first housing member 5110 is coupled to the second housing member 5140.

The inner surface 5146 of the second housing member 5140 includes a medicament container holder 5157. The inner surface further includes protrusions that define a transfer member groove 5147, piston portion grooves 5148, and a bias portion groove 5149. The medicament container holder 5157 is configured to receive a body 5210 of the medicament container 5200 (e.g., a prefilled syringe). Moreover, the medicament container holder 5157 is configured to be coupled to a portion of the medicament container holder 5127 of the first housing member 5110 to define a space in which the medicament container 5200 is disposed. The medicament container holder 5157 includes a proximal end surface 5164. The proximal end surface 5164 is configured to contact a portion of the medicament container 5200 (either directly or via intervening structure) when the medicament container 5200 is in the second position, as described in further detail herein.

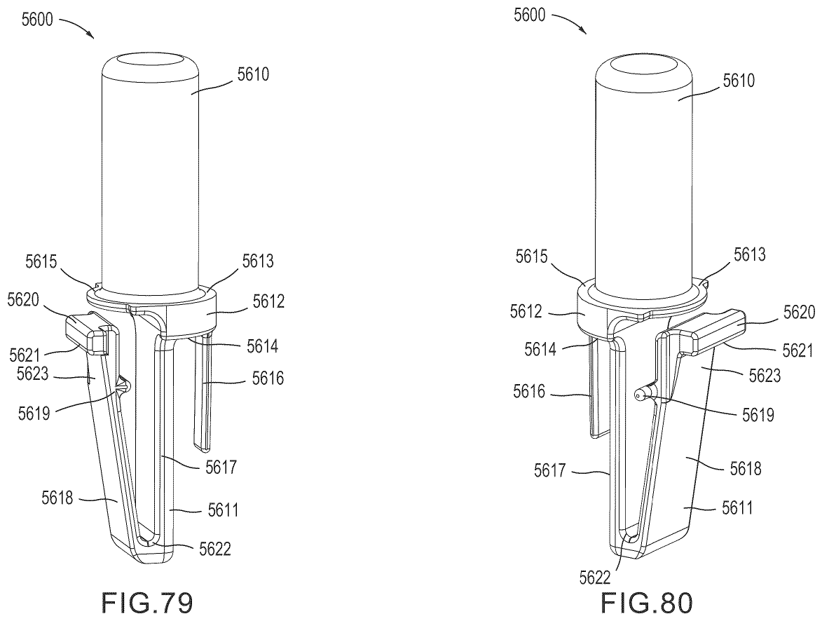

The transfer member groove 5147 receives a latch 5620 of the transfer member 5600 (see FIGS. 79 and 80). The latch 5620 of the transfer member 5600 and the transfer member groove 5147 defined by the inner surface 5146 of the second housing member 5140 engage each other in a way that allows the latch 5620 of the transfer member 5600 to slide in a proximal and/or distal direction within the transfer member groove 5147 while limiting lateral movement of the guide protrusion 5619. Similarly, the piston portion grooves 5148 are configured to receive the guide protrusions 5302 of the piston portion 5330 of the medicament delivery mechanism 5300. The bias portion groove 5149 is configured to receive the guide protrusion 5354 of the bias portion 5350 of the medicament delivery mechanism 5300. In this manner, the piston portion grooves 5148 and the bias member groove 5149 engage the guide protrusions 5302 of the piston portion 5330 and the guide protrusion 5354 of the bias portion 5350, respectively, to prevent the medicament delivery mechanism 5300 from moving in a lateral direction with respect to the housing 5100 and/or rotating within the housing 5100.

The second housing member 5140 further includes a set of tab latches 5163 and defines a set of openings 5159. The second housing member 5140 can include any number of tab latches 5163 such that the number of tab latches 5163 correspond to the number of tabs 5128 of the first housing member 5110. Collectively, the tabs 5128 of the first housing member 5110 and the tab latches 5163 of the second housing member 5140 couple the first housing member 5110 to the second housing member 5140. Similarly stated, the tabs 5128 are configured to engage the tab latches 5163 to define a lock fit. Moreover, a surface of the tabs 5128 is in contact with a surface of the tab latches 5163 to define a lock fit such that the first housing member 5110 and the second housing member 5140 couple together to define the housing 5100. The openings 5129 of the first housing member 5110 and the openings 5159 of the second housing member 5140 allow access to the tabs 5128 of the first housing member 5110 and the tab latches 5163 of the second housing member 5140, respectively. In this manner, the first housing member 5110 can be decoupled from the second housing member 5140.

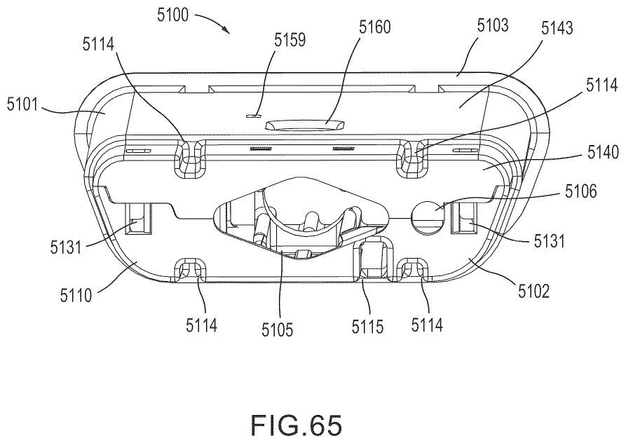

As shown in FIG. 65, when the first housing member 5110 and the second housing member 5140 are assembled, the distal end portion 5102 of the housing 5100 defines a needle aperture 5105, a transfer member access opening 5106 and base lock openings 5131. Similarly stated, the first housing member 5110 and the second housing member 5140 collectively define the needle aperture 5105, the transfer member access opening 5106 and the base lock openings 5131. The needle aperture 5105 is configured to allow the needle 5216 (see e.g., FIGS. 74, 92 and 93) to exit the housing 5100 when the medical injector 5000 is actuated, as described in further detail herein.

The transfer member access opening 5106 is configured to provide access to the transfer member 5600 when the transfer member 5600 is disposed within the housing 5100. For example, in some embodiments, the transfer member 5600 can be disengaged from the medicament delivery mechanism 5300 without moving the medicament delivery mechanism 5300 in the distal direction. In this manner, the medical injector 5000 can be disabled such that the medicament delivery mechanism 5300 cannot engage the medicament container 5200 to convey a medicament 5220. For example, in some embodiments, a user, manufacturer and/or operator can disengage the transfer member 5600 from the medicament delivery mechanism 5300, via the transfer member access opening 5106, to safely dispose of an unused medical injector 5000 whose medicament 5220 expired. In other embodiments, an operator can manipulate the transfer member within the housing 5100 via the transfer member access opening 5106 during the assembly of the medical injector 5000.

The base lock openings 5131 are configured to receive the base locks 5515 and the safety lock protrusions 5702, as shown in the cross-sectional view of FIG. 73. The base lock openings 5131 receive the base locks 5515 and the safety lock protrusions 5702 such that the base locks 5515 of the base 5510 are in contact with the base lock protrusions 5126 of the first housing member 5110 when the safety lock protrusions 5702 are disposed within the base lock openings 5131. In this manner, the safety lock protrusions 5702 and the base lock protrusion 5126 prevent the base from moving in a proximal direction by placing the a proximal surface of the base locks 5515 in contact with a distal surface of the base lock protrusions 5126. When the safety lock protrusions 5702 are removed from the base lock openings 5131, the proximal surface of the tapered surface of the base locks 5515 allow movement in a proximal direction past the corresponding tapered surfaces of the base lock protrusions 5126 when the base 5510 is moved in the proximal direction.

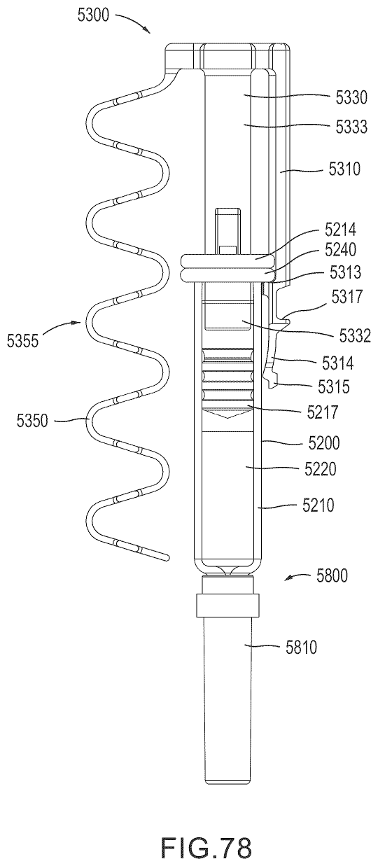

FIGS. 71-80 show the medicament container 5200, the system actuator 5500, the transfer member 5600 and the medicament delivery mechanism 5300 of the medical injector 5000. The medicament container 5200 has a body 5210 with a distal end portion 5213 and a proximal end portion 5212. The body 5210 defines a volume 5211 that contains (i.e., is filled with or partially filled with) a medicament 5220 (see, e.g., FIG. 74). The distal end portion 5213 of the medicament container 5200 includes a neck 5215 that is coupled to the needle 5216, as described below. The proximal end portion 5212 of the medicament container 5200 includes an elastomeric member 5217 (i.e., a plunger) that seals the medicament 5220 within the body 5210. The elastomeric member 5217 is configured to move within the body 5210 to inject the medicament 5220 from the medicament container 5200. More particularly, as shown in FIG. 78, the elastomeric member 5217 receives a piston rod 5333 of a piston portion 5330 included in the medicament delivery mechanism 5300. The proximal end portion 5212 includes a flange 5214 and a damping member 5240 (see FIG. 78) configured to engage the piston portion 5330 and the latch portion 5310 of the medicament delivery mechanism 5300. The flange 5214 and the damping member 5240 are also configured to engage and/or contact the medicament container holders 5127 and 5157 of the housing 5100.

The elastomeric member 5217 can be of any design or formulation suitable for contact with the medicament 5220. For example, the elastomeric member 5217 can be formulated to minimize any reduction in the efficacy of the medicament 5220 that may result from contact (either direct or indirect) between the elastomeric member 5217 and the medicament 5220. For example, in some embodiments, the elastomeric member 5217 can be formulated to minimize any leaching or out-gassing of compositions that may have an undesired effect on the medicament 5220. In other embodiments, the elastomeric member 5217 can be formulated to maintain its chemical stability, flexibility and/or sealing properties when in contact (either direct or indirect) with the medicament 5220 over a long period of time (e.g., for up to six months, one year, two years, five years or longer). In some embodiments, the elastomeric member 5217 is similar to the elastomeric member 3217 of the medical injector 3000, described with reference to FIG. 22.

The medicament container 5200 can have any suitable size (e.g., length and/or diameter) and can contain any suitable volume of the medicament 5220. Moreover, the medicament container 5200 and the piston portion 5330 can be collectively configured such that the piston portion 5330 travels a desired distance within the medicament container 5200 (i.e., the “stroke”) during an injection event. In this manner, the medicament container 5200, the volume of the medicament 5220 within the medicament container 5200 and the piston portion 5330 can be collectively configured to provide a desired fill volume and delivery volume. For example, the medicament container 5200, as shown in FIG. 74, is a prefilled syringe and can be purchased and/or acquired with a given fill volume. In this manner, the piston portion 5330 can be configured to provide a desired delivery volume.

Moreover, the length of the medicament container 5200 and the length of the piston portion 5330 can be configured such that the medicament delivery mechanism 5300 can fit in the same housing 5100 regardless of the fill volume, the delivery volume and/or the ratio of the fill volume to the delivery volume. In this manner, the same housing and production tooling can be used to produce devices having various dosages of the medicament 5220. For example, in a first embodiment (e.g., having a fill volume to delivery volume ratio of 0.4), the medicament container has a first length and the second movable member has a first length. In a second embodiment (e.g., having a fill volume to delivery volume ratio of 0.6), the medicament container has a second length shorter than the first length, and the second movable member has a second length longer than the first length. In this manner, the stroke of the device of the second embodiment is longer than that of the device of the first embodiment, thereby allowing a greater dosage. The medicament container of the device of the second embodiment, however, is shorter than the medicament container of the device of the first embodiment, thereby allowing the components of both embodiments to be disposed within the same housing and/or a housing having the same length.

As shown in FIGS. 71-74, the system actuator 5500 includes the base 5510 and a release member 5530, and is configured to move in the proximal and distal direction relative to the housing 5100. Although the base 5510 and the release member 5530 are shown as being monolithically constructed to form the system actuator 5500, in other embodiments the system actuator 5500 can include a base that is constructed separately from (and later joined to) a release member. As described above, when the medical injector 5000 is in its first configuration (i.e., the storage configuration), the base locks 5515 and the safety lock protrusions 5702 are disposed within the base lock opening 5131 such that the base locks 5515 are urged by the safety lock protrusions 5702 into contact with the base lock protrusions 5126. Therefore, the system actuator 5500 and/or the base 5510 cannot move in the proximal direction to actuate the medicament delivery mechanism 5300. Similarly stated, as shown in FIG. 73, when the medical injector 5000 is in its first configuration (i.e., the storage configuration), the safety lock protrusions 5702 and the base lock protrusions 5126 cooperatively limit the proximal movement of the base 5510.

The release member 5530 has a proximal end portion 5531 and a distal end portion 5532. The release member 5530 extends from a proximal surface 5511 of the base 5510. The proximal end portion 5531 of the release member 5530 is configured to engage that latch portion 5310 of the medicament delivery mechanism 5300 when the medical injector is in its first (or storage) configuration. More particularly, as shown in FIG. 72, the proximal end portion 5531 of the release member 5530 maintains a first latch protrusion 5315 of the latch portion 5310 in contact with the engagement surface 5109 of the latch member notch 5120 of the housing 5100. When the engagement surface 5109 is in contact with the first latch protrusion 5315, the engagement surface 5109 applies a reaction force to the first latch protrusion 5315 in response to the force applied by the spring 5420, which urges the transfer member 5600 and the medicament delivery mechanism 5300 in a distal direction. Similarly stated, when the first latch protrusion 5315 is in contact with the engagement surface 5109, the engagement surface 5109 limits distal movement of the first latch protrusion 5315, and thus, the medicament delivery mechanism 5300. In this manner, when the system actuator 5500 is in a first position (i.e., coupled to the distal end portion of the housing 5100), the release member 5530 maintains the first latch protrusion 5315 within the latch member notch 5120 and maintains the medical injector 5000 in the first configuration (e.g., non-actuated configuration).

The medicament delivery mechanism 5300 (all or portions of which can also be referred to as a “first movable member”) includes the latch portion 5310, the piston portion 5330 and the bias portion 5350 (see e.g., FIGS. 75-78). The latch portion 5310 is operably coupled to the spring 5420 via the transfer member 5600 (i.e., the second movable member 5600). The medicament delivery mechanism 5300 includes a proximal end portion 5301. The proximal end portion 5301 includes the guide protrusions 5302, described above with reference to FIGS. 67-70.

The latch portion 5310 includes a proximal end portion 5311 and a distal end portion 5312. The proximal end portion 5311 is disposed at and/or joined with the proximal end portion 5301 of the medicament delivery mechanism 5300. Similarly stated, the latch portion 5310 is configured to extend from the proximal end portion 5301 of the medicament delivery mechanism 5300 in the distal direction. The distal end portion 5312 of the latch portion 5310 includes a latch arm 5314 having a first latch protrusion 5315, a second latch protrusion 5317, and a second shoulder 5313, and defines a channel 5316. As described above, the first latch protrusion 5315 is configured to engage the release member 5530 and the engagement surface 5109 of the latch member notch 5120. In particular, as shown in FIG. 72, the release member 5530 urges, bends and/or deforms the latch arm 5314 to maintain the first latch protrusion 5315 within the latch member notch 5120. Thus, the latch arm 5314 can be constructed from a flexible material such that the release member 5530 can urge, bend and/or deform the latch arm 5314 to engage the first latch protrusion 5315 with the latch member notch 5120.

The channel 5316 of the latch portion 5310 is defined between a surface of the distal end portion 5312 of the latch portion 5310 and a proximal surface 5318 of the second latch protrusion 5317. The channel 5316 is configured to receive the latch 5620 of the transfer member 5600. More particularly, when the medical injector 5000 is in the first configuration, the proximal surface 5318 of the second latch protrusion 5317 is in contact with a distal surface 5621 of the latch 5620 of the transfer member 5600. In this manner, the transfer member 5600 can transfer a force produced by the actuation of the spring 5420 to the latch portion 5310 of the medicament delivery mechanism 5300 to move the medicament delivery mechanism 5300 in the distal direction. Similarly stated, this arrangement allows the medicament delivery mechanism 5300 to move with and/or remain coupled to the transfer member 5600 (which can be referred to as a “second movable member”) during the insertion and/or injection operation.

The piston portion 5330 includes a proximal end portion 5331 and a distal end portion 5332 and defines a piston rod 5333 therebetween. The proximal end portion 5331 is disposed at and/or joined with the proximal end portion 5301 of the medicament delivery mechanism 5300. Similarly stated, the piston portion 5330 is configured to extend from the proximal end portion 5301 of the medicament delivery mechanism 5300 in the distal direction. The distal end portion 5332 is configured to be disposed at least partially within the proximal end portion 5212 of the medicament container 5200. The piston rod 5333 defines recesses 5334.

The piston portion 5330 includes two engagement members 5336 that have a first shoulder 5335 and a deformable portion 5338. The engagement members 5336 are at least partially disposed within the recesses 5334 defined by the piston rod 5333, and extend in a lateral direction relative to the piston portion 5330. Similarly stated, the engagement members 5336 extend from the corresponding recess 5334 and are substantially perpendicular to a longitudinal axis defined by the piston portion 5330 between the proximal end portion 5331 and the distal end portion 5332. In this manner, as described in more detail herein, when the engagement members 5336 are deformed (e.g., at the deformable portion 5338), the engagement members 5336 fold into and/or are contained within the recesses 5334. The engagement members 5336 can be any suitable size or shape. In some embodiments, the engagement members 5336 can be monolithically formed with the piston portion 5330. In other embodiments, the engagement members 5336 can be formed separately from a brittle material and later coupled to the piston portion 5330. In still other embodiments, the engagement members 5336 can be formed separately from a flexible material and coupled to the piston portion 5330. In some embodiments, for example, the engagement members 5336 can be a single pin that is disposed through an opening within the piston portion 5330 such that the ends of the pins protrude from the recesses 5334.

The first shoulder 5335 of the engagement member 5336 is disposed at a distal surface of the engagement member 5336. As shown in FIG. 91, the first shoulder 5335 is configured to engage a proximal surface of the flange 5214 of the medicament container 5200. In this manner, the piston portion 5330 of the medicament delivery mechanism 5300 is configured to move the medicament container 5200 in response to a force applied by the spring 5420 when the medical injected 5000 is actuated. Similarly stated, when the release member 5530 actuates the medical injector 5000, the transfer member 5600 transfers a force from the spring 5420 to the medicament delivery mechanism 5300 such that the first shoulder 5335 of the piston portion 5330 moves the medicament container 5200 from the first position to the second position.

The deformable portion 5338 of the engagement member 5336 is configured to deform during and/or to initiate an injection event. The deformable portion 5338 can be any suitable structure that deforms (e.g., either plastically or elastically, including bending, breaking, stretching or the like) when the force applied thereto exceeds a value. For example, in some embodiments, the deformable portion 5338 can include a fillet configured to act as a stress concentration riser configured to deform under a given force. In use within the medical injector 5000, the deformable portion 5338 is configured to deform during and/or to initiate an injection event when the medicament container 5200 is in the second position. After deformation of the deformable portion 5338 and/or movement of the engagement members 5336, the first shoulder 5335 is no longer in contact with the flange 5214 of the medicament container 5200 and the piston portion 5330 is allowed to move in a distal direction, relative to the medicament container 5200.

The bias portion 5350 includes a proximal end portion 5352 and a distal end portion 5353. The proximal end portion 5352 is disposed at and/or joined with the proximal end portion 5301 of the medicament delivery mechanism 5300. Similarly stated, the bias portion 5350 is configured to extend from the proximal end portion 5301 of the medicament delivery mechanism 5300 in the distal direction.

The bias portion 5350 includes a serpentine portion 5355 constructed from any suitable material and having suitable dimensions such that the bias portion 5350 and/or the serpentine portion 5355 produce a force when the serpentine portion 5355 is compressed (see e.g., FIG. 95). As described above, the bias portion 5350 includes guide protrusions 5354 (see e.g., FIG. 76) configured to engage the bias member grooves 5119 defined by the first housing member 5110 and the bias member grooves 5149 defined by the second housing member 5140 to prevent the bias portion 5350 from moving in a lateral direction with respect to the housing 5100 and/or rotating within the housing 5100. The distal end portion 5353 of the bias portion 5350 is configured to engage the lower bias plate 5124. In this manner, a proximal surface of the lower bias plate 5124 prevents the distal end portion 5353 of the bias portion 5350 from moving in the distal direction as the medicament delivery device 5300 moves in the distal direction in response to the distal force applied by the spring 5420 when the medical injector 5000 is actuated. Therefore, the serpentine portion 5355 of the bias portion 5350 is compressed between the proximal end portion 5352 and the distal end portion 5353.

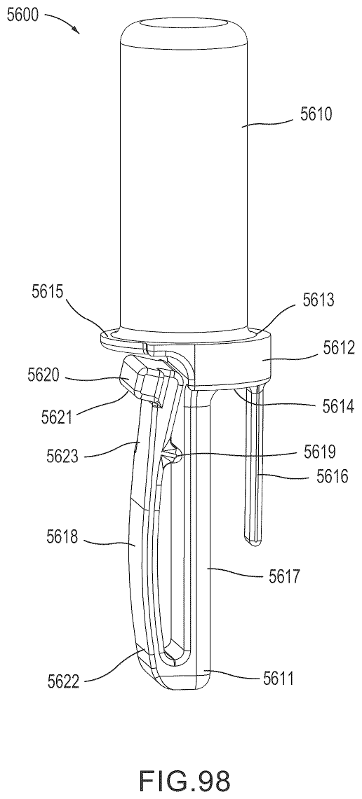

The transfer member 5600 (also referred to as the “second movable member”) includes a proximal end portion 5610 and a distal end portion 5611, and is configured to move between a first configuration (see e.g., FIGS. 79 and 80) and a second configuration (see e.g., FIGS. 97 and 98). The proximal end portion 5610 is substantially cylindrical and is configured to engage and/or contact the spring 5420. Moreover, the transfer member 5600 includes a ring protrusion 5612 that includes a proximal surface 5613 defining a spring seat 5615. As shown in FIG. 72, the distal end portion 5422 of the spring 5420 is disposed about the proximal end portion 5610 of the transfer member 5600, and is configured to engage the spring seat 5615 defined by the ring protrusion 5612.

The transfer member 5600 further includes a guide arm 5616 and the latch extension 5617 that extends from a distal surface 5614 of the ring protrusion 5612. The guide arm 5616 is configured to guide the transfer member 5600 as it moves in the distal direction and provide support to the latch extension 5617 when the transfer member 5600 is placed in the second configuration, as described in further detail herein.

The latch extension 5617 includes the latch arm 5618 and a bendable portion 5622. The latch arm 5618 includes the guide protrusion 5619 and the latch 5620. As described above, the latch extension 5617 extends in a distal direction from the ring protrusion 5612 of the transfer member 5600. The latch arm 5618 is configured to extend from the distal end portion 5611 of the transfer member 5610. Similarly stated, the latch arm 5618 extends from a distal end portion of the latch extension 5617. Moreover, the latch arm 5618 extends from the distal end portion of the latch extension 5617 at a suitable angle such that the latch 5620 is received within the channel 5316 (see e.g., FIG. 72). For example, in some embodiments, the latch arm 5618 extends from the distal end portion of the latch extension 5617 at an acute angle. The guide protrusion 5619 is configured to engage the transfer member groove 5117, as described above.

The latch 5620 extends from a proximal end portion 5623 of the latch arm 5618. The latch 5620 is configured to engage the second latch protrusion 5317 of the latch portion 5310 of the medicament delivery mechanism 5300. As described above, the distal surface 5621 of the latch 5620 is configured to be in contact with a proximal surface 5318 of the second latch protrusion 5317 when the transfer member 5600 is in the first configuration. In this manner, the transfer member 5600 transfers a force from the actuation of the spring 5420 to the medicament delivery mechanism 5300 via the transfer member 5600 to move the medicament delivery mechanism 5300 in the distal direction within the housing 5100. Therefore, the force produced by the spring 5420 results in both the insertion of the needle 5216 and injection of the medicament 5220 within the medicament container 5200, which occur as separate and distinct operations, as described herein.

Furthermore, when the transfer member 5600 has moved a desired distance in the distal direction, in response to the force produced by the actuation of the spring 5420, the latch arm 5618 engages the transfer member release protrusion 5121 of the housing 5100 (see e.g., FIG. 67) to place the transfer member 5600 in the second configuration. Similarly stated, the latch arm 5618 engages and/or contacts the transfer member release protrusion 5121 when the transfer member 5600 is in the second position. The bendable portion 5622 of the latch extension 5617 is configured to bend, relative to the latch extension 5617. Thus, when the latch arm 5618 engages the transfer member release protrusion 5121, the bendable portion 5622 of the transfer member 5600 bends, thereby placing the transfer member 5600 in its second configuration (see FIGS. 97 and 98). When the transfer member 5600 is in its second configuration, the latch 5620 is disengaged from the second latch protrusion 5317 of the medicament delivery mechanism 5300. Said another way, when the latch arm 5618 engages the transfer member release protrusion 5121, the bendable portion 5622 of the transfer member bends such that the angle between the latch arm 5618 and the latch extension 5617 is reduced, thus disengaging the transfer member 5600 from the medicament delivery mechanism 5300. Said yet another way, when the transfer member 5600 is in its second configuration, the medicament delivery mechanism 5300 is isolated and/or no longer operably coupled to the spring 5420. In this manner, as described below, the retraction force exerted by the biasing portion 5350 moves the medicament delivery mechanism 5300 proximally within the housing 5100 to retract the needle 5216.

FIGS. 81 and 82 show the cover 5190 of the medical injector 5000. The cover 5190 includes a proximal end portion 5191 and a distal end portion 5192, and defines a cavity 5196. The cavity 5196 of the cover 5190 is configured to receive at least a portion of the housing 5100. Thus, when the portion of the housing 5100 is disposed within the cover 5190, the cover 5190 blocks an optical pathway between the medicament container 5200 and a region outside of the housing 5100. Similarly stated, when the portion of the housing 5100 is disposed within the cover 5190, the cover 5190 is obstructs the first status indicator aperture 5130 and/or the second status indicator aperture 5160 of the housing 5100 to reduce the amount of light transmitted to the medicament 5220 within the medicament container 5200. In this manner, the life of the medicament 5220 can be extended by the prevention and/or reduction of degradation to the medicament 5220 that may be caused by ultra-violet radiation.

The proximal end portion 5191 of the cover 5190 defines apertures 5193. The apertures 5193 configured to receive the cover retention protrusions 5104 of the housing 5100 (shown in FIGS. 10 and 12). In this manner, the apertures 5193 and the cover retention protrusions 5104 of the housing 5100 removably retain the cover 5190 about at least a portion of the housing 5100. Said another way, the apertures 5193 and the cover retention protrusions 5104 of the housing 5100 are configured such that the cover 5190 can be removed from a portion of the housing 5100 and then replaced about the portion of the housing 5100.

The cover 5190 can be any suitable configuration and can include any suitable feature. For example, the cover 5190 includes openings 5195 and notches 5194. In some embodiments, the openings 5195 can receive inserts (not shown). The inserts can be a flexible inserts and can be configured to increase friction between the cover 5190 and a surface. For example, the inserts can increase the friction between the cover 5190 and a surface on which the medical injector 5000 is placed, to prevent sliding. The notches 5194 are disposed at the proximal end of the cover 5190. In some embodiments, the notches 5194 can be used to reduce the material needed to manufacture the cover 5190.

FIGS. 83-87 show the safety lock 5700 of the medical injector 5000. The safety lock 5700 of the medical injector 5000 includes a proximal surface 5730, a distal surface 5740 opposite the proximal surface 5730 and a needle sheath 5810. The safety lock 5700 defines a needle sheath aperture 5703. The proximal surface 5730 of the safety lock 5700 includes two safety lock protrusions 5702, two opposing pull-tabs 5710 and an engagement portion 5720. As described above, when the safety lock 5700 is in a first (locked) position, the safety lock protrusions 5702 are configured to be disposed through the safety lock protrusion apertures 5514 defined by the base 5510 (see e.g., FIG. 88) and within the base lock openings 5131 defined by the distal end portion 5102 of the housing 5100 (see e.g., FIG. 73). Accordingly, the safety lock protrusions 5702 are configured to prevent the base locks 5515 of the base 5510 from moving past the base lock protrusion 5126 of the first housing member 5110, thereby preventing proximal movement of the base 5510 and/or delivery of the medicament 5220. Similarly stated, when the medical injector 5000 is in its first configuration (i.e., the storage configuration), the safety lock protrusions 5702 are disposed adjacent and/or in contact with the base lock protrusions 5126, thereby preventing lateral deformation (e.g., a outward flexing motion) of the base lock protrusions 5126. Thus, the arrangement of the safety lock protrusions 5702 prevents the system actuator 5500 and/or the base 5510 from moving in the proximal direction to actuate the medicament delivery mechanism 5300.

The pull-tabs 5710 of the safety lock 5700 include a grip portion 5712. The grip portion 5712 of the pull-tabs 5710 provides an area for the user to grip and/or remove the safety lock 5700 from the rest of the medicament delivery system 5700. In some embodiments, the pull-tabs 5710 can include indicia, such as, for example, an indicia similar to that included in the pull tabs 3710 of the safety lock 3700, described with reference to FIG. 43.

The engagement portion 5720 of the safety lock 5700 includes engagement members 5721. The engagement members 5721 extend in a proximal direction from the proximal surface 5730. The engagement members 5721 have tabs 5722 that extend from a surface of the engagement members 5721. The tabs 5722 are configured to engage an outer surface 5815 of a distal end portion 5812 of the needle sheath 5810.

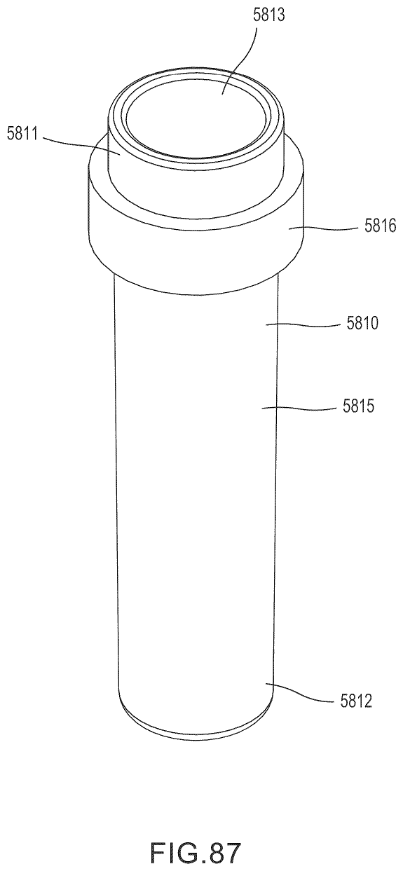

As shown in FIGS. 86 and 87, the needle sheath 5810 includes the distal end portion 5812, a proximal end portion 5811 and a rib 5816. The needle sheath 5810 further includes a contoured portion 5814 that defines a bore 5813. The bore 5813 of the needle sheath 5810 is configured to receive the needle 5216 and/or a distal end portion of the 5213 of the medicament container 5200. The contoured portion 5814 of the needle sheath 5810 defines a friction fit with the distal end portion 5213 of the medicament container 5200. In this manner, the needle sheath 5810 can protect the user from the needle 5216 and/or can keep the needle 5216 sterile before the user actuates the medical injector 5000. The proximal end portion 5811 of the needle sheath is configured to contact the body 5210 of the medicament container 5200.

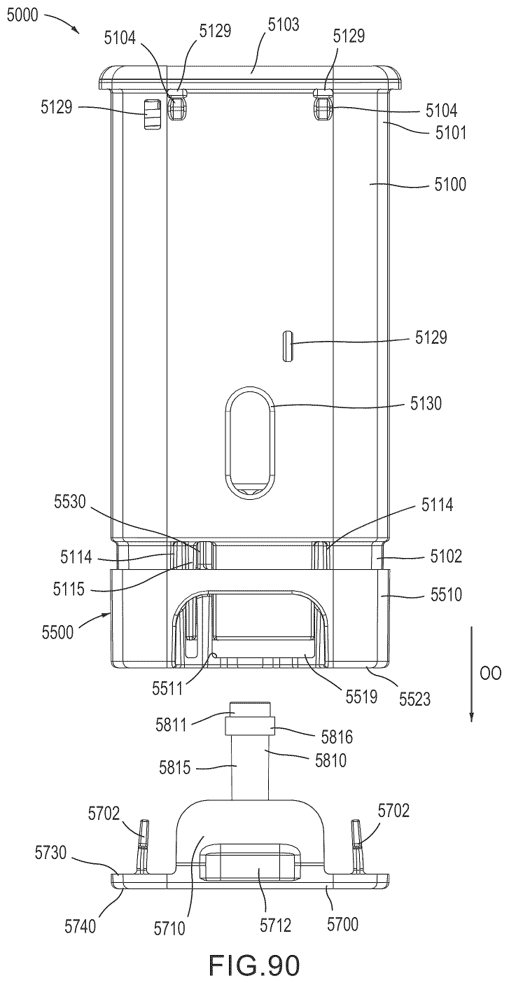

The distal end portion 5812 of the needle sheath 5810 is configured to be inserted into a space defined between the tabs 5722 of the engagement members 5721 of the safety lock 5700. The tabs 5722 are angled and/or bent towards the distal direction to allow the distal end portion 5812 of the needle sheath 5810 to move between the engagement members 5721 in a distal direction, but not in a proximal direction. Similarly stated, the tabs 5722 include an edge that contacts the outer surface 5815 of the needle sheath 5810 to prevent the safety lock 5700 from moving in a distal direction relative to the needle sheath 5810. Said another way, the needle sheath 5810 is removed from the needle 5216 when the safety lock 5700 is moved in a distal direction with respect to the housing 5100 (see e.g., FIG. 90).

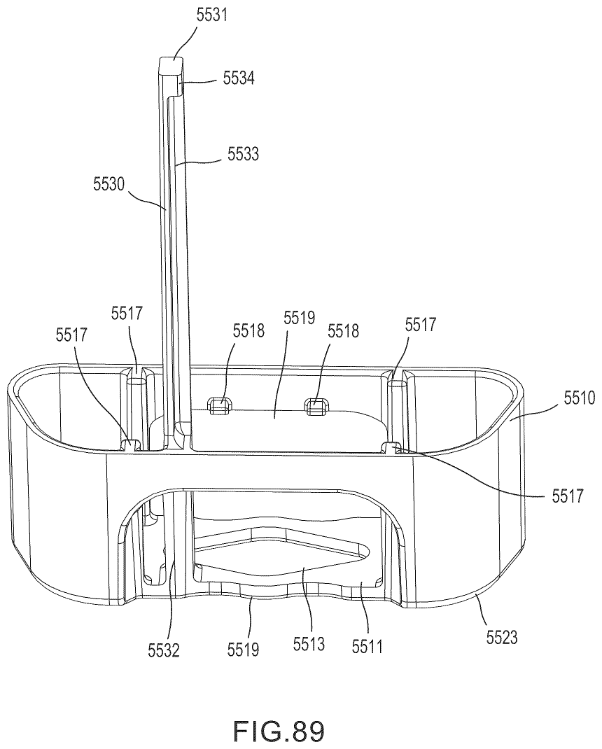

FIGS. 88 and 89 show the base 5510 (or actuator) of the medical injector 5000. The base 5510 includes the proximal surface 5511, a distal surface 5523 and base connection knobs 5518. The base 5510 defines a needle aperture 5513, safety lock protrusion apertures 5514, transfer member access opening 5516 and pull-tab openings 5519. The needle aperture 5513 is configured to receive the needle 5216 when the medical injector 5000 is actuated. The safety lock protrusion apertures 5514 of the base 5510 receive the safety lock protrusions 5702 of the safety lock 5700 when the medical injector 5000 is in the first configuration, as described above. The transfer member access opening 5516 provides access to the transfer member 5600 when the transfer member 5600 is disposed within the housing 5100. The pull-tab openings 5519 are configured to receive the pull-tabs 5710 of the safety lock 5700 when the medical injector 5000 is in the first configuration.

The proximal surface 5511 of the base 5510 includes and/or is coupled to the release member 5530, guide members 5517 and base locks 5515. The release member 5530 includes a proximal end portion 5531 and a distal end portion 5532 and defines a channel 5533 between a system lock surface 5534 and the distal end portion 5532 (see e.g., FIG. 89). As shown in FIG. 71, the system lock surface 5534 is disposed at the proximal end portion 5531 and is configured to engage the first latch protrusion 5315 of the medicament delivery mechanism 5300. Moreover, the system lock surface 5534 engages the first latch protrusion 5315 such that the system lock surface 5534 maintains the engagement of the first latch protrusion 5315 and the latch member notch 5120, as described above and shown in FIG. 72. Similarly stated, the system lock surface 5534 of the release member 5530 applies a force to the first latch protrusion 5315 to maintain the first latch protrusion 5315 within the latch member notch 5120. When the system actuator 5500 is moved in a proximal direction, as described in further detail herein, the system lock surface 5534 moves in the proximal direction to disengage the first latch protrusion 5315. In response, the first latch protrusion 5315 moves within the channel 5533 of the release member 5530 in a distal direction, as described in further detail herein. Similarly stated, upon actuation of the medicament injector 5000, a portion of the medicament delivery mechanism 5300 moves within the release member 5530.

The guide members 5517 of the base 5510 are configured to engage and/or slide within the base rail grooves 5114 of the housing 5100, as described above. The base locks 5515 of the base 5510 are configured to engage the base lock protrusions 5126 of the first housing member 5110. As described in further detail herein, when the safety lock 5700 is removed and the base 5510 is moved in a proximal direction with respect to the housing 5100, the base locks 5515 of the base 5510 are configured to disengage from the base lock protrusions 5126 and move in the proximal direction, relative to the base lock protrusions 5126. As described above, the base connection knobs 5518 are configured to engage the base retention recesses 5134A, 5134B in a way that allows proximal movement of the base 5510 but limits distal movement of the base 5510.

The medical injector 5000 is first enabled by moving the medicament delivery device 5000 from a first configuration to a second configuration by moving the cover 5190 from a first position to a second position. The cover 5190 is moved from the first position to the second position by moving it with respect to the housing 5100 in the distal direction. For example, the cover 5190 can be moved similarly to the cover 3190 of the medical injector 3000 described with reference to FIG. 49.

After the cover 5190 is removed from the housing 5100, the medical injector 5000 can be moved from the second configuration to a third configuration by moving the safety lock 5700 from a first position to a second position. The safety lock 5700 is moved from a first position to a second position by moving the safety lock 5700 with respect to the housing 5100 in the direction shown by the arrow OO in FIG. 90. Similarly stated, the medical injector 5000 can be moved from the second configuration to a third configuration by removing the safety lock 5700 from the distal end portion 5102 of the housing 5100. When the safety lock 5700 is moved from the first position to the second position, the safety lock protrusions 5702 are removed from within the base lock openings 5131 of the first housing member 5110, thereby enabling the system actuator 5500 and/or the base 5510. Similarly stated, when the safety lock 5700 is in the second position, the safety lock protrusions 5702 no longer maintain the engagement of the base locks 5515 with the base lock protrusions 5126 and/or the base locks 5515 can slide proximally relative to the base lock protrusion 5126 of the housing 5100. In this manner, the base 5510 can be moved from a first position to a second position. Moreover, with the safety lock 5700 removed, the needle sheath 5810 is removed from the medicament container 5200, as shown in FIG. 91.

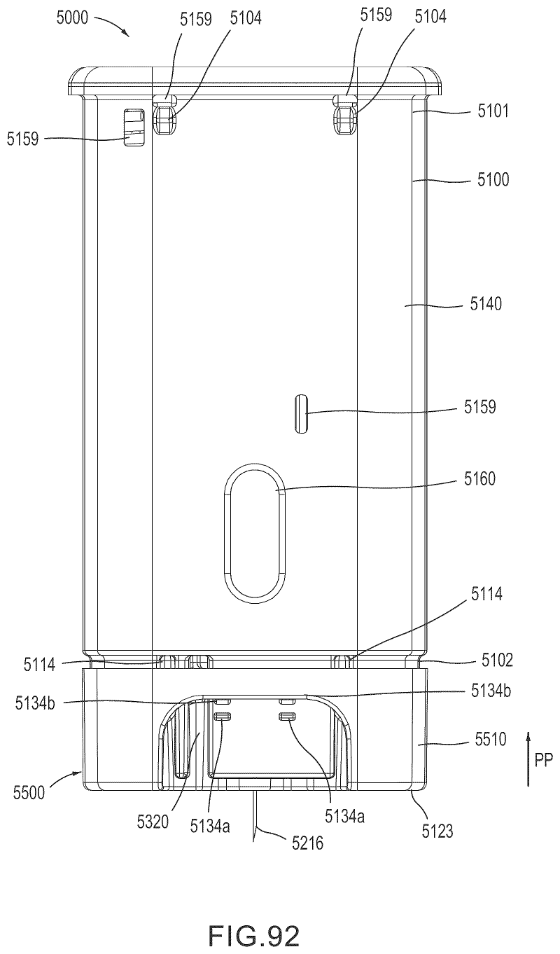

After the safety lock 5700 is moved from the first position to the second position, the medical injector 5000 can be moved from the third configuration to a fourth configuration (i.e., the needle insertion configuration) by moving the base 5510 from the first position to the second position. Similarly stated, the medical injector 5000 can be actuated by the system actuator 5500 by moving the base 5510 proximally relative to the housing 5100. The base 5510 is moved from its first position to its second position by placing the medical injector 5000 against the body of the patient and moving the base 5510 with respect to the housing 5100 in the direction shown by the arrow PP in FIG. 92. With the base locks 5515 disengaged from the base lock protrusions 5126, the system actuator 5500 can move in the proximal direction causing the base locks 5515 move proximally past the base lock protrusions 5126.

When the base 5510 is moved from the first position to the second position, the system actuator 5500 actuates the medicament delivery mechanism 5300, thereby placing the medical injector 5000 in its fourth configuration (i.e., the needle insertion configuration), as shown in FIGS. 92-94. More specifically, the proximal movement of the system actuator 5500 and/or the base 5510 moves the release member 5530 in the proximal direction within the housing 5100, thereby allowing the first latch protrusion 5315 to be disengaged from the system lock surface 5534 of the proximal end portion 5533 of the release member 5530. Similarly stated, when the system actuator 5500 is moved in the proximal direction, the system lock surface 5534 disengages the first latch protrusion 5315. Moreover, when the system lock surface 5534 moves in the proximal direction relative to the first latch protrusion 5315, the first latch protrusion 5315 moves into the channel 5533 defined by the release member 5530.

When the first latch protrusion 5315 is disposed within the channel 5533, the force applied by the system lock surface 5534 of the base 5510 to maintain the first latch protrusion 5315 within the latch member notch 5120 is removed and the first latch protrusion 5315 is allowed to disengage the latch member notch 5120. Therefore, the engagement surface 5109 of the latch member notch 5120 no longer applies the reaction force to the first latch protrusion 5315; thus, the spring 5420 is allowed to expand. As described above, the proximal end portion 5421 of the spring 5420 is in contact with the upper spring plate 5122 of the first housing member 5110 such that the spring 5420 expands in the direction shown be the arrow QQ in FIG. 93. With the distal end portion 5422 of the spring 5420 in contact with the spring seat 5615 of the transfer member 5600, a force F₄ produced by the expansion of the spring 5420 is applied to the transfer member 5600, which moves the transfer member 5600 in the direction shown by the arrow QQ. In this manner, the latch 5620 of the transfer member 5600 transfers at least a portion of the force F₄ to the second latch protrusion 5317 of the latch portion 5310 of the medicament delivery mechanism 5300 such that the portion of the force moves the medicament delivery mechanism 5300 in the distal direction, shown by the arrow QQ in FIG. 93. Thus, the medicament delivery mechanism 5300 (the first movable member) and the transfer member 5600 (the second movable member) move together distally within the housing.

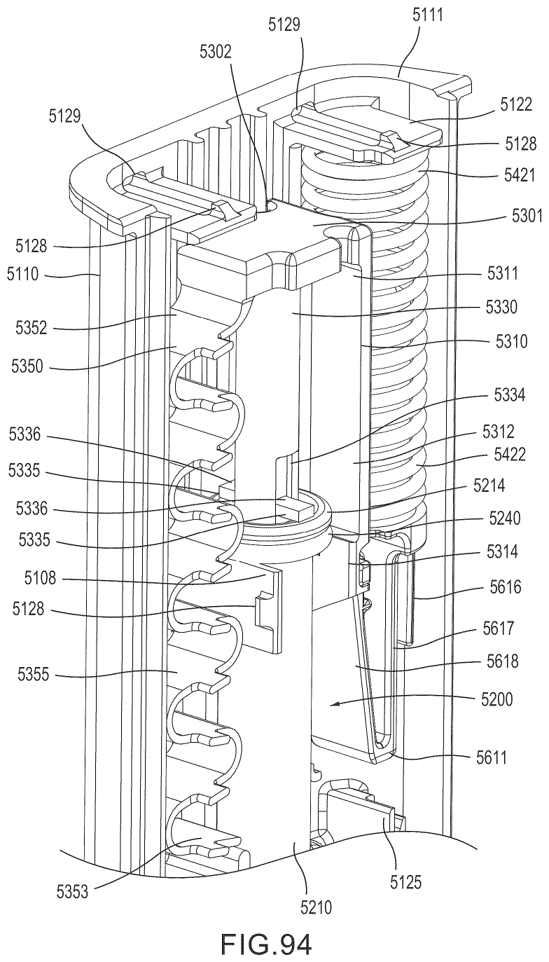

When the medicament delivery mechanism 5300 is moving distally, the piston portion 5330 of the medicament delivery mechanism 5300 applies a portion of the force F₄ to the medicament container 5200. More specifically, as shown in FIG. 94, the first shoulder 5335 of each engagement member 5336 contacts the flange 5214 of the medicament container 5200. The movement of the medicament delivery mechanism 5300 moves the piston portion 5330 in the distal direction. Therefore, with the first shoulder 5335 of each engagement member 5336 in contact with the flange 5214 of the medicament container 5200, the first shoulder 5335 transfers a portion of the force F₄ to the medicament container 5200 to move the medicament container 5200 in the distal direction. The movement of the medicament container 5200 within the housing 5100 results in the needle insertion operation.

As shown in FIG. 78, the distance between the end surface of the piston rod 5333 and the engagement members 5336 is such that when the first shoulder 5335 of each engagement member 5336 contacts the flange 5214, the distal end portion 5332 of the piston rod 5333 is spaced apart from the elastomeric member 5217 within the medicament container 5200. This arrangement prevents any portion of the force F₄ from being applied or transferred to the plunger 5217. Said another way, during the needle insertion operation (i.e., when the medical injector is being moved to its fourth configuration) the plunger 5217 is isolated from the piston portion 5330. Accordingly, this arrangement reduces and/or eliminates leakage and/or injection of medicament 5220 from the medicament container 5200 during the needle insertion operation.

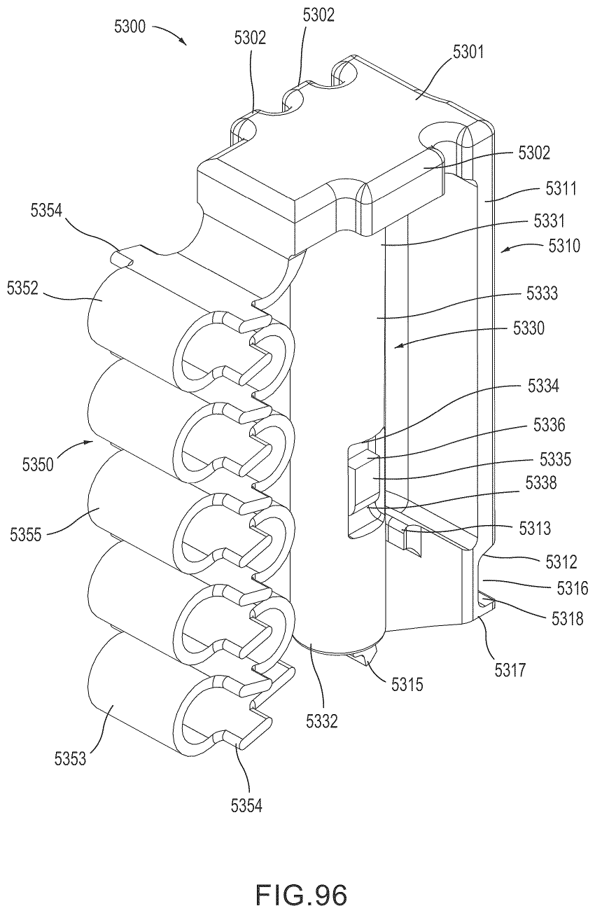

After the transfer member 5600, the medicament delivery mechanism 5300 and the medicament container 5200 move in the distal direction a given distance, the damping member 5240 of the medicament container 5200 contacts the proximal surface 5108 of the medicament container holder 5127 and 5157 of the first housing portion 5110 and the second housing portion 5140, respectively. The proximal surface 5108 prevents the medicament container 5200 from moving further in the distal direction. Thus, when the flange 5214 and/or the damping member 5240 contact the proximal surface 5108, the needle 5216 is fully inserted into the target location of a patient. At this point, the medical injector 5000 can be moved from the fourth configuration to the fifth configuration (i.e., the medicament delivery configuration), shown in FIGS. 95 and 96.

When the damping member 5240 of the medicament container 5200 is in contact with the proximal surface 5108 of the medicament container holders 5127 and 5157, the medicament container 5200 is prevented from moving in the distal direction. The portion of the force F₄ applied by the spring 5420, however, continues to urge the transfer member 5600 and the medicament delivery mechanism 5300 in the direction shown by the arrow RR in FIG. 95. More specifically, when the medicament container 5200 is in contact with the medicament container holders 5127 and 5157, the force F₄ applied by the spring 5420 moves the transfer member 5600 and the medicament delivery mechanism 5300 in the distal direction, relative to the medicament container 5200. In this manner, the portion of the force F₄ applied to the medicament delivery mechanism 5300 causes the deformable portion 5338 of the engagement members 5336 to deform and/or bend inward (see e.g., FIG. 96). Similarly stated, the deformable portion 5338 of each of the engagement members 5336 is configured to deform when the damping member 5240 of the medicament container 5200 is in contact with the proximal surface 5108 of the medicament container holders 5127 and 5157. When the deformable portion 5338 is deformed, the engagement members 5336 are disposed within the recesses 5334 defined by the piston rod 5333 (see e.g., FIG. 96). In this manner, the piston rod 5333 is configured to move within the medicament container 5200 into contact with the elastomeric member 5217 to deliver the medicament 5220. Similarly stated, the piston portion 5330 is moved from its first configuration, in which the engagement members 5336 collectively have a size that is greater than the size (i.e., diameter) of the inner bore of the medicament container 5200 to its second configuration, in which the engagement members 5336 collectively have a size that is less than the size (i.e., diameter) of the inner bore of the medicament container 5200. This decrease in size (or diameter) allows the piston rod 5333 to move within the medicament container 5200.

When the medicament delivery mechanism 5300 moves in the distal direction to move the elastomeric member 5217 and inject the medicament 5220, the serpentine portion 5355 and/or the bias portion 5350 is also compressed. More specifically, a portion of the force F₄ compresses the serpentine portion 5355 and/or the bias portion 5350 between the proximal end portion 5301 of the medicament delivery mechanism 5300 and the lower bias plate 5124. Similarly stated, the bias portion 5350 is configured to compress as the serpentine portion 5355 elastically deforms (e.g., bending, squeezing, or compressing such that the bias portion 5350 returns to a non-deformed configuration when the deforming force is removed). In this manner, the space defined between adjacent portions of the serpentine portion 5355 is reduced.

As the spring 5420 fully expands, the medicament delivery mechanism 5300 moves in the distal direction to fully inject the medicament 5220 within the medicament container 5200 through the needle 5216. Additionally, when the spring 5420 is fully expanded and/or when the medicament delivery mechanism 5300 has moved a desired distance within the housing 5100, the latch arm 5618 of the transfer member 5600 engages the transfer member release protrusion 5121 of the housing 5100. As described above, the transfer member release protrusion 5121 contacts the latch arm 5618 of the transfer member 5600 such that the bendable portion 5622 disposed at the distal end of the latch extension 5617 bends. In this manner, the latch 5620 of the latch arm 5618 is disengaged from the second latch protrusion 5318 of the latch portion 5310 of the medicament delivery mechanism 5300 (see e.g., FIGS. 97 and 98). Similarly stated, the spring 5240 and/or the transfer member 5600 are decoupled from the medicament delivery mechanism 5300. With the latch arm 5618 disengaged from the latch portion 5310, the medical injector 5000 can be moved from the fifth configuration to the sixth configuration (i.e., the retraction configuration).

As shown in FIG. 98, the transfer mechanism 5600 is deformed such that the transfer member 5600 and/or the spring 5420 are no longer engaged with the medicament delivery mechanism 5300. Therefore, the medicament delivery mechanism 5300 is configured to move within the housing 5100 in the direction shown by the arrow SS in FIG. 97 in response to the force produced by the bias portion 5350. Similarly stated, with the medicament delivery mechanism 5300 disengaged from the transfer member 5600 and/or the spring 5420, the force F₄ is no longer applied to the medicament delivery mechanism 5300. In this manner, the bias portion 5350 is configured to expand in the direction of the arrow SS shown in FIG. 97 to apply a retraction force to the medicament delivery mechanism 5300. Similarly stated, with the portion of the force F₄ configured to compress the bias portion 5350 removed, the bias portion 5350 expands, returning to its uncompressed (i.e., non-deformed) configuration.

During the retraction operation, the second shoulder 5313 included in the latch portion 5310 is configured to engage a distal surface of the damping member 5240 and/or the flange 5214. The second shoulder 5313 is further configured to transmit the retraction force produced by the expansion of the bias portion 5350 to the flange 5214, thereby moving the medicament container 5200 proximally. Similarly stated, the medicament container 5200 is moved in the proximal direction towards the first position of the medicament container 5200. This motion, removes the needle 5216 from the target location of the patient and retracts the needle into the housing 5100, as shown in FIG. 97.

While various embodiments of the invention have been described above, it should be understood that they have been presented by way of example only, and not limitation. Where methods described above indicate certain events occurring in certain order, the ordering of certain events may be modified. Additionally, certain of the events may be performed concurrently in a parallel process when possible, as well as performed sequentially as described above.

Although the first surface 3341 of the piston member 3330 is shown as being substantially parallel to the second surface 3342 of the piston member 3330, in other embodiments, the first surface of a movable member can be at any suitable angular orientation to a second surface of the movable member.

Although the carrier 3370 is shown as substantially surrounding the medicament container 3200, in other embodiments, a carrier and/or the contact shoulders (analogous to the first shoulder 3377 and the second shoulder 3381) need not substantially surround the medicament container 3200. For example, in some embodiments, a carrier can be a single piece member that only partially surrounds the flange 3214 of the medicament container 3200. Similarly stated, in some embodiments, a carrier need not be movable between an opened configuration and a closed configuration, but rather can receive and/or retain the medicament container in a single configuration.

Although the carrier 4370 is described above as being configured to accommodate an o-ring or other suitable damping member to reduce the forces exerted on the medicament container 4200 during insertion and/or injection, in other embodiments, any suitable mechanisms or structures for reducing the energy, impulse and/or forces applied to the carrier, the medicament container, the housing and/or the actuation member can be employed. For example, in some embodiments, a carrier can include a deformable portion (e.g., a “crush rib”) configured to deform when contacting the housing during an insertion event. In this manner, the deformable portion can absorb at least a portion of the energy and/or force generated during the impact, thereby reducing the magnitude of the energy, impulse and/or force applied to the medicament container. Similarly, in some embodiments, a portion of a medicament delivery mechanism, such as medicament delivery mechanism 4300 can include a crush rib or an impact portion configured to plastically and/or elastically deform to absorb and/or dampen the forces from the needle insertion operation.

In some embodiments, the outer surface 3815 of the needle sheath 3810 can include a cap or cover that has different material properties than the remainder of the needle sheath 3810. For example, in some embodiments, the outer surface 3815 can be constructed of a material having greater hardness and/or rigidity than the remainder of the needle sheath 3810. This arrangement allows for sufficient structural rigidity to assembly the needle sheath 3810 within the engagement portion 3720 of the safety lock 3700. In other embodiments, however, any of the needle sheaths described herein need not include an outer cover or cap. The use of a cap-less design can reduce manufacturing and/or assembly costs.

Although the medical injector 3000 is shown above as including a gas container 3410 that is actuated by a puncturer that moves within the housing 3100 with the release member 3550, in other embodiments a system actuation assembly 3500 can include a puncturer that is substantially fixed within the housing and a gas container that moves within the housing into contact with the puncturer upon actuation of the device.

Although the medicament delivery mechanism 5300 is shown above as being a monolithically constructed member (i.e., a “first movable member”), in other embodiments, the medicament delivery mechanism 5300 can include multiple members that are separately constructed and/or that are coupled together. For example, in some embodiments, a medicament delivery mechanism can include a first member that corresponds to the latch portion 5310 and the piston portion 5330, and a second, separately constructed member that produces a retraction force (e.g., similar to the function of the bias portion 5350. In such embodiments, for example, second member can be a separately constructed coil spring or the like.

Any of the devices and/or medicament containers shown and described herein can be constructed from any suitable material. Such materials include glass, plastic (including thermoplastics such as cyclic olefin copolymers), or any other material used in the manufacture of prefilled syringes containing medications.

Any of the devices and/or medicament containers shown and described herein can include any suitable medicament or therapeutic agent. In some embodiments, the medicament contained within any of the medicament containers shown herein can be a vaccine, such as, for example, an influenza A vaccine, an influenza B vaccine, an influenza A (H1N1) vaccine, a hepatitis A vaccine, a hepatitis B vaccine, a haemophilus influenza Type B (HiB) vaccine, a measles vaccine, a mumps vaccine, a rubella vaccine, a polio vaccine, a human papilloma virus (HPV) vaccine, a tetanus vaccine, a diphtheria vaccine, a pertussis vaccine, a bubonic plague vaccine, a yellow fever vaccine, a cholera vaccine, a malaria vaccine, a smallpox vaccine, a pneumococcal vaccine, a rotavirus vaccine, a varicella vaccine, a rabies vaccine and/or a meningococcus vaccine. In other embodiments, the medicament contained within any of the medicament containers shown herein can be a catecholamine, such as epinephrine. In other embodiments, the medicament contained within any of the medicament containers shown herein can be an opioid receptor antagonist, such as naloxone, including any of the naloxone formulations described in U.S. Pat. No. 8,627,816 entitled, “Medicament Delivery Device for Administration of Opioid Antagonists Including Formulation for Naloxone,” filed on Feb. 28, 2011. In yet other embodiments, the medicament contained within any of the medicament containers shown herein can include peptide hormones such as insulin and glucagon, human growth hormone (HGH), erythropoiesis-stimulating agents (ESA) such as darbepoetin alfa, monoclonal antibodies such as denosumab and adalimumab, interferons, etanercept, pegfilgrastim, and other chronic therapies, or the like. In yet other embodiments, the medicament contained within any of the medicament containers shown herein can be a placebo substance (i.e., a substance with no active ingredients), such as water.

Although the medical injector 3000 includes the electronic circuit system cavity 3153, the gas cavity 3154 and/or the medicament cavity 3157 that are shown and described as being fluidically and/or physically isolated from each other, in other embodiments, any of the electronic circuit system cavity 3153, the gas cavity 3154 and/or the medicament cavity 3157 can be fluidically coupled to and/or share a common boundary with each other. In some embodiments, for example, a housing can define a single cavity within which a medicament container, an energy storage member and an electronic circuit system are disposed.

The medicament containers and/or medicament delivery devices disclosed herein can contain any suitable amount of any medicament. For example, in some embodiments, a medicament delivery device as shown herein can be a single-dose device containing an amount medicament to be delivered of approximately 0.4 mg, 0.8 mg, 1 mg, 1.6 mg or 2 mg. As described above, the fill volume can be such that the ratio of the delivery volume to the fill volume is any suitable value (e.g., 0.4, 0.6 or the like). In some embodiments, an electronic circuit system can include “configuration switch” (similar to the configuration switch 3974 shown and described above) that, when actuated during the assembly of the delivery device, can select an electronic output corresponding to the dose contained within the medicament container.

Although the electronic circuit system 3900 is shown and described above as having two irreversible switches (e.g., switch 3972 and switch 3973), in other embodiments, an electronic circuit system can have any number of switches. Such switches can be either reversible or irreversible.

Although the electronic circuit system 3900 is shown and described above as producing an electronic output in response to the actuation of two switches (e.g., switch 3972 and switch 3973), in other embodiments, an electronic circuit system can produce an electronic output in response to any suitable input, command or prompt. Suitable input for prompting an output can include, for example, an audible input by the user (e.g., the user's response to a voice prompt produced by the electronic circuit system), an input from a “start button” depressed by the user, an input from a sensor (e.g., a proximity sensor, a temperature sensor or the like), movement of (e.g., shaking) of the medicament delivery device, or the like. In some embodiments, an electronic circuit system can include a microphone and/or a voice recognition module to detect a user's vocal input.

Although medical devices having two LEDs and an audio output device have been shown, in other embodiments the medical device might have any number of LEDs and/or audio output devices. Additionally, other types of output devices, such as haptic output devices, can be used. In some embodiments, outputs from an electronic circuit system can include, for example, an audible or visual output related to the composition of the medicament (e.g., an indication of the expiration date, the symptoms requiring treatment with the medicament or the like), the use of the medicament delivery device, and/or post-administration procedures (e.g., a prompt to call 911, instructions for the disposal of the device or the like).

Any of the medicament delivery devices shown and described herein can include any of the electronic circuit systems shown and described herein. For example, although the medical injector 5000 is shown as being devoid of an electronic circuit system, in other embodiments, the medical injector 5000 can include an electronic circuit system similar to the electronic circuit system 3900 shown and described above with reference to FIGS. 29-39. Moreover, although the electronic circuit systems (e.g., the electronic circuit system 3900) are shown and described herein as being coupled the housing of the medicament delivery device, in other embodiments, all or a portion of an electronic circuit system can be coupled to a removable cover (e.g., cover 3190). For example, in some embodiments, the cover can include an electronic circuit system (the “master ECS”) including an audible output device, and the electronic circuit system can be configured to receive on or more signals from an electronic circuit system (the “slave ECS”) coupled to the medicament delivery device. In this manner, the master ECS can receive indications of when the safety tab has been removed, when the device has been actuated or the like, and can produce an audible output as described herein. In some such embodiments, the master ECS and the slave ECS can be similar to the electronic circuit systems shown and described in U.S. Pat. No. 8,172,082 entitled, “Devices, Systems and Methods for Medicament Delivery,” filed on Feb. 5, 2007, which is incorporated herein by reference in its entirety.

Although the electronic circuit system 3900 is shown and described above as producing an electronic output in response to the removal of the safety lock 3700 and/or movement of the base 3510, in other embodiments, any suitable component within a medicament delivery device can function to actuate the electronic circuit system. For example, in some embodiments, a carrier (similar to the carrier 3370) can include a protrusion configured to engage a portion of an electronic circuit system such that the electronic circuit system produces an output in response to movement of the carrier. In other embodiments, an electronic circuit system can produce an electronic output in response to the deformation of a portion of a movable member (e.g., the engagement portion 3379 of the carrier 3370). In such embodiments, the deformable portion may be configured to engage a portion of the electronic circuit system or may be configured such that a portion of the electronic circuit system is disposed therein (e.g., a copper trace) to activate the electronic circuit system.

In some embodiments, the electronic circuit system 3900 of the types shown and described herein can be used in either an actual medicament delivery device or a simulated medicament delivery device. A simulated medicament delivery device can, for example, correspond to an actual medicament delivery device and can be used, for example, to train a user in the operation of the corresponding actual medicament delivery device.

The simulated medicament delivery device can simulate the actual medicament delivery device in any number of ways. For example, in some embodiments, the simulated medicament delivery device can have a shape corresponding to a shape of the actual medicament delivery device, a size corresponding to a size of the actual medicament delivery device and/or a weight corresponding to a weight of the actual medicament delivery device. Moreover, in some embodiments, the simulated medicament delivery device can include components that correspond to the components of the actual medicament delivery device. In this manner, the simulated medicament delivery device can simulate the look, feel and sounds of the actual medicament delivery device. For example, in some embodiments, the simulated medicament delivery device can include external components (e.g., a housing, a needle guard, a sterile cover, a safety lock or the like) that correspond to external components of the actual medicament delivery device. In some embodiments, the simulated medicament delivery device can include internal components (e.g., an actuation mechanism, a compressed gas source, a medicament container or the like) that correspond to internal components of the actual medicament delivery device.

In some embodiments, however, the simulated medicament delivery device can be devoid of a medicament and/or those components that cause the medicament to be delivered (e.g., a needle, a nozzle or the like). In this manner, the simulated medicament delivery device can be used to train a user in the use of the actual medicament delivery device without exposing the user to a needle and/or a medicament. Moreover, the simulated medicament delivery device can have features to identify it as a training device to prevent a user from mistakenly believing that the simulated medicament delivery device can be used to deliver a medicament. For example, in some embodiments, the simulated medicament delivery device can be of a different color than a corresponding actual medicament delivery device. Similarly, in some embodiments, the simulated medicament delivery device can include a label clearly identifying it as a training device.

Although various embodiments have been described as having particular features and/or combinations of components, other embodiments are possible having a combination of any features and/or components from any of embodiments where appropriate. For example, any of the devices shown and described herein can include an electronic circuit system as described herein. For example, although the medicament delivery device 4000 shown in FIGS. 56 and 57 is not shown as including an electronic circuit system, in other embodiments, a medicament delivery device similar to the device 4000 can include an electronic circuit system similar to the electronic circuit system 3900 shown and described above.

Any of the medicament containers described herein can include any of the elastomeric members described herein. For example, the medicament container 5200 can include an elastomeric member 5217 that is formulated to be compatible with the medicament contained therein. Although the medical injector 5000 includes a single elastomeric member 5217, in other embodiments, any number of elastomeric members 5217 can be disposed within the medicament container 5200. For example, in some embodiments, a medicament container can include a dry portion of a medicament and a fluid portion of the medicament, configured to be mixed before injection. The piston portion 5330 of the medicament delivery mechanism 5300 can be configured to engage multiple elastomeric members 5217 associated with the portions of the medicament. In this manner, multiple elastomeric members 5217 can be engaged to mix the dry portion with the fluid portion of the medicament before the completion of an injection event. In some embodiments, for example, any of the devices shown and described herein can include a mixing actuator similar to the mixing actuators shown and described in U.S. Patent Publication No. 2013/0023822 entitled, “Devices and Methods for Delivering Medicaments from a Multi-Chamber Container,” filed Jan. 25, 2012, which is incorporated herein by reference in its entirety.

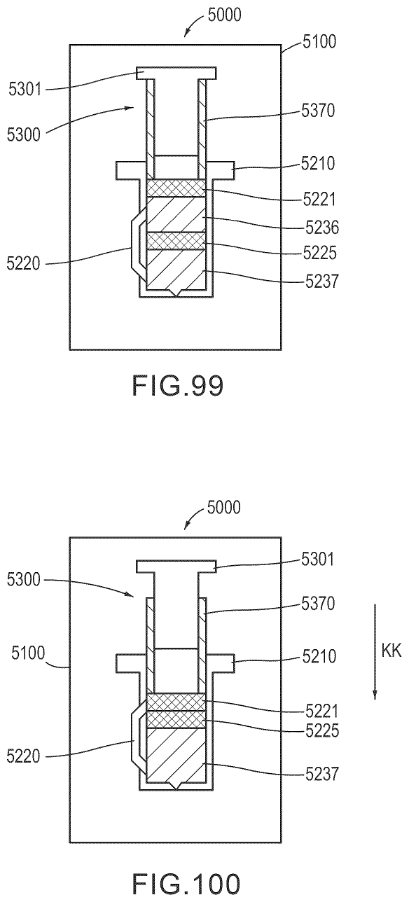

While the medicament containers described above include a single plunger, in some embodiments, any of the medicament containers described herein can include any number of plungers and/or can define multiple volumes therein that contain different medicament constituents. For example, as shown in FIGS. 99-102, a medicament delivery device 5000 includes a housing 5100, a medicament container 5210, and a movable assembly 5300. The housing 5100 can be any suitable size, shape, or configuration and can be made of any suitable material. For example, in some embodiments, the housing 5100 is an assembly of multiple parts formed from a plastic material and defines a substantially rectangular shape when assembled.

The medicament container 5210 is disposed within the housing 5100, and includes a first plunger 5221, a second plunger 5225, and a bypass 5220. The medicament container 5210 defines a first volume 5236, and a second volume 5237. Expanding further, the first volume 5236 is defined between a distal end surface of the first plunger 5221, a portion of the medicament container 5120 and a proximal end surface of the second plunger 5225, and can contain a first substance, such as any suitable diluent, as described in further detail herein. Similarly, the second volume 5236 is defined between a distal end surface of the second plunger a distal end portion of the medicament container 5210, and can contain a second substance, such as any suitable medicament (e.g., a lyophilized medicament). In this manner, the diluent contained within the first volume 5236 can be stored separately from with the medicament within the second volume 5237. Upon actuation the diluent can be mixed with the medicament such that the combination of the diluents and the medicament reconstitute the medicament for delivery into, for example, the body of a patient.

The movable assembly 5300 includes a first movable member 5301 and a second movable member 5370, and is configured to move between a first configuration, a second configuration, and a third configuration. The first movable member 5301 and the second movable member 5370 are movably coupled such that the second movable member 5370 can move with and/or relative to the first movable member 5301. As shown, in some embodiments, the second movable member 5370 can substantially surround the first movable member 5301. In some embodiments, the second movable member 5370 can define a substantially annular and/or cylindrical shape such that at least a portion of the first movable member 5301 is disposed therein.

As shown in FIG. 99, the second movable member 5370 engages the first plunger 5221 disposed within the medicament container 5210 and when the movable assembly 5300 is in the first configuration (FIG. 99). In other embodiments, the second movable member 5370 can be spaced apart from the plunger 5221 when the movable assembly 5300 is in the first configuration. The second movable member 5370 can be moved, relative to the first movable member 5301 to move the movable assembly 5300 from the first configuration to the second configuration. For example, in some embodiments, the second movable member 5370 can be moved by a force exerted by an energy storage member (e.g., such as those described herein). When the second movable member 5370 moves relative to the first movable member 5301, a distal end portion of the second movable member 5370 moves the first plunger 5221 in the distal direction within the medicament container 5210, as shown by the arrow KK in FIG. 100. The distal motion of the plunger 5221 can facilitate, for example, a mixing of diluents and the medicament contained within the medicament container 5210. For example, in some embodiments, the distal movement of the first plunger 5221 can cause the second plunger 5225 to move past the bypass 5220 and urge the diluent, contained within the first volume 5236 to move within the bypass 5220 and enter the second volume 5237.

The bypass 5220 can be any suitable bypass (external or internal) configured to define a pathway between the first volume 5236 and the second volume 5237. In some embodiments, the bypass 5220 can include a one way valve such that when a pressure within the first volume 5236 increases (e.g., as induced by the distal movement of the first plunger 5221), the one way valve opens to allow a flow of the diluent through the bypass 5220 to the mixing volume 5237. In other embodiments, the bypass 5220 can include a frangible seal configured to break under the increase pressure. In this manner, when first plunger 5221 is moved, the first volume 5236 is reduced and the distal end surface of the first plunger 5221 can contact the proximal end surface of the second plunger 5255. Accordingly, as the volume defined by the first volume 5236 is reduced, the volume of the second volume 5237 increases. In this manner, the distal end surface of the first plunger 5221 contacts the proximal end surface of the second plunger 5225 at a position within the medicament container 5210 such that the first plunger 5221 of the second plunger 5225 substantially seals an opening of the bypass 5220, thereby preventing potential backflow.

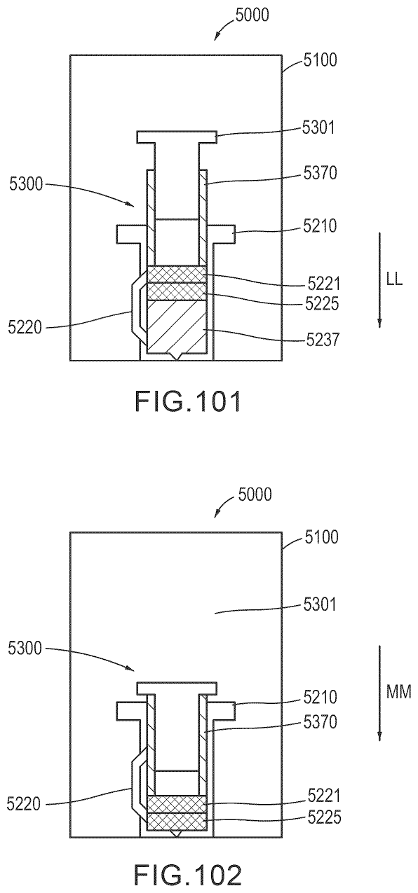

The movable assembly 5300 is configured to move from a first position (e.g., FIG. 99) to a second position within the housing 5100, as shown by the arrow LL in FIG. 101. In some embodiments, the movable assembly 5300 can move in the direction LL (e.g., the distal direction) in response to a portion of a force exerted, for example, by the energy storage member (described above). The distal movement of the movable assembly 5300 moves the medicament container 5210 within the housing 5100 from a first container position (e.g., FIG. 100) to a second container position (e.g., FIG. 101). In some embodiments, the distal movement (e.g., in the direction of the arrow LL shown in FIG. 101) can facilitate the insertion of a needle (not shown in FIGS. 99-102), disposed at the distal end portion of the medicament container 5210, into a target location (e.g., the body of a patient).

When the medicament container 5210 is in the second container position within the housing 5100, the first movable member 5301 moves distally to engage the second movable member 5370. In this manner, the first movable member 5301 and the second movable member 5370 can move together in the distal direction, as shown by the arrow MM in FIG. 102. Thus, the movable assembly 5300 moves in the distal direction and moves the first plunger 5221 and the second plunger 5225 within the medicament container 5210 such that the medicament disposed within the second volume 5237 is delivered to a volume substantially outside the medicament container 5210 (e.g., into the body of the patient via the needle).

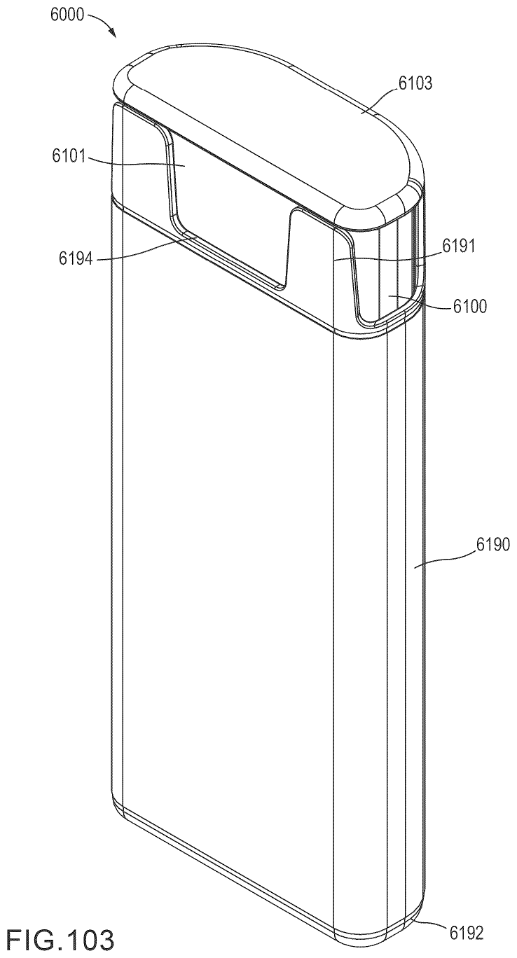

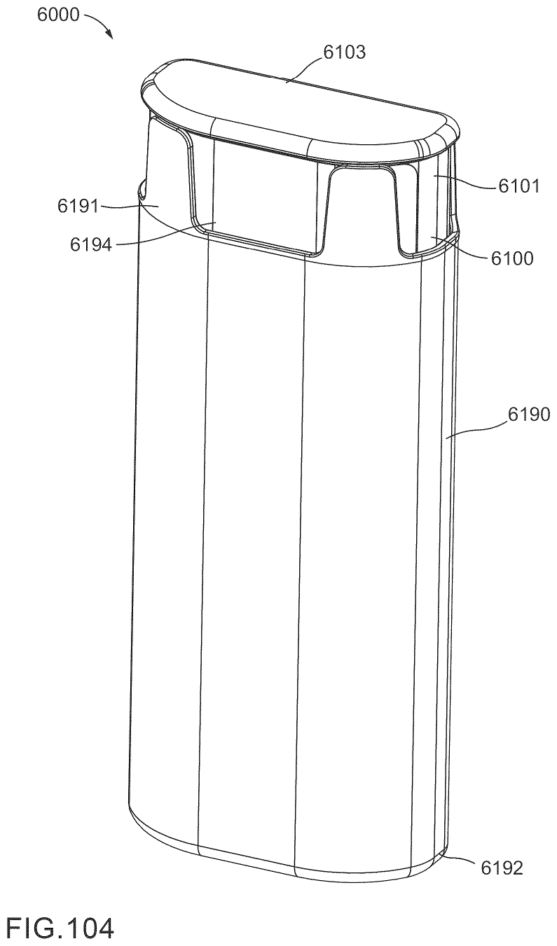

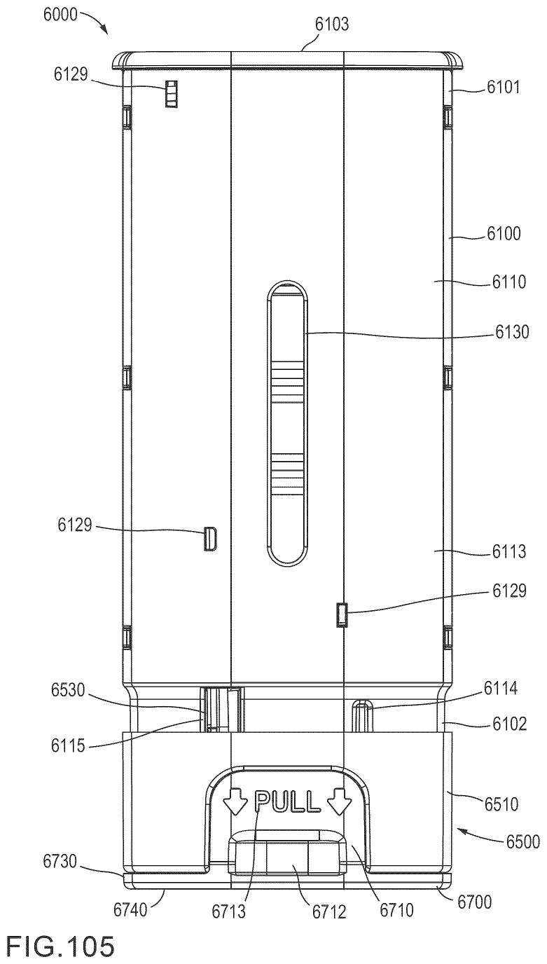

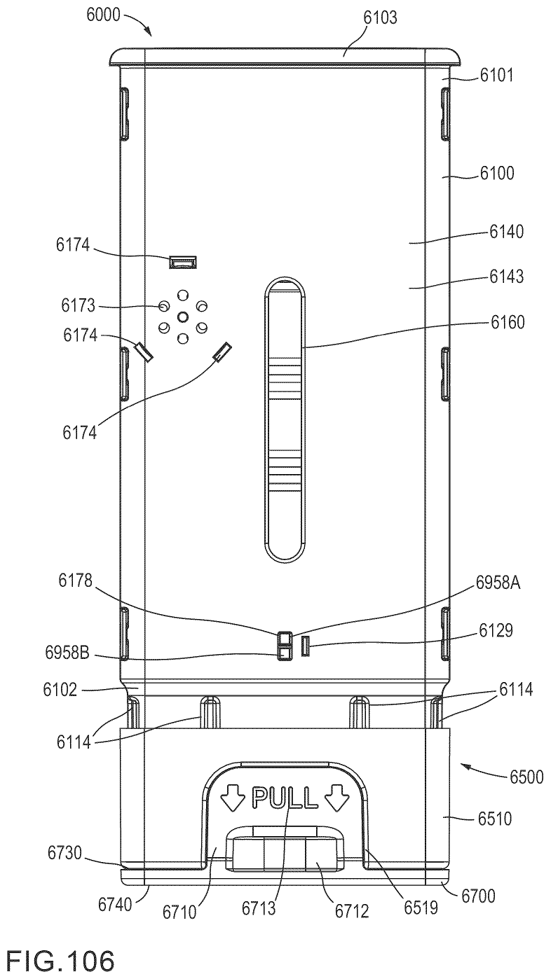

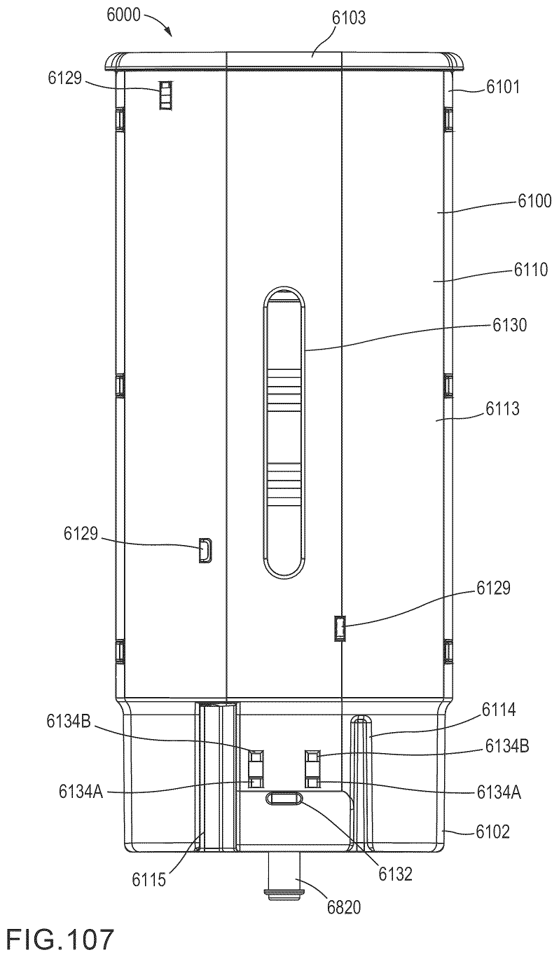

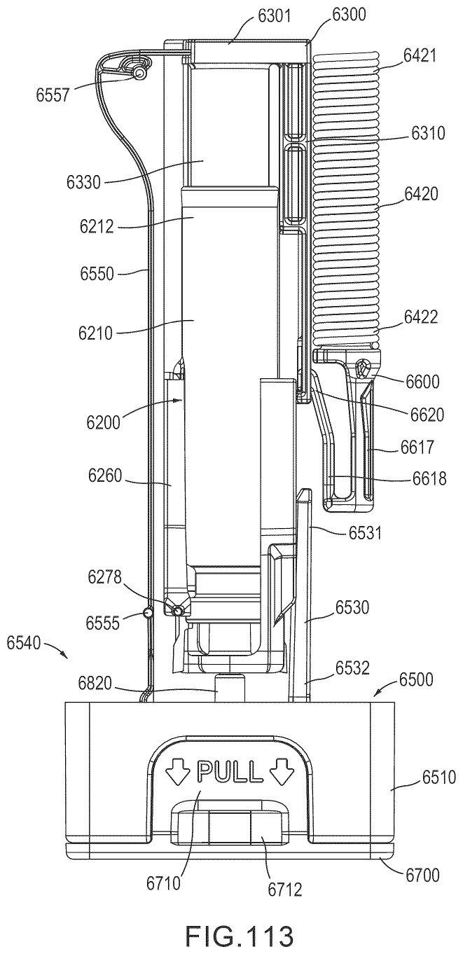

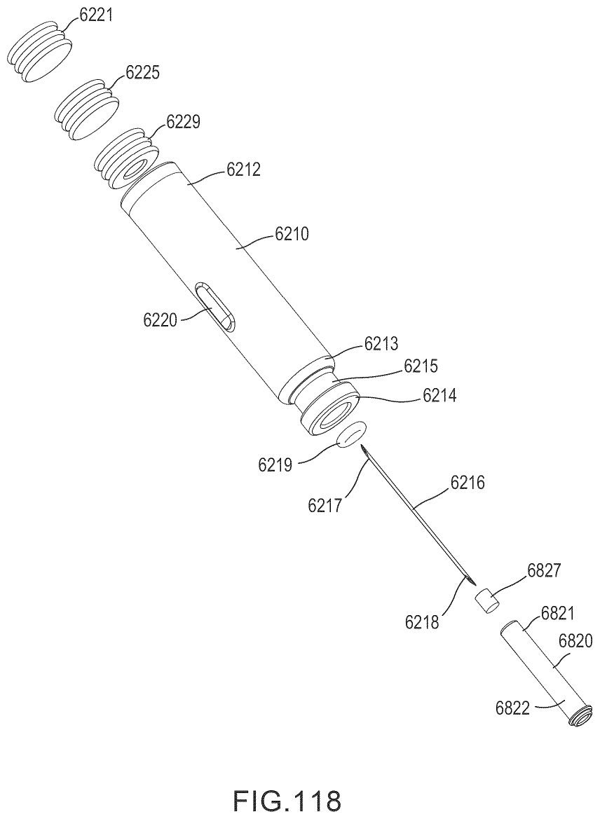

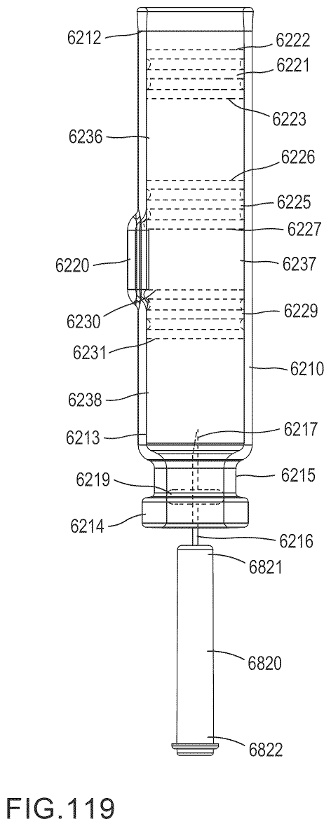

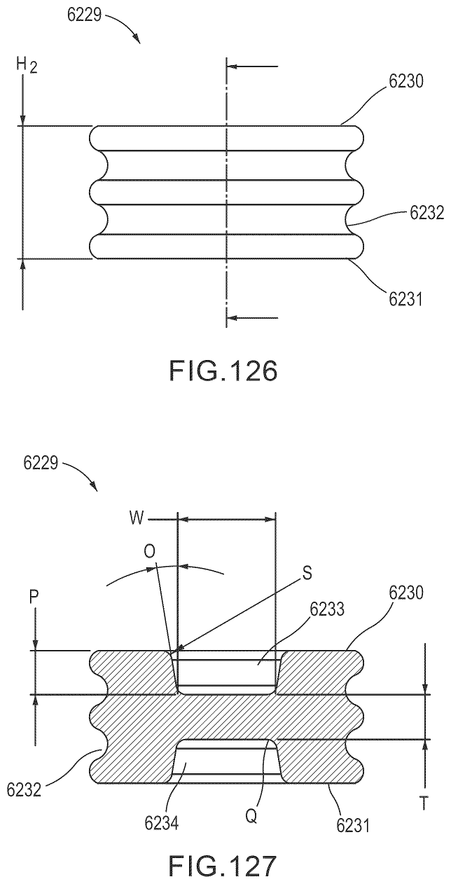

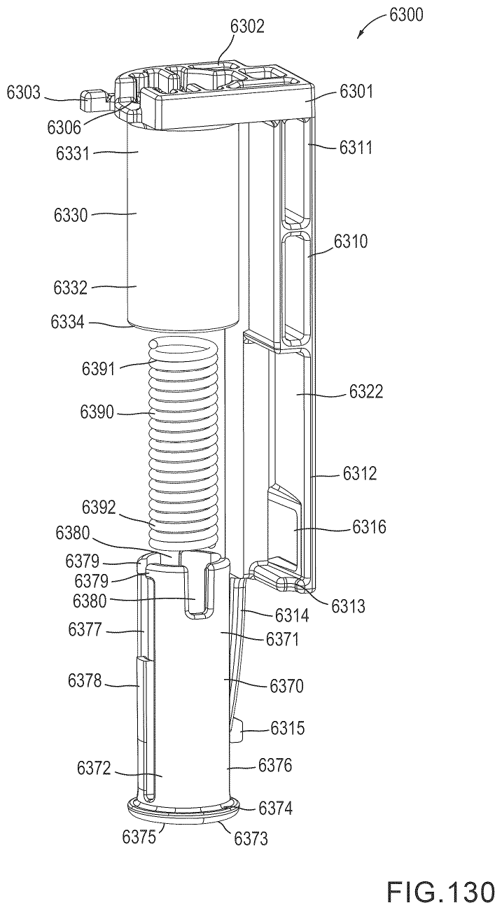

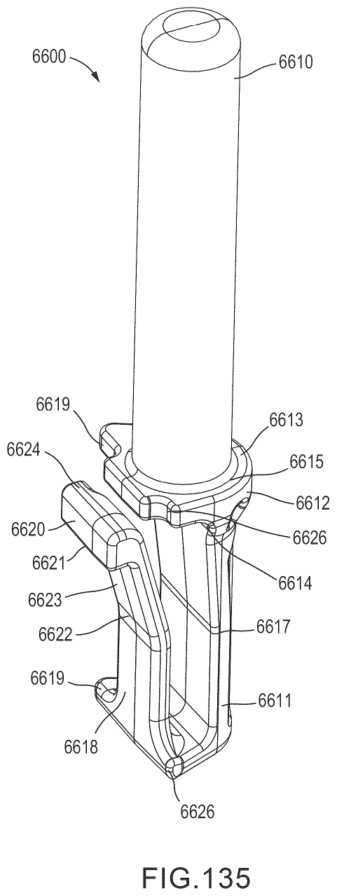

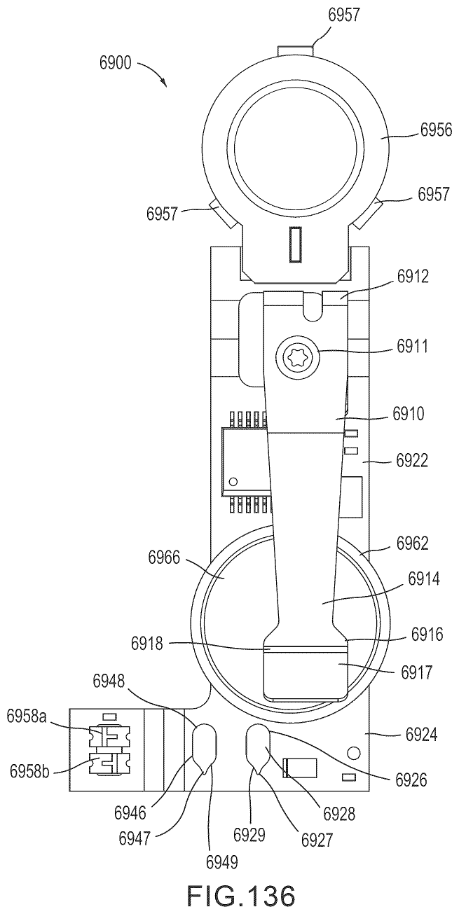

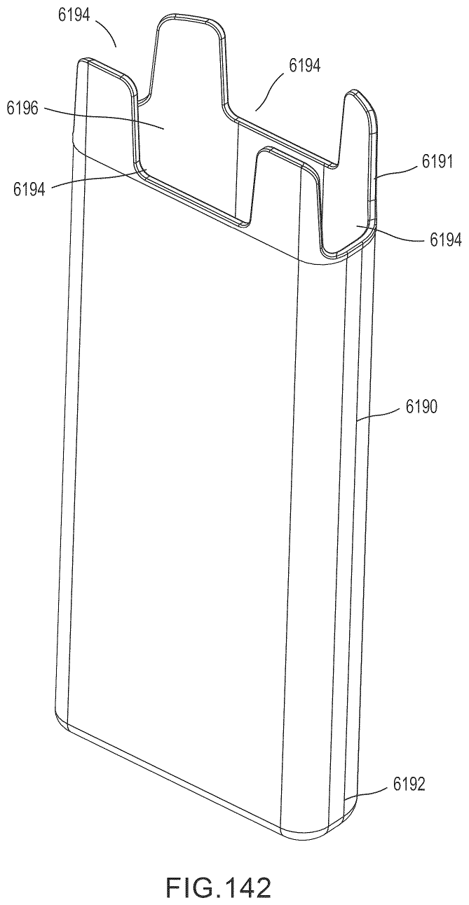

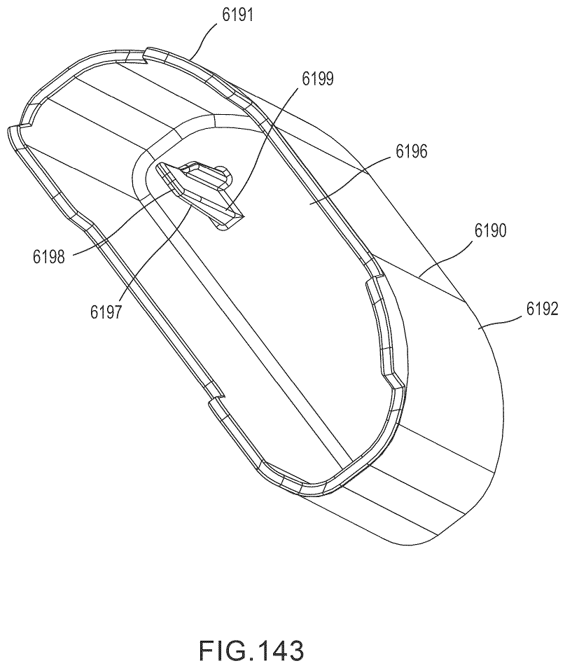

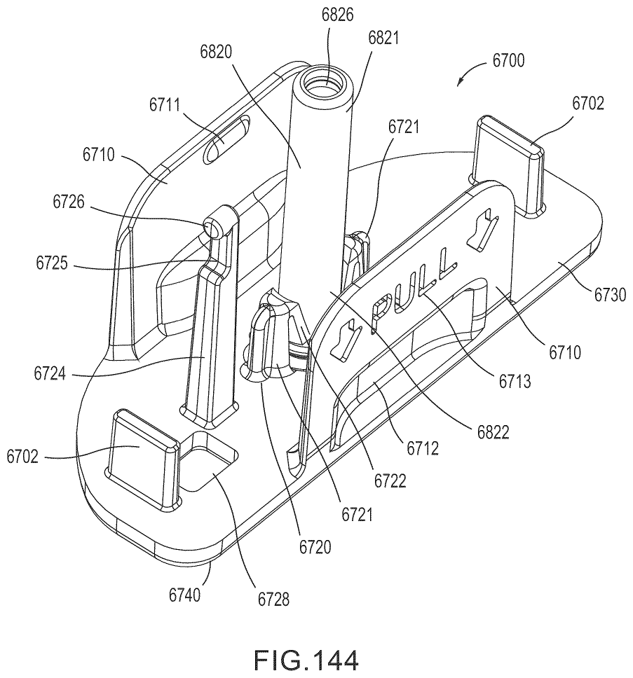

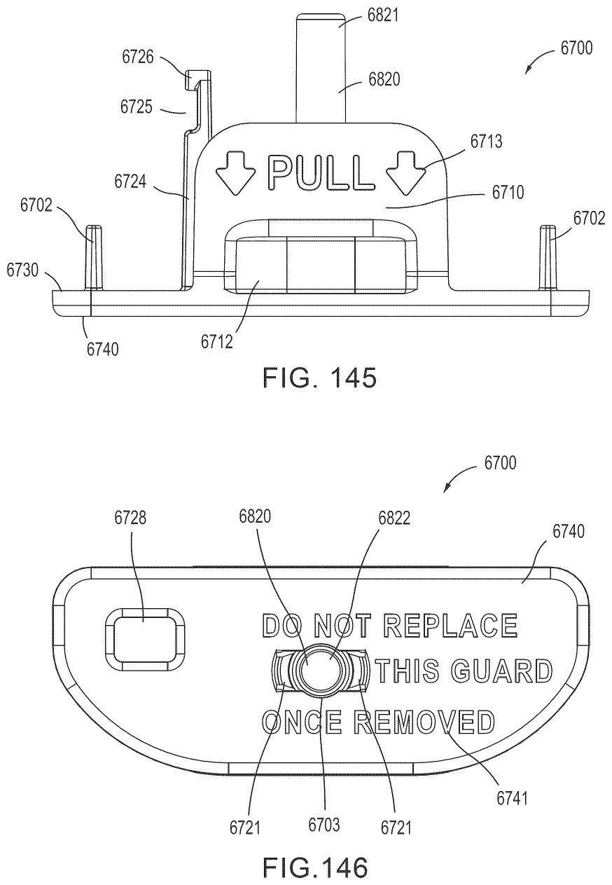

In some embodiments, the medicament delivery device can be a medical injector configured to automatically mix and deliver a medicament contained within a medicament container. For example, FIGS. 103-161 show various views of a medical injector 6000, according to an embodiment in various different configurations (or stages of operation). FIGS. 103-104 are perspective views of the medical injector 6000 in a first configuration (i.e., prior to use). The medical injector 6000 includes a housing 6100 (see e.g., FIGS. 105-112), a system actuator assembly 6500 (see e.g., FIGS. 113-117 and 149-151), a medicament container assembly 6200 containing a medicament 6240 (see e.g., FIGS. 118-119), a movable assembly 6300 (see e.g., FIGS. 130-134), a transfer member 6600 (see e.g., FIG. 135), an electronic circuit system 6900 (see e.g., FIGS. 136-141), a cover 6190 (see e.g., FIGS. 142 and 143), and a safety lock 6700 (see e.g., FIGS. 144-148). A discussion of the components of the medical injector 6000 will be followed by a discussion of the operation of the medical injector 6000.

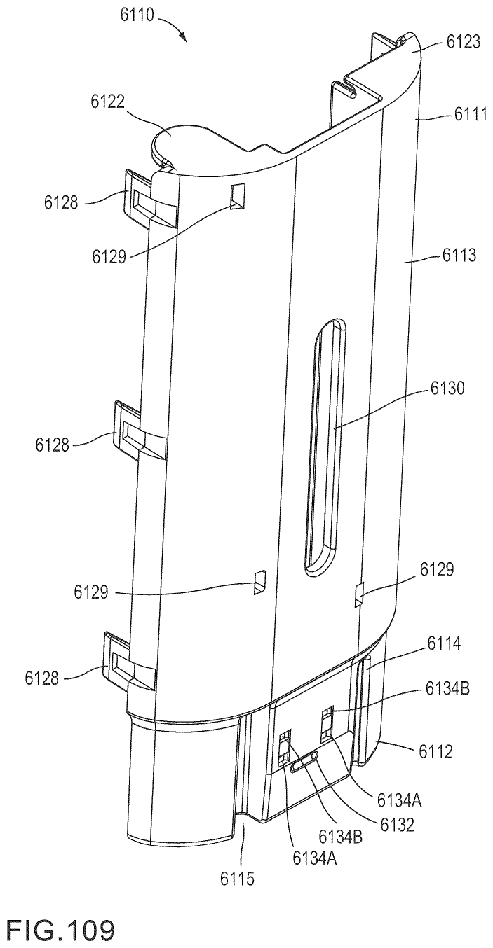

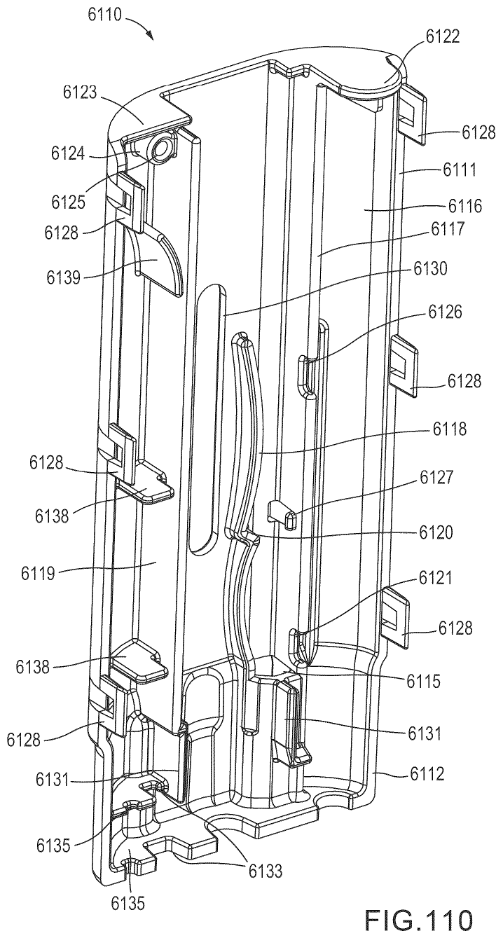

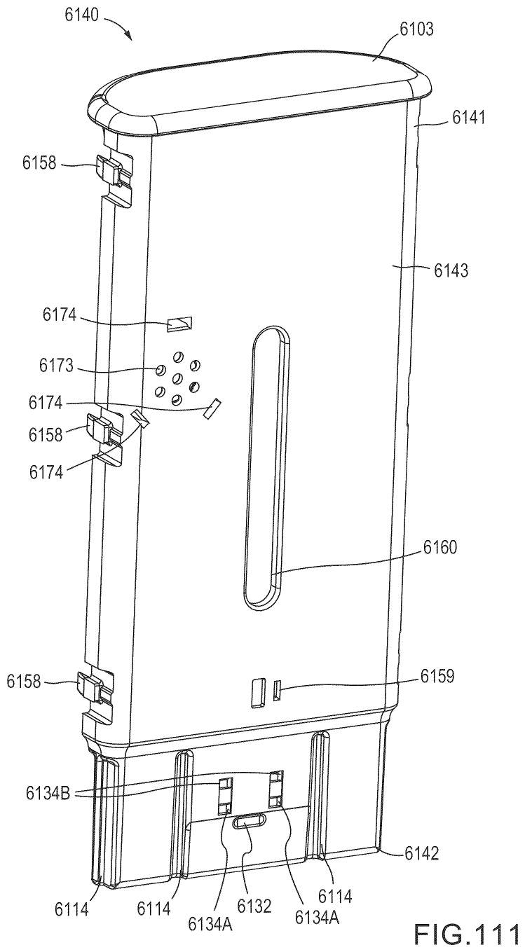

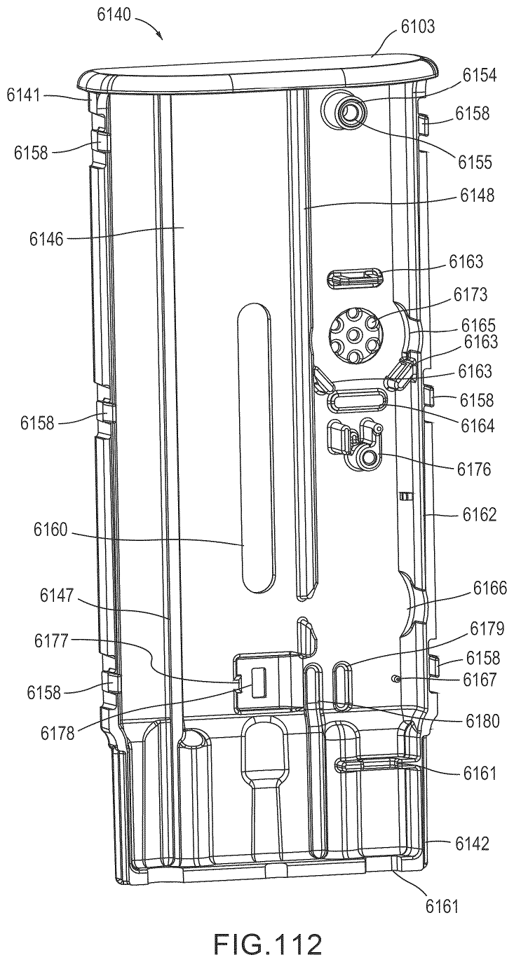

As shown in FIGS. 105-112, the housing 6100 includes a first housing member 6110 (FIGS. 109 and 110) and a second housing member 6140 (FIGS. 111 and 112) that can couple to form the housing 6100. The housing 6100 has a proximal end portion 6101 and a distal end portion 6102. The housing 6100 defines a first status indicator aperture 6130 (defined by the first housing member 6110) and a second status indicator aperture 6160 (defined by the second housing member 6140). The status indicator apertures 6130, 6160 can allow a patient to monitor the status and/or contents of the medicament container 6210 contained within the housing 6100. For example, by visually inspecting the status indicator aperture 6130 and/or 6160, a patient can determine whether the medicament container 6210 contains a medicament 6240 and/or whether the medicament 6240 has been dispensed.

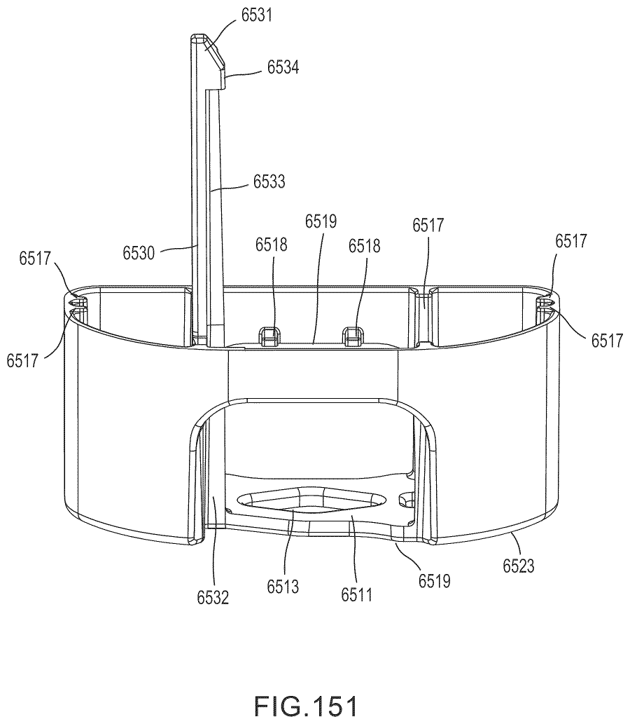

As shown in FIGS. 109-110, the first housing member 6110 includes an outer surface 6113 and an inner surface 6116, and a proximal end portion 6111 and a distal end portion 6112. The outer surface 6113 defines base retention recesses 6134A and 6134B, an activation rod groove 6115, and a base rail groove 6114, at the distal end portion 6112 of the first housing member 6110. The distal base retention recesses 6134A are configured to receive base connection knobs 6518 of an actuator 6510 (also referred to herein as “base 6510,” see e.g., FIG. 151) when the base 6510 is in a first (i.e., pre-actuated) position relative to the housing 6100. The proximal base retention recesses 6134B are configured to receive the base connection knobs 6518 of the base 6510 when the base 6510 is in a second (i.e., actuated) position relative to the housing 6100. The base retention recesses 6134A, 6134B have a tapered proximal sidewall and a non-tapered distal sidewall. This arrangement allows the base retention recesses 6134A, 6134B to receive the base connection knobs 6518 such that the base 6510 can move proximally relative to the housing 6100, but cannot move distally relative to the housing 6100. Said another way, the distal base retention recesses 6134A are configured to prevent the base 6510 from moving in the distal direction when the base 6510 is in the first position and the proximal base retention recesses 6134B are configured to prevent the base 6510 from moving in the distal direction when the base 6510 is in the second position. Similarly stated, the proximal base retention recesses 6134B and the base connection knobs 6518 cooperatively lock the base 6510 to prevent undesirable movement of the base 6510 after the medical injector 6000 is actuated, and to further visually indicate to the user that the medical injector has been actuated.

The activation rod groove 6115 is configured to receive an activator 6530 (also referred to herein as “release member 6530,” and/or “rod 6530” see e.g., FIG. 151) of the base 6510. As described in more detail herein, the release member 6530 of the base 6510 is configured to engage a portion of the movable assembly 6300 (also referred to herein as “medicament delivery mechanism 6300”) when the base 6510 is moved with respect to the housing 6100 to actuate the medical injector 6000. The base rail groove 6114 is configured to receive a guide member 6517 of the base 6510. The guide member 6517 of the base 6510 and the base rail groove 6114 of the housing 6100 engage each other in a way that allows the guide member 6517 of the base 6510 to slide in a proximal and/or distal direction within the base rail groove 6114 while limiting lateral movement of the guide member 6517 and/or base 6510 with respect to the housing 6100.

The inner surface 6116 of the first housing member 6110 includes a transfer member guide 6117, a movable member guide 6118, a mixing actuator guide 6119, an upper spring plate 6122, an upper mixing actuator plate 6123, and a mixing actuator pivot protrusion 6124 (see e.g., FIG. 110). The transfer member guide 6117 is configured to engage a guide surface 6619 and a guide protrusion 6624 of the transfer member 6600 (see FIG. 135). The guide surface 6619 and the guide protrusion 6624 of the transfer member 6600 and the transfer member guide 6117 of the first housing member 6110 engage each other in a way that allows the guide surface 6619 and the guide protrusion 6624 of the transfer member 6600 to slide in a proximal and/or distal direction along the transfer member guide 6117 while limiting lateral movement of the transfer member 6600 within the housing 6100.

The transfer member guide 6117 defines an upper notch 6126 and a lower notch 6121. The upper notch 6126 defined by the transfer member guide 6117 can receive the guide protrusion 6624 of the transfer member 6600 during assembly of the medical injector 6000. Similarly stated, the guide protrusion 6624 is inserted through the upper notch 6126 and is disposed on an opposite side of the transfer member guide 6117 than the guide surface 6619 of the transfer member 6600. This arrangement allows the transfer member 6600 to move in a proximal and/or distal direction with respect to the housing 6100 but prevents the transfer member 6600 from moving in a lateral direction with respect to the housing 6100. Furthermore, the guide protrusion 6624 can be moved through the upper notch 6126 to disengage the transfer member 6600 from the medicament delivery device 6300 without moving the medicament delivery device 6300. For example, in some embodiments, the medicament 6240 disposed within the medicament container 6210 can expire. In such embodiments, the guide protrusion 6624 can be moved through the upper notch 6126 to disengage from the medicament delivery device 6300, thereby disarming the medical injector 6000 (e.g., rendering the medical injector 6000 incapable of completing an injection event in the designed manner). The lower notch 6121 receives the guide protrusion 6624 to facilitate a retraction event, as described in further detail herein.

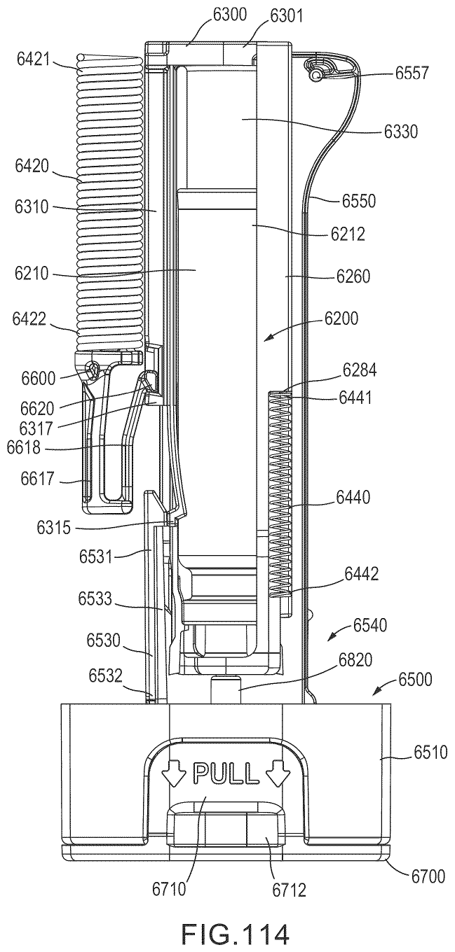

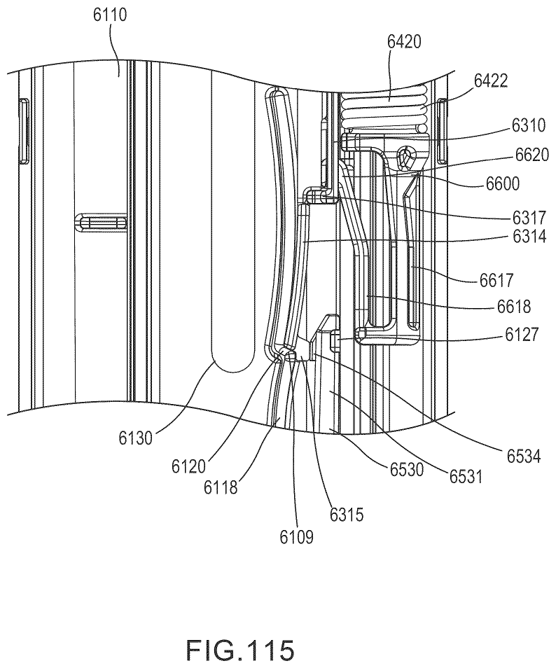

Similarly, the movable member guide 6118 is configured to engage a first latch protrusion 6315 included in a first movable member 6301 of the medicament delivery mechanism 6300 (see e.g., FIGS. 114, 115 and 132). As described in more detail below, the movable member guide 6118 defines a latch member notch 6120 that includes an engagement surface 6109 (see FIG. 115) against which the first latch protrusion 6315 of the latch portion 6310 of the first movable member 6301 is disposed when the medical injector 6000 is in the first configuration.

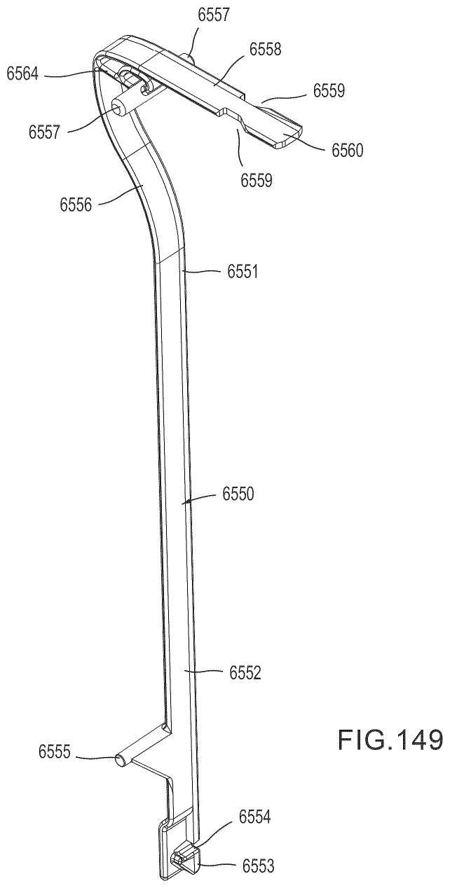

The mixing actuator guide 6119 engages a mixing actuator member 6550 included in the system actuation assembly 6500 (see e.g., FIG. 149). Furthermore, the inner surface 6116 of the first housing portion 6110 includes lower retention protrusions 6138 and an upper retention protrusion 6139. The arrangement of the mixing actuator guide 6119 and the upper and lower retention protrusions, 6139 and 6138 respectively, defines a channel or track within which the mixing actuator member 6550 is disposed. Similarly stated, the mixing actuator member 6550 is slidably disposed against and between the mixing actuator guide 6119 and the upper and lower retention protrusions, 6139 and 6138. In this manner, the mixing actuator guide 6119, the lower retention protrusions 6138, and upper retention protrusion 6139 act to guide the mixing actuator member 6550 when the mixing actuator member 6550 is moved within the housing 6100. For example, as described in further detail herein, the mixing actuator member 6550 is disposed in a space defined between the lower retention protrusions 6138 and the mixing actuator guide 6119, thereby limiting the motion of the mixing actuator member to the proximal and distal direction (i.e., limiting lateral movement of the mixing actuator member 6550). Furthermore, the mixing actuator member 6550 can engage and/or slide against the upper retention protrusion 6139 such that the upper retention protrusion 6139 facilitates a deformation of the mixing actuator member 6550 (e.g., the mixing actuator member 6550 deforms, bends, curves, or otherwise reconfigures) when the mixing actuator member 6550 is moved within the housing 6100.

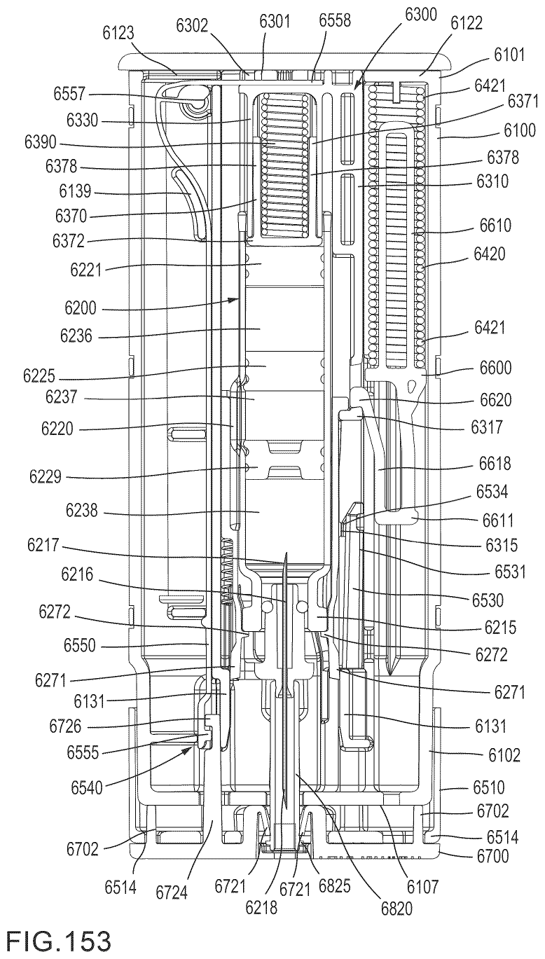

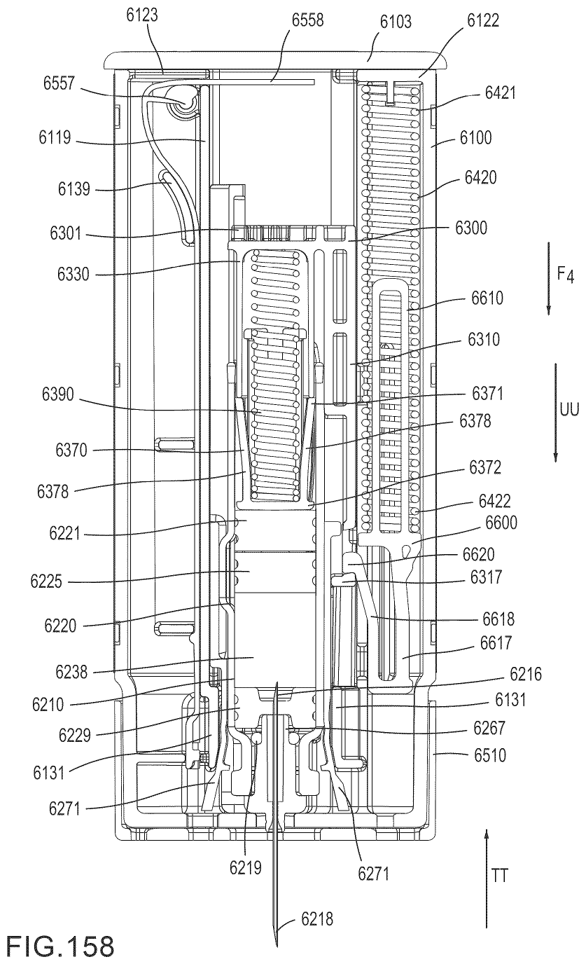

The upper spring plate 6122 is disposed at the proximal end portion 6111 of the first housing member 6110. The upper spring plate 6122 extends from the inner surface 6116 and is configured to contact a proximal end portion 6421 of an energy storage member 6420 (also referred to herein as a “insertion spring 6420” and/or “spring 6420”, see FIG. 153). In this manner, when the medical injector 6000 is activated, the upper spring plate 6122 limits proximal movement of the spring 6420 such that the spring 6420 expands distally to move the medicament delivery mechanism 6300 and/or the transfer member 6600 in a distal direction (see e.g., FIG. 158). Similarly stated, the upper spring plate 6122 receives a force from the spring 6420 and applies an equal and opposite reaction force to the proximal end portion 6421 of the spring 6420 such that a distal end portion 6422 of the spring 6420 expands in a distal direction, as described in further detail herein.

The upper mixing actuator plate 6123 is disposed at the proximal end portion 6111 of the first housing member 6110 and extends from the inner surface 6116. The upper mixing actuator plate 6123 is configured to selectively engage the mixing actuator member 6550 of the system actuator assembly 6500 (see FIG. 153). In this manner, the upper mixing actuator plate 6123 is configured to limit the proximal movement of the mixing actuator member 6550, as described in further detail herein. The mixing actuator pivot protrusion 6124 defines an aperture 6125 that receives a pivot protrusion 6557 of the mixing actuator member 6550. In this manner, the mixing actuator member 6550 can pivot about the pivot protrusion 6557 when the mixing actuator member 6550 is moved within the housing 6100.

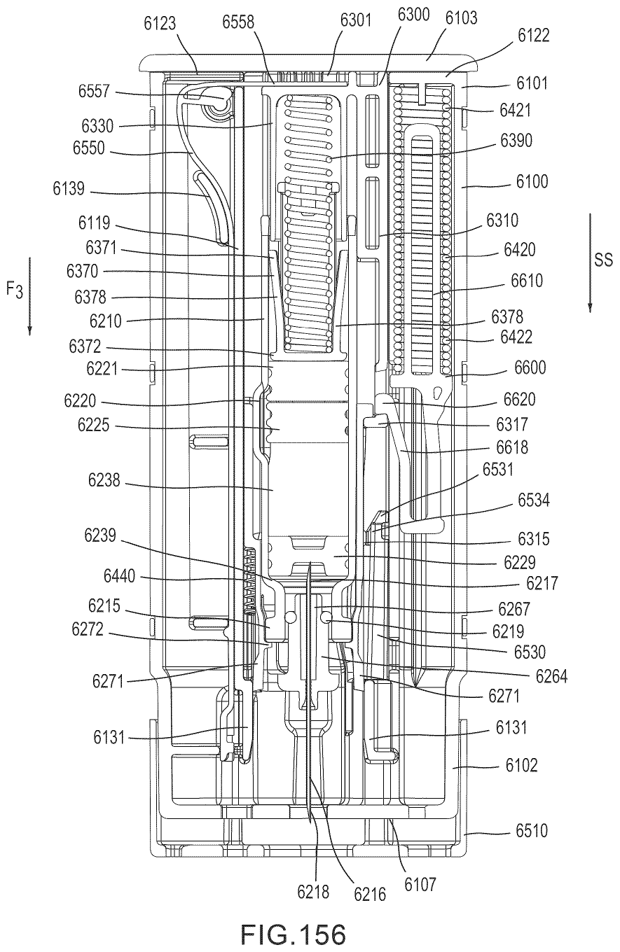

The inner surface 6116 of the first housing member 6110 further includes carrier engagement protrusions 6131 (see e.g., FIG. 110), and defines actuator grooves 6133 and battery isolation protrusion grooves 6135. The carrier engagement protrusions 6131 selectively engage a set of tabs 6271 included in a carrier 6260 of the medicament container assembly 6200 (see FIG. 156). The actuator grooves 6133 receive a portion of a safety lock actuator 6724 of the safety lock 6700 and the mixing actuator member 6550 of the system actuator assembly 6500. Similarly, the battery isolation protrusion grooves 6135 receive a portion of a battery isolation protrusion 6197 included in the cover 6190 when the medical injector 6000 is in the first configuration.

The first housing member 6110 further includes a set of latches 6128 and a set of openings 6129. The latches 6128 extend from portions of the inner surface 6116 of the first housing member 6110. The first housing member 6110 can include any number of latches 6128 that can have any suitable shape or size. For example, in some embodiments, the latches 6128 vary in size. The latches 6128 are configured to engage portions of the second housing member 6140 to couple the first housing member 6110 to the second housing member 6140, as described in further detail herein.

As shown in FIGS. 111 and 112, the second housing member 6140 includes an outer surface 6143 and an inner surface 6146 and a proximal end portion 6141, a proximal cap 6103, and a distal end portion 6142. The second housing member 6140 further includes a set of tabs 6158 and defines a set of openings 6159. The second housing member 6140 can include any number of tabs 6158 such that the number of tabs 6158 corresponds to the number of latches 6128 of the first housing member 6110. Collectively, the latches 6128 of the first housing member 6110 and the tabs 6158 of the second housing member 6140 couple the first housing member 6110 to the second housing member 6140. Similarly stated, the latches 6128 are configured to engage the tabs 6158 to define a lock fit. Moreover, a surface of each of the latches 6128 is in contact with a surface of the corresponding tab 6158 to define a lock fit such that the first housing member 6110 and the second housing member 6140 collectively define the housing 6100. The openings 6129 of the first housing member 6110 and the openings 6159 of the second housing member 6140 allow access to the internal latches of the second housing member 6140 and the internal tabs of the first housing member 6110, respectively. In this manner, the first housing member 6110 can be decoupled from the second housing member 6140.

The outer surface 6143 defines base retention recesses 6134A and 6134B and base rail grooves 6114, at the distal end portion 6142 of the second housing member 6140. The distal base retention recesses 6134A are configured to receive base connection knobs 6518 of the base 6510 when the base 6510 is in a first (prior to actuation) position relative to the housing 6100. The proximal base retention recesses 6134B are configured to receive the base connection knobs 6518 of the base 6510 when the base 6510 is in a second (actuated) position relative to the housing 6100. The base retention recesses 6134A, 6134B have a tapered proximal sidewall and a non-tapered distal sidewall. This arrangement allows the base retention recesses 6134A, 6134B to receive the base connection knobs 6518 such that the base 6510 can move proximally relative to the housing 6100, but cannot move distally relative to the housing 6100. Said another way, the distal base retention recesses 6134A are configured to prevent the base 6510 from moving distally when the base 6510 is in a first position and the proximal base retention recesses 6134B are configured to prevent the base 6510 from moving distally when the base 6510 is in a second position. Similarly stated, the proximal base retention recesses 6134B and the base connection knobs 6518 cooperatively lock the base 6510 to prevent undesirable movement of the base 6510 after the medical injector 6000 is actuated, and to further visually indicate to the user that the medical injector has been actuated.

The base rail grooves 6114 are configured to receive guide members 6517 of the base 6510. The guide members 6517 of the base 6510 and the base rail grooves 6114 of the second housing member 6140 engage each other in a way that allows the guide members 6517 of the base 6510 to slide in a proximal and/or distal direction within the base rail grooves 6114 while limiting lateral movement of the guide members 6517. This arrangement allows the base 6510 to move in a proximal and/or distal direction with respect to the housing 6100 but prevents the base 6510 from moving in a lateral direction with respect to the housing 6100.

The proximal cap 6103 extends from the proximal end portion 6141 of the second housing member 6140 and encloses the proximal end portion 6101 of the housing 6100 when the first housing member 6110 is coupled to the second housing member 6140.

The inner surface 6146 of the second housing member 6140 includes a transfer member guide 6147 and a movable member guide 6148. The transfer member guide 6147 is configured to engage a second guide surface 6626 of the transfer member 6600 (see FIG. 135). The second guide surface 6626 of the transfer member 6600 and the transfer member guide 6147 of the second housing member 6140 engage each other such that the second guide surface 6626 of the transfer member 6600 slides in a proximal and/or distal direction along a surface of the transfer member groove 6147 while limiting lateral movement of the transfer member 6600. Similarly, the movable member guide 6148 is configured to engage a top portion 6302 of the first movable member 6301 included in the medicament delivery mechanism 6300.

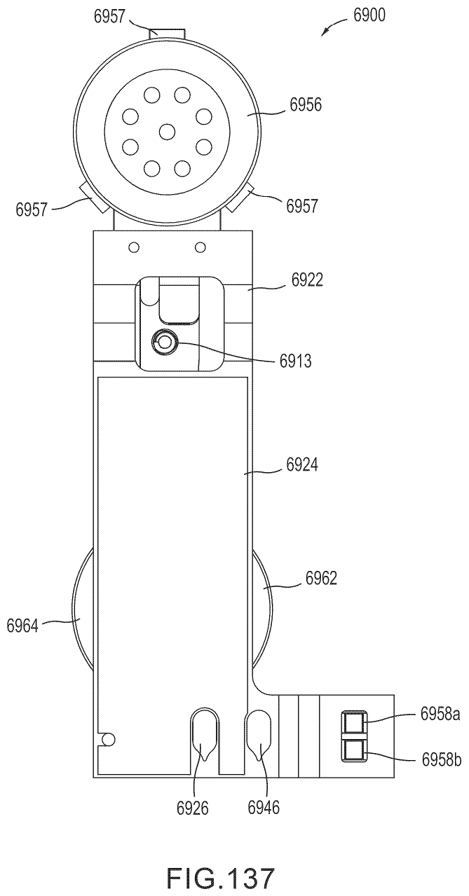

The inner surface 6146 of the second housing member 6140 further includes a mixing actuator pivot protrusion 6154, latches 6163, and a battery clip protrusion 6176. The mixing actuator protrusion 6154 defines and aperture 6155 that receives a pivot protrusion 6557 of the mixing actuator member 6550 (e.g., similar to the pivot protrusion 6124 of the first housing member 6110 described above). The latches 6163 are configured to receive tabs 6957 (see e.g., FIG. 137) included in the electronic circuit system 6900 adjacent the audible output device 6956. The battery clip protrusion 6176 is configured to be coupled to the battery clip 6910. In this manner, the latches 6163 can engage the tabs 6957 of electronic circuit system 6900 and the battery clip 6910 can engage the battery clip protrusion 6176 to collectively couple the electronic circuit system 6900 to the housing 6100. In other embodiments, the electronic circuit system 6900 can be coupled to the housing 6100 by other suitable means such as an adhesive, a clip, a label and/or the like.

The inner surface 6146 of the second housing portion 6140 defines an audible output device recess 6165, a battery recess 6166, multiple sound apertures 6173, an LED aperture 6178, a first actuator groove 6179 and a second actuator groove 6180. A battery 6962 is disposed within the battery recess 6166 when the electronic circuit system 6900 is coupled to the second housing portion 6140. Similarly, an audible output device 6956 is disposed within an audible output device recess 6165 such that a front face of the audible output device 6956 is disposed adjacent the sound apertures 6173. In this manner, the sound apertures 6173 are configured to allow sound produced by the audio output device 6956 to pass from the audio output device 6956 to a region outside of the housing 6100. The LED aperture 6178 is configured to receive LEDs 6958A and 6958B included in the electronic circuit system 6900 such that a user can view the LEDs 6958A, 6958B, which are described in more detail herein.

The inner surface 6146 includes a circuit board retention tab 6177 and a circuit board alignment protrusion 6167. The circuit board retention tab 6177 is configured to engage a portion of a circuit board 6922 included in the electronic circuit system 6900 such that the LEDs 6958A and 6958B are maintained within the LED aperture 6178. With the electronic circuit system 6900 coupled to the second housing portion 6140 (as described above) the circuit board alignment protrusion 6167 can engage the circuit board to ensure alignment of the electronic circuit system 6900 relative to the second housing portion 6140.

The first actuator groove 6179 defined by the inner surface 6146 of the second housing portion 6140 is configured to be disposed adjacent the safety lock actuator groove 6133 defined by the inner surface 6116 of the first housing portion 6110. As described above, the safety lock actuator groove 6133 of the first housing portion 6110 receives the safety lock actuator 6724 of the safety lock 6700 such that the safety lock actuator 6724 can engage the mixing actuator member 6550. In use, the safety lock actuator 6724 moves the mixing actuator member 6550 in the distal direction and a protrusion 6555 of the mixing actuator member 6550 moves in the distal direction within the first actuator groove 6179 to engage a portion of the electronic circuit system 6900, as described in more detail herein. Similarly, the second actuator groove 6180 defined by the inner surface 6146 of the second housing portion 6140 is configured to receive an actuator protrusion 6279 included in the carrier 6260. In use, the carrier 6260 moves in the distal direction such that the actuator protrusion 6279 moves in the distal direction within the second actuator groove 6180 to engage a portion of the electronic circuit system 6900, as further described herein.

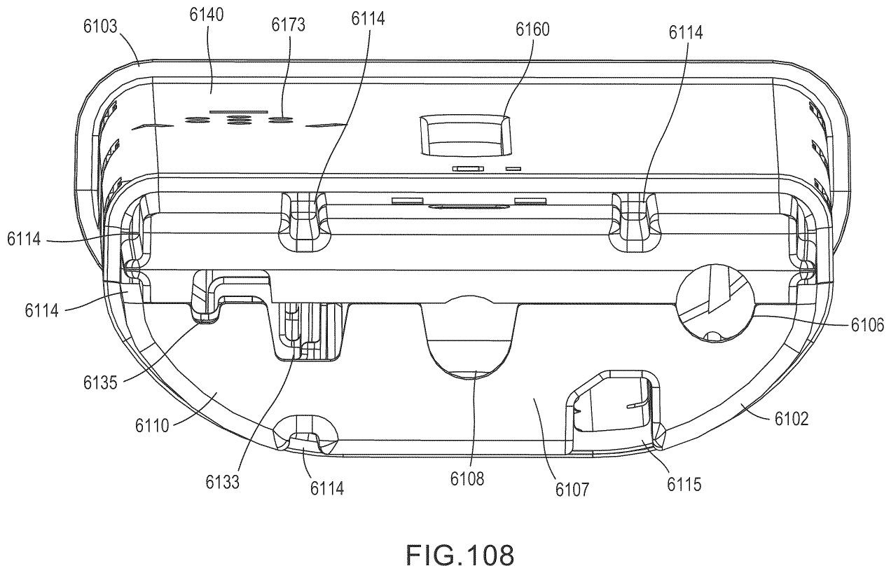

As shown in FIG. 108, when the first housing member 6110 and the second housing member 6140 are assembled, the distal end portion 6102 of the housing 6100 defines a needle aperture 6108 and a transfer member access opening 6106. Similarly stated, the first housing member 6110 and the second housing member 6140 collectively define the needle aperture 6108 and the transfer member access opening 6106. The needle aperture 6108 is configured to allow a needle 6216 (see e.g., FIGS. 158, 159, and 160) to exit the housing 6100 when the medical injector 6000 is actuated, and be retracted back into the housing 6100 upon completion of the injection, as described in further detail herein.

The transfer member access opening 6106 is configured to provide access to the transfer member 6600 when the transfer member 6600 is disposed within the housing 6100. For example, in some embodiments, the transfer member 6600 can be disengaged from the medicament delivery mechanism 6300 without moving the medicament delivery mechanism 6300 in the distal direction. In this manner, the medical injector 6000 can be disabled such that the medicament delivery mechanism 6300 cannot engage the medicament container 6210 to convey a medicament 6240. For example, in some embodiments, a user can disengage the transfer member 6600 from the medicament delivery mechanism 6300, via the transfer member access opening 6106, to safely dispose of an unused medical injector 6000 in which the medicament 6240 has expired. In such embodiments, the user can engage the guide protrusion 6624, via the transfer member access opening 6106, and move the guide protrusion 6624 through the upper notch 6126, as described above. In other embodiments, an operator can manipulate the transfer member within the housing 6100 via the transfer member access opening 6106 during the assembly of the medical injector 6000.

FIGS. 113-135 show the medicament container assembly 6200, the system actuator assembly 6500, the transfer member 6600 and the medicament delivery mechanism 6300 of the medical injector 6000. As shown in FIGS. 113-117, the system actuator assembly 6500 includes the base 6510, a release member 6530, and a mixing actuator assembly 6540. Although the base 6510 and the release member 6530 are shown as being monolithically constructed to form a portion of the system actuator assembly 6500, in other embodiments the system actuator assembly 6500 can include a base that is constructed separately from (and later joined to) a release member. The release member 6530 has a proximal end portion 6531 and a distal end portion 6532. The release member 6530 extends from a proximal surface 6511 of the base 6510.

As shown in FIGS. 114 and 115, the proximal end portion 6531 of the release member 6530 is configured to engage the latch portion 6310 of the medicament delivery mechanism 6300 when the medical injector 6000 is in its first (or storage) configuration. In this manner, the proximal end portion 6531 of the release member 6530 maintains a first latch protrusion 6315 of the latch portion 6310 in contact with the engagement surface 6109 of the latch member notch 6120 of the housing 6100. When the engagement surface 6109 is in contact with the first latch protrusion 6315, the engagement surface 6109 applies a reaction force to the first latch protrusion 6315 in response to the force applied by the spring 6420, which urges the transfer member 6600 and the medicament delivery mechanism 6300 in a distal direction. Similarly stated, when the first latch protrusion 6315 is in contact with the engagement surface 6109, the engagement surface 6109 limits distal movement of the first latch protrusion 6315, and thus, the medicament delivery mechanism 6300. In this manner, when the base 6510 is in a first position (i.e., before actuation of the medical injector 6000), the release member 6530 maintains the first latch protrusion 6315 within the latch member notch 6120 and maintains the medical injector 6000 in the first configuration (e.g., non-actuated configuration). Furthermore, as shown in FIGS. 110 and 115, the first portion 6110 of the housing 6100 includes a retention protrusion 6127 that engages the release member 6530. The retention protrusion contacts the release member 6530 to limit lateral deformation and/or movement of the release member 6530, thereby ensuring that the first latch protrusion is maintained within the latch member notch 6120. Similar stated, the retention protrusion maintains the alignment of the first latch protrusion 6315 and the release member 6530 is maintained.

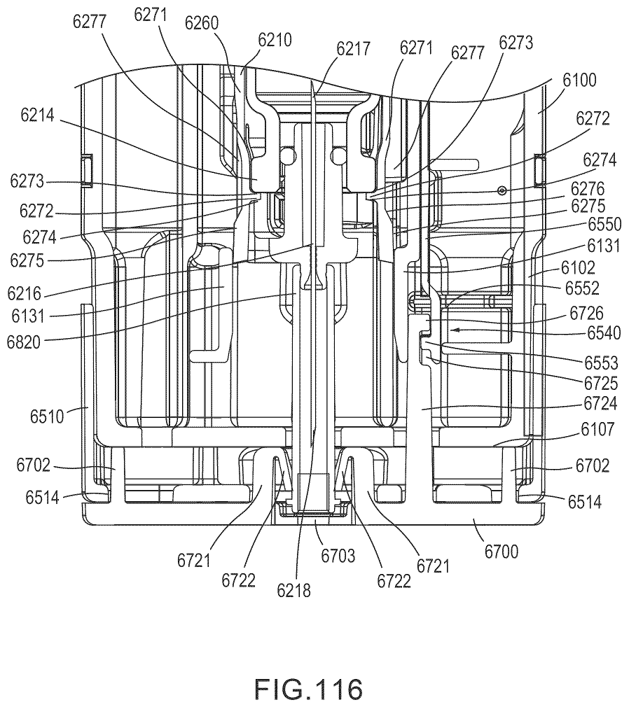

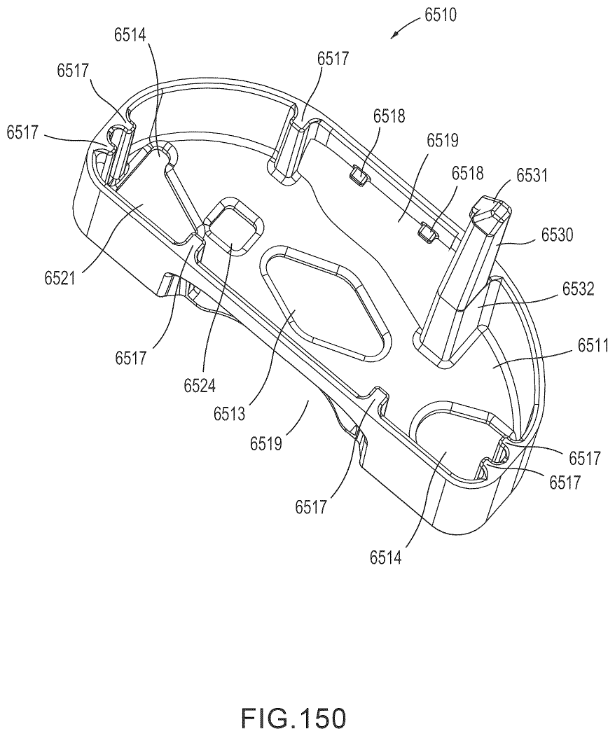

As shown in FIG. 116, when the medical injector 6000 is in its first configuration (i.e., the storage configuration), the safety lock protrusions 6702 are disposed within the safety lock protrusion openings 6514 of the base 6510 (see also FIG. 150), and engage a distal surface 6107 of the housing 6100. In this manner, movement of the safety lock 6700 in the proximal direction is prevented. Therefore, the system actuator assembly 6500 and/or the base 6510 cannot move in the proximal direction to actuate the medicament delivery mechanism 6300. Similarly stated, as shown in FIG. 116, when the medical injector 6000 is in its first configuration (i.e., the storage configuration), the safety lock protrusions 6702 engage the distal surface 6107 of the housing 6100 to limit the proximal movement of the base 6510.

The mixing actuator assembly 6540 includes the mixing actuator member 6550 and the safety lock 6700. As shown in FIGS. 116, 144 and 145 the safety lock 6700 includes the safety lock actuator 6724. The safety lock actuator 6724 includes a protrusion 6726 and defines a channel 6725. The channel 6725 receives a catch 6553 included in the mixing actuator member 6550 such that the protrusion 6726 can engage the catch 6553. In this manner, when the safety lock 6700 is moved in the distal direction to be removed from the medical injector 6000, the protrusion 6726 contacts the catch 6553 of the mixing actuator member 6550 such that the removal of the safety lock 6700 moves a distal portion 6552 of the mixing release member 6550 in the distal direction, as described in further detail herein.

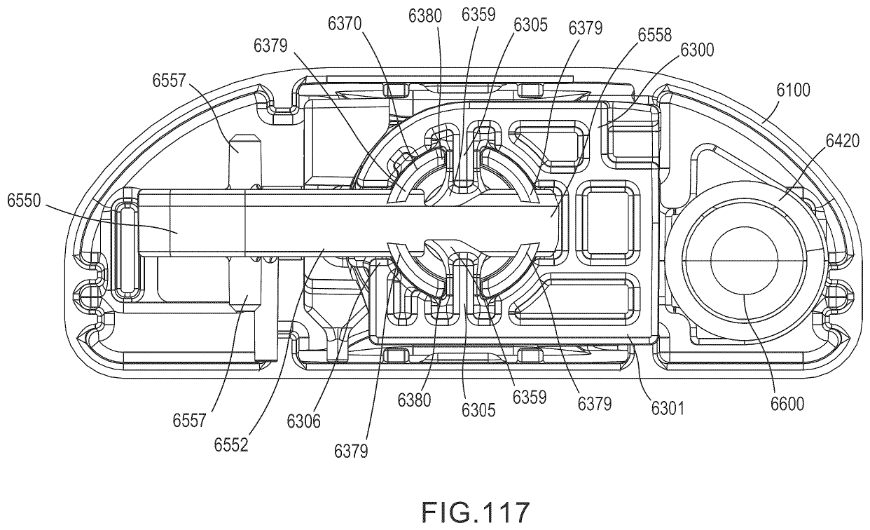

As shown in FIGS. 117, 149 and 155, the mixing actuator member 6550 includes a proximal end portion 6551 configured to engage the first movable member 6301 and the mixing piston 6370. More specifically, the mixing actuator member 6550 includes a retention portion 6558 movably disposed within an actuator member channel 6306 defined by the first movable member 6301. The retention portion 6558 is configured to move within the actuator member channel 6306 between a first position (e.g., the locked position) and a second position (e.g., the mixing position). As described in more detail herein (see e.g., FIG. 130), the mixing piston 6370 is disposed within the piston portion 6330 of the first movable member 6301 such that a proximal end portion 6371 of the mixing piston 6370 can extend through a proximal end portion 6331 of the piston portion 6330 to engage the mixing actuator 6550. In this manner, when the mixing actuator 6550 is in the first position, a set of retention protrusions 6379 of the mixing piston 6370 can engage the retention portion 6558 of the mixing actuator 6550 such that the medical injector 6000 is maintained in the first configuration. Furthermore, when the safety lock 6700 is moved in the distal direction (e.g., removed from the medical injector 6000), the retention portion 6558 is moved to the second position such that the mixing piston 6370 is actuated to urge a mixing event, as described in further detail herein.

The medicament container assembly 6200 includes a medicament container 6210, the needle 6216, and the carrier 6260. The medicament container 6210 includes a proximal end portion 6212, a distal end portion 6213, and a bypass 6220. The bypass 6220 can be a singular channel bypass or can define multiple channels. Although the bypass 6220 is shown in FIGS. 118 and 119 as an external bypass, in other embodiments, the bypass 6220 can be internal to the medicament container and/or a part of the elastomeric member 6225. Said another way, in some embodiments the bypass can be configured such that the outer diameter of the medicament container 6210 is substantially constant. The bypass 6220 is configured to facilitate the mixing and/or injection of a medicament contained within the medicament container 6210, as described in further detail herein. In particular, the bypass 6220 is configured to place various volumes within the medicament container 6210 in fluid communication with each other.

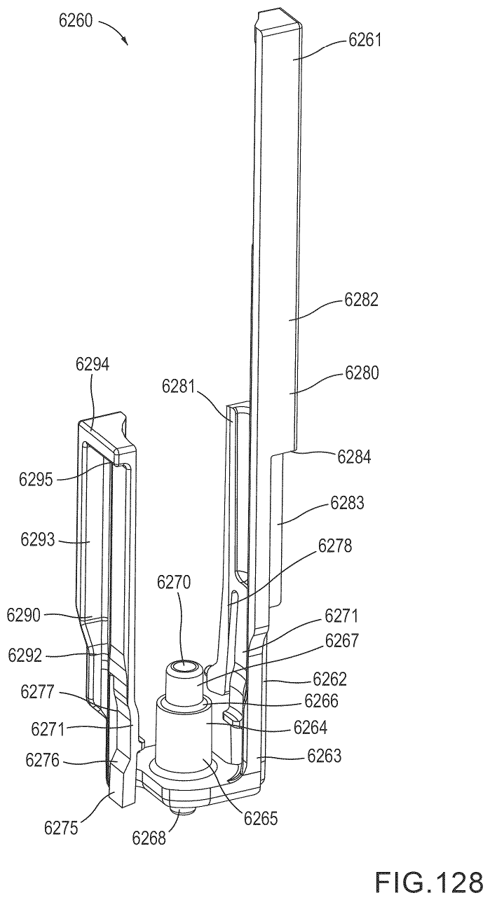

As shown in FIGS. 118 and 119, the distal end portion 6213 of the medicament container 6210 includes a neck 6215 and a flanged end 6214 configured to engage at least a portion of the carrier 6260 and the needle 6216, as described below. Furthermore, the distal end portion 6213 of the medicament container 6210 includes a sealing member 6219. The sealing member 6219 can be any suitable member, such as, for example, an o-ring. In this manner, the sealing member 6219 is configured to engage an inner surface of the medicament container 6210 and a portion of a needle hub 6264 included in the carrier 6260 (see e.g., FIG. 128) to define a fluidic seal, as described in further detail herein.

The proximal end portion 6212 of the medicament container 6210 receives a first elastomeric member 6221, a second elastomeric member 6225, and a third elastomeric member 6229. In some embodiments, the first elastomeric member 6221, the second elastomeric member 6225, and the third elastomeric member 6229 are placed within the medicament container 6210 during the fill process, as further described herein, to define a diluent volume 6236, a dry medicament volume 6237, and a void volume 6238 (see, e.g., FIG. 119). Said another way, the diluent volume 6236 is a volume disposed within the medicament container 6210 defined between a distal surface 6223 of the first elastomeric member 6221 and a proximal surface 6226 of the second elastomeric member 6225. The dry medicament volume 6237 is a volume disposed within medicament container 6210 defined between a distal surface 6227 of second elastomeric member 6225 and a proximal surface 6230 of third elastomeric member 6229 and the void volume 6238 is a volume disposed within the medicament container 6210 defined between a distal surface 6231 of the third elastomeric member 6229 and the distal end portion 6213 of the medicament container 6210.

As shown in FIG. 119, the diluent volume 6236, the dry medicament volume 6237, and the void volume 6238 are defined by the positions of the first elastomeric member 6221, the second elastomeric member 6225, and the third elastomeric member 6229, relative to and/or within the medicament container 6210. In some embodiments, the diluent volume 6236 can contain a medicament diluent, such as, for example, water. In some embodiments, the dry medicament volume 6237 can contain a lyophilized medicament (e.g., any suitable medicament produced via any suitable lyophilizing process) including any of the formulations and/or compositions described herein.

As shown in FIGS. 113 and 114, the proximal end portion 6212 of the medicament container 6210 is coupled to and/or receives a portion of the medicament delivery mechanism 6300 such that medicament delivery mechanism 6300 can move the first elastomeric member 6221, the second elastomeric member 6225, and/or the third elastomeric member 6229 to mix and/or inject the medicament disposed therein. More specifically, the proximal end portion 6212 of the medicament container 6210 can receive a piston portion 6330 of the first movable member 6301 and a second movable member 6370 (also referred to herein as a “mixing piston 6370” and shown, for example, in FIGS. 130 and 134).

The medicament container 6210 can have any suitable size (e.g., length and/or diameter). Moreover, the medicament container 6210, the piston portion 6330, and/or the mixing piston 6370 can be collectively configured such that the piston portion 6330 and/or the mixing piston 6370 travels a desired distance within the medicament container 6210 (i.e., the “stroke”) during an injection event. In this manner, the medicament container 6210, the diluent contained within the diluent volume 6236, the lyophilized medicament contained within the dry medicament volume 6237, the void volume 6238, the piston portion 6330, and the mixing piston 6370 can be collectively configured to provide a desired fill volume and delivery volume.

The length of the medicament container 6210 and the length of the piston portion 6330 and/or the mixing piston 6370 can be configured such that the medicament delivery mechanism 6300 can fit in the same housing 6100 regardless of the fill volume, the delivery volume and/or the ratio of the fill volume to the delivery volume. In this manner, the same housing and production tooling can be used to produce devices having various dosages of the medicament. For example, in a first embodiment (e.g., having a fill volume to delivery volume ratio of 0.4), the medicament container has a first length and the second movable member has a first length. In a second embodiment (e.g., having a fill volume to delivery volume ratio of 0.6), the medicament container has a second length shorter than the first length, and the second movable member has a second length longer than the first length. In this manner, the stroke of the device of the second embodiment is longer than that of the device of the first embodiment, thereby allowing a greater dosage. The medicament container of the device of the second embodiment, however, is shorter than the medicament container of the device of the first embodiment, thereby allowing the components of both embodiments to be disposed within the same housing and/or a housing having the same length.

The first elastomeric member 6221, the second elastomeric member 6225, and the third elastomeric member 6229 can be of any design or formulation suitable for contact with the medicament (e.g., the diluent contained in the diluent volume 6236 and/or a lyophilized medicament contained in the dry medicament volume 6237). For example, the elastomeric members 6221, 6225, and 6229 can be formulated to minimize any reduction in the efficacy of the medicament that may result from contact (either direct or indirect) between the elastomeric members 6221, 6225, and 6229 and the medicament. For example, in some embodiments, the first elastomeric member 6221, the second elastomeric member 6225, and the third elastomeric member 6229 can be formulated to minimize any leaching or out-gassing of compositions that may have an undesired effect on the medicament. In other embodiments, the elastomeric members 6221, 6225, and 6229 can be formulated to maintain its chemical stability, flexibility and/or sealing properties when in contact (either direct or indirect) with the medicament over a long period of time (e.g., for up to six months, one year, two years, five years or longer).

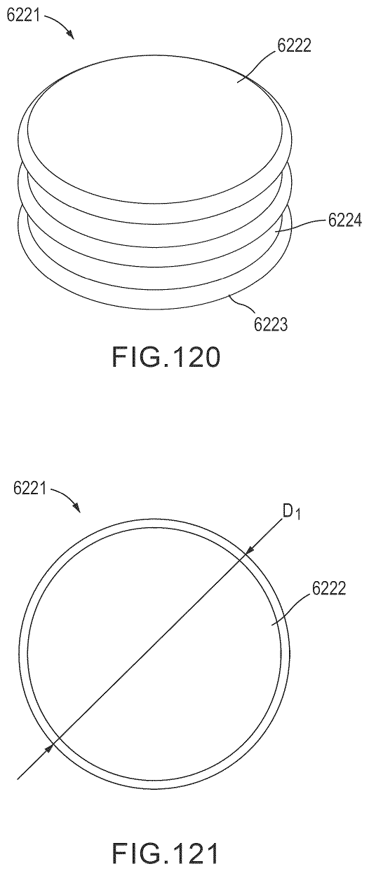

As shown in FIGS. 120-123, the first elastomeric member 6221 includes a proximal surface 6222, the distal surface 6223, and a set of grooves 6224. The grooves 6224 can be configured (e.g., have a size and/or shape) to allow expansion of the first elastomeric member 6221 within a medicament container 6210. Furthermore, the first elastomeric member 6221 has a diameter D₁ and a height H₁. The radius R₁ (FIG. 104) can be any suitable radius. For example, in some embodiments, the diameter D₁ is directly related to the inner diameter (e.g., diameter of an inner surface) of the walls of the medicament container 6210. In such embodiments, the diameter D₁ can be configured to be slightly larger than the inner diameter of the medicament container 6210. In this manner, the sides of the first elastomeric member 6221 can engage the inner surface of the medicament container 6210 to define a fluid seal. Expanding further, with the diameter D₁ of the first elastomeric member 6221 slightly larger than the inner radius of the medicament container 6210, the grooves 6224 define a void such that the side of the first elastomeric member 6221 can deform (e.g., be flattened) to occupy a portion of the void when disposed within or moved within the medicament container 6210. Similarly stated, the grooves 6224 allow the sides of the first elastomeric member 6221 to deform such that the diameter D₁ can be reduced to be substantially similar to the inner diameter of the medicament container 6210.

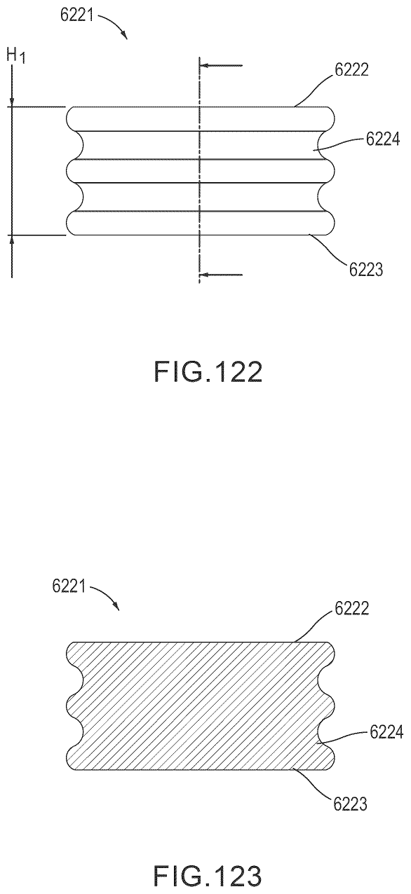

The height H₁ (FIG. 122) of the first elastomeric member 6221 can be any suitable height. In some embodiments, the height H₁ of the first elastomeric member 6221 can be used to control the fill volume and/or the delivery volume. In this manner, the first elastomeric member 6221 can further be configured to control the stroke length of the piston portion 6330 of the first movable member 6301 and/or the mixing piston 6370. In some embodiments, the height H₁ of the first elastomeric member 6221 can be such that, in use, the first elastomeric member 6221 does not substantially deform in a longitudinal direction (e.g., proximal and distal direction). Thus, the height H₁ of the first elastomeric member 6221 can be such that the first elastomeric member 6221 does not substantially deform when engaged by the first movable member 6301 and/or the second movable member 6370. In some embodiments, the second elastomeric member 6225 can be substantially similar to the first elastomeric member 6221; therefore, the second elastomeric member 6225 is not shown or described in detail herein.

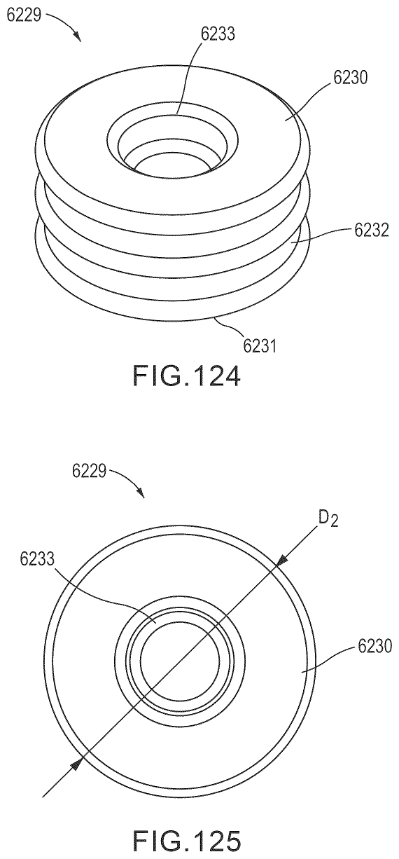

As shown in FIGS. 124-127 the third elastomeric member 6229 includes a proximal surface 6230, a distal surface 6231, a set of grooves 6232, a proximal counter bore 6233, and a distal counter bore 6234. The grooves 6232 can be configured (e.g., have a size and/or shape) to allow expansion of the third elastomeric member 6229 within a medicament container 6210. Furthermore, the third elastomeric member 6229 has a diameter D₂, a height H₂, and a thickness T. The diameter D₂ (FIG. 125) can be any suitable diameter. For example, in some embodiments, the diameter D₂ is directly related to the inner diameter (e.g., diameter of an inner surface) of the walls of the medicament container 6210. In such embodiments, the diameter D₂ can be configured to be slightly larger than the inner radius of the medicament container 6210. In this manner, the sides of the third elastomeric member 6229 can engage the inner surface of the medicament container 6210 to define a fluid seal. Expanding further, the grooves 6232 allow the sides of the third elastomeric member 6229 to deform such that the diameter D₂ can be reduced to be substantially similar to the inner diameter of the medicament container 6210. In some embodiments, the diameter D₂ of the third elastomeric member 6229 can be substantially similar to the diameter D₁ of the first elastomeric member 6221. Similarly, in some embodiments, the height H₂ of the third elastomeric member 6229 can be substantially similar to the height H₁ of the first elastomeric member.

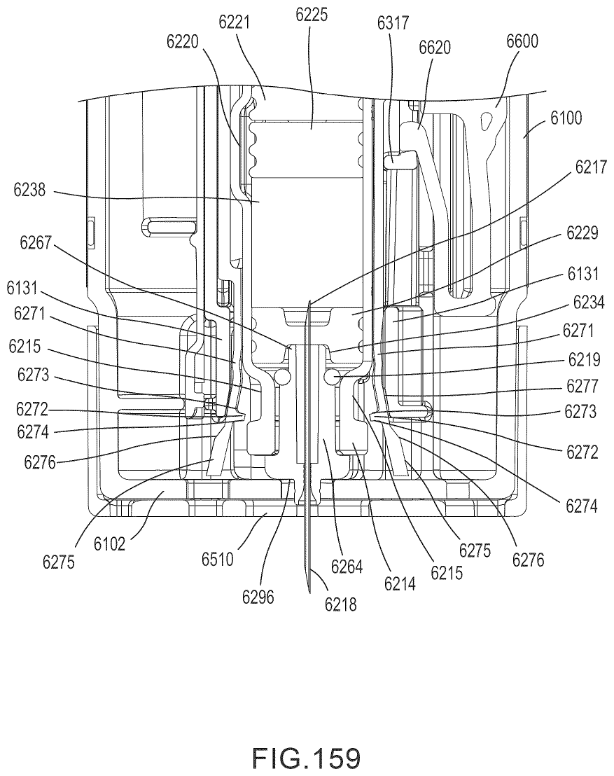

The proximal counter bore 6233 and the distal counter bore 6234 define a depth P, a width W, an angle O, an external radius S, and an internal radius Q. In use, the third elastomeric member 6229 is configured to engage a portion of the carrier 6260 and the needle 6216. More specifically, the distal counter bore 6234 receives a portion of the needle hub 6264, as shown in FIGS. 158 and 159, described in further detail herein. The width W and depth D of the distal counter bore 6234 can be such that an upper portion 6267 of the needle hub 6264 can be disposed within the distal counter bore 6234 when the medicament container 6210 is moved to a second container position (in which the needle 6216 is placed into fluid communication with the dry medicament volume 6237). Furthermore, the distal counter bore 6234 and the proximal counter bore 6233 reduces the thickness T of the portion of the third elastomeric member 6229 through which the needle 6216 penetrates, as further described herein.

In some embodiments a first elastomeric member, a second elastomeric member, and/or a third elastomeric member of an injector can be similar to first elastomeric member 6221 or third elastomeric member 6229. Said another way, in some embodiments, a medicament container can include three elastomeric members similar to the first elastomeric member 6221. In other embodiments, a medicament container can include three elastomeric members similar to the third elastomeric member 6229. For example, in such embodiments, the first elastomeric member and the second elastomeric member can define a proximal counter bore and a distal counter bore and can be configured to further control the fill volume and/or delivery volume of a diluent and/or lyophilized medicament disposed within the medicament container.

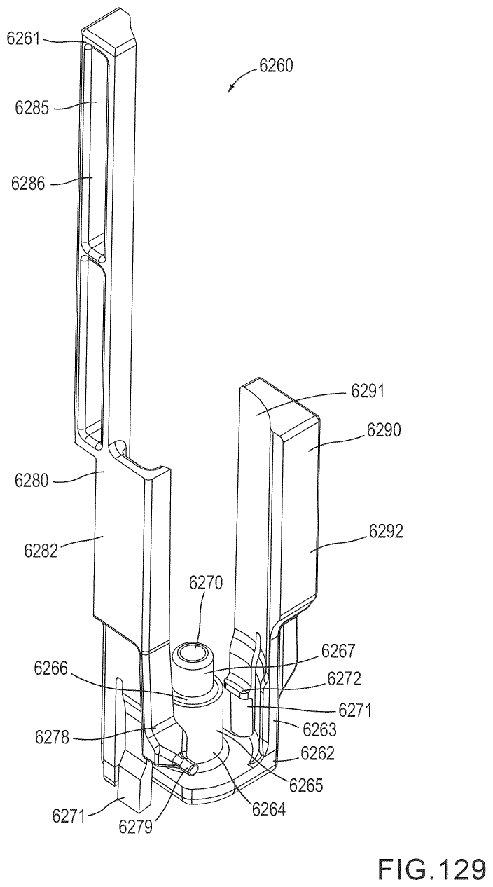

As described above, the medicament container 6210 is configured to engage and/or be coupled to the carrier 6260 (see e.g., FIGS. 113 and 114). Referring to FIGS. 128 and 129, the carrier 6260 includes a proximal end portion 6261, a distal end portion 6262, a needle hub 6264, an electronics engagement portion 6278, a first retention arm 6280, and a second retention arm 6290. The first retention arm 6280 and the second retention arm 6290 extend, in the proximal direction, from a container-mounting portion 6263 disposed at the distal end portion 6262 of the carrier 6260. The container-mounting portion 6263 is configured to selectively engage the flanged end 6214 of the medicament container 6210. More specifically, the carrier 6260 includes the set of tabs 6271 that include a container shoulder 6272. As described above, the set of tabs 6271 are configured to selectively engage the container engagement protrusions 6131 of the housing 6100 (see e.g., FIG. 116). The arrangement of the tabs 6271, the container engagement protrusions 6131, and the container shoulders 6272 are such that the flanged end 6214 of the medicament container 6210 can selectively engage the container shoulder 6272 when moving between the first container position and the second container position, as described in further detail herein.

The needle hub 6264 includes a base portion 6265, an upper portion 6267, and a lower needle port 6268. The base portion 6265 includes a proximal surface 6266 from which the upper portion 6267 extends in the proximal direction. The lower needle port 6268 is configured to extend from the base portion 6265 in the distal direction. The needle hub 6264 defines a needle passageway 6270 that receives a proximal end portion 6217 of the needle 6216 (see e.g., FIG. 116). Expanding further, the needle passageway 6270 can include an inner surface (not shown) that includes any suitable feature to couple the needle 6216 within the needle hub 6264. For example, in some embodiments, the inner surface defining the needle passageway 6270 can include a set of protrusions configured to define a friction fit with the needle 6216. In other embodiments, an adhesive can be applied to the inner surface defining the needle passageway 6270 to couple the needle 6216 to the needle hub 6264. The needle hub 6264 is configured to engage a portion of the medicament container 6210 when the medicament container 6210, as shown in FIG. 116.

The electronics engagement portion 6278 includes an activator protrusion 6279. The electronics engagement portion 6278 extends from a surface of the first retention arm 6280 and is configured to engage the electronic circuit system 6900. More specifically, the activator protrusion 6279 of the electronics engagement portion 6278 is disposed within a second actuation portion 6946 of the electronic circuit system 6900 when the carrier 6260 is in the first position. When the carrier 6260 is moved to the second position (i.e., during the injection event), the activator protrusion 6279 moves in the distal direction to actuate the second actuation portion 6946 of the electronic circuit system 6900 as described in further detail herein.

The first retention arm 6280 includes an inner surface 6281 and an outer surface 6282. The inner surface 6281 engages the medicament container 6210 when the medicament container 6210 is disposed within and/or is coupled to the container-mounting portion 6263. The outer surface 6282 defines a channel 6283 and includes a retraction spring surface 6284. The channel 6283 receives a retraction spring 6440 (FIG. 114) such that a proximal end portion 6441 of the retraction spring 6440 is in contact with the retraction spring surface 6284. The outer surface 6282 further defines a slot 6285. The slot 6285 is configured to receive a guide protrusion 6303 of the first movable member 6301. In this manner, the set of walls 6286 that define the slot 6285 can engage the guide protrusion 6303 of the first movable member 6301 such that the top portion 6302 of the first movable member 6301 is aligned with the carrier 6260 when the first movable member 6301 moves relative to the carrier 6260. Furthermore, during a retraction event, a distal surface of the wall 6286 defining the slot 6285 can engage the guide protrusion 6303 to transfer a portion of a retraction force, exerted by the retraction member 6440, on the first movable member 6301 such that the first movable member 6301 moves in the proximal direction when the carrier 6260 is retracted.

The second retention arm 6290 includes an inner surface 6291 and an outer surface 6292. Similar to the first retention arm 6280, the inner surface 6291 of the second retention arm 6290 engages the medicament container 6210 when the medicament container 6210 is disposed within and/or is coupled to the container-mounting portion 6263. In this manner, the container-mounting portion 6263, the inner surface 6281 of the first retention arm 6280, and the inner surface 6291 of the second retention arm 6290 act to couple the medicament container 6210 to the carrier 6260. The outer surface 6292 defines a channel 6293, and includes a latch 6294. The channel 6293 receives a protrusion 6313 included in the latch portion 6310 of the first movable member 6301. In this manner, the protrusion 6313 can move within the channel during an injection event.

The medicament delivery mechanism 6300 (all or portions of which can also be referred to as a “movable assembly”) includes the first movable member 6301, the second movable member 6370 (the mixing piston 6370), and a mixing spring 6390 (see e.g., FIGS. 130-134). The first movable member 6301 includes the top portion 6302, the latch portion 6310, and the piston portion 6330. The top portion 6302 includes the guide protrusion 6303 and the actuator member channel 6306 (for receiving the mixing actuator member 6550), as described above.

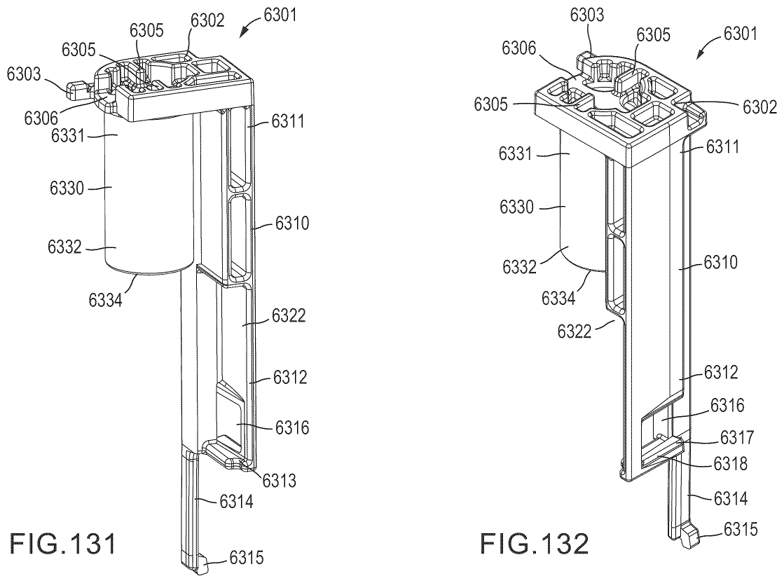

The latch portion 6310 includes a proximal end portion 6311 and a distal end portion 6312 (see e.g., FIGS. 131 and 132). The proximal end portion 6311 is disposed at and/or is joined with the top portion 6302 of the first movable member 6301. Similarly stated, the latch portion 6310 is configured to extend from the top portion 6302 of the first movable member 6301 in the distal direction. The distal end portion 6312 of the latch portion 6310 includes a latch arm 6314 having a first latch protrusion 6315, a second latch protrusion 6317, and a protrusion 6313, and defines an opening 6316 and channel 6322. As described above, the first latch protrusion 6315 is configured to engage the release member 6530 of the base 6510 and the engagement surface 6109 of the latch member notch 6120. In particular, as shown in FIG. 115, the release member 6530 urges, bends and/or deforms the latch arm 6314 to maintain the first latch protrusion 6315 within the latch member notch 6120. Thus, the latch arm 6314 can be constructed from a material having sufficient flexibility such that the release member 6530 can urge, bend and/or deform the latch arm 6314 to engage the first latch protrusion 6315 with the latch member notch 6120.

The opening 6316 of the latch portion 6310 is defined between a surface of the distal end portion 6312 of the latch portion 6310 and a proximal surface 6318 of the second latch protrusion 6317 (see e.g., FIG. 132). The opening 6316 is configured to receive the latch 6620 of the transfer member 6600 (see e.g., FIGS. 135 and 114). More particularly, when the medical injector 6000 is in the first configuration (i.e., prior to actuation), the proximal surface 6318 of the second latch protrusion 6317 is in contact with a distal surface 6621 of the latch 6620 of the transfer member 6600. In this manner, the transfer member 6600 can transfer a force produced by the spring 6420 to the latch portion 6310 of the first movable member 6300 to move the medicament delivery mechanism 6300 in the distal direction when the medical injector 6000 is actuated. Similarly stated, this arrangement allows the medicament delivery mechanism 6300 and/or the first movable member 6301 to move with and/or remain coupled to the transfer member 6600 during the insertion and/or injection operation. The channel 6322 receives the second retention arm 6290 of the carrier 6260. In this manner, the second retention arm 6290 can move within the channel 6322 between the first position and the second position. Similarly stated, this arrangement allows at least a portion of the carrier 6260 to move within the first movable member 6301 when the movable member 6301 moves relative to the carrier 6260 (e.g., during an injection event).

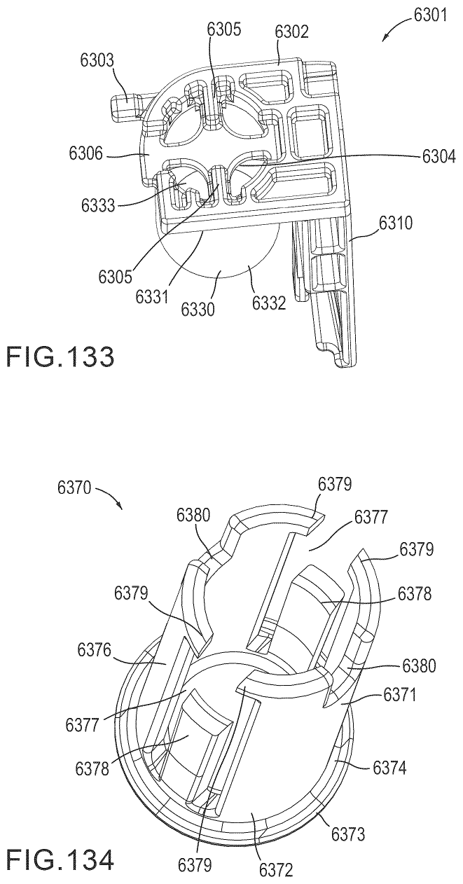

The piston portion 6330 includes a proximal end portion 6331 and a distal end portion 6332 and defines an opening 6333 (see e.g., FIG. 133). More specifically, the proximal end portion 6331 is disposed at and/or joined with a bottom surface 6304 of the top portion 6302 of the first movable member 6301. Expanding further, the piston portion 6330 extends from the bottom surface 6304 of the top portion 6302 and defines an annular shape. Thus, the opening 6333 is defined by the inner walls of the piston portion 6330. The distal end portion 6332 is configured to be disposed at least partially within the proximal end portion 6212 of the medicament container 6210 (see e.g., FIG. 153).

As shown in FIG. 130, the piston portion 6330 is configured to receive at least a portion of the mixing spring 6390 and the mixing piston 6370. More specifically, the medicament delivery mechanism 6300 is configured such that when the medical injector 6000 is in the first configuration (e.g., the storage configuration), the mixing spring 6390 is disposed within the piston portion 6330 and the mixing piston 6370 in a first (e.g., compressed) configuration (see e.g., FIG. 153). Furthermore, the mixing piston 6370 (e.g., the second movable member 6370) is disposed within the piston portion 6330 such that a proximal end portion 6371 of the mixing piston 6370 extends, in the proximal direction, through the piston portion 6330 of the first movable member 6301. Similarly stated, when the mixing piston 6370 is in a first position (the storage position), the proximal end portion 6371 extends through the proximal end portion of the first movable member 6301 such that the mixing piston 6370 can be retained within the piston portion 6330 of the first movable member 6301, as described below.

The mixing piston 6370 includes the proximal end portion 6371 and a distal end portion 6372. The distal end portion 6372 includes a base 6373 with a proximal surface 6374 and a distal surface 6375. The proximal surface 6374 of the base 6373 defines a spring seat that receives a distal end portion 6392 of the mixing spring 6390. The distal surface 6375 of the base 6373 is configured to engage the proximal surface 6222 of the first plunger 6221, as described above. The mixing piston 6370 further includes a set of walls 6376 that extend in the proximal direction from the proximal surface 6374 of the base 6373. The walls 6376 define channels 6377 and include tabs 6378 that selectively engage the piston portion 6330 of the first movable member 6301. The tabs 6378 are configured to move between a first configuration (e.g., a retracted configuration) and a second configuration (e.g., an extended configuration). In some embodiments, the tabs 6378 can define a pre-stress load such that the tabs 6378, without an external force applied, are in the extended configuration. In some embodiments, the tabs 6378 can be maintained in the first configuration by the inner surface of the piston portion 6330. In such embodiments, the tabs 6378 can be moved to the second configuration when the mixing piston 6370 is moved in the distal direction to the second position, as described in further detail herein. As described herein, the tabs 6378 (also referred to as a retention portion or retention members) are configured to contact and/or engage the distal end surface 6334 to limit proximal movement of the mixing piston 6370 relative to the first movable member 6301 (i.e., retraction of the mixing piston 6370 into the piston portion 6330) after the mixing piston 6370 has been actuated. This arrangement prevents retraction of the mixing piston 6370 when the force produced by the spring 6420, which can exceed the force produced by the mixing spring 6390, is applied to the first movable member 6301 via the transfer member 6600.

The proximal end portion 6371 includes retention protrusions (or portions) 6379 and alignment grooves 6380. The retention grooves 6379 extend laterally from a surface of the walls 6376 that define the channels 6377. Similarly stated, as shown in FIG. 134, the retention protrusions 6379 extend into the channels 6377. As described above, the proximal end portion 6371 of the mixing piston 6370 extends through the proximal end portion 6331 of the piston portion 6330. In this manner, the alignment grooves 6380 receive alignment protrusions 6305 included in the top portion 6302 of the first movable member 6301 (see e.g., FIG. 117). Furthermore, when the medical injector 6000 is in the first configuration, the retention protrusions 6379 engage the retention portion 6558 of the mixing actuator member 6550. Thus, when the medical injector 6000 is in the first configuration, the mixing piston 6370 is in a first (e.g., locked) position, in which the movement of the mixing piston 6370 relative to the first movable member 6301 is limited and/or prevented.

The arrangement of the first movable member 6301, the mixing piston 6370, and the mixing actuator member 6550 is such that when the mixing actuator member 6550 is moved to actuate a mixing event, the mixing spring 6390 expands to move the mixing piston 6370 in the distal direction. More particularly, when the retention protrusions 6379 are in contact with the retention portion 6558 of the mixing actuator member 6550, a lock surface 6560 (see e.g., FIG. 149) of the retention portion 6558 applies a reaction force to a distal surface of the retention protrusions 6379 equal to the force exerted by the mixing spring 6390. Therefore, when the mixing actuator member 6550 is moved to the second position (e.g., no longer in contact with the retention protrusions 6379) the reaction force is removed and the mixing spring 6390 expands. Furthermore, the bottom surface 6304 of the top portion 6302 of the first movable member 6301 engages the proximal end portion 6391 of the mixing spring 6390 such that when the mixing spring 6390 expands, the distal end portion 6392 moves in the distal direction. Thus, the expansion of the mixing spring 6390 is such that the mixing spring 6390 exerts a force on the mixing piston 6370 to move the mixing piston 6370 in the distal direction, as further described herein.

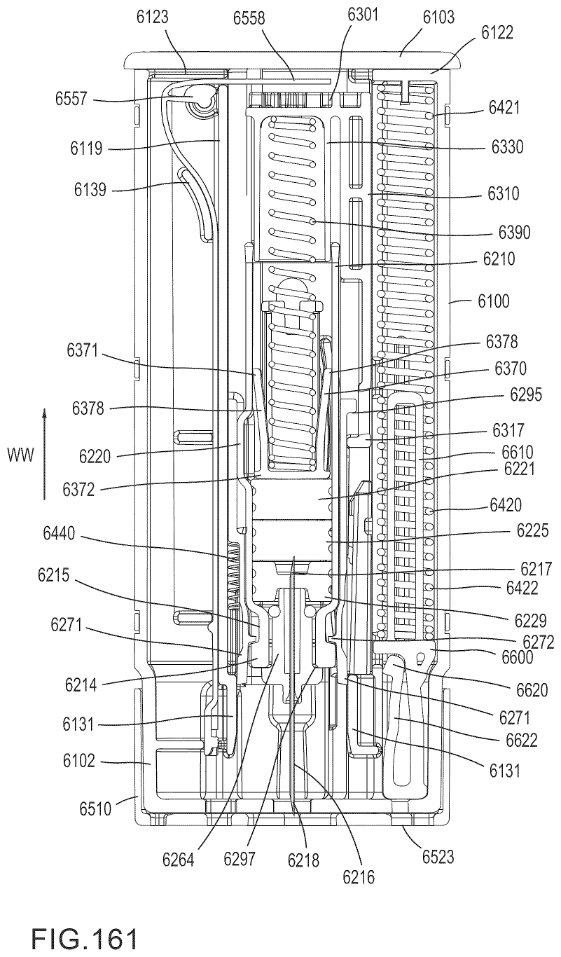

Referring to FIG. 135, the transfer member 6600 includes a proximal end portion 6610 and a distal end portion 6611, and is configured to move between a first configuration (see e.g., FIGS. 135 and 153, in which the transfer member 6600 is engaged to the first movable member 6301) and a second configuration (see e.g., FIG. 161, in which the transfer member 6600 is disengaged from the first movable member 6301). The proximal end portion 6610 is substantially cylindrical and is configured to engage and/or contact the spring 6420. Moreover, the transfer member 6600 includes a ring protrusion 6612 that includes a proximal surface 6613 defining a spring seat 6615. The distal end portion 6422 of the spring 6420 is disposed about the proximal end portion 6610 of the transfer member 6600, and is configured to engage the spring seat 6615 defined by the ring protrusion 6612.

The transfer member 6600 further includes a latch extension 6617 that extends from a distal surface 6614 of the ring protrusion 6612. The latch extension 6617 includes the latch arm 6618 and a bendable portion 6622. The latch arm 6618 includes the first guide surface 6619, the latch 6620, the guide protrusion 6624, and the second guide surface 6626. As described above, the latch extension 6617 extends in a distal direction from the ring protrusion 6612 of the transfer member 6600. The latch arm 6618 is configured to extend from the distal end portion 6611 of the transfer member 6610. Similarly stated, the latch arm 6618 extends from a distal end portion of the latch extension 6617. Moreover, the latch arm 6618 extends from the distal end portion of the latch extension 6617 at a suitable angle such that the latch 6620 is received within the opening 6316 of the first movable member 6301 (see e.g., FIGS. 131 and 132). For example, in some embodiments, the latch arm 6618 extends from the distal end portion of the latch extension 6617 at an acute angle. The first guide surface 6619, the second guide surface 6626, and the guide protrusion 6624 engage the transfer member guide 6117 of the housing 6100, as described above.

The latch 6620 extends from a proximal end portion 6623 of the latch arm 6618. The latch 6620 is configured to engage the second latch protrusion 6317 of the latch portion 6310 of the first movable member 6301. As described above, the distal surface 6621 of the latch 6620 is configured to be in contact with a proximal surface 6318 of the second latch protrusion 6317 when the transfer member 6600 is in the first configuration. In this manner, the transfer member 6600 transfers a force from the actuation of the spring 6420 to the first movable member 6301 and/or the medicament delivery mechanism 6300 to move the medicament delivery mechanism 6300 in the distal direction within the housing 6100. In this manner, the force produced by the spring 6420, which is offset from the medicament delivery mechanism 6300 and/or the medicament container 6210, results in both the insertion of the needle 6216 and injection of the medicament within the medicament container 6210. Although, as described below, the mixing spring 6390 produces a force to mix a diluent and a lyophilized medicament, in other embodiments, a portion of the force produced by the spring 6420 can be used to facilitate the mixing process.

Furthermore, when the transfer member 6600 has moved a desired distance in the distal direction in response to the force produced by the actuation of the spring 6420 (e.g., upon completion of the medicament injection), the guide protrusion 6624 of the latch 6620 aligns with the lower notch 6121 of the housing 6100 (see e.g., FIG. 110) to allow the transfer member 6600 to be moved to the second configuration (see e.g., FIG. 161). Expanding further, when the guide protrusion 6624 is aligned with the lower notch 6121 the guide protrusion 6624 moves through the lower notch 6121 thereby placing transfer member 6600 in the second configuration. In this manner, the latch 6620 can be disengaged from the second latch protrusion 6317. Similarly stated, when the transfer member 6600 is in its second configuration, the late 6620 is disengaged from the first movable member 6301, and the force produced by the spring 6420 is no longer transferred to the medicament delivery mechanism 6300. In particular, the bendable portion 6622 of the latch extension 6617 is configured to bend, relative to the latch extension 6617. In some embodiments, the bendable portion 6622 can define a pre-stress load such that when the transfer member 6600 is in the first configuration, the bendable portion 6622 is in a bent or deformed position. Thus, when the guide protrusion 6624 is aligned with the lower notch 6121, the bendable portion 6622 of the transfer member 6600 bends (e.g., returns to an undeformed position), thereby placing the transfer member 6600 in its second configuration (see FIG. 161).

When the transfer member 6600 is in its second configuration, the latch 6620 is disengaged from the second latch protrusion 6317 of the first movable member 6301. Said another way, when the guide protrusion 6624 of the latch 6620 is aligned with the lower notch 6121, the bendable portion 6622 of the transfer member 6600 bends (e.g., returns to the undeformed configuration) such that the angle between the latch arm 6618 and the latch extension 6617 is reduced, thus disengaging the transfer member 6600 from the medicament delivery mechanism 6300. Said yet another way, when the transfer member 6600 is in its second configuration, the medicament delivery mechanism 6300 is isolated and/or no longer operably coupled to the spring 6420. In this manner, as described below, the retraction force exerted by the retraction spring 6440 moves the medicament delivery mechanism 6300 and/or the medicament container assembly 6200 proximally within the housing 6100 to retract the needle 6216.

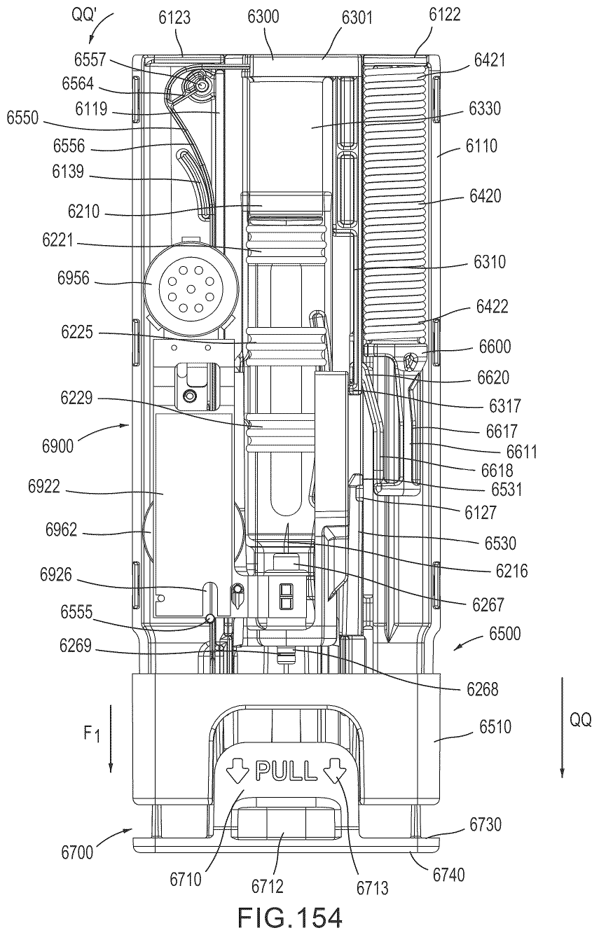

FIGS. 136-141 show the electronic circuit system 6900. The electronic circuit system 6900 of the medical injector 6000 includes a printed circuit board 6922, a battery assembly 6962, an audio output device 6956, two light emitting diodes (LEDs) 6958A, 6958B and a battery clip 6910. The electronic circuit system 6900 is disposed within the housing 6100 (see e.g., FIG. 154). As described herein, the electronic circuit system 6900 is configured to output an electronic output associated with the use of the medical injector 6000.

As described above, the electronic circuit system 6900 is coupled to the second housing member 6140 of the housing 6100. In some embodiments, the electronic circuit system 6900 can be coupled to the housing 6100 by any suitable means such as an adhesive, a clip, a label and/or the like. As described in more detail herein, the battery clip protrusion 6176 of the second housing member 6140 is configured to hold the battery clip 6910 in place. Similarly stated, the battery clip protrusion 6176 of the second housing member 6140 is configured to exert a force on the battery clip 6910 to ensure that electrical contact between the battery assembly 6962 and the battery clip 6910 is maintained when the battery isolation protrusion 6197 of the cover 6190 is removed.

As shown and described above with respect to FIG. 111, the second housing member 6140 defines the sounds apertures 6173, the LED aperture 6178, the first actuator groove 6179, and the second actuator groove 6180. The audible output device 6956 is disposed within the recess 6165 defined by the inner surface 6146 of the second housing member 6140 such that the front face of the audible output device 6956 is disposed adjacent the sound apertures 6173. In this manner, the sound apertures 6173 are configured to allow sound produced by the audio output device 6956 to pass from the audio output device 6956 to a region outside of the housing 6100. Furthermore, the audio output device 6956 includes the tabs 6957 configured to engage the latches 6163 of the second housing member 6140.

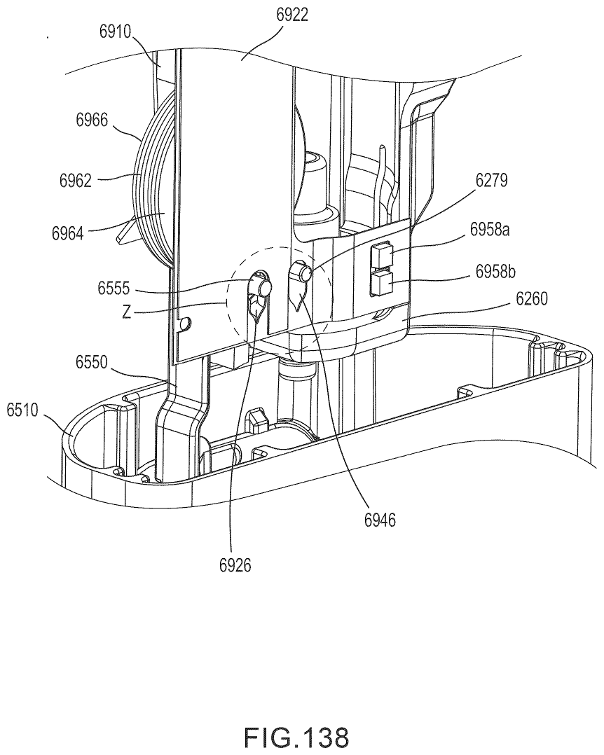

The first actuator groove 6179 defined by the second housing member 6140 is disposed adjacent the safety lock actuator groove 6133 defined by the first housing member 6110. In this manner, the first actuator groove 6179 of the second housing member 6140 and the safety lock actuator groove 6133 of the first housing member 6110 collectively receive the protrusion 6555 of the mixing actuator member 6550 (see e.g., FIGS. 138 and 149), which is described in more detail herein. The second actuator groove 6180 of the second housing member 6140 is configured to receive the protrusion 6279 of the electronic engagement portion 6278 included in the carrier 6260 (see e.g., FIGS. 129 and 138), which is described in more detail herein.

The printed circuit board 6922 of the electronic circuit system 6900 includes a substrate 6924, a first actuation portion 6926 and a second actuation portion 6946. The substrate 6924 of the printed circuit board 6922 includes the electrical components for the electronic circuit system 6900 to operate as desired. For example, the electrical components can be resistors, capacitors, inductors, switches, microcontrollers, microprocessors and/or the like. The printed circuit board 6922 may also be constructed of materials other than a flexible substrate such as a FR4 standard board (rigid circuit board).

As shown in FIGS. 138-141, the first actuation portion 6926 includes a first electrical conductor 6934 and defines an opening 6928 having a boundary 6929. The opening 6928 of the first actuation portion 6926 is configured to receive the protrusion 6555 of the mixing actuator member 6550. The boundary 6929 of the opening 6928 has a discontinuous shape, such as, for example, a teardrop shape, that includes a stress concentration riser 6927. The discontinuity and/or the stress concentration riser 6927 of the boundary 6929 can be of any suitable shape to cause the substrate 6924 to deform in a predetermined direction when the protrusion 6555 of the mixing actuator member 6550 is moved relative to the opening 6928, as shown by the arrow NN in FIG. 140.

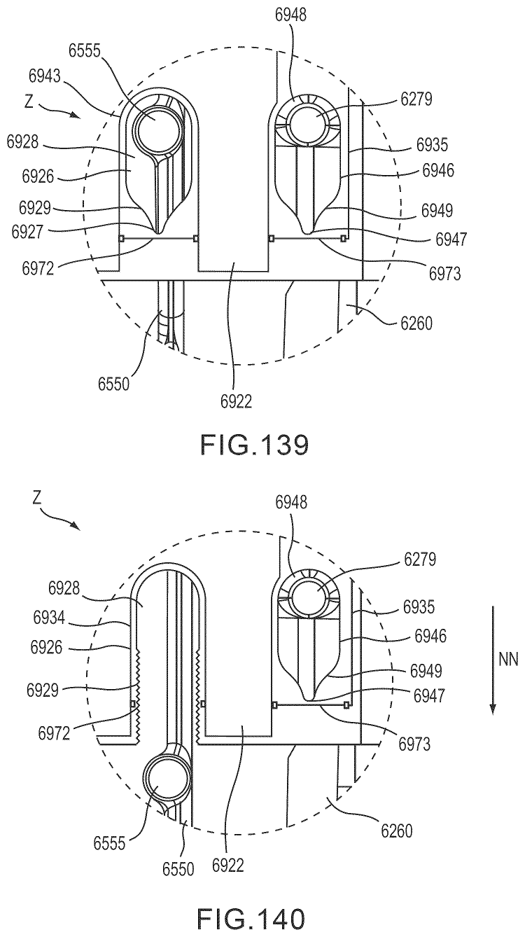

The opening 6928 is defined adjacent the first electrical conductor 6934 that electronically couples the components included in the electronic circuit system 6900. The first electrical conductor 6934 includes a first switch 6972, which can be, for example a frangible portion of the first electrical conductor 6934. In use, when the safety lock 6700 is moved in the distal direction from the first position to the second position, the protrusion 6726 of the actuator 6724 engages the catch 6553 of the mixing actuator member 6550 and moves the distal end portion 6551 of the mixing actuator member 6550 in the distal direction. In this manner, the protrusion 6555 of the mixing actuator member 6550 moves from a first position (see e.g., FIG. 139) to a second position (see e.g., FIG. 140). The movement of the mixing actuator member 6550 causes the protrusion 6555 to move within the first opening 6928, as indicated by the arrow NN in FIG. 140. The movement of the protrusion 6555 tears the first actuation portion 6926 of the substrate 6924, thereby separating the portion of the first electrical conductor 6934 including the first switch 6972. Said another way, when the safety lock 6700 is moved from its first position to its second position (see e.g., FIG. 154), the mixing actuator member 6550 is actuated and the protrusion 6555 moves the first switch 6972 from a first state (e.g., a state of electrical continuity) to a second state (e.g., a state of electrical discontinuity). Said yet another way, when the safety lock 6700 is moved from its first position to its second position, the mixing actuator member 6550 disrupts the first electrical conductor 6934.

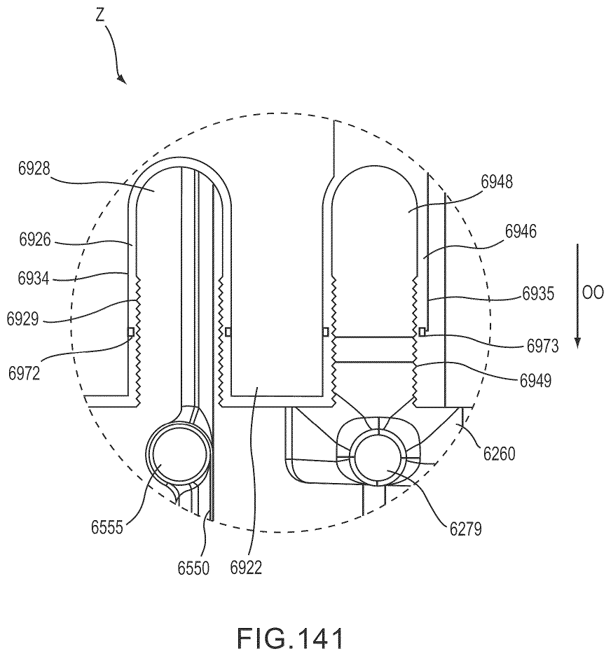

The second actuation portion 6946 includes a second electrical conductor 6935 and defines an opening 6945 having a boundary 6949. As shown in FIGS. 138-141, the opening 6945 of the second actuation portion 6946 is configured to receive the protrusion 6279 of the electronic engagement portion 6278 of the carrier 6260. The boundary 6949 of the opening 6945 has a discontinuous shape that includes a stress concentration riser 6947. The discontinuity and/or the stress concentration riser 6947 of the boundary 6949 can be of any suitable shape to cause the substrate 6924 to deform in a predetermined direction when the protrusion 6279 of the carrier 6260 is moved in a proximal direction relative to the opening 6945, as shown by the arrow OO in FIG. 141.

The second electrical conductor 6935 includes a second switch 6973, which can be, for example, a frangible portion of the second electrical conductor 6935. In use, when the carrier 6260 is moved from its first position to its second position (see e.g., FIG. 158), the protrusion 6555 moves in a distal direction, substantially parallel to a plane defined by a surface of the second actuation portion 6946 of the substrate 6924. The distal movement of the protrusion 6555 tears the second actuation portion 6946 of the substrate 6924, thereby separating the portion of the second electrical conductor 6935 including the second switch 6973. Said another way, when the carrier 6260 is moved from its first position to its second position, the protrusion 6555 moves the second switch 6973 from a first state (e.g., a state of electrical continuity) to a second state (e.g., a state of electrical discontinuity). In some embodiments, other portions the medical injector 6000 can engage the first electrical conductor 6934 or the second electrical conductor 6935 to actuate the electronic circuit system 6900. For example, in some embodiments, a base can include an actuator such that the proximal movement of the base can urge an actuator to move in the proximal direction to actuate the electronic circuit system.

In some embodiments, the safety lock 6700, the mixing actuator member 6550 and/or other portions of the medical injector 6000 can be configured to interact with mechanical and/or optical switches to produce an electronic output in a reversible manner. For example, in some embodiments, the electronic circuit system 6900 can include one or more optical switches configured to change states based on the sensed position of one of the plungers within the medicament container 6210. In some such embodiments, the electronic circuit system 6900 can produce an output when the mixing event has ended based at least in part upon the location of a plunger within the medicament container.

The battery assembly 6962 of the electronic circuit system 6900 includes two batteries stacked on top of one another. In other embodiments, the electronic circuit system can include any number of batteries and/or any suitable type of power source. In some embodiments, for example, the battery assembly can include Lithium batteries such as, for example, CR61616, CR62016s, type AAA or the like. The battery assembly 6962 has a first surface 6964 and a second surface 6966. The first surface 6964 of the battery assembly 6962 can contact an electrical contact (not shown) disposed on the substrate 6924. The second surface 6966 of the battery assembly 6962 is configured to contact a contact portion 6918 of a distal end portion 6916 of a battery clip 6910. When both the electrical contact of the substrate 6924 and the contact portion 6918 of the distal end portion 6916 of the battery clip 6910 contact the battery assembly 6962, the batteries of the battery assembly 6962 are placed in electrical communication with the electronic circuit system 6900. Said another way, when the electrical contact of the substrate 6924 and the contact portion 6918 of the distal end portion 6916 of the battery clip 6910 contact the battery assembly 6962, the battery assembly 6962 is configured to supply power to the electronic circuit system 6900.

The battery clip 6910 (shown in FIG. 136) includes a proximal end portion 6912 and a distal end portion 6916. The proximal end portion 6912 defines a retention aperture (not shown). The retention aperture is configured to receive a screw 6911 to couple the battery clip 6910 to the battery clip protrusion 6176 of the second housing member 6140. In this manner, the battery clip protrusion 6176 maintains the position of the battery clip 6910 with respect to the electronic circuit system housing 6170 and/or the battery assembly 6962.

The distal end portion 6916 of the battery clip 6910 includes a contact portion 6918 and an angled portion 6917. As described above, the contact portion 6918 is configured to contact the second surface 6966 of the battery assembly 6962 to place the battery assembly 6962 in electrical communication with the electronic circuit system 6900. The angled portion 6917 of the distal end portion 6916 of the battery clip 6910 is configured to allow a proximal end portion 6236 of a battery isolation protrusion 6197 (see e.g., FIG. 143) to be disposed between the second surface 6966 of the battery assembly 6962 and the contact portion 6918 of the distal end portion 6916 of the battery clip 6910. When the battery isolation protrusion 6197 is disposed between the second surface 6966 of the battery assembly 6962 and the contact portion 6918 of the distal end portion 6916 of the battery clip 6910, the electrical path between the battery assembly 6962 and the remainder of the electrical circuit system 6900 is disrupted, thereby removing power from the electronic circuit system 6900. The contact portion 6918 of the distal end portion 6916 of the battery clip 6910 is biased such that when the battery isolation protrusion 6197 is removed, the contact portion 6918 will move into contact the second surface 6966 of the battery assembly 6962, thereby restoring electrical communication between the battery assembly 6962 and the electronic circuit system 6900. In some embodiments, the battery isolation protrusion 6197 can be repeatedly removed from between the second surface 6966 of the battery assembly 6962 and the contact portion 6918 of the distal end portion 6916 of the battery clip 6910 and reinserted. Said another way, the battery isolation protrusion 6197 and the battery clip 6910 collectively form a reversible on/off switch.

The audio output device 6956 of the electronic circuit system 6900 is configured to output audible sound to a user in response to use of the medical injector 6000. In some embodiments, the audible output device 6956 can be a speaker. In some embodiments, the audible sound can be, for example, associated with a recorded message and/or a recorded speech. In other embodiments, the audible instructions can be an audible beep, a series of tones and/or or the like.

In some embodiments, the medical injector 6000 can have a network interface device (not shown) configured to operatively connect the electronic circuit system 6900 to a remote device (not shown) and/or a communications network (not shown). In this manner, the electronic circuit system 6900 can send information to and/or receive information from the remote device. The remote device can be, for example, a remote communications network, a computer, a compliance monitoring device, a cell phone, a personal digital assistant (PDA) or the like. Such an arrangement can be used, for example, to download replacement processor-readable code from a central network to the electronic circuit system 6900. In some embodiments, for example, the electronic circuit system 6900 can download information associated with a medical injector 6000, such as an expiration date, a recall notice, updated use instructions or the like. Similarly, in some embodiments, the electronic circuit system 6900 can upload compliance information associated with the use of the medical injector 6000 via the network interface device.

FIGS. 142 and 143 show the cover 6190 of the medical injector 6000. The cover 6190 includes a proximal end portion 6191 and a distal end portion 6192, and defines a cavity 6196. The cavity 6196 of the cover 6190 is configured to receive at least a portion of the housing 6100. Thus, when the portion of the housing 6100 is disposed within the cover 6190, the cover 6190 blocks an optical pathway between the medicament container 6210 and a region outside of the housing 6100. Similarly stated, when the portion of the housing 6100 is disposed within the cover 6190, the cover 6190 is obstructs the first status indicator aperture 6130 and/or the second status indicator aperture 6160 of the housing 6100 to reduce the amount of light transmitted to the medicament within the medicament container 6210. In this manner, the life of the medicament can be extended by the prevention and/or reduction of degradation to the medicament that may be caused by ultra-violet radiation. In some embodiments, for example, when the medicament is not degraded by ultra-violet radiation, the cover 6190 can include status indicator apertures similar to the status indicator aperture 6130 and/or 6160.

As described above, the electronic circuit system 6900 can be actuated when the housing 6100 is at least partially removed from the cover 6190. More particularly, the distal end portion 6192 of the cover 6190 includes the battery isolation protrusion 6197. The battery isolation protrusion 6197 includes a proximal end portion 6198 and a distal end portion 6199. The proximal end portion 6198 of the battery isolation protrusion 6197 is configured to be removably disposed between the second surface 6966 of the battery assembly 6962 and the contact portion 6918 of the distal end portion 6916 of the battery clip 6910, as described above.

The cover 6190 can be any suitable configuration and can include any suitable feature. For example, the cover 6190 includes notches 6194 disposed at the proximal end of the cover 6190. In some embodiments, the notches 6194 can be used to reduce the material needed to manufacture the cover 6190. In some embodiments, the cover 6190 can include openings that can receive inserts (not shown). The inserts can be a flexible inserts and can be configured to increase friction between the cover 6190 and a surface. For example, the inserts can increase the friction between the cover 6190 and a surface on which the medical injector 6000 is placed, to prevent sliding.

FIGS. 144-148 show the safety lock 6700 of the medical injector 6000. The safety lock 6700 of the medical injector 6000 includes a proximal surface 6730, a distal surface 6740 opposite the proximal surface 6730 and a needle sheath 6820. The safety lock 6700 defines a needle sheath aperture 6703 and a battery isolation protrusion aperture 6728. The battery isolation protrusion aperture 6728 is configured to receive the battery isolation protrusion 6197 of the cover 6190 such that the battery isolation protrusion 6197 can be disposed within the housing 6100 and/or in engagement with the electronic circuit system 6900, as described above. Similarly stated, the battery isolation protrusion aperture 6728 of the safety lock 6700 is aligned with the battery isolation protrusion aperture 6135 of the housing 6100, such that the battery isolation protrusion 6197 can be disposed within the housing 6100 when the cover 6190 is disposed about a portion of the housing 6100.

The proximal surface 6730 of the safety lock 6700 includes the safety lock protrusions 6702, the actuator 6724, two opposing pull-tabs 6710, and an engagement portion 6720. As described above, when the safety lock 6700 is in a first (locked) position, the safety lock protrusions 6702 are disposed in the safety lock protrusion opening 6514 defined by the base 6510 and in contact with a distal surface 6107 of the housing 6100 (see e.g., FIGS. 116 and 153). Accordingly, the safety lock protrusions 6702 are configured to prevent the proximal movement of the base 6510 and/or delivery of the medicament.

The actuator 6724 of the safety lock 6700 defines the channel 6725 and includes the protrusion 6726. The actuator 6724 can extend from the proximal surface 6730 of the safety lock 6700 and through a safety lock actuator opening 6524 of the base 6510 (see e.g., FIG. 150). As described above, the channel 6725 receives a catch 6553 of the mixing actuator member 6550 such that the protrusion 6726 can engage the catch 6553. The protrusion 6726 extends in a direction substantially transverse to the actuator 6724 and/or substantially parallel to the proximal surface 6730 of the safety lock 6700. As described above, the protrusion 6726 can engage the catch 6553 to move the mixing actuator member 6550 in the distal direction when the safety lock 6700 is moved distally to remove the needle sheath 6820 and/or prepare the medical injector 6000 for use.

The pull-tabs 6710 of the safety lock 6700 include a grip portion 6712 and indicia 6713. The grip portion 6712 of the pull-tabs 6710 provides an area for the user to grip and/or remove the safety lock 6700 from the rest of the medicament delivery system 6700. The indicia 6713 provide instruction on how to remove the safety lock 6700. The distal end surface 6740 also includes indicia 6741 (see e.g., FIG. 146). In some embodiments, for example, indicia can indicate the direction the user should pull the safety lock 6700 to remove the safety lock 6700. In other embodiments, indicia can indicate the medical injector 6000 is a trainer (e.g., that the medical injector 6000 is devoid of a needle and/or an active medicament).

The engagement portion 6720 of the safety lock 6700 includes engagement members 6721. The engagement members 6721 extend in a proximal direction from the proximal surface 6730. The engagement members 6721 have tabs 6722 that extend from a surface of the engagement members 6721. The tabs 6722 are configured to engage a rib 6825 disposed at a distal end portion 6822 of the needle sheath 6820. In this manner, distal movement of the safety tab 6700 results in distal movement (e.g., removal of) the needle sheath 6820.

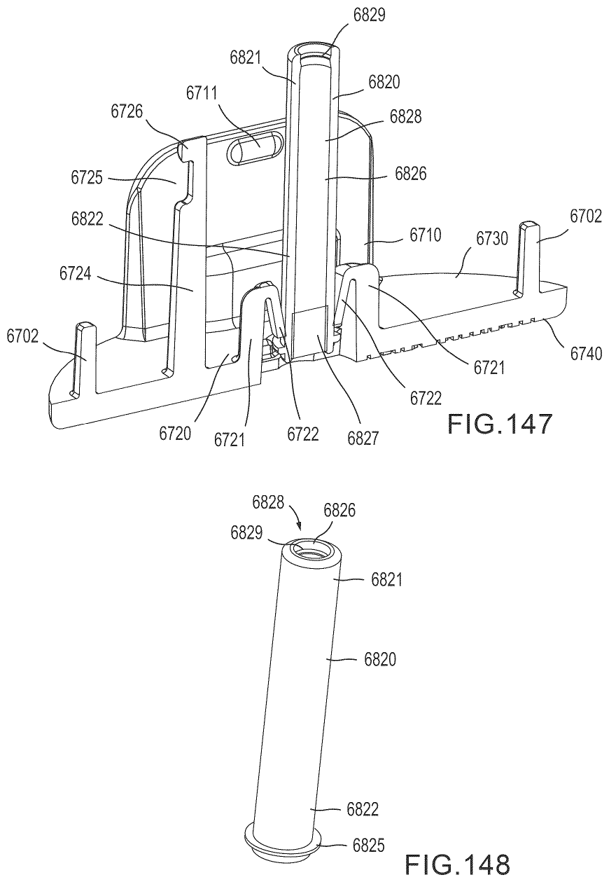

As shown in FIGS. 147 and 148, the needle sheath 68210 includes the distal end portion 6822, a proximal end portion 6821, the rib 6825, and a needle plug 6827. The needle sheath 6820 also defines a bore 6828. The bore 6828 is defined by an inner surface 6826 of the needle sheath 6820 and is configured to receive the needle 6216 and/or a distal end portion of the 6213 of the medicament container 6200. The needle plug 6827 is disposed within the bore 6828 at the distal end portion 6822 of the needle sheath 6820. The needle plug 6827 can be any suitable material configured to engage a proximal end portion 6218 of the needle 6216. For example, in some embodiments, the needle plug can be a cork material or any other suitable porous material (e.g., any suitable Porex™ material) to allow for exposure to ethylene oxide during a sterilization operation.

The inner surface 6826 further define an annular protrusion 6829 disposed at the proximal end portion 6281 of the needle sheath 6820 and is configured to engage an annular notch 6269 defined by the lower needle port 6268 of the carrier 6260. The annular protrusion 6829 defines a friction fit with the annular notch 6269 of the carrier 6260. In this manner, the needle sheath 6820 can be coupled to the carrier 6260 and can protect the user from the needle 6216 and/or can keep the needle 6216 sterile before the user actuates the medical injector 6000.

The distal end portion 6822 of the needle sheath 6820 is configured to be inserted into a space defined between the tabs 6722 of the engagement members 6721 of the safety lock 6700. The tabs 6722 are angled and/or bent towards the distal direction to allow the distal end portion 6822 of the needle sheath 6810 to move between the engagement members 6721 in a distal direction, but not in a proximal direction. Similarly stated, the tabs 6722 include an edge that contacts the rib 6825 of the needle sheath 6820 to prevent the safety lock 6700 from moving in a distal direction relative to the needle sheath 6820. In this manner, the needle sheath 6820 is removed from the needle 6216 when the safety lock 6700 is moved in a distal direction with respect to the housing 6100 (see e.g., FIG. 154).

FIG. 149 shows the mixing actuator member 6550 of the medical injector 6000. The mixing actuator member 6550 includes the proximal end portion 6551, the distal end portion 6552, and the engagement portion 6558. The distal end portion 6552 includes the protrusion 6555 and the catch 6553 having an engagement surface 6554. As described above, the catch 6553 is configured to engage the protrusion 6726 of the actuator 6724 included in the safety lock 6700. In this manner, when the safety lock 6700 is moved in the distal direction, the protrusion 6726 contacts the engagement surface 6554 of the catch 6553 and moves the distal end portion 6552 of the mixing actuator member 6550 in the distal direction. Thus, when the distal end portion 6552 of the mixing actuator member 6550 is moved in the distal direction, the protrusion 6555 is moved in the distal direction to actuate a portion of the electronic circuit system 6900, as described above.

The proximal end portion 6551 defines a curved portion 6556 and includes the pivot protrusions 6557. As described above, the pivot protrusions 6557 are disposed within the pivot protrusion apertures 6125 and 6155 of the housing 6100, such that the mixing actuator member 6550 can pivot about the pivot protrusions 6557 when actuated. Furthermore, the proximal end portion 6551 includes a stiffening arm 6564 configured to facilitate the pivot motion of the mixing actuator member 6550. Expanding further, the stiffening arm 6564 can be configured to transfer and/or amplify of a portion of a force exerted on the catch 6553 by the distal movement of the safety lock 6700 to move the retention portion 6558 in a lateral direction (e.g., a direction substantially perpendicular to the distal direction), as described in further detail herein. More particularly, the stiffening arm 6564 is configured such that the curved portion 6556 of the mixing release member 6550 is spaced apart from the pivot protrusions 6557 by a first distance and the retention portion 6558 is spaced apart from the pivot protrusions 6557 by a second distance, less than the first distance. In this manner the force exerted by the retention portion 6558 during rotation of a portion of the mixing release member 6550 is greater than the force applied to the distal end portion 6552 of the mixing release member 6550.

The retention portion 6558 extends in a substantially normal direction from distal end portion 6552 of the mixing release member 6550. Similarly stated, the retention portion 6558 is substantially perpendicular to a portion of the mixing release member 6550 defined between the proximal end portion 6551 and the distal end portion 6552. The retention portion 6558 includes a lock surface 6560 and defines a set of notches 6559. As described above, the lock surface 6560 can selectively engage the retention protrusions 6379 of the mixing piston 6370 to maintain the mixing piston 6370 in the first (e.g., locked) configuration. The notches 6559 are configured to receive a set of the retention protrusions 6379 when the retention portion 6558 is moved to the second position (e.g., when the safety lock 6700 is removed from the housing 6100).

FIGS. 150 and 151 show the base 6510 of the medical injector 6000. The base 6510 includes a proximal surface 6511, a distal surface 6523 and base connection knobs 6518. The base 6510 defines a needle aperture 6513, the safety lock protrusion apertures 6514, the battery isolation protrusion aperture 6521, the safety lock actuator opening 6524, and pull-tab openings 6519. The needle aperture 6513 is configured to receive the needle 6216 when the medical injector 6000 is actuated. The safety lock protrusion apertures 6514 of the base 6510 receive the safety lock protrusions 6702 of the safety lock 6700 when the safety lock 6700 is coupled to the housing 6100 and/or the base 6510. The battery isolation protrusion aperture 6521 of the base 6510 receives the battery isolation protrusion 6197 of the cover 6190. The safety lock actuator opening 6524 receives the actuator 6724 of the safety lock 6700 when the safety lock 6700 is coupled to the housing 6100 and/or the base 6510. The pull-tab openings 6519 are configured to receive the pull-tabs 6710 of the safety lock 6700 when the safety lock 6700 is coupled to the housing 6100 and/or the base 6510.

The proximal surface 6511 of the base 6510 includes and/or is coupled to the release member 6530 and the guide members 6517. The release member 6530 includes a proximal end portion 6531 and a distal end portion 6532 and defines a channel 6533 between a system lock surface 6534 and the distal end portion 6532 (see e.g., FIG. 151). The system lock surface 6534 is disposed at the proximal end portion 6531, and is configured to engage the first latch protrusion 6315 of the medicament delivery mechanism 6300 when the medical injector 6000 is in the first configuration. Moreover, the system lock surface 6534 engages the first latch protrusion 6315 such that the system lock surface 6534 maintains the engagement of the first latch protrusion 6315 and the latch member notch 6120, as described above and shown in FIGS. 114 and 115. Similarly stated, the system lock surface 6534 of the release member 6530 applies a force to the first latch protrusion 6315 to maintain the first latch protrusion 6315 within the latch member notch 6120. In this manner, distal movement of the first movable member 6301 and/or the medicament delivery mechanism 6300 is limited. When the base 6510 is moved in a proximal direction, as described in further detail herein, the system lock surface 6534 moves in the proximal direction to disengage from the first latch protrusion 6315. In response, the first latch protrusion 6315 moves within the channel 6533 of the release member 6530 in a distal direction, as described in further detail herein. Similarly stated, upon actuation of the medicament injector 6000, a portion of the medicament delivery mechanism 6300 moves within the release member 6530.

The guide members 6517 of the base 6510 are configured to engage and/or slide within the base rail grooves 6114 of the housing 6100, as described above. As described above, the base connection knobs 6518 are configured to engage the base retention recesses 6134A, 6134B in a way that allows proximal movement of the base 6510 but limits distal movement of the base 6510 relative to the housing 6100.

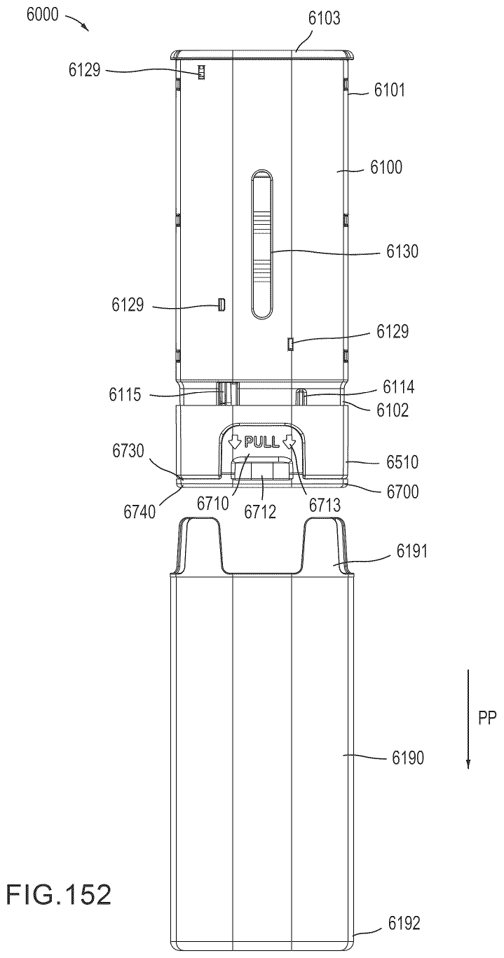

As shown in FIG. 152, the medical injector 6000 is first enabled by moving the medicament delivery device 6000 from the first configuration to the second configuration by moving the cover 6190 from a first position to a second position. The cover 6190 is moved from the first position to the second position by moving it with respect to the housing 6100 in the direction shown by the arrow PP in FIG. 152. When the cover 6190 is moved with respect to the housing 6100 in the direction PP, the battery isolation protrusion 6197 is removed from the area between the battery clip 6910 and the second surface 6966 of the battery assembly 6962. In this manner, the battery assembly 6962 is operatively coupled to the electronic circuit system 6900 when the cover 6190 is removed, thereby providing power to the electronic circuit system 6900. Similarly stated, this arrangement allows the electronic circuit system 6900 to be actuated when the cover 6190 is removed.

When power is provided, as described above, the electronic circuit system 6900 can output one or more predetermined electronic outputs. For example, in some embodiments, the electronic circuit system 6900 can output an electronic signal associated with recorded speech to the audible output device 6956. Such an electronic signal can be, for example, associated with a. WAV file that contains a recorded instruction, instructing the user in the operation of the medical injector 6000. Such an instruction can state, for example, “Remove the safety tab near the base of the auto-injector.” The electronic circuit system 6900 can simultaneously output an electronic signal to one and/or both of the LEDs 6958A, 6958B thereby causing one and/or both of the LEDs 6958A, 6958B to flash a particular color. In this manner, the electronic circuit system 6900 can provide both audible and visual instructions to assist the user in the initial operation of the medical injector 6000.

In other embodiments, the electronic circuit system 6900 can output an electronic output associated with a description and/or status of the medical injector 6000 and/or the medicament contained therein. For example, in some embodiments, the electronic circuit system 6900 can output an audible message indicating the symptoms for which the medicament should be administered, the expiration date of the medicament, the dosage of the medicament or the like.

In yet other embodiments, the electronic circuit system 6900 can output a wireless signal to a cell phone, computer, compliance tracking device, emergency dispatch system or the like. For example, in some embodiments, the electronic circuit system 6900 can output an wireless signal to a compliance tracking device, which receives the signal and monitors the activity (e.g., the arming of, the use of or the like) of the medical injector 6000.

In some embodiments, the medical injector 6000 can be repeatedly moved between the first configuration and the second configuration when the cover 6190 is moved repeatedly between the first position and the second position, respectively. Said another way, in some embodiments, the cover 6190 can be removed and replaced about the housing 6100 any number of times. When the cover 6190 is moved from the second position to the first position, the battery isolation protrusion 6197 is inserted between the battery clip 6910 and the second surface 6966 of the battery assembly 6962, deactivating the electronic circuit system 6900. When the cover is moved from the first position to the second position a second time, the electronic circuit system 6900 is once again activated. In other embodiments, the cover 6190 is configured to be removed from the housing only one time and the electronic circuit system 6900 is therefore configured output a single electronic output in response thereto, which, for example, can warn the user about the compromised sterility of the needle 6216.

After the cover 6190 is removed from the housing 6100, the medical injector 6000 is in the second configuration. As shown in FIG. 153, the medical injector 6000 is in a locked or pre-actuated position while in the second configuration. Thus, the safety lock protrusions 6702 of the safety lock 6700 are disposed within the safety lock protrusion openings 6514 of the base, and in contact with the distal surface 6107 of the housing 6100. With the safety lock 6700 coupled to the housing 6100 and/or the base 6510, the mixing actuator member 6550 is in a first position and/or configuration. As described above, the lock surface 6560 of the retention portion 6558 included in the mixing actuator member 6550 exerts a reaction force on the retention protrusions 6379 of the mixing piston 6370 (e.g., the second movable member 6370). In this manner, the mixing spring 6390 is maintained in the compressed configuration and the mixing piston 6370 remains in a first position, relative to the piston portion 6330 of the first movable member 6301. Therefore, the medicament container assembly 6200 remains in a first configuration (e.g., a pre-mixed configuration). In this configuration, the diluent volume 6236 is separated and/or fluidically isolated from the dry medicament volume 6237. Similarly, the dry medicament volume 6237 is substantially separated from the void volume 6238. The proximal end portion 6217 of the needle 6216 is disposed within the void volume 6238, and is therefore substantially isolated from the medicament. Furthermore, the distal end portion 6218 of the needle 6216 is disposed within the needle sheath 6820 such that a user is protected from a sharp point defined by the distal end of the needle 6216, and the sterility of the needle 6216 is maintained.

The medical injector 6000 can be moved from the second configuration (FIGS. 152 and 153) to a third configuration (FIGS. 154-157) by moving the safety lock 6700 from a first position to a second position. The safety lock 6700 is moved from a first position to a second position by moving the safety lock 6700 with respect to the housing 6100 in the direction shown by the arrow QQ in FIG. 154. When the safety lock 6700 is moved from the first position to the second position, the safety lock protrusions 6702 are no longer in contact with the distal surface 6107 of the housing 6100, and are removed from safety lock protrusion openings 6514 of the base, thereby enabling the medicament delivery mechanism 6300. Additionally, when the safety lock 6700 is removed from and/or moved relative to the housing 6100, the actuator 6724 of the safety lock 6700 also moves in the direction QQ to actuate the mixing actuator member 6550. More specifically, as described above (e.g., with respect to FIGS. 116, 147 and 149) the protrusion 6726 of the actuator 6724 is in contact with the engagement surface 6554 of the catch 6553; therefore, when the actuator 6724 is moved in the direction QQ, the protrusion 6726 exerts a first force F₁ on the engagement surface 6554 of the catch 6553 to move at least the distal end portion 6552 of the mixing actuator member 6550 in the direction QQ.

With the distal end portion 6552 of the mixing actuator member 6550 moved in the direction QQ, the protrusion 6555 of the mixing actuator member 6550 moves with relation to the first actuation portion 6926 of the electronic circuit system 6900, thereby moving the first switch 6972 from a first state (e.g., a state of electrical continuity) to a second state (e.g., a state of electrical discontinuity). When the protrusion 6555 moves the first switch 6972 of the electronic circuit system 6900 to the second state, the electronic circuit system 6900 can output one or more predetermined electronic outputs. For example, the protrusion 6555 can irreversibly move the first switch 6972 to the second state such that a processor (not shown) can output an electronic signal associated with recorded speech to the audible output device 6956. Such an electronic signal can be, for example, associated with a recorded message notifying the user of the status of the medical injector 6000. Such a status message can state, for example, “The needle guard has been removed and the mixing operation is no ongoing.” The electronic circuit system 6900 can also simultaneously output an electronic signal to one and/or both of the LEDs 6958A, 6958B, thereby causing one and/or both of the LEDs 6958A, 6958B to start flashing, stop flashing, change color, or the like.

In some embodiments, the first actuation portion 6926 and the protrusion 6555 can be configured such that the protrusion 6555 must move a predetermined distance before the protrusion 6555 engages the boundary 6929 of the opening 6928. For example, in some embodiments, the protrusion 6555 must move approximately 0.62 inches before the protrusion 6555 engages the boundary 6929 of the opening 6928. In this manner, the safety lock 6700 can be moved slightly without irreversibly moving the first switch 6972 of the electronic circuit system 6900 to the second state. Accordingly, this arrangement will permit the user to inadvertently and/or accidentally move the safety lock 6700 without actuating the electronic circuit system 6900.

In some embodiments, the electronic circuit system 6900 can be configured to output the status message for a predetermined time period, such as, for example, five seconds. After the predetermined time period has elapsed, the electronic circuit system 6900 can output an audible message further instructing the user in the operation of the medical injector 6000. Such an instruction can state, for example, “The mixing operation is now complete. Place the base of the auto-injector against the patient's thigh. To complete the injection, press the base firmly against the patient's thigh.” In some embodiments, the electronic circuit system 6900 can simultaneously output an electronic signal to one and/or both of the LEDs 6958A, 6958B, thereby causing one and/or both of the LEDs 6958A, 6958B to flash a particular color. In this manner, the electronic circuit system 6900 can provide both audible and/or visual instructions to assist the user in the placement and actuation of the medical injector 6000. In some embodiments, the electronic circuit system 6900 can be configured to repeat the instructions after a predetermined time period has elapsed.

In other embodiments, the output associated with the completion of the mixing operation (or any other operations described herein) need not be based on an elapsed time. For example, as described above, some such embodiments, the electronic circuit system 6900 can produce an output when the mixing event has ended based at least in part upon the location of a plunger within the medicament container.

As described above, in other embodiments, the medical injector 6000 can have a network interface device (not shown) configured to operatively connect the electronic circuit system 6900 to a remote device (not shown) and/or a communications network (not shown). In this manner, the electronic circuit system 6900 can send a wireless signal notifying a remote device that the safety lock 6700 of the medical injector 6000 has been removed and that the medical injector 6000 has been armed. In other embodiments, the electronic circuit system 6900 can send a wireless signal (e.g., a wireless 911 call) notifying an emergency responder that the medical injector 6000 has been armed.

The actuation of the mixing actuator member 6550 also actuates the mixing piston 6370. As described above, the protrusion 6726 of the actuator 6724 exerts the first force F₁ on the engagement surface 6554 of the catch 6553 such that at least a portion of the first force F₁ moves the mixing actuator member 6550 in the direction QQ. The mixing actuator guide 6119, the lower retention protrusions 6138, the upper retention protrusion 6139, and the upper mixing actuator plate 6123 of the first housing member 6110 (see e.g., FIG. 110) engage portions of the mixing actuator member 6550 to facilitate a desired motion, bending, flexing, or reconfiguration of at least a portion of the mixing actuator member 6550. This arrangement allows at least a portion of the mixing actuator member 6550 to pivot about the pivot protrusions 6557 disposed within the pivot protrusion apertures 6125 and 6155 of the housing, as shown by the arrow QQ′ in FIG. 154. Furthermore, the upper retention protrusion 6139 defines a curved shape configured to engage the curved portion 6556 of the mixing actuator member 6550. Thus, the housing 6100 and/or the upper retention protrusion 6139 define a channel and/or track within which and/or against which a portion of the mixing actuator member 6550 can move, flex, and/or bend in a non-linear manner.

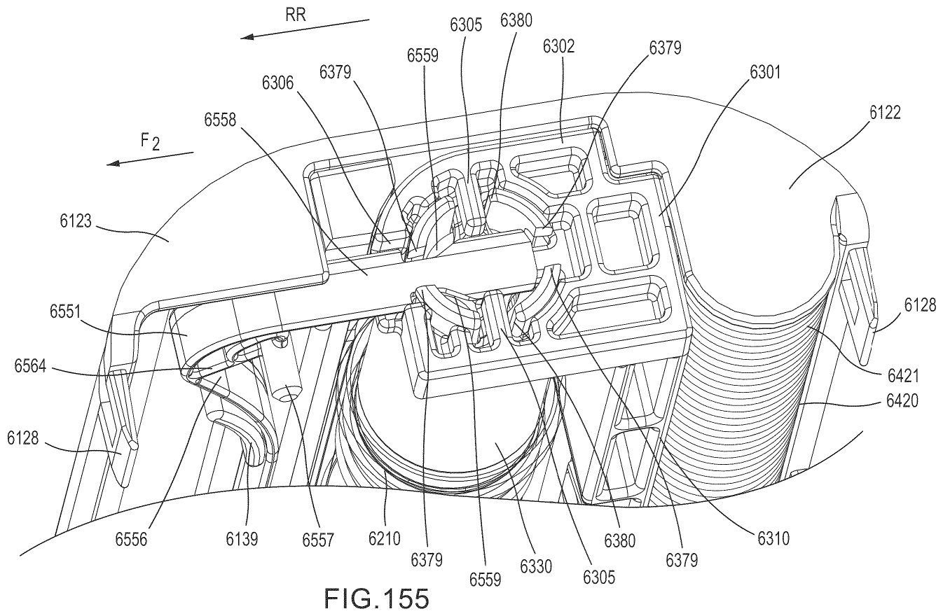

The arrangement of the portion of the mixing actuator member 6550 that defines the curved path 6556, the stiffening arm 6564, and the upper mixing actuator plate 6123 facilitate a transferring of a portion of the first force F₁ in the direction QQ into a second force F₂ in the direction RR, as shown in FIG. 155. Similarly stated, mixing actuator member 6550 is configured such that at least a portion of the first force F₁ exerted on the catch 6553 by the protrusion 6726 of the safety lock 6700 moves the retention portion 6558 in the direction RR. In some embodiments, the position of the pivot protrusions 6557, relative to the rest of the mixing actuator member 6550 and the stiffening arm 6564 are such that the transferring of the first force F₁ includes amplifying the first force F₁. More specifically, the length of the stiffening arm 6564 defines a first moment arm and the distance defined between the pivot protrusions 6557 and the retention portion 6558 defines a second moment arm, substantially smaller than the first moment arm. In this manner, a torque produced from the rotation about the pivot protrusions 6557 results in the amplification of the first force F₁ by the ratio of the length of the first moment arm to the length of the second moment arm.

By way of example, in some embodiments, the length of the first moment arm (e.g., the length of the stiffening arm 6564) can be four times as long as the length of the second moment arm (e.g., the length defined between the pivot protrusions 6557 and the retention portion 6558). Therefore, as a first force is applied in the direction QQ, the pivot motion of the mixing actuator member 6550 about the pivot protrusions 6557 results in a second force in the direction RR that is four times greater than the first force. Furthermore, this arrangement reduces the lateral translation of the retention portion 6558 (e.g., the translation of the portion of the mixing actuator member 6550 in the direction QQ is greater than the translation of the retention portion 6558 in the direction RR. In this manner, in some embodiments, the retention portion 6558 can be configured move in the direction RR with the second force F₂, resulting in a relatively fast movement of the retention portion 6558.

As shown in FIG. 155, the lateral motion of the retention portion 6558 disengages the lock surface 6560 from the retention protrusions 6379 of the mixing piston 6370. More specifically, with reference to FIG. 155, the retention portion 6558 is moved within the retention portion channel 6306 in the direction RR such that the retention protrusions 6379 disposed to the left of the alignment protrusion 6305 are at least momentarily positioned in alignment with the notches 6559 and the retention protrusions 6379 disposed to the right of the alignment protrusions 6305 are at least momentarily positioned adjacent to an end of the retention portion 6558. In this manner, the lock surface 6560 no longer exerts the reaction force on the distal surface of the retention protrusions 6379 to maintain the mixing spring 6390 in the first configuration (e.g., the compressed configuration). Therefore, when the retention portion 6558 moves laterally, the mixing spring 6390 expands to the second configuration and exerts a force F₃ to move the mixing piston 6370 in the distal direction, as indicated by arrow SS in FIG. 156.

With the mixing spring 6390 in the second configuration (e.g., the expanded configuration), much of the proximal end portion 6371 of the mixing piston 6370 is disposed outside of the opening 6333 defined by the piston portion 6330 of the first movable member 6301. Similarly stated, the proximal end portion 6371 of the mixing piston 6370 is disposed in a distal position relative to the distal end 6334 of the piston portion 6330 of the first movable member 6301. As described above, with the mixing piston 6370 outside of the piston portion 6330, the tabs 6378 (i.e., retention members or portions) included in the walls 6376 of the mixing piston 6370 expand to an undeformed position, as shown in FIG. 156. In this manner, the tabs 6378 can engage the distal end 6334 of the piston portion 6330 after the first movable member 6301 is moved to a second position, as described in further detail herein. More particularly, as shown in FIG. 158, the tabs 6378 (i.e., retention members or portions) are configured to engage the distal end 6334 of the piston portion 6330 to limit movement of the mixing piston 6370 relative to the first movable member 6301 during the needle insertion and/or injection operations.

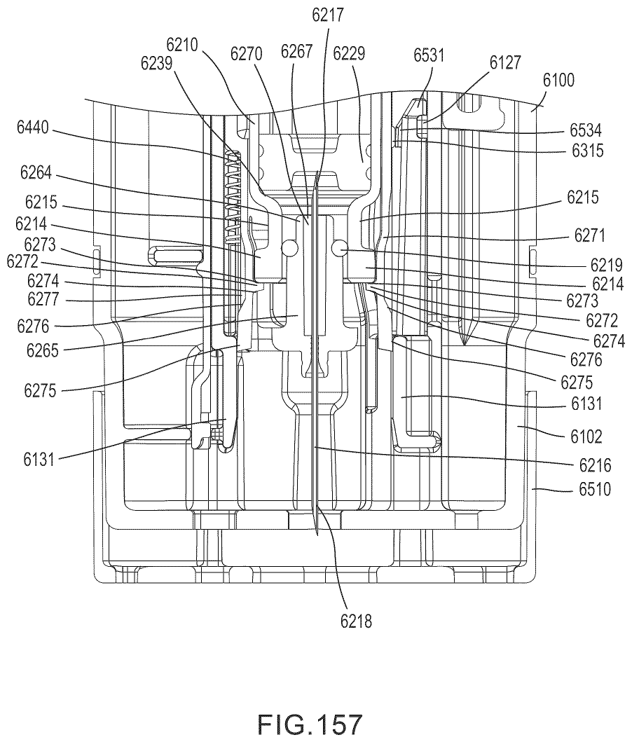

The distal movement of the mixing piston 6370 begins the mixing event, as shown in FIGS. 156 and 157. More specifically, the distal surface 6375 of the mixing piston 6370 engages the proximal surface 6222 of the first elastomeric member 6221 and transfers a portion of the force F₃ exerted by the mixing spring 6390 to move at least the first elastomeric member 6221 in the distal direction. The arrangement of the elastomeric members within the medicament container 6210 is such that the portion of the force F₃ exerted on the first elastomeric member 6221 moves the first elastomeric member 6221, the second elastomeric member 6225, and the third elastomeric member 6229 in the distal direction. Expanding further, the constituents in the diluent volume 6236, the dry medicament volume 6237, and the void volume 6238 are such that when the force F₃ is applied, the volume of the void volume 6238 is reduced. For example, in some embodiments, at a portion of a gas within the void volume 6238 is evacuated from the void volume 6238. In some embodiments, the gas can exit the void volume 6238 via the distal end portion 6213 of the medicament container 6210. In some embodiments, the gas can exit the void volume 6238 via the needle 6216. In this manner, the third elastomeric member 6229 is moved in the distal direction to contact a distal shoulder 6239 of the medicament container 6210. As shown in FIG. 157, the distal shoulder 6239 engages the distal surface 6231 of the third elastomeric member 6229 to stop the distal movement of the third elastomeric member 6229 such that the proximal end portion 6217 of the needle 6216 does not substantially puncture through the thickness T of the third elastomeric member 6229.

Concurrently, the application of the force F₃ results in the distal movement of the first elastomeric member 6221 and the second elastomeric member 6225. Therefore, as shown in FIG. 156, the diluent volume 6236 and the dry medicament volume 6237 are placed in fluid communication via the bypass 6220 such that the diluent within the diluent volume 6236 is transferred to the dry medicament volume 6237. In this manner, the diluent can mix with the lyophilized medicament disposed within the dry medicament volume 6237 to reconstitute the medicament for injection.

After the mixing event, the medical injector 6000 can be moved from the third configuration (FIG. 154-157) to a fourth configuration (FIGS. 158 and 159) by moving the base 6510 from a first position to a second position. Similarly stated, the medical injector 6000 can be actuated by the system actuator assembly 6500 by moving the base 6510 proximally relative to the housing 6100. The base 6510 is moved from its first position to its second position by placing the medical injector 6000 against the body of the patient and moving the base 6510 with respect to the housing 6100 in the direction shown by the arrow TT in FIG. 158.

When the base 6510 is moved from the first position to the second position, the system actuator assembly 6500 actuates the medicament delivery mechanism 6300, thereby placing the medical injector 6000 in its fourth configuration (i.e., the needle insertion configuration), as shown in FIGS. 158 and 159. More specifically, the proximal movement of the system actuator assembly 6500 and/or the base 6510 moves the release member 6530 in the proximal direction within the housing 6100, thereby allowing the first latch protrusion 6315 to be disengaged from the system lock surface 6534 of the proximal end portion 6533 of the release member 6530. Similarly stated, when the system actuator assembly 6500 is moved in the proximal direction, the system lock surface 6534 disengages the first latch protrusion 6315. Moreover, when the system lock surface 6534 moves in the proximal direction relative to the first latch protrusion 6315, the first latch protrusion 6315 moves and/or deforms substantially laterally into the channel 6533 defined by the release member 6530.

When the first latch protrusion 6315 is disposed within the channel 6533, the force applied by the system lock surface 6534 of the base 6510 to maintain the first latch protrusion 6315 within the latch member notch 6120 is removed and the first latch protrusion 6315 is allowed to disengage the latch member notch 6120. Therefore, the engagement surface 6109 of the latch member notch 6120 no longer applies the reaction force to the first latch protrusion 6315; thus, the spring 6420 is allowed to expand. As described above, the proximal end portion 6421 of the spring 6420 is in contact with the upper spring plate 6122 of the first housing member 6110 such that the spring 6420 expands in the direction shown be the arrow UU in FIG. 158. With the distal end portion 6422 of the spring 6420 in contact with the spring seat 6615 of the transfer member 6600, a force F₄ produced by the expansion of the spring 6420 is applied to the transfer member 6600, which moves the transfer member 6600 in the direction shown by the arrow UU. In this manner, the latch 6620 of the transfer member 6600 transfers at least a portion of the force F₄ to the second latch protrusion 6317 of the latch portion 6310 of the first movable member 6301 such that the portion of the force moves the medicament delivery mechanism 6300 in the distal direction, shown by the arrow UU in FIG. 158. Thus, the first movable member 6301 and the transfer member 6600 move together distally within the housing 6100.

When the medicament delivery mechanism 6300 is moving distally, the piston portion 6330 of the first movable member 6301 applies a portion of the force F₄ to the medicament container 6210. More specifically, the distal end 6334 of the piston portion 6330 engages the latches 6378 of the mixing portion 6370. With the latches 6378 in the extended position (described above), the piston portion 6330 can transfer a portion of the force F₄ to the mixing piston 6370 such that the mixing piston 6370 further transfers a portion of the force F₄ to the first elastomeric member 6221. With the first elastomeric member 6221, the second elastomeric member 6225, and the third elastomeric member 6229 in their respective second positions, the portion of the force F₄ transferred to the first elastomeric member 6221 moves the medicament container assembly 6200 within the housing 6100 to a third configuration. Expanding further, the mixed medicament contained within the mixed volume 6237 is such that the medicament is a substantially incompressible liquid; thus the portion of the force F₄ acts to move the medicament container assembly 6200 in the distal direction rather than moving the first elastomeric member 6221, the second elastomeric member 6225, and/or the third elastomeric member 6229 within the medicament container 6210.

As described above, the portion of the force F₄ exerted by the piston portion 6330 and/or the mixing piston 6370 moves the medicament container assembly 6200 in the distal direction. As shown in FIG. 157, when the medicament container assembly 6200 is in the first position (e.g., prior to being moved by the portion of the force F₄), an engagement surface 6275 of the needle insertion tabs 6271 included in the carrier 6260 are in contact with the carrier engagement surface 6131 included in the first housing member 6110. In this configuration, the flanged end 6214 of the medicament container 6210 is disposed on a proximal surface 6273 of the container shoulder 6272. Therefore, when the portion of the force F₄ is exerted on the first elastomeric member 6221, the force is transferred through the medicament container 6210 to the proximal surface 6273 of the container shoulder 6272. Thus, a portion of the force F₄ is exerted on the container shoulder 6272 to move the carrier 6260 in the distal direction (FIGS. 158 and 159).

As described above, when the carrier 6260 and/or medicament container assembly 6200 moves to the second position, the protrusion 6279 of the electronic engagement portion 6278 actuates the electronic circuit 6900 to trigger a predetermined output or sequence of outputs. When the protrusion 6279 is moved in the distal direction relative to the opening 6945, the second switch 6973 is moved from a first state (e.g., a state of electrical continuity) to a second state (e.g., a state of electrical discontinuity). When the protrusion 6279 moves the second switch 6973 of the electronic circuit system 6900 to the second state, the electronic circuit system 6900 can output one or more predetermined electronic outputs.

For example, in some embodiments, the electronic circuit system 6900 can output an electronic signal associated with recorded speech to the audible output device 6956. Such an electronic signal can be, for example, associated with an audible countdown timer, instructing the user on the duration of the injection procedure. Said another way, if it takes, for example, ten seconds to complete an injection, an audible countdown timer can count from ten to zero ensuring that the user maintains the medical injector 6000 in place for the full ten seconds. In other embodiments, the electronic signal can be, for example, associated with a recorded message notifying the user that the injection is complete, instructing the user on post-injection disposal and safety procedures, instructing the user on post-injection medical treatment or the like. Such a status message can state, for example, “The injection is now complete. Please seek further medical attention from a doctor.” The electronic circuit system 6900 can also simultaneously output an electronic signal to one and/or both LEDs 6958A, 6958B, thereby causing one and/or both LEDs 6958A, 6958B to stop flashing, change color or the like, to provide a visual indication that the injection is complete. In other embodiments, the electronic circuit system 6900 can send a wireless signal notifying a remote device that the injection is complete. In this manner, a patient's compliance can be monitored.

As shown in FIG. 159, the carrier 6260 moves to a second position within the housing 6100 during the needle insertion operation. With the carrier 6260 in the second position, a distal surface 6296 of the carrier 6260 contacts the housing 6100, thereby limiting the distal movement of the carrier 6260. Furthermore, with the carrier 6260 in the second position, the carrier engagement surface 6131 is disposed within a recesses 6277 defined by the needle insertion tabs 6271. With the carrier engagement surface 6131 disposed within the recesses 6277, the needle insertion tabs 6271 return to an undeformed configuration, as described above. In the undeformed configuration, the needle insertion tabs 6271 extend such that the flanged end 6214 is no longer in contact with the container shoulders 6272. Thus, the portion of the force F₄ applied to the first elastomeric member 6221 moves the medicament container 6210 in the distal direction, relative to the carrier 6260.

When the medicament container 6210 moves in the distal direction relative to the carrier 6260, the medicament container 6210 moves distally about the needle hub 6264 such that the upper portion 6267 of the needle hub 6264 is disposed within the distal counter bore 6234 of the third elastomeric member 6229. In this manner, the proximal end portion 6217 of the needle 6216 punctures through the thickness T of the third elastomeric member 6229 and the medical injector 6000 can be placed in a fifth configuration (i.e., the medicament delivery configuration).

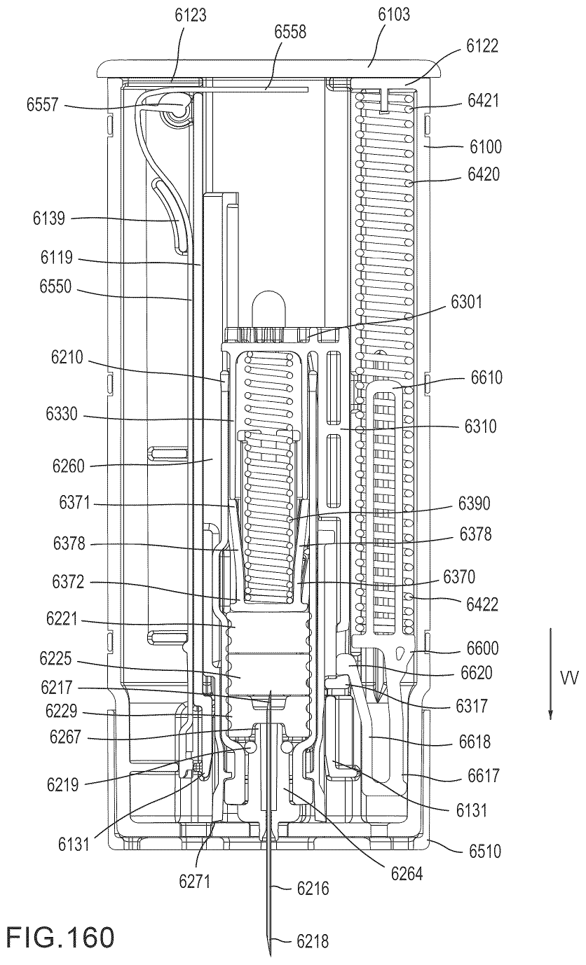

The medical injector 6000 is placed in the fifth configuration when the proximal end portion 6217 of the needle 6216 is disposed within the mixing volume 6237 and a portion of the force F₄ is exerted on the first elastomeric member 6221, as shown in FIG. 160. With the medicament container 6210 and the carrier 6260 in the second position within the housing 6100 (e.g., moved in the distal direction), the portion of the force F₄ exerted on the first elastomeric member 6221 can move the first elastomeric member 6221 and the second elastomeric member 6225 from the second position to a third position within the medicament container 6210. More specifically, the mixing piston 6370 and/or piston portion 6330 exerts the portion of the force F₄ on the proximal surface 6222 of the first elastomeric member 6221 as indicated by arrow VV in FIG. 160 to move the first elastomeric member 6221 and the second elastomeric member 6225 to the third position. In this manner, the medicament disposed within the dry medicament volume 6237 (e.g., the volume defined between the distal surface 6227 of the second elastomeric member 6225 and the proximal surface 6230 of the third elastomeric member 6229) is transferred to the needle 6216 and injected into the body of the patient.

When the spring 6420 fully expands, the medicament delivery mechanism 6300 moves in the distal direction to fully inject the medicament within the medicament container 6210. Additionally, when the spring 6420 is fully expanded and/or when the medicament delivery mechanism 6300 has moved a desired distance within the housing 6100, the guide protrusion 6624 of the transfer member 6600 engages the lower notch 6121 of the housing 6100 (see e.g., FIG. 110) to place the transfer member 6600 in the second configuration. Expanding further, the guide protrusion 6624 is aligned with the lower notch 6121 such that the guide protrusion 6624 moves through the lower notch 6121 to move the transfer member 6600 to the second position. As described above, when the guide protrusion 6624 moves through the lower notch 6121, the bendable portion 6622 of the transfer member 6600 bends (e.g., returns to an undeformed position), thereby placing the transfer member 6600 in its second configuration, as shown in FIG. 161. In this manner, the latch 6620 can be disengaged from the second latch protrusion 6317. Similarly stated, the spring 6420 and/or the transfer member 6600 are decoupled from the medicament delivery mechanism 6300. With the latch arm 6618 disengaged from the latch portion 6310, the medical injector 6000 can be moved from the fifth configuration to the sixth configuration (i.e., the retraction configuration).

With the transfer member 6600 disengaged from the medicament delivery mechanism 6300, the medicament container assembly 6200 and the medicament delivery mechanism 6300 are configured to move within the housing 6100 in the direction shown by the arrow WW in FIG. 161 in response to a force exerted by the retraction member 6440 (e.g., the retraction spring). Similarly stated, with the medicament delivery mechanism 6300 disengaged from the transfer member 6600 and/or the spring 6420, the force F₄ is no longer applied to the medicament delivery mechanism 6300. In this manner, the retraction member 6440 is configured to expand in the direction of the arrow WW to apply a retraction force to the medicament container assembly 6200. Similarly stated, with the portion of the force F₄ configured to compress the retraction spring 6440 removed, the retraction member 6440 expands, returning to its uncompressed (i.e., non-deformed) configuration.

During the retraction operation, the retraction spring 6440 exerts a retraction force on the retraction spring surface 6284 to move the carrier 6260 in the direction WW. The proximal movement of the carrier 6260 (e.g., the retraction) places the carrier engagement surface 6131 in contact with an angled surface 6276 of the needle insertion tabs 6271. In this manner, the angled surface 6276 is configured to slide relative to the carrier engagement surface 6131 as the carrier 6260 moves in the proximal direction in response to the retraction force exerted by the retraction member 6440. As the carrier 6260 continues to move in the proximal direction the engagement surface 6275 is placed into contact with the carrier engagement surface 6131 such that the needle insertion tabs 6271 are placed in the deformed configuration (e.g., non-extended configuration). Therefore, the container shoulders 6272 move closer together to maintain the flanged end 6214 of the medicament container 6210 between a distal surface 6274 of the container shoulder 6272 and a proximal surface 6297 of the container-mounting portion 6263. In this manner, the medicament container 6210 is coupled to the carrier 6260 and a portion of the retraction force moves the medicament container 6210 in the proximal direction. This motion, removes the needle 6216 from the target location of the patient and retracts the needle into the housing 6100, as shown in FIG. 161.

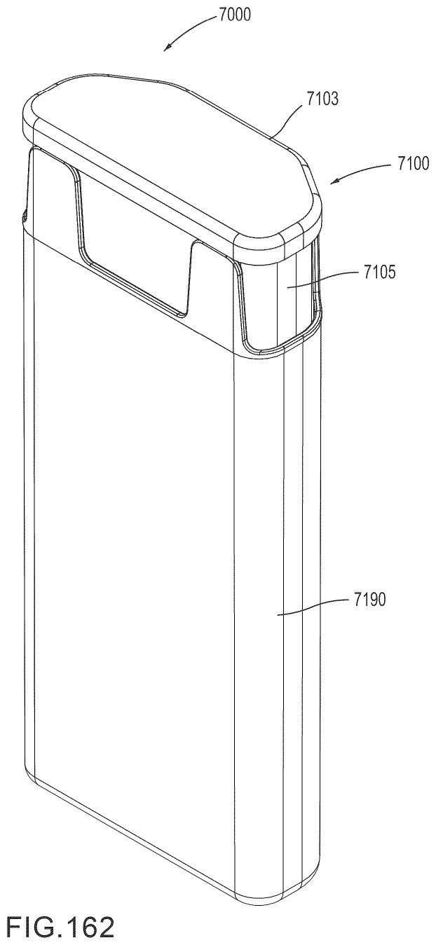

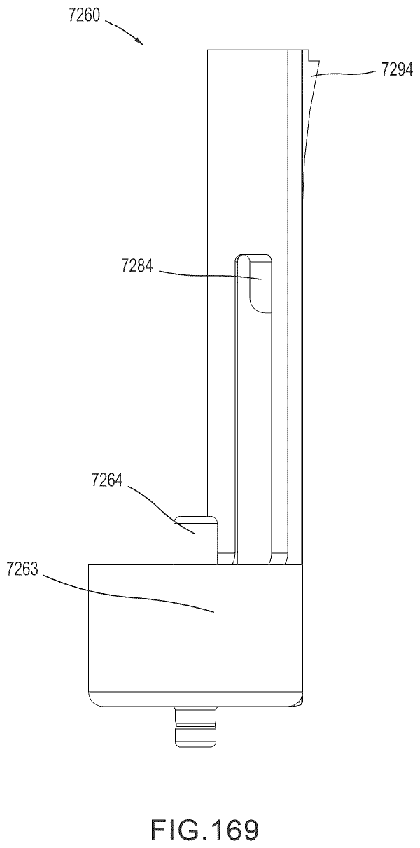

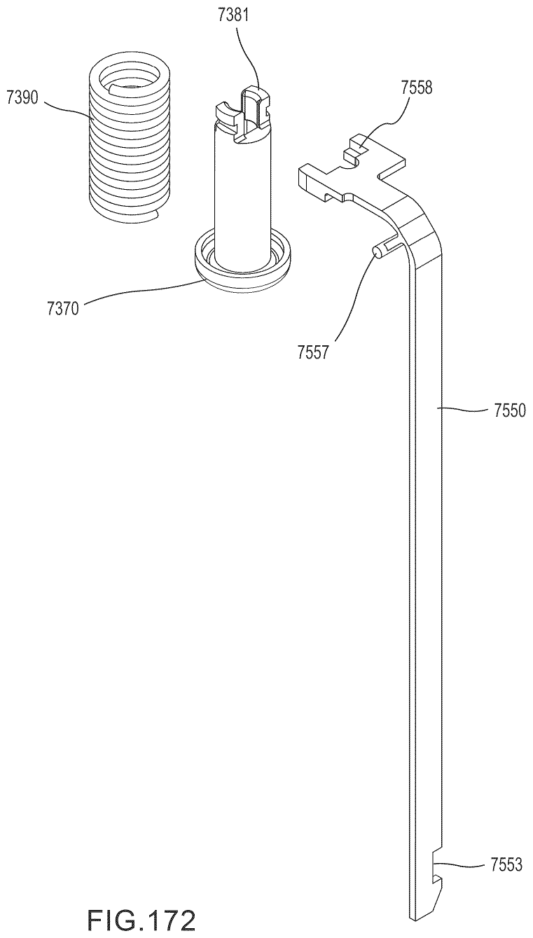

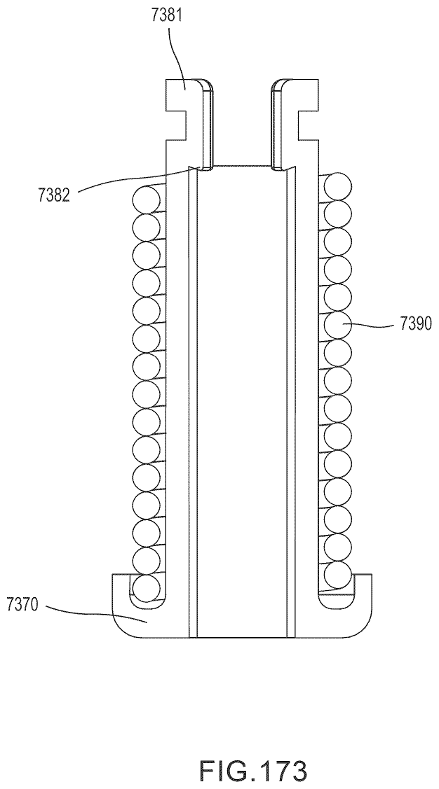

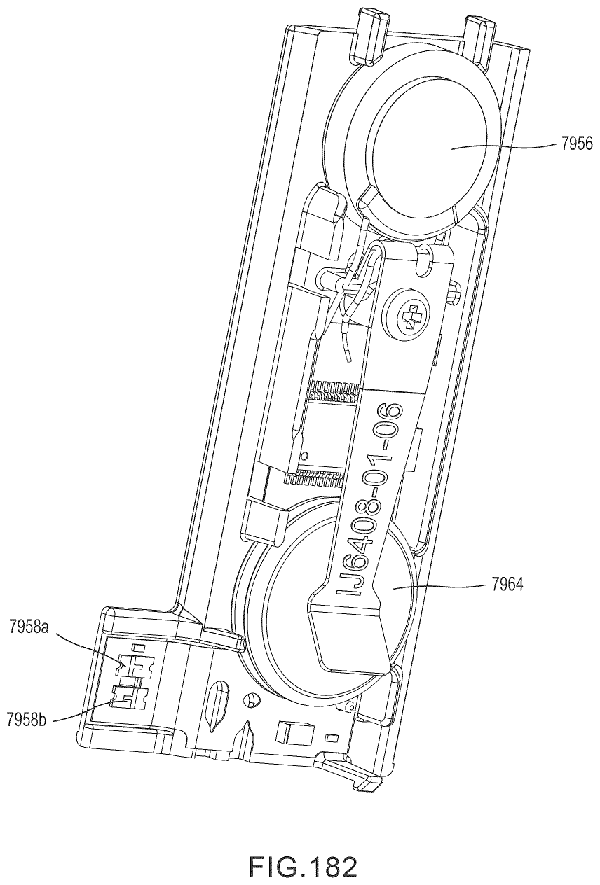

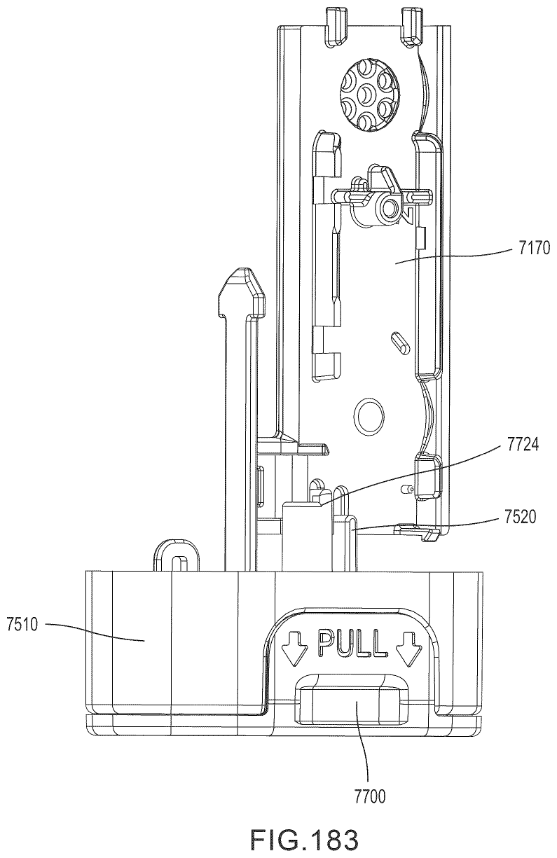

While specific components are discussed above with respect to the medical injector 6000, in other embodiments, any of the medicament delivery devices and/or medical injectors described herein can include components that are modified and/or removed from those shown and described above with respect to the medical injector 6000. Similarly stated, in other embodiments, a medical injector can include different, more or fewer components than are shown in the medical injector 6000 without substantially changing the mixing and/or medicament injection event. For example, FIGS. 162-190 show a medical injector 7000, according to an embodiment.

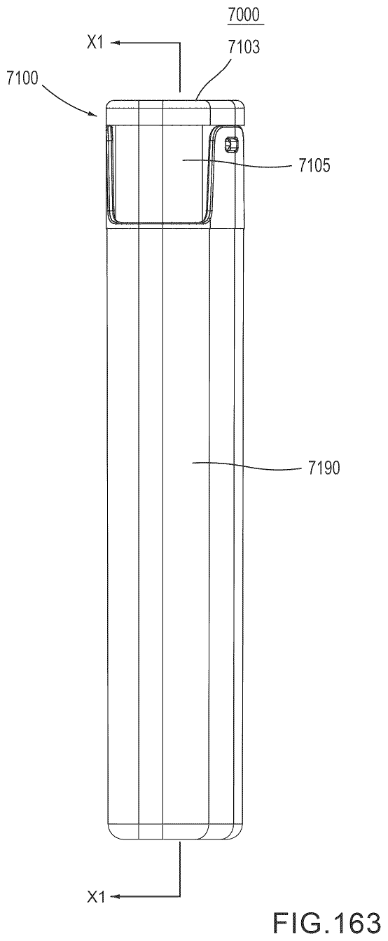

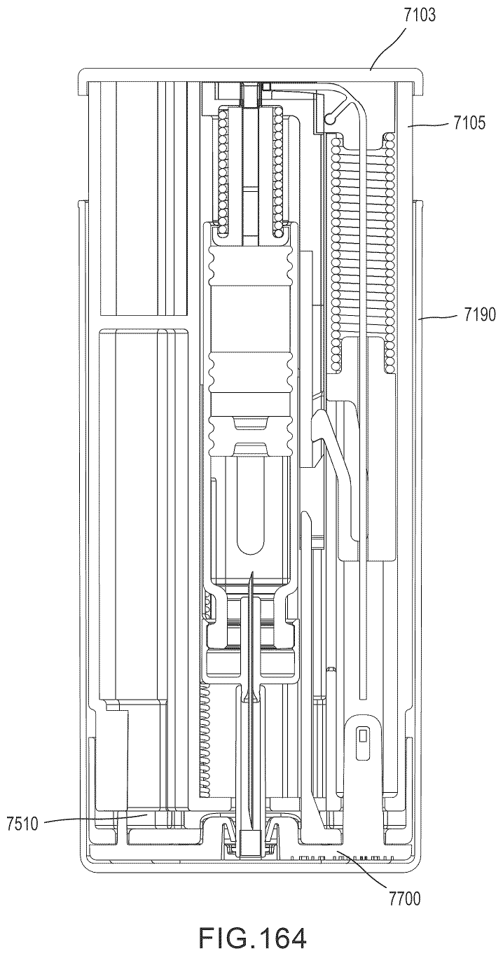

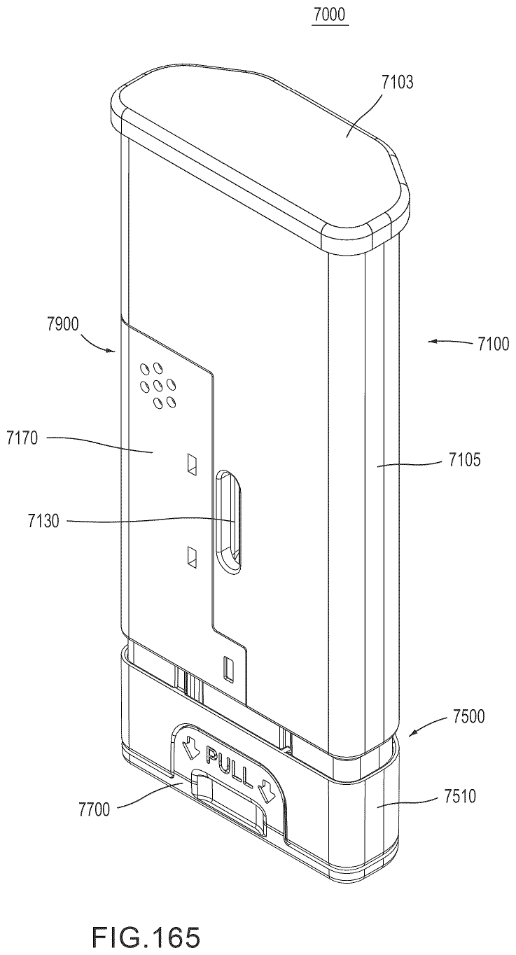

FIG. 162 is a perspective view, FIG. 163 is a side view, and FIG. 164 is a cross-sectional view taken along line X1-X1, of an injector 7000 in a first configuration. The injector 7000 includes a housing 7100 including a body 7105 and a proximal cap 7103. The injector 7000 includes a case 7190 and a safety lock 7700 (shown in FIG. 165). The case 7190 and the safety lock 7700 can be configured to prevent damage to the injector 7000, to prevent the accidental actuation of the injector, to identify the contents of the injector 7000, and/or to initiate an electronic output during operation of the injector, as described in further detail herein.

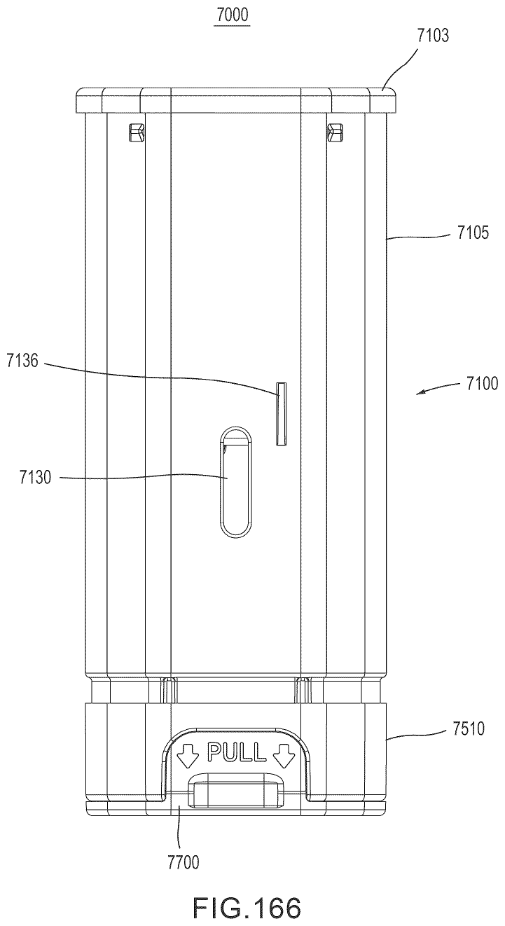

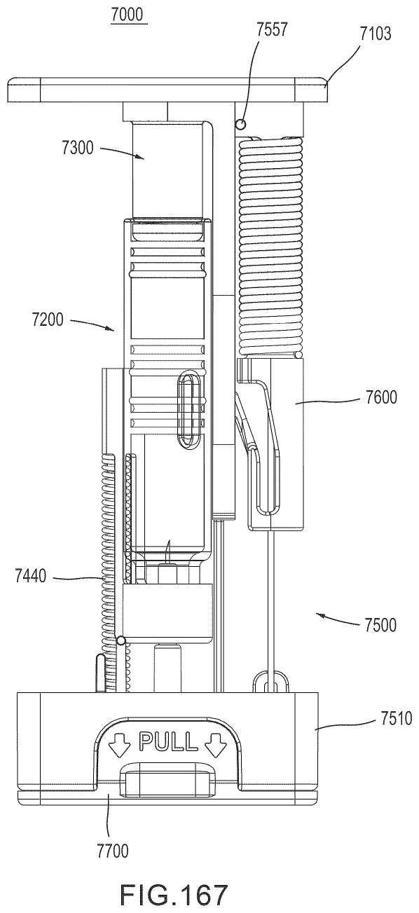

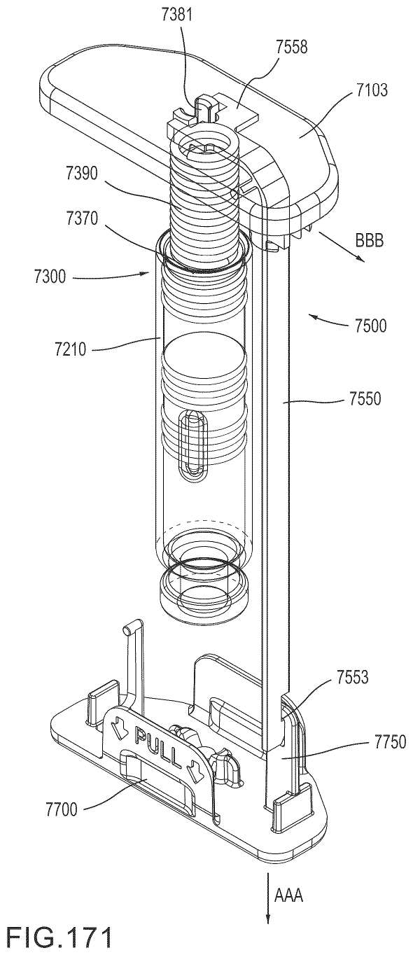

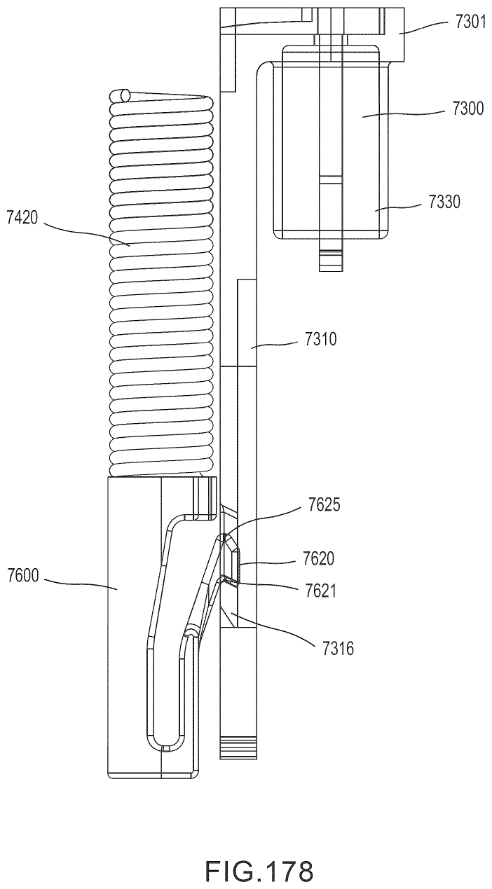

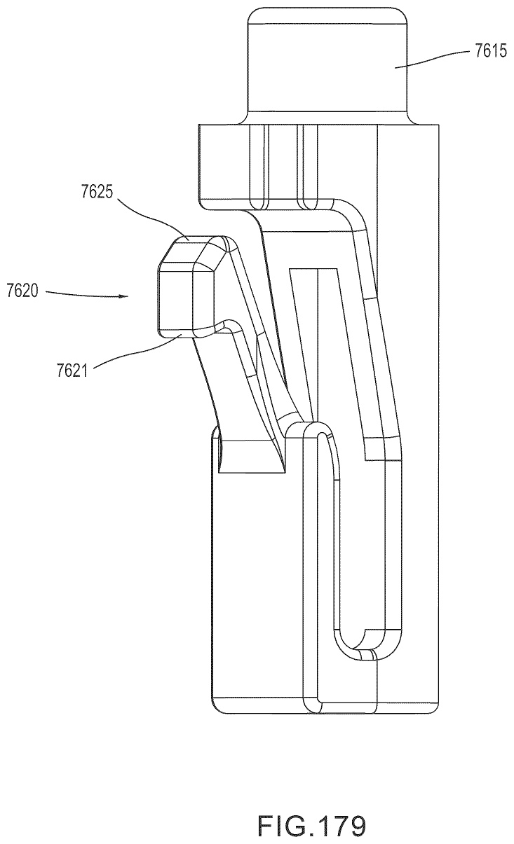

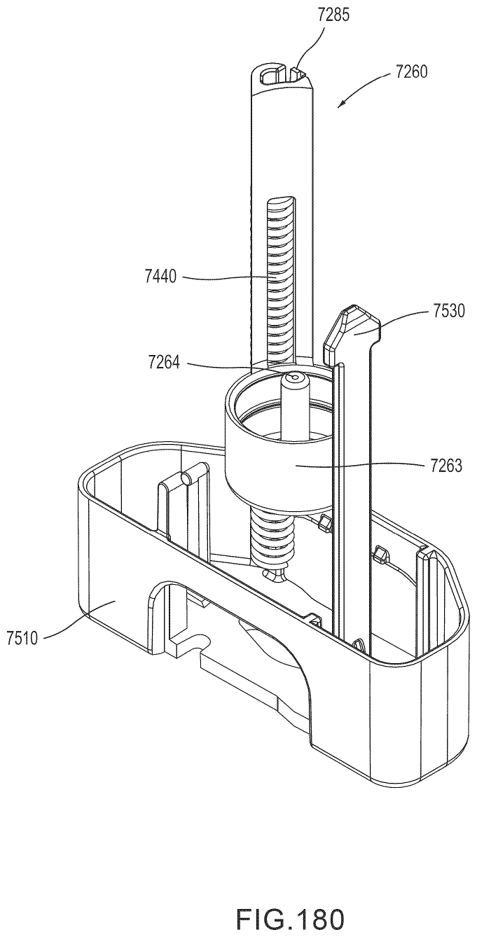

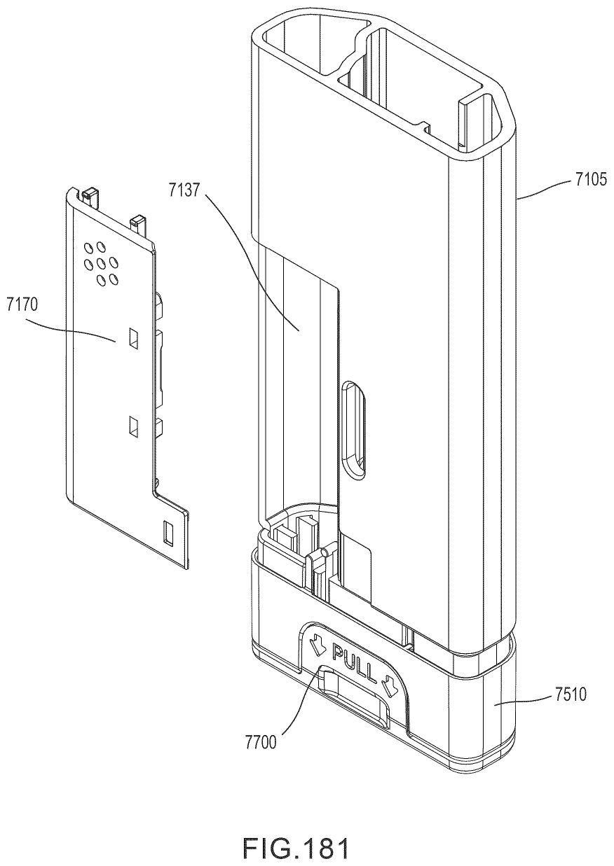

FIG. 165 is a rear perspective view, and FIG. 166 is a rear view, of the injector 7000 in a second configuration (e.g., with the case 7190 removed). FIG. 167 is a front view of the injector 7000, shown without the body 7105 to more clearly show the components disposed within the housing 7100, as described below. The injector 7000 includes a system actuator assembly 7500 having components (including the base 7510) configured to initiate an injection and/or mixing of a medicament contained within the injector 7000. The injector 7000 includes an electronic assembly 7900 (as shown in FIGS. 181-184) configured to provide at least one electronic output associated with injection and/or mixing. The housing 7100 can include openings configured to provide an indication of a status to a user of the injector 7000 and/or to interact with the internal components of injector 7000, specifically, the housing 7100 includes a status window 7130 and a catch 7136. The injector 7000 includes a medicament container assembly 7200 (as shown in FIGS. 167-171), a medicament delivery mechanism 7300 (as shown in FIGS. 171-178), a transfer member 7600 (as shown in FIGS. 178 and 179), and a retraction member 7440 (as shown in FIG. 180).

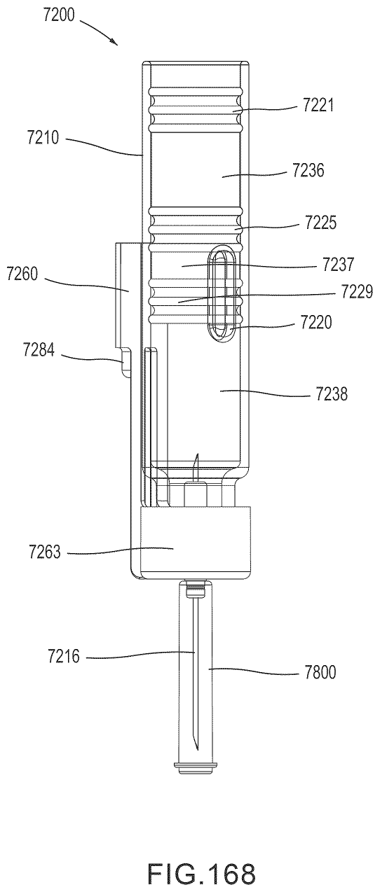

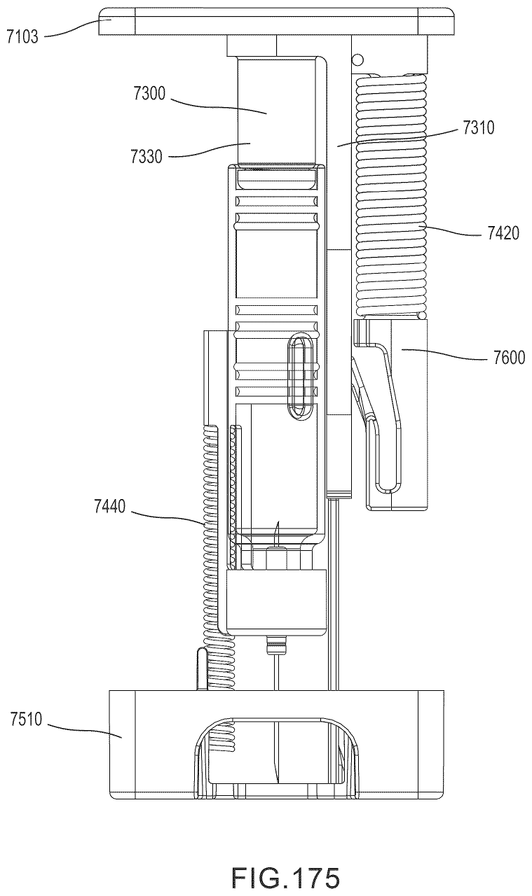

FIGS. 168-170 show components of the medicament container assembly 7200 of the injector 7000. The medicament container assembly 7200 includes components configured to store a medicament, segregate stored medicament components, and mix medicament components. The medicament container assembly 7200 includes a carrier 7260 and a medicament container 7210. The carrier 7260 includes a retraction member protrusion 7284, a container mounting portion 7263, a needle hub 7264, and a latch 7294. The retraction member protrusion 7284 receives a proximal end portion of the retraction member 7440 (as shown in FIG. 175), such that the retraction member 7440 can move the carrier 7260 in the proximal direction. The container mounting portion 7293 receives a distal end portion 7213 of the medicament container 7210. The needle hub 7264 can receive, hold, and/or contain at least a portion of a needle 7216 and can be configured to receive a needle guard 7800. A plug (similar to the plug 6827 shown and described above) can be disposed within the needle guard 7800. In some embodiments, the needle 7216 penetrates the plug during injection. In other embodiments, the needle guard 7800 can be coupled to the safety lock 7700 such that the needle guard 7800 is removed from the needle 7216 when the safety lock 7700 is removed from the housing 7100. The latch 7294 presses against and/or is disposed within the catch 7136 of the housing (shown in FIG. 166) to prevent and/or limit proximal movement of the medicament container 7210 when the injector 7000 is in the first configuration, the second configuration, a third configuration (e.g., the mixing configuration), and a fourth configuration (e.g., the needle insertion configuration).

The medicament container 7210 includes a first elastomeric member 7221, a second elastomeric member 7225, and a third elastomeric member 7229. The first elastomeric member 7221, the second elastomeric member 7225, and the third elastomeric member 7229 are placed within the medicament container 7210 during the fill process, as described below, to define a diluents volume 7236, a mixing volume 7237, and a void volume 7238. Said another way, the diluents volume 7236 is the volume within the medicament container 7210 between a distal surface of first elastomeric member 7221 and a proximal surface of second elastomeric member 7225, the mixing volume 7237 is the volume within medicament container 7210 between a distal surface of second elastomeric member 7225 and a proximal surface of third elastomeric member 7229, and the void volume 7238 is the volume within the medicament container 7210 distal to the distal surface of the third elastomeric member 7229.

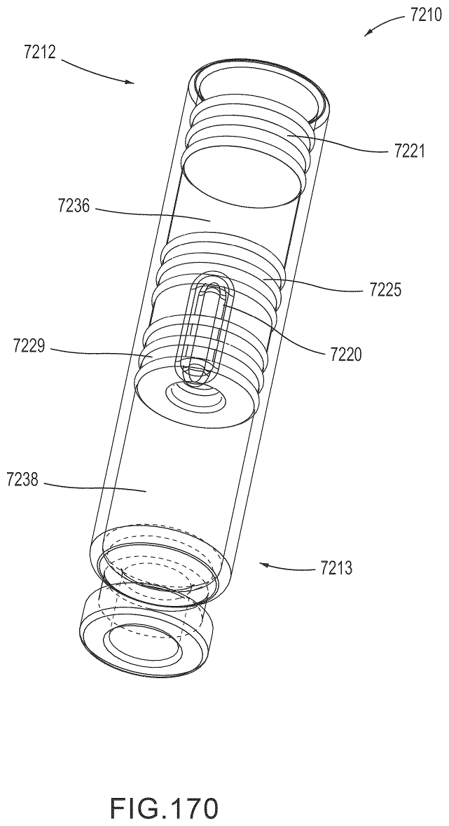

The medicament container 7210 includes a bypass 7220, a proximal end portion 7212, and a distal end portion 7213. The bypass 7220 can be a singular channel bypass or can define multiple channels. Although the bypass 7220 is shown as an external bypass, alternatively, in some embodiments, the bypass 7220 can be internal (e.g., defined by an internal structure of the container) and/or defined by the second elastomeric member 7225. Said another way, in some embodiments the bypass can be configured such that the outer diameter of the medicament container 7210 is substantially constant. As shown in FIGS. 168 and 170, the diluents volume 7236, the mixing volume 7237, and the void volume 7238 are defined by the relative positions of the first elastomeric member 7221, the second elastomeric member 7225, and the third elastomeric member 7229. The diluents volume 7236 can contain medicament diluents, such as, for example, water; the mixing volume 7237 can contain a lyophilized medicament. The second elastomeric member 7225 and a sidewall of the medicament container 7210 can collectively produce a fluid tight seal between the diluents volume 7236 and the mixing volume 7237 to prevent premature mixing of the diluents and the lyophilized medicament.

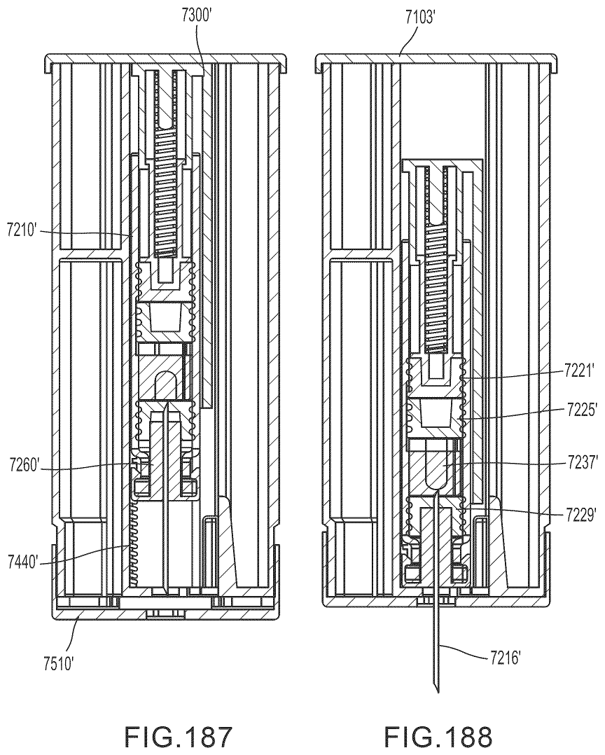

The proximal end 7212 of the medicament container 7210 receives a second movable member 7370 (i.e., a mixing piston, as shown in FIG. 171). In use, when the system actuator assembly 7500 is actuated, the second movable member 7370 moves in the distal direction within the proximal end portion 7212 of the medicament container 7210, which moves the first elastomeric member 7221 in the in the distal direction (see e.g., FIGS. 186 and 187). The distal movement of the first elastomeric member 7221 causes the diluents in diluents volume 7236 to move the second elastomeric member 7225 in the distal direction (see e.g., FIG. 186). In some embodiments, the movement can be substantially simultaneous (e.g. when the diluent is an incompressible fluid). As shown in FIG. 186, distal movement of the second elastomeric member 7225 can cause the substantially dry, solid and/or lyophilized medicament in mixing volume 7237 to move the third elastomeric member 7229 in the distal direction. When the first elastomeric member 7221, the second elastomeric member 7225, and the third elastomeric member 7229, move in the in the distal direction, the volume and location of the diluents volume 7236, the mixing volume 7237, and the void volume 7238, can change. By way of example, when the first elastomeric member 7221, second elastomeric member 7225, and third elastomeric member 7229, move in the in the distal direction, the volume of the diluents volume 7236 and the mixing volume 7237 can initially remain substantially unchanged while the volume of the void volume 7238 can be reduced (see e.g., FIG. 186). When the proximal end of the second elastomeric member 7225 moves in the distal direction past the proximal end portion of the bypass 7220, the diluents volume 7236 can be placed in fluid communication with mixing volume 7237. Thus, continued distal movement of first elastomeric member 7221 can cause the diluents in diluents volume 7236 to flow into mixing volume 7237 via the bypass 7220 and can cause the diluents and the substantially dry, solid and/or lyophilized medicament to mix in the mixing volume 7238, forming a reconstituted medicament within the mixing volume 7237. In some embodiments, when the diluents volume 7236 and the mixing volume 7237 are in fluid communication, the movement of the second elastomeric member 7225 can slow or stop.

As shown and described below with respect to FIG. 187, when the diluents flows into the mixing volume 7237, the volume of the diluents volume 7236 can be reduced, the volume of the mixing volume 7238 can increase, and the volume of the void volume 7238 can be reduced. In some embodiments, when the volume of the void volume 7238 is decreased, air within the void volume 7238 can escape via the needle 7216. The first elastomeric member 7221 can contact the second elastomeric member 7225 and can continue to move in the distal direction as shown in FIG. 187. The distal movement of the first elastomeric member 7221 and the second elastomeric members 7225 can cause the medicament to move the third elastomeric member 7229 in the distal direction, thereby forcing air within the void volume 7238 to escape from medicament container 7210.

In some embodiments, the distal movement of third elastomeric member 7229 during the mixing operation can cause the third elastomeric member 7229 to contact needle 7216. Furthermore, in some embodiments, the distal movement of third elastomeric member 7229 can cause the third elastomeric member 7229 to contact needle 7216 such that the needle 7216 penetrates through only a portion of the third elastomeric member 7229, thus preventing fluid communication between the needle 7216 and the mixing volume 7237 (and the medicament therein) until injection. In this manner, the needle 7216 remains fluidly isolated from the mixing volume 7237 until after the needle insertion event as described below. After completion of the mixing event and/or the insertion event, continued movement of the third elastomeric member 7229 within the medicament container 7210 can cause the needle 7216 to substantially penetrate through the third elastomeric member 7229 and allow the needle 7216 to be placed in fluid communication with mixing volume 7237 and the medicament.

As shown in FIG. 170, in some embodiments, a distal surface of the third elastomeric member 7229 includes a counter bore to allow the third elastomeric member 7229 to move about needle hub 7264 during the mixing operation, as shown in FIGS. 186-188. This configuration effectively reduces the thickness of the portion of the third elastomeric member 7229 through which the needle 7216 penetrates. In some embodiments, the distal surface and a proximal surface of the third elastomeric member 7229 can include a counter bore to reduce the thickness of the portion of the third elastomeric member 7229 through which the needle 7216 penetrates, as described above with respect to FIGS. 158 and 159. In some embodiments, this arrangement can also be used to increase the volume of the mixing volume 7237. In some embodiments, the proximal and/or distal sides of any of the elastomeric members 7221, 7225, or 7229, can be shaped to increase or decrease the volumes of the diluents volume 7236, the mixing volume 7237, and/or the void volume 7238, respectively. Thus, the flow of a fluid to or from the diluents volume 7236, the mixing volume 7237, and/or the void volume 7238 and/or the mixing of medicament formulations within the mixing volume 7237 can be controlled.

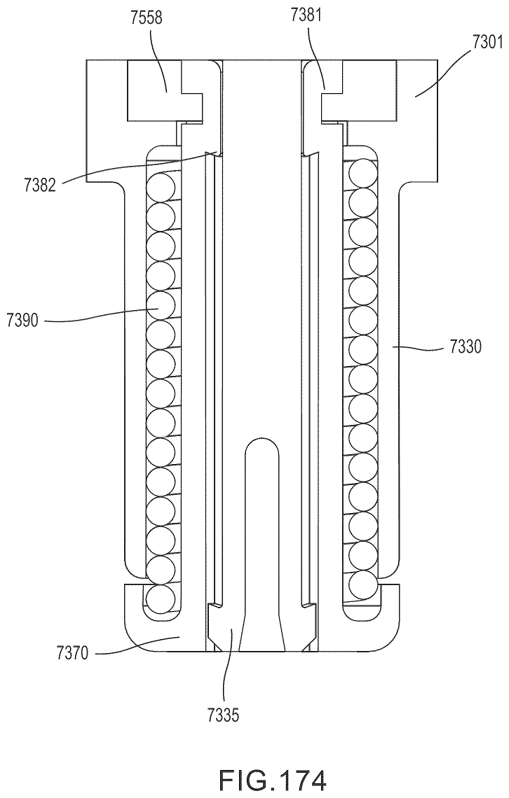

FIGS. 171-175 depict portions of the injector 7000 including the medicament delivery mechanism 7300 and the transfer member 7600. The injector 7000 includes components configured to store, transport, and mix medicaments such as the safety lock 7700, the proximal cap 7103, medicament container assembly 7200, and the medicament delivery mechanism 7300. The medicament delivery mechanism 7300 can be activated by portions of the system actuator assembly 7500 and includes a first movable member 7301, the second movable member 7370 and a mixing spring 7390. When the injector 7000 is in the first and second configurations, the second movable member 7370 and the mixing spring 7390 are disposed within a piston portion 7330 of the first movable member 7301 (as shown in FIG. 174). A mixing activator member 7550 is operatively coupled to the safety lock 7700 via a hook portion 7553. The mixing activator member 7550 includes a pivot protrusion 7557 operatively coupled to proximal cap 7103 (see e.g., FIG. 167) that allows the mixing activator member 7550 to pivot about the pivot protrusion 7557.

As shown in FIGS. 171, 172 and 174, the mixing activator member 7550 includes a retention portion 7558 that is operatively engaged with an external retention portion 7381 of the second movable member 7370 when the injector 7000 is in the first configuration (e.g., before actuation of the second movable member 7370). The second movable member 7370 includes an internal retention shoulder 7382 that engages a portion of the first movable member 7301 upon completion of the mixing operation. More particularly, the internal retention shoulder 7182 engages a mixing retainer 7335 included in the piston portion 7330 of the first movable member 7301 (shown in FIG. 174) to stop the distal movement of second movable member 7370 upon completion of the mixing event. The mixing spring 7390 is configured exert a force to move the second movable member 7370 in the distal direction when moving from a compressed configuration (e.g., when the injector is in the first configuration) to an uncompressed configuration (e.g., when the injector is in the second configuration) in the distal direction such that the second movable member 7370 contacts the first elastomeric member 7221.

The medicament delivery mechanism 7300 is configured such that when the safety lock 7700 is removed from the injector 7000, a force acting in the distal direction (as shown by the arrow AAA in FIG. 171) is applied to the hook portion 7553 prior to the hook portion 7553 being disengaged from a retention portion 7750 of the safety lock 7700. The distal force causes the mixing activator member 7550 to freely rotate about pivot protrusion 7557 thereby moving the retention portion 7558 in the direction of the arrow BBB shown in FIG. 171. When the retention portion 7558 is disengaged from the external retention portion 7381, the mixing spring 7390 moves from the compressed configuration to the uncompressed configuration and exerts a force on the second movable member 7370, thereby moving the second movable member 7370 in the distal direction. The distal movement of the second movable member 7370 causes the external retention portion 7381 to act against the retention portion 7558 of the mixing activator member 7550, causing the mixing activator member 7550 to further rotate. The mixing activator member 7550 rotates such that the external retention portion 7381 of the second movable member 7370 is no longer operatively coupled to the retention portion 7558 of the mixing activator member 7550, and the mixing spring 7390 moves the second movable member 7370 into contact with the first elastomeric member 7221. Continued movement of the second movable member 7370 moves the first elastomeric member 7221, the second elastomeric member 7225 and/or the third elastomeric member 7229, as described above.

The first movable member 7301 includes the piston portion 7330 and a latch portion 7310. The piston portion 7330 is operatively coupled to the injection spring 7420 via the transfer member 7600. In this manner, expansion of the injection spring 7420 moves transfer member 7600 in the distal direction, thereby moving the piston portion 7330 in the distal direction to move the medicament container 7210.

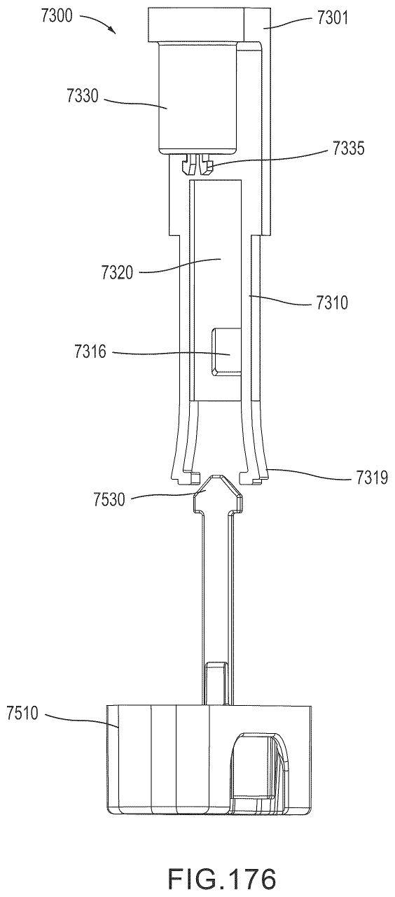

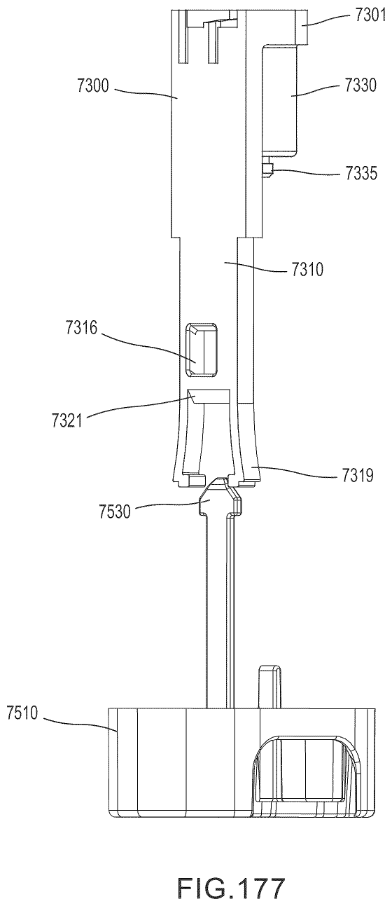

As shown in FIGS. 176-178, the latch portion 7310 of the first movable member 7301 includes a release portion 7319 and a ramp 7321 and defines a channel 7320 and an opening 7316. The injection spring 7420 can move between a compressed configuration and an uncompressed configuration to exert an insertion force against the proximal cap 7103 and the transfer member 7600. When the injector 7000 is in the first, second and third configuration (i.e., prior to actuation of the insertion spring 7420), the release portion 7319 rests on and/or engages a rod 7530 of the base 7510 (as shown in FIGS. 176 and 177). The release portion 7319, the base 7510, and the injection spring 7420 are collectively configured such that injection spring 7420 cannot produce enough force to deform the release portion 7319 to move the release portion 7319 in the distal direction over the rod 7530. The release portion 7319, however, is configured (e.g., flexible) such that pushing the base 7510 in the proximal direction deforms the release portion 7319, thereby allowing the rod 7530 to move in the proximal direction through release portion 7319. The channel 7320 allows the first movable member 7301 to move about the rod 7530 during injection and retraction. The opening 7316 receives a latch 7620 of the transfer member 7600.

With the movement of the rod 7530 past the release portion 7319, the insertion spring 7420 is released from the compressed configuration (e.g., allowed to expand). The arrangement of the proximal cap 7103 is such that the proximal cap 7103 exerts a reaction force equal to and in an opposite direction of the portion of the insertion force exerted on the proximal cap 7103 by the insertion spring 7420. In this manner, the distal end portion of the insertion spring 7420 is configured to extend in the distal direction. Thus, the expansion of the insertion spring 7420 can move the transfer member 7600 and therefore the first movable member 7301 in the distal direction. The mixing retainer 7335 can be configured to engage the internal retention shoulder 7382 of the second movable member 7370 to limit the distal movement of the second movable member 7370, as described above. In some embodiments, the second movable member 7370 can include a retention portion configured to limit movement of the second movable member 7370 relative to the first movable member 7301 in a proximal direction (i.e., to limit and/or prevent retraction of the second movable member 7370 back into the piston portion 7330).

FIG. 178 depicts the first movable member 7301, the transfer member 7600, and the injection spring 7420 (the first movable member 7301 is shown translucent to better show the interaction between the transfer member 7600 and the latch portion 7310 of the first movable member 7301). The latch 7620 of the transfer member 7600 includes a top surface 7625, and a bottom surface 7621 configured to engage the distal end portion of the sidewall defining the opening 7316. The transfer member 7600 also includes an injection spring seat 7615.

FIG. 180 depicts the retraction member 7440 and other components that interact with retraction member 7440 to retract the needle 7216 back into the housing 7100 after injection of the medicament. The retraction member 7440 is seated about the retraction member protrusion 7284 of the carrier 7260 and can be seated about a spring seat (not shown) on the base 7510 and/or a sidewall of the housing 7100. The retraction member 7440 can be disposed substantially uncompressed (i.e., substantially expanded) between the retraction member protrusion 7284 and the base 7510 and/or the sidewall of the housing 7100. In some embodiments, the retraction member 7440 can be disposed partially compressed between the retraction member protrusion 7284 and the base 7510. In those embodiments in which the retraction member 7440 is disposed in an at least partially compressed state, the latch 7294 interacts and/or engages with the catch 7136 of the housing 7100 to limit the proximal movement of the carrier 7260. In this manner, the force exerted on the carrier 7260 by the retraction member 7440 is not transferred to the medicament container 7210, the medicament delivery mechanism 7300, the system actuator assembly 7500, and/or the transfer member 7600. When the carrier 7260 moves in the distal direction during injection, the retraction member 7440 is compressed between the base 7510 and the carrier 7260. In this manner, as described in more detail below, the retraction spring 7440 exerts a proximal (or retraction) force on the carrier 7260 upon completion of the injection operation.

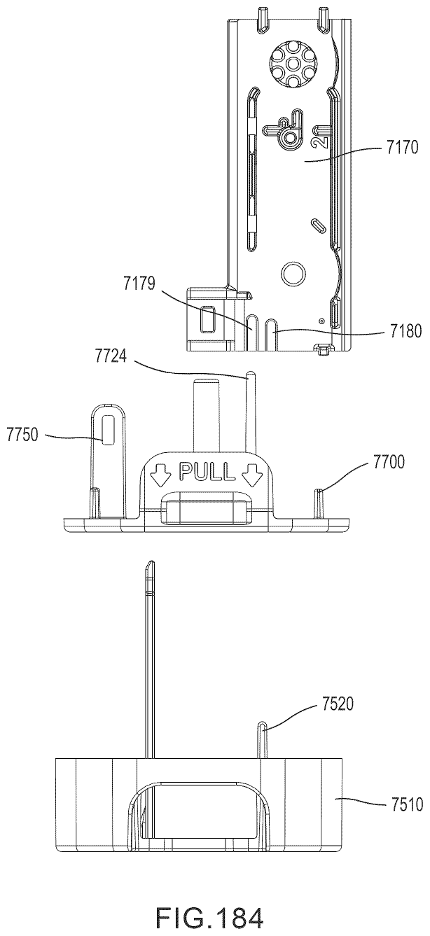

FIGS. 181-184 depict the electronic assembly 7900, and other components of the injector 7000 that interact with the electronic assembly 7900. The electronic assembly 7900 includes any suitable electronic components operatively coupled to produce and/or output an electronic output and/or to perform the functions described herein. The electronic assembly 7900 can be similar to the electronic circuit systems described in U.S. Pat. No. 7,731,686, entitled “Devices, Systems and Methods for Medicament Delivery,” filed Jan. 9, 2007, and/or U.S. Patent Application Publication Number 2008/0269689, entitled “Medicament Delivery Device Having an Electronic Circuit System,” filed May 12, 2009, both of which are incorporated herein by reference in their entirety. As shown in FIG. 181, the housing 7100 of the injector 7000 defines a volume 7137 configured to receive an electronic circuit housing 7170. The electronic assembly 7900 can include an audio output device 7956, (e.g. a speaker), a battery assembly 7964, and at least one visual output device 7958 (e.g., an LED light). As shown in FIG. 184, the electronic circuit housing 7170 defines a first actuation groove 7179 and a second actuation groove 7180. The safety lock 7700 includes first switch actuator 7724 disposed within the first actuation groove 7179 such that removal of the safety lock 7700 from the injector 7000 engages a first switch of the electronic assembly 7900 to initiate an electronic output of the electronic assembly 7900. The base 7510 includes second switch actuator 7520 disposed within second switch actuation groove 7180 such that actuation of the base 7510 engages a second switch of the electronic assembly 7900 to initiate an electronic output of the electronic assembly 7900.

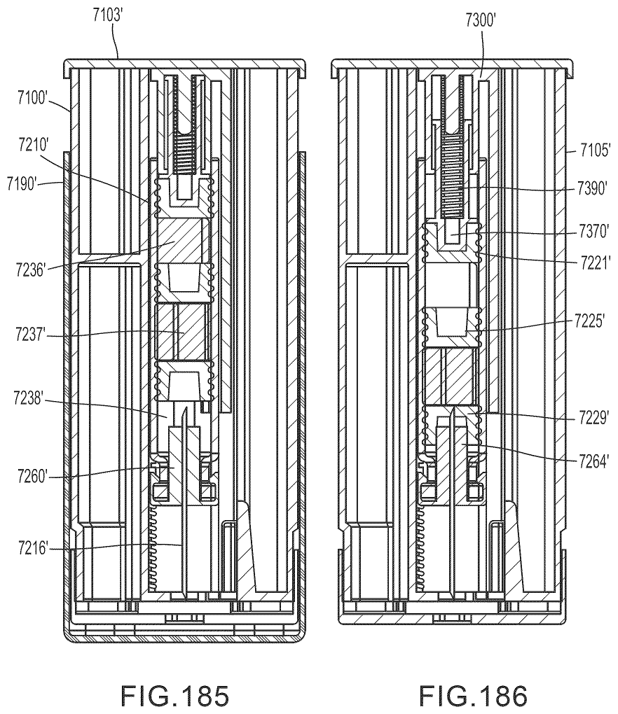

The operation of the injector 7000 can be described as follows with reference to an injector 7000′ shown in FIGS. 185-190. The injector 7000′ is similar to, and can have similar components as the injector 7000. Accordingly, similar components can perform similar functions. By way of example, the first elastomeric member 2217′ of the injector 7000 can be similar in configuration to the first elastomeric member 7221 of the injector 7000. Furthermore, any component described in relation to injector 7000, can be included in injector 7000′. By way of example, the injector 7000′ can have an electronic assembly 7900 (not shown in FIGS. 185-190). Thus, the following description of the injector 7000′ includes references to components described above with regards to injector 7000.

FIG. 185 depicts injector 7000′ with the case 7190′ disposed about the housing 7100 (e.g., the first configuration). As depicted in FIG. 186, the case 7190′ can be removed from the injector 7000′ (e.g., the second configuration). Removing the case 7190′ can actuate the electronic assembly 7900 to produce an electronic output, such as, for example, an output indicating the status of injector 7000′ or providing instructions for operation. Removing the case 7190′ can actuate the electronic assembly 7900 by engaging a portion of the electronic assembly 7900, placing the battery assembly 7964 in electronic communication with a processor, or the like.

The safety lock 7700′ can be removed from the injector 7000′ to place the injector 7000′ in a third configuration (e.g., the initiation of the mixing operation). Removing the safety lock 7700 can cause the first switch actuator 7724 to activate a first switch to produce a second electronic output and/or continue producing the current electronic output. Removing the safety lock 7700 also initiates the mixing operation (e.g., the third configuration). Specifically, removing the safety lock 7700 causes the retention portion 7558 to disengage from the release portion 7553 of the mixing activator member 7550 and can allow the mixing activator member 7550 to rotate freely about the pivot protrusion 7557. As described above with reference to medicament delivery mechanism 7300, force from the mixing spring 7390′ acts on the second movable member 7370′ and causes the second movable member 7370′ to move in the distal direction. The distal movement of the second movable member 7370′ causes the external retention portion 7381 to act against the retention portion 7558 of the mixing activator member 7550 causing the mixing activator member 7550 to rotate. The mixing activator member 7550 rotates such that the external retention portion 7381 of the second movable member 7370′ is disengaged from the retention portion 7558 of the mixing activator member 7550, thereby allowing the mixing spring 7390′ to move the second movable member 7370′ into the medicament container 7210′ and subsequently into contact with the first elastomeric member 7221′.

The distal movement the second movable member 7370′ within the medicament container 7210′ moves the first elastomeric member 7221′, the second elastomeric member 7225′, and/or the third elastomeric member 7229′ within the medicament container 7210′, as described above, and as shown in FIGS. 186 and 187. In this manner, actuation of the medicament delivery mechanism 7300 produces the mixing operation as described above.

As described above, the distal movement of the third elastomeric member 7229′ during the mixing operation causes the third elastomeric member 7229′ to contact the needle 7216′, such that the needle 7216′ penetrates through a portion of the third elastomeric member 7229′, as shown in FIGS. 186 and 187. When the second movable member 7370′ has moved a predetermined distance within the medicament container 7210, the internal retention shoulder 7382 contacts the mixing retainer 7335 of the first movable member 7301′ to stop the distal movement of the second movable member 7370′. In some embodiments, this arrangement can prevent the needle 7216′ from being placed in fluid communication with the mixing volume 7237 and/or the medicament. At this point, mixing of the medicament is substantially completed and the injector 7000′ is in the fourth configuration (see e.g., FIG. 187). In some embodiments, however, the needle 7216′ can penetrate through the third elastomeric member 7229′ before the internal retention shoulder 7382 stops the distal movement of the second movable member 370′ and can allow the needle 7216′ to be in fluid communication with the mixing volume 7237′ and the medicament.

During the mixing operation, the electronic assembly 7900 can output a countdown timer to alert the user to refrain from activating the insertion spring 7420 of the injector 7000′ until the mixing is complete and/or can instruct the user to activate the injector 7000′ after the mixing is complete. The electronic assembly 7900 can provide the user with instructions for activating the injector 7000′, such as, for example, identifying where to inject the medicament and/or how to begin injection (e.g., by pressing the base 7510′ against the body).

The user can move the base 7510′ (and any of the other bases shown and described herein) using any suitable motion and/or operation. For example, in some embodiments, the user can grasp the sides of the housing 7110′ and push against the proximal portion thereof. Moving the base 7510′ in the proximal direction can start the insertion and injection process (e.g., a fifth configuration and sixth configuration, respectively). When the base 7510′ is moved in the proximal direction, movement of the rod 7530 deforms the release portion 7319 such that the rod 7530 moves in the proximal direction within the channel 7320 of the first movable member 7301′. The injection spring 7420 acts against the proximal cap 7103 and the transfer member 7600, as described above, to cause the transfer member 7600 to move in the distal direction. The latch 7620 of the transfer member 7600 acts within the opening 7316 and moves the first movable member 7301′ in the distal direction. The medicament delivery mechanism 7300′, the carrier 7260′, and the medicament container 7210′ can move substantially together, as shown in FIG. 187, to insert the needle 7216′. This operation also causes retraction spring 7440 to compress. A distal end portion of the carrier 7260′ contacts the base 7510′ and/or the housing 7100 to stop movement of the carrier 7260 (e.g., the sixth configuration as shown in FIG. 188).

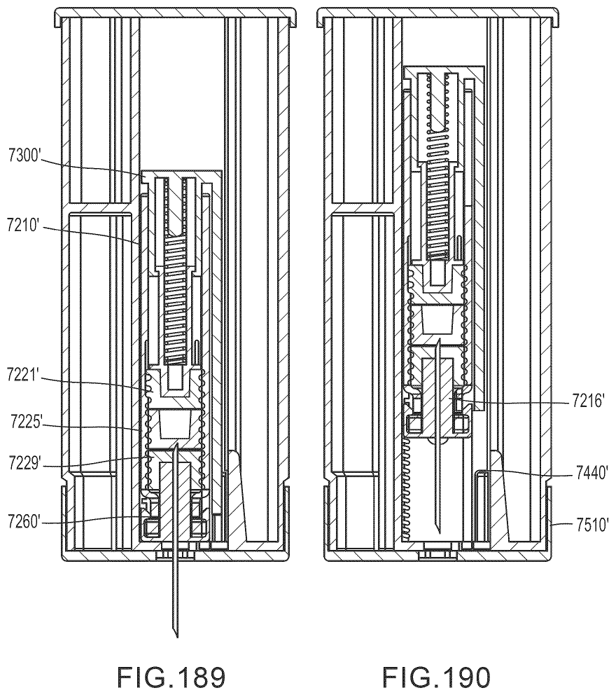

Upon completion of the needle insertion operation, the injection spring 7420 continues to move the transfer member 7600, the first movable member 7301′, the mixing spring 7390′ and the second movable member 7370′ in the distal direction. Said in another way, the medicament delivery mechanism 7300′ moves relative to the carrier 7260′. The second movable member 7370′ causes the first elastomeric member 7221′ and the second elastomeric member 7225′ to move in the distal direction and causes the medicament within the mixing volume 7237′ to move the third elastomeric member 7229′ in the distal direction. The third elastomeric member 7229′ moves in the distal direction such that the needle 7216′ penetrates through the third elastomeric member 7229′ thus placing the needle 7216′ in fluid communication with the mixing volume 7237′ and the medicament (see, e.g. FIG. 188). The third elastomeric member 7229′ contacts the distal end portion 7213 of the medicament container 7210′ to stop its distal movement. Continued movement of the first elastomeric member 7221′ and the second elastomeric member 7229′ causes medicament within the mixing volume 7237′ to flow into needle 7216′ and out of injector 7000′. The first elastomeric member 7221′ and the second elastomeric member 7225′ continue to move in the distal direction and into contact with the third elastomeric member 7229′, thereby stopping the flow of medicament from the mixing volume 237′ through the needle 7216′ and out of the injector 7000′ (e.g., a seventh configuration as shown in FIG. 189).

Upon completion of the injection operation, the disengagement rod (not shown) of the base 7510′ contacts the ramp 7321 of the first movable member 7301′ and causes the latch 7620 of the transfer member 7600 to move out of the opening 7316 of the first movable member 7301′. Said another way, upon completion of the insertion, the transfer member 7600 is disengaged from the first movable member 7301′, thereby removing the force of the injection spring 7420 from the medicament delivery mechanism 7300′. The retraction member 7440, which has been compressed by the injection operation between the retraction member protrusion 7284 and the base 7510′, expands and moves the carrier 7260′ and the needle 7216′ in the proximal direction within the injector 7000′ (e.g., an eighth configuration as shown in FIG. 190). Although described as being a portion of the base 7510′, in other embodiments, the disengagement rod can be coupled to any suitable portion of the injector, such as for example, the housing 7100′. Moreover, in some embodiments, the retraction member 7440′ can expand fully. In some embodiments the carrier 7260′ can move in the proximal direction and the latch 7294 can contact the catch 7136 of body 7100 to stop the proximal movement of the carrier 7260 and the needle 7216.

Other embodiments can include any suitable mechanism for disengaging the transfer member 7600 from the medicament delivery mechanism 7300′. For example, in some embodiments, when the transfer member 7600 and the medicament delivery mechanism 7300′ reach the distal end portion of the housing 7100, a disengagement member (not shown) of the base 7510′ can limit the travel of the medicament delivery mechanism 7300′ at a predetermined distance from the base 7510′ (e.g., towards the end of the travel of the medicament delivery mechanism 7300′). Thus, when the base 7510′ is pulled away from the injection site, the force of the injection spring 7420′ can push the base 7510′ and/or the medicament delivery mechanism 7300′ in the distal direction. This movement can allow the latch 7620 of the transfer member 7600 to align with a slot (not shown) in a retention member (not shown) of the housing 7100′, thereby allowing the latch 7620 to become disengaged from the first movable member 7301′. After the latch 7620 is disengaged, retraction can occur, as described above. In this manner, because the base 7510′ remains stationary while the injector 7000′ is pressed firmly against the patient (e.g., the base cannot move in the distal direction), the retraction operation is prevented until the pressure is released (i.e., until the base 7510′ is removed). This can provide time for the entire dose to be delivered through the needle 7216′ before retraction occurs.

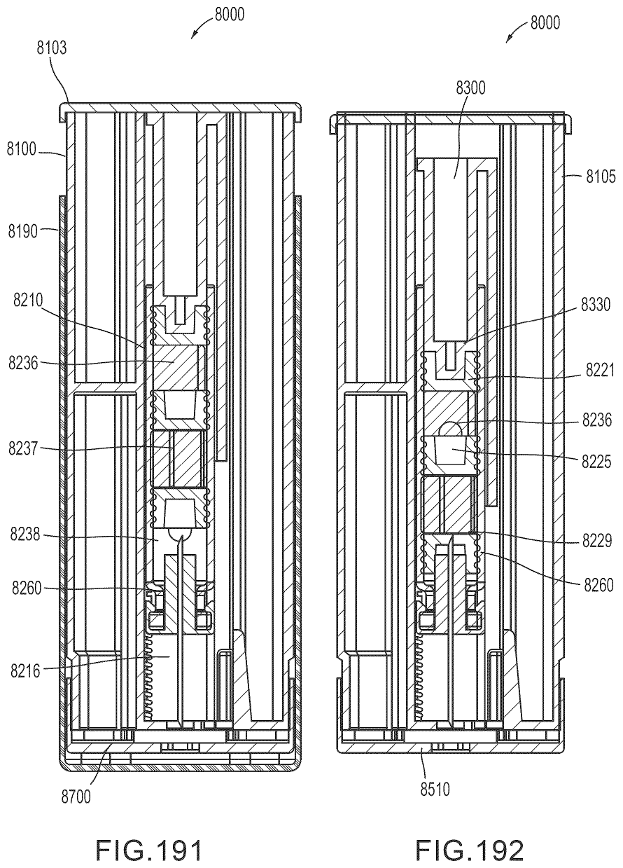

Although the injector 7000 is shown and described as having a second movable member 7370 that is separate (e.g., has a separate spring, can be separately actuated, etc.) from the first movable member 7301, in other embodiments, a second movable member and a first movable member can share common components and/or can be actuated by a single energy storage member. For example, FIGS. 191-196 depict an injector 8000. Certain components within the injector 8000 can be similar to and have similar functions as the corresponding components in the injector 7000. By way of example, the first elastomeric member 8221 of the injector 8000 can be similar in configuration to the first elastomeric member 7221 of the injector 7000. The injector 8000 differs from injector 7000, however, in that injector 8000 does not include a separately actuated mixing assembly (e.g., the second movable member 7370 and/or the mixing spring 7590 included in the injector 7000).

The injector 8000 includes a housing 8100, a proximal cap 8103, a case 8190, a base 8510, a medicament container 8210, and a first, second, and third elastomeric member 8221, 8225, 8229, respectively, that define a diluents volume 8236, a mixing volume 8237, and a void volume 8238, as described above. The injector 8000 also includes a needle 8216, and a movable member 8300. As described below, the movable member 8300 effectuates both the mixing and the injection of the medicament.

FIG. 191 depicts the injector 8000 with the case 8190 disposed about the housing 8100 (e.g., a first configuration). As depicted in FIG. 187, the case 8190 can be removed from the injector 8000 to place the injector 8000 in a second configuration. Removing the case 8190 can actuate an electronic assembly (not shown in FIGS. 191-196) to produce an electronic output, such as, for example, an output indicating the status of the injector 8000 or providing instructions for operation. Removing the case 8190 can actuate the electronic assembly by engaging a portion of the electronic assembly, placing a battery assembly in electronic communication with a processor, or the like, as described above.

A safety lock 8700 can be removed from injector 8000 to place the injector 8000 in a third configuration (e.g., initiation of the mixing operation). Removing safety lock 8700 can cause an actuator (similar to the first switch actuator 7724) to activate a first switch, to produce a second electronic output and/or continue producing a current electronic output. Removing the safety lock 8700 exposes the base 8510 of the injector 8000.

The base 8510 can be moved in the proximal direction thereby causing an injection spring (not shown in FIGS. 191-196) to act against the proximal cap 8103 (e.g., a distal end portion of the injection spring moves in the distal direction), which moves the movable member 8300 in the distal direction as described above with reference to injector 7000. The initial movement of the movable member 8300 starts the mixing operation (e.g., the third configuration, see FIG. 192). More specifically, the base 8510 can be moved in the proximal direction and can cause a release rod to deform a release portion to actuate the injection assembly, in a similar manner as described above. The injection spring can act against the proximal cap 8103 and a transfer member (e.g., similar to the transfer member 7600) to move the transfer member, in the distal direction, and therefore the movable member 8300. The distal movement of the movable member 8300 causes a piston portion 8330 of the movable member 8300 to contact the first elastomeric member 8221 and to move the first elastomeric member 8221 in the distal direction.

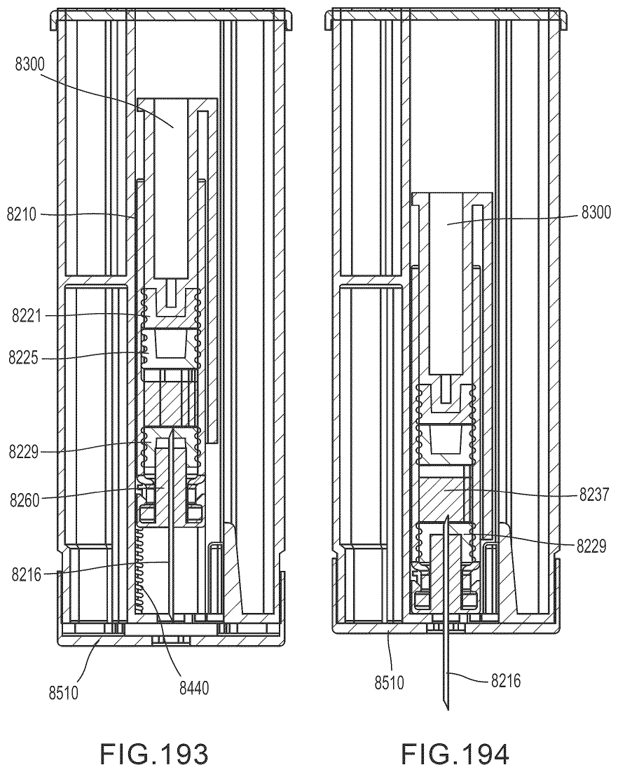

The distal movement of the first elastomeric member 8221 moves the second elastomeric member 8225 and/or the third elastomeric member 8229 within the medicament container 8210, as described above, and as shown in FIGS. 192 and 193. In this manner, actuation of the injection spring produces the mixing operation.

As described above, the distal movement of third elastomeric member 8229 during the mixing operation causes the third elastomeric member 8229 to contact needle 8216, such that the needle 8216 penetrates through a portion of the third elastomeric member 8229. At this point, mixing of the medicament is substantially complete and the injector 8000 is in a fourth configuration (see e.g., FIG. 193). In some embodiments, however, the needle 8216 can penetrate through the third elastomeric member 8229 thereby allowing the needle 8216 to be in fluid communication with the mixing volume 8237 and the medicament.

Continued movement of the movable member 8300 starts the insertion and injection processes (e.g., a fifth configuration). The movable member 8300, the carrier 8260, and the medicament container 8210 can move substantially together, and can cause the retraction member 8440 to compress. In this manner, the needle 8216 is inserted as shown in FIG. 193. A distal end portion of the carrier 8260 contacts the base 8510 and/or the housing 8100 to stop the distal movement of the carrier 8260 and the medicament container 8210 (e.g., a sixth configuration, see FIG. 194).

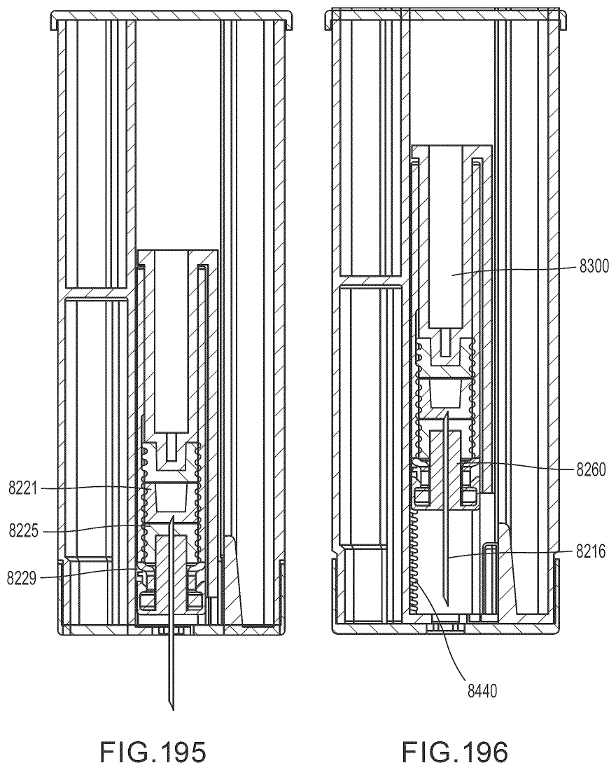

Upon completion of the needle insertion operation, the injection spring continues to move the movable member 8300 in the distal direction. Said in another way, the movable member 8300 moves relative to carrier 8260 and within the medicament container 8210, as shown in FIGS. 193-195. The piston portion 8330 of the movable member 8300 moves the first elastomeric member 8221 and the second elastomeric member 8225 in the distal direction, thereby causing the medicament within the mixing volume 8237 to move the third elastomeric member 8229 in the distal direction. The third elastomeric member 8229 moves in the distal direction such that the needle 8216 penetrates through the third elastomeric member 8229 thus placing the needle 8216 in fluid communication with the mixing volume 8237 and the medicament (see, e.g. FIG. 193). The third elastomeric member 8229 contacts the distal end portion 8213 of the medicament container 8210 to stop its distal movement. Continued movement of the first elastomeric member 8221 and the second elastomeric member 8229 causes the medicament within the mixing volume 8237 to flow into the needle 8216 and out of the injector 8000. The first elastomeric member 8221 and the second elastomeric member 8225 can continue to move in the distal direction and into contact with the third elastomeric member 8229, thereby stopping the flow of medicament from the mixing volume 8237 through the needle 8216 and out of the injector 8000 (e.g., a seventh configuration, see FIG. 195).

Upon completion of the injection operation, the movable member 8300 disengages from the injection spring. The movable member 8300 can disengage from the injection spring by any suitable mechanism. For example, in some embodiments the injector 8000 can include a transfer member similar to the transfer member 7600 described above. After the movable member 8300 is disengaged from the injection spring, the retraction member 8440, which has been compressed by the injection operation between the movable member 8300 and the base 8510, can expand and can move the carrier 8260 and the needle 8216 in the proximal direction within injector 8000 (e.g., an eighth configuration, see FIG. 196). In some embodiments, retraction member 8440 can expand fully.

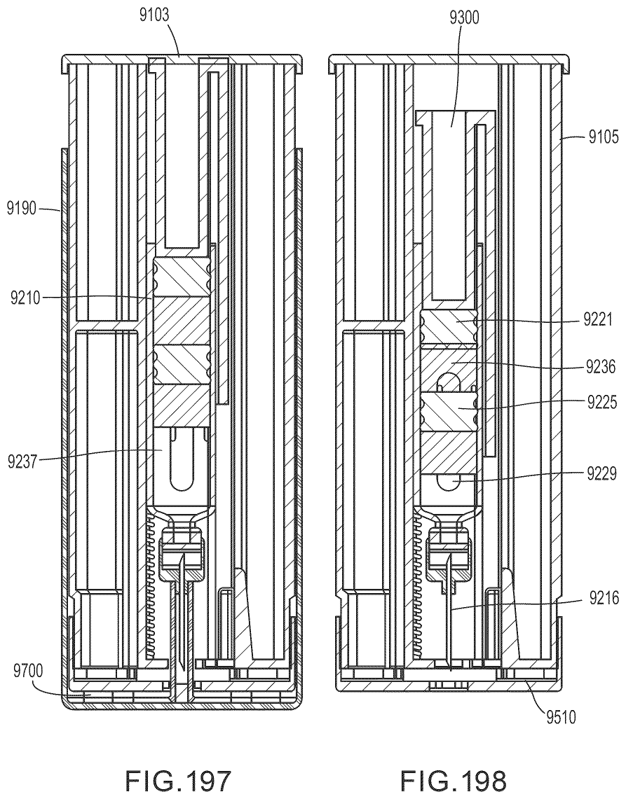

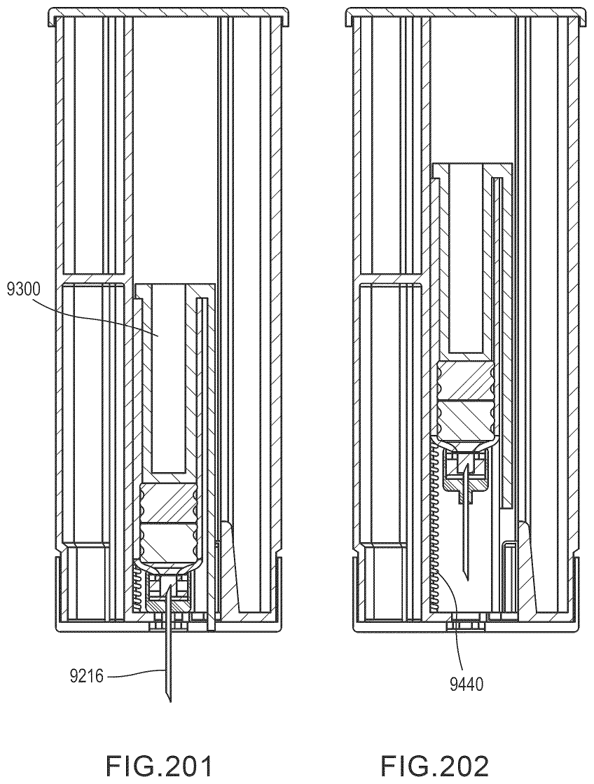

Although the injector 7000 is shown as described as having a first elastomeric member, a second elastomeric member, and a third elastomeric member within the medicament container. In other embodiments, the injector 7000 can include only a first elastomeric member and a second elastomeric member within the medicament container. For example, FIGS. 197-202 depict an injector 9000 that does not include a third elastomeric member within the medicament container. Other components within injector 9000, however, can be similar to and have similar functions as the components corresponding in the injector 7000 and the injector 8000. By way of example, first elastomeric member 9221 of the injector 9000 can be similar in configuration to first elastomeric member 7221 of the injector 7000.

The injector 9000 includes a housing 9100, a proximal cap 9103, a case 9190, a base 9510, a medicament container 9210, a first elastomeric member 9221 and a second elastomeric member 9225 that define a diluents volume 9236 and a mixing volume 9237, a needle 9216, and a movable member 9300. In some embodiments, the movable member 9300 can effectuate both the mixing and the injection of the medicament.

FIG. 197 depicts the injector 9000 with the case 9190 disposed about the housing 9100 (e.g., a first configuration). As depicted in FIG. 198, the case 9190 can be removed from the injector 9000 to place the injector 9000 in a second configuration. Removing the case 9190 can actuate an electronic assembly (not shown in FIGS. 197-202) to produce an electronic output, such as, for example, an output indicating the status of the injector 9000 or providing instructions for operation. Removing the case 9190 can actuate the electronic assembly by engaging a portion of the electronic assembly, placing a battery assembly in electronic communication with a processor, or the like.

The safety lock 9700 can be removed from the injector 9000 to place the injector 9000 in a third configuration (e.g., initiation of the mixing operation). Removing the safety lock 9700 can cause a first switch actuator 9724 to activate a first switch of the electronic assembly to produce a second electronic output and/or continue producing a current electronic output.

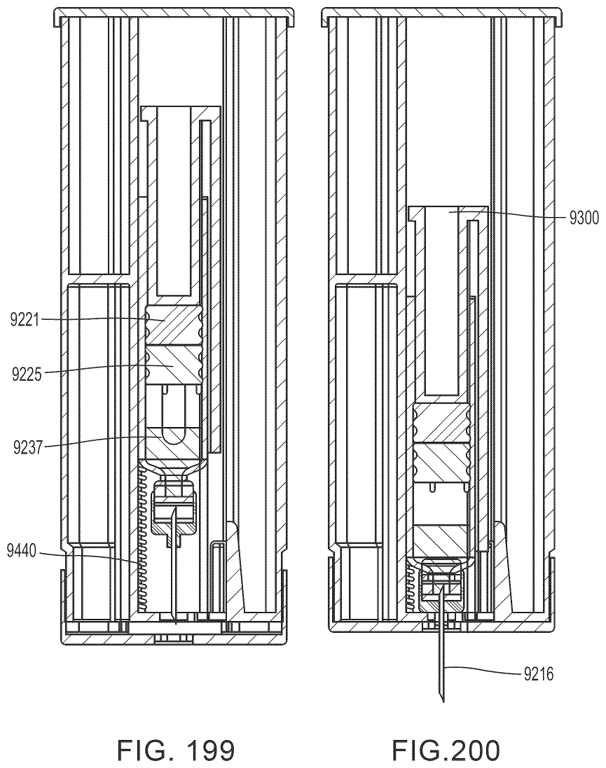

Removing the safety lock 9700 exposes the base 9510 of injector 9000. The base 9510 can be moved in the proximal direction thereby causing an injection spring to act against the proximal cap 9103, which moves the movable member 9300 in the distal direction. The initial movement of the movable member 9300 starts the mixing operation as described above with reference to the injector 8000 (e.g., the third configuration, see FIG. 198). More specifically, the base 9510 can be moved in the proximal direction and can cause a release rod to deform release portion to actuate the injection assembly, in a similar manner as described above. An injection spring acts against the proximal cap 9103 and a transfer mechanism (e.g., similar to the transfer member 7600), and causes the injection latch, and therefore the piston portion 9330, to move in the distal direction. The distal movement of the movable member 9300 causes a piston portion 9330 of the movable member 9300 to contact the first elastomeric member 9221. The distal movement of the first elastomeric member 9221 within the medicament container 9210 can move the second elastomeric member 9225 within the medicament container 9210 as described above, and as shown in FIGS. 198 and 199. In this manner, actuation of the injection spring produces the mixing operation, as described above.

As the diluent flows into the mixing volume 9237, the volume of the diluents volume 9236 can be reduced, and the volume of the mixing volume 9237 can remain substantially the same. The first elastomeric member 9221 can contact the second elastomeric member 9225 and can continue to move in the distal direction. In this manner, mixing of the medicament can be substantially complete (e.g., a fourth configuration). The distal movement of the first elastomeric member 9221 and the second elastomeric member 9225 can cause the volume of the mixing volume 9237 to be reduced and can cause any air within the mixing volume 9237 to vent out of the injector 9000.

The injector 9000, and any other injectors described herein (including the injector 7000 and 7000′), can use any suitable mechanism for venting the air within the mixing volume 9237. For example, in some embodiments, the mixing mechanism can include a “two-step” mixing actuator. The initial actuation (or first step) of the mixing mechanism results in the mixing operation, as described above. In such embodiments, the injector can include a protrusion or other member to limit the further movement of the spring. When mixing of the medicament is substantially complete, a user can orient the injector 9000 upwards, and can press a “vent” button, which actuates a release mechanism to allow the spring to expand further. Continued pressure exerted by the spring, can cause the container to move, such that the needle 9216 pierces a crimp seal. In this manner, air within the mixing volume 9237 can escape via needle 9216. In some embodiments, the continued pressure exerted by the spring can increase the turbulence of the diluent flowing within the medicament container, thereby enhancing the mixing operation.

In a three-plunger design (e.g., injectors 7000, 7000′ and 8000), upon pressing the “vent” button, continued distal movement of the first elastomeric member 7221′, the second elastomeric member 7225′ and the third elastomeric member 225′ within the medicament container 7210 causes the needle 7216 to pierce the third elastomeric member 7225′. In this manner, air within the mixing volume 7237′ can escape via needle 7216.

After venting, the user can push an injection button (not shown) and can allow a mixing spring (not shown) to continue to push the elastomeric members toward the distal end of the medicament container 7210 and can begin the injection process as described below.

Continued movement of the movable member 9300 starts the injection process (e.g., a fifth configuration). The movable member 9300, the carrier 9260, and the medicament container 9210 can move substantially together, and can cause retraction member 9440 to compress. In this manner, needle 9216 is inserted. A distal end portion of carrier 9260 can contact the base 9510 and/or the housing 9100 to stop the distal movement of the carrier 9260 and the medicament container 9210 (e.g., a sixth configuration).

The injection spring can continue to move the injection latch and movable member 9300. Said in another way, the movable member 9300 can begin to move relative to the carrier 9260. A piston portion 9330 of the movable member 9300 can cause the first elastomeric member 9221 and the second elastomeric member 9225 to move in the distal direction, causing medicament within the mixing volume 9237 to flow into the needle 9216 out of the injector 9000. The first elastomeric member 9221 and the second elastomeric member 9225 can continue to move in the distal direction into contact with the distal end portion 9213 of the medicament container 9210. At this point, the flow of medicament from the mixing volume 9237 through the needle 9216 and out of the injector 9000 is stopped (a seventh configuration).

As the transfer member and the movable member 9300 near the base 9510, the transfer member can be decoupled from the movable member 9300 by any suitable mechanism, thereby removing the force of the injection spring (not shown) from movable member 9300. The retraction member 9440, which has been compressed by the injection operation between the retraction member protrusion 9284 and the base 9510, can expand and can move the carrier 9260 and the needle 9216 in the proximal direction within the injector 9000 (e.g., an eighth configuration). In some embodiments, the retraction member 9440 can expand fully. In some embodiments, the carrier 9260 can move in the proximal direction and a latch included in the carrier 260 can contact a catch of the housing 9100 to stop the proximal movement of the carrier 9260 and the needle 9216.

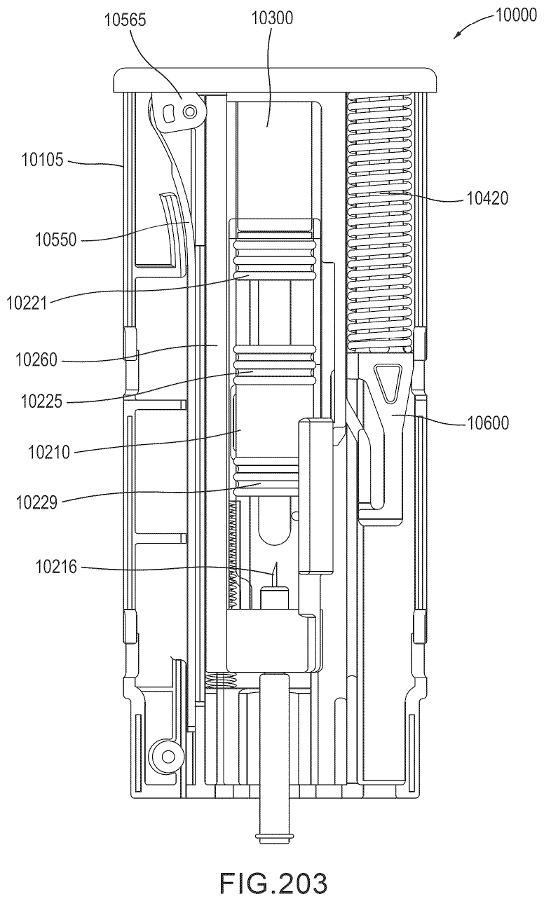

Although the injector 7000 is shown and described as having a mixing activator member 7550 that is partially disposed within the injection spring 7420, in other embodiments, a mixing activator release member and an injection spring (and/or injection assembly) can be disposed on opposing sides within an injector. Said another way, a mixing activator release member may not be disposed within the injection spring. For example, FIGS. 203-218 depict an injector 10000. Certain components within the injector 10000 can be similar to and have similar functions as the corresponding components in the injector 10000. By way of example, first elastomeric member 10221 of the injector 10000 can be similar in configuration to first elastomeric member 7221 of the injector 7000. The injector 10000 at least differs from injector 7000, however, in that a mixing activator member 10550 of the injector 10000 is not disposed within an injection spring 10420.

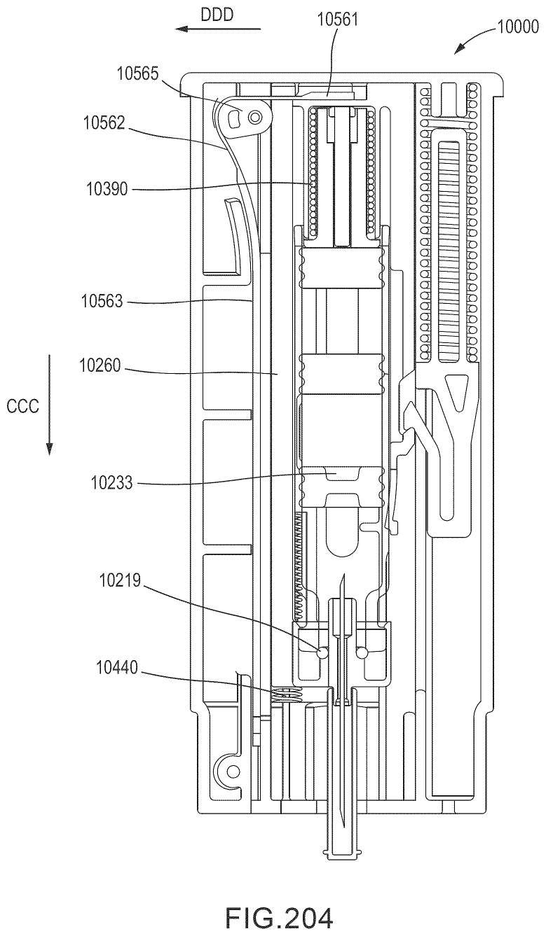

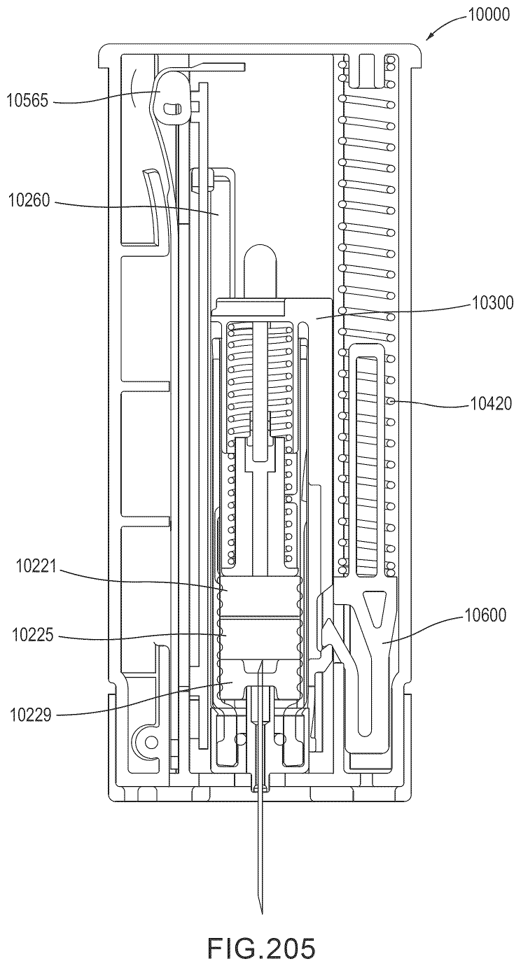

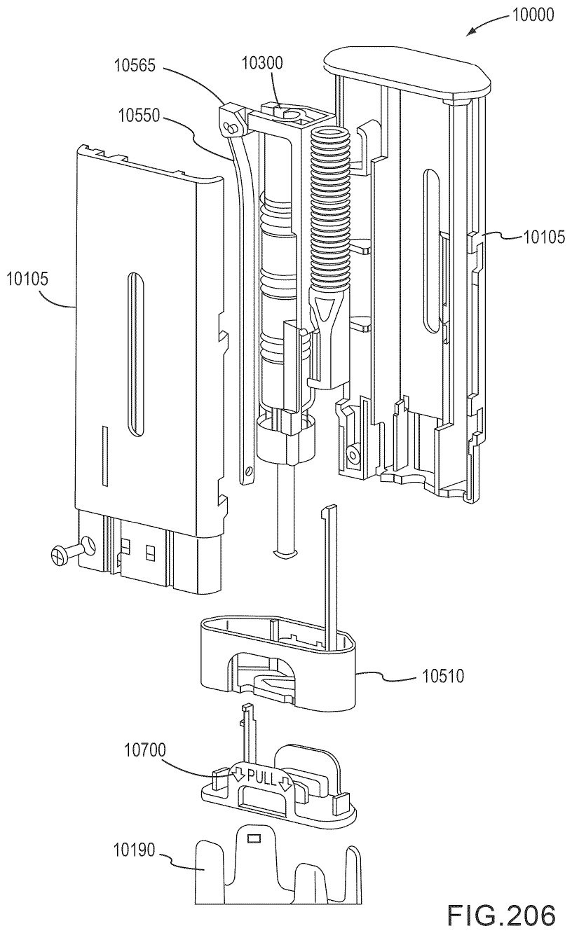

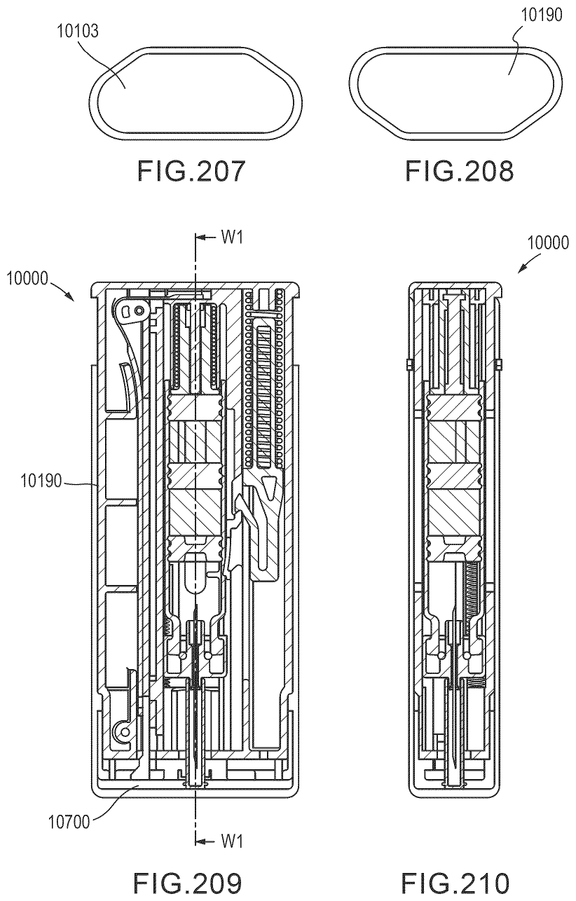

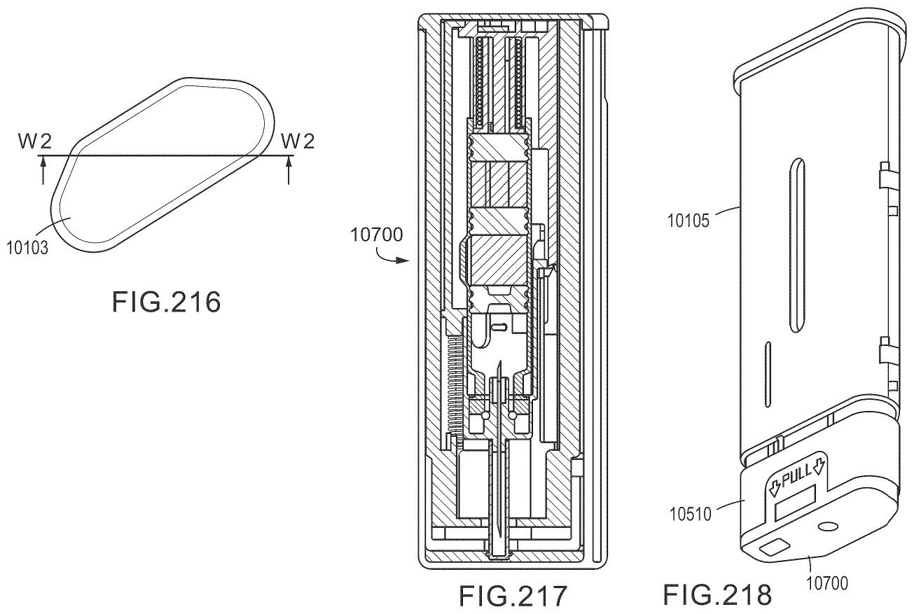

FIG. 203 is a front view of the injector 10000 in a second configuration, FIG. 204 is a cross-sectional view of the injector 10000 in the second configuration, FIG. 205 is a cross-sectional view of the injector 10000 in the seventh configuration, and FIG. 206 is an exploded perspective view of the injector 10000. FIG. 207-FIG. 215 depict the operation of the injector 10000. FIG. 216 depicts a top view of the injector 10000, and FIG. 217 depicts a cross-sectional view the injector 10000 taken along line W2-W2. FIG. 218 depicts a view of the injector 10000 in the second configuration. The injector 10000 includes a body 10105, a carrier 10260, a medicament container 10210, a first elastomeric member 10221, a second elastomeric member 10225, a third elastomeric member 10229, a mixing activator member 10550, a mixing latch guide 10565, a mixing spring 10390, a medicament delivery mechanism 10300, an injection spring 10420, an transfer member 10600, an energy-absorbing member 10219, and a retraction member 10440. As shown in FIGS. 203-206, the mixing activator member 10550 includes a first thickness 10561, a second thickness 10562, and third thickness 10563. In this manner, the rigidity of the mixing activator member 10550 can vary spatially. Said another way, the mixing activator member 10550 can be less rigid at certain points (e.g., the second thickness 10562) to allow the mixing activator member 10550 to deform and/bend more easily during the operation of the injector 10000, e.g. about the mixing latch guide 10565. In some embodiments, however, mixing activator member 10550 can be a substantially uniform thickness. In some embodiments, mixing activator member 10550 can include more or fewer than three thicknesses. In some embodiments, other injectors shown in described herein can include a mixing latch with varying thicknesses as described above. The mixing latch guide 10565 can act as a cam for the mixing activator member 10550 during movement of the mixing activator member 10550. In this manner, a vertical portion of the mixing activator member 10550 can move twice as far in the direction CCC as a horizontal portion of the mixing activator member 10550 moves in the direction DDD. Said another way, the mixing latch guide 10565 imparts a mechanical advantage on the mixing activator member 10550. In other embodiments, the mixing latch guide 10565 can be configured such that the vertical portion of the mixing activator member 10550 can move a shorter distance in the direction CCC than the horizontal portion of the mixing activator member 10550 moves in the direction DDD.

The injector 10000 also includes the energy-absorbing member 10219. The energy-absorbing member 10219 can absorb, deflect and/or redirect energy, and/or otherwise reduce the energy transferred into certain components of the injector 10000 during operation. More specifically, the energy-absorbing member 10219 can reduce the energy transferred to the medicament container during operation. In this manner, a medicament container including fragile materials can be less likely to deform and/or otherwise break during injection.

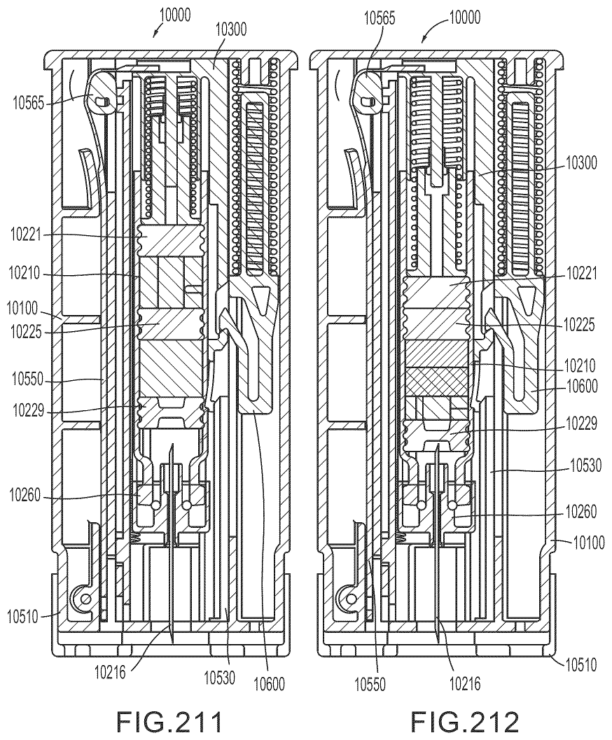

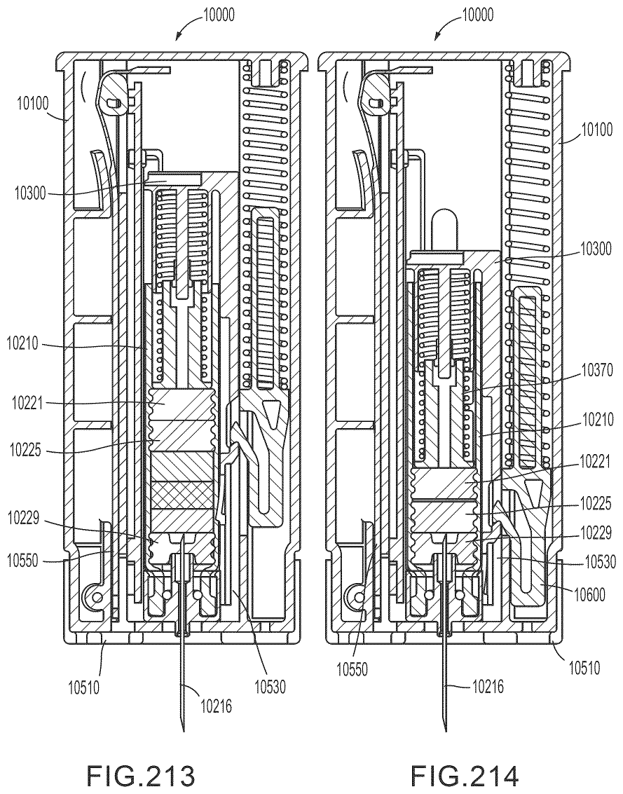

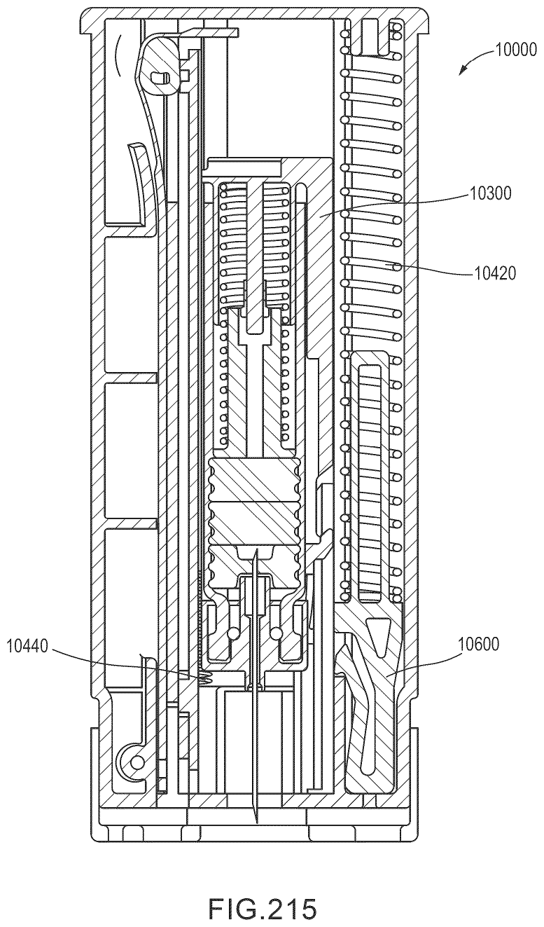

FIG. 207-FIG. 215 depict the operation of the injector 10000. The operation of injector 10000 can be similar to the operation of the injector 7000 and the injector 7000′ as described above. FIG. 207 is a top view of the injector 10000, FIG. 208 is a bottom view of the injector 10000, FIG. 209 is a cross-sectional view of the injector 10000 in the first configuration (i.e. prior to removal of a case 10190), and FIG. 210 is a cross-sectional view of the injector 10000 taken along line W1-W1. FIG. 211 is a cross-sectional view of the injector 10000 in the third configuration (e.g., mixing start). FIG. 212 is a cross-sectional view of the injector 10000 in the fourth configuration (e.g., mixing end). FIG. 213 is a cross-sectional view of the injector 10000 at the end of the fifth configuration (e.g., insertion) and the beginning of the sixth configuration (e.g., injection start). FIG. 214 is a cross-sectional view of the injector 10000 in the seventh configuration (e.g., injection end). FIG. 215 is a cross-sectional view of the injector 10000 in the eighth configuration (e.g. retraction). As shown in FIG. 215, after the injection process ends, the latch 10620 is decoupled from the medicament delivery mechanism 10300. In this manner, the retraction member 10440 does not have to overcome the force of the injection spring 10420.

Any of the devices and/or medicament containers shown and described herein can include any suitable medicament or therapeutic agent. For example, although the medical injectors described above are shown and described is including a multi-chamber medicament container (e.g., medicament container 6210) that includes a substantially dry medicament (e.g., contained within the dry medicament volume 6237) and a diluent (e.g., contained within the diluent volume 6237), in other embodiments, any of the medicament delivery devices disclosed herein can include a multi-chamber container that is filled with any suitable substances. For example, in some embodiments, any of the medicament delivery devices disclosed herein can include a medicament container (e.g., a cartridge) that separately stores and mixes, upon actuation, two liquid substances. For example in some embodiments, any of the devices shown and described herein can include a medicament container filled with (in separate chambers) epinephrine and at least one antihistamine (e.g., epinephrine and diphenhydramine, epinephrine and hydroxyzine, epinephrine and cetirizine); an antipsychotic medicament and a benzodiazepine (e.g. haloperidol and diazepam, haloperidol and midazolam, haloperidol and lorazepam); insulin and a GLP-1 analog or incretin mimetic (e.g. insulin and exenatide, insulin and lixisenatide); an NSAID and an opiode (e.g., ketorolac and buprenorphine). Other suitable compositions that can be included in any of the medicament containers and/or devices described herein include pralidoxime chloride and atropine; obidoxime chloride and atropine; epinephrine and atropine; methotrexate and etanercept; methotrexate and adalimumab; and methotrexate and certolizumab.

### Glucagon Formulation

In some embodiments, a composition can include glucagon and/or any pharmaceutically acceptable constituents for use in the medicament delivery devices disclosed herein. In some embodiments, the glucagon formulation can be prepared and/or filled according to any of the methods described herein (e.g., method 11000). A composition according to an embodiment can be formulated such that the target concentration of glucagon in the solution, either before lyophilization (see e.g., operation 11010 shown and described above with reference to FIG. 134) and/or after being reconstituted upon actuation of the device, is approximately 1 mg/mL. In other embodiments, the target concentration of glucagon in the solution, either before lyophilization and/or after being reconstituted, can be approximately 2 mg/mL, approximately 1.5 mg/mL, approximately 0.5 mg/mL (e.g., a pediatric dose) or approximately 0.25 mg/mL. In other embodiments a composition can be formulated such that the target concentration of glucagon in the solution, either before lyophilization and/or after being reconstituted upon actuation of the device, is between approximately 0.25 mg/mL and 2 mg/mL, between approximately 0.5 mg/mL and 1 mg/mL, or between approximately 0.8 mg/mL and 1.2 mg/mL.

In certain embodiments, the concentration (either before lyophilization or upon reconstitution) of glucagon in a glucagon formulation is about 1 mg/mL and the total solute concentration is about 50 mg/mL. For example, in some embodiments, a composition can include glucagon and any suitable bulking agents to increase the total solute concentration in the glucagon formulation. In this manner, the glucagon formulation can be more effectively lyophilized and/or reconstituted. For example, in some embodiments, as described below, certain bulking agents can be used to improve the stability, solubility and/or efficacy of the composition when reconstituted in any of the devices shown and described herein. In some embodiments, certain bulking agents can be used to produce a visual indicia when the composition is reconstituted (e.g., such agents can allow the reconstituted medicament to be more easily detected by the user).

In some embodiments, a composition can include a peptide, such as, for example, glucagon and a carbohydrate. In this manner, the stability of the peptide (e.g., glucagon) can be increased during lyophilization and subsequent storage. In particular, the stability of peptides, such as glucagon, can be increased in an amorphous (i.e. non-crystalline) environment. It is believed that carbohydrates undergoing dehydration create a solid-state environment that is amorphous and exhibits high viscosity when maintained below the glass transition temperature. In addition, carbohydrates contain multiple hydroxyl groups that may form hydrogen bonds with polar groups on a protein or peptide surface in an amorphous solid-state environment. Without being bound by any particular mechanism, when water is removed during lyophilization, such carbohydrates may maintain the hydrogen bonds and preserve the native-like solid state of the polypeptide structure. In certain embodiments, therefore, the glucagon formulations include other excipients, such as, but not limited to carbohydrates. Suitable carbohydrates include, but are not limited to, lactose, trehalose, mannitol, and combinations thereof.

Additionally, the solubility of glucagon increases below a pH of 4. In certain embodiments, the glucagon formulations, prior to lyophilization and/or after reconstitution, have a pH of less than about pH 5.0, including less than about pH 4.5, less than about pH 4.0, less than about pH 3.5, less than about pH 3.0, less than about pH 2.5, less than about pH 2.0. In other embodiments of the invention, the glucagon formulations, prior to lyophilization and/or after reconstitution, have a pH range of about pH 1.5 to about pH 5.0, inclusive of all ranges and subranges therebetween, e.g., about pH 2.0 to about pH 4.5, about pH 2.0 to about pH 4.0, about pH 2.0 to about pH 3.5, about pH 2.0 to about pH 3.0, about pH 2.0 to about pH 2.5, about pH 2.5 to about pH 4.5, about pH 2.5 to about pH 4.0, about pH 2.5 to about pH 3.5, about pH 2.5 to about pH 3.0, about pH 3.0 to about pH 4.5, about pH 3.0 to about pH 4.0, about pH 3.0 to about pH 3.5, about pH 3.5 to about pH 4.5, and about pH 3.5 to about pH 4.0. In certain embodiments, the pH of the glucagon formulation is adjusted prior to lyophilization by the addition of a suitable acid, such as hydrochloric acid or citric acid.

The lyophilized formulations of the present invention may be reconstituted by any suitable diluent or combination of diluent, including, but not limited to, water, sterile water, glycerin, or hydrochloric acid.

As described above, in some embodiments, a glucagon formulation can include any suitable bulking agents and/or excipients. Table 1 lists the formulations investigated for lyophilization. The formulations set for the below include a concentration of glucagon in the solution, either before lyophilization and/or after being reconstituted, of approximately 1 mg/mL.

TABLE 1

| Formulation | Excipients and Concentration | Medicament |
|---|---|---|
| 1 | Lactose - 49 mg/mL | 1 mg/mL glucagon |
| 2 | Trehalose - 40 mg/mL | 1 mg/mL glucagon |
| | Mannitol - 20 mg/mL | |
| 3 | Trehalose - 40 mg/mL | 1 mg/mL glucagon |
| | Mannitol - 20 mg/mL | |
| | Citric acid - 1.8 mg/mL | |
| | Sodium citrate - 0.35 mg/mL | |
| 4 | Glycine - 20 mg/mL | 1 mg/mL glucagon |
| 5 | Mannitol - 40 mg/mL | 1 mg/mL glucagon |
| | Ascorbic acid - 5 mg/mL | |

Formulation 1 included lactose, which is a known animal-derived excipient. Lactose, which is used in the commercially available glucagon formulations, is a reducing sugar that may destabilize glucagon. Accordingly, Formulations 2 through 5 are lactose-free formulations. Formulation 2 utilized trehalose and mannitol as carbohydrate bulking agents. Formulation 3 included a buffer system of citric acid and sodium citrate, in addition to the carbohydrate bulking agents. Formulation 4 was carbohydrate free, containing only glycine as the bulking agent. Formulation 5 utilized only mannitol as a bulking agent and included ascorbic acid. All formulations except Formulation 3 employed hydrochloric acid to reduce the solution pH to approximately 3 before lyophilization.

Trehalose, however, is a non-reducing sugar, and without being bound by any particular mechanism, may potentially increase the stability of glucagon, prior to lyophilization, during lyophilization, in storage, and/or after reconstitution. In addition to the improved properties of Formulation 3, the absence of any animal-based excipients, such as lactose, make it particularly appealing from a regulatory standpoint, as the FDA has strict guidelines regarding animal-based excipients.

All five formulations listed in Table 1 were successfully reconstituted with water and resulted in solutions suitable for use in the multi-chambered container closure system of the present invention.

In some embodiments, the medicament contained within any of the medicament containers shown herein can be a vaccine, such as, for example, an influenza A vaccine, an influenza B vaccine, an influenza A (H1N1) vaccine, a hepatitis A vaccine, a hepatitis B vaccine, a haemophilus influenza Type B (HiB) vaccine, a measles vaccine, a mumps vaccine, a rubella vaccine, a polio vaccine, a human papilloma virus (HPV) vaccine, a tetanus vaccine, a diphtheria vaccine, a pertussis vaccine, a bubonic plague vaccine, a yellow fever vaccine, a cholera vaccine, a malaria vaccine, a smallpox vaccine, a pneumococcal vaccine, a rotavirus vaccine, a varicella vaccine and/or a meningococcus vaccine. In other embodiments, the medicament contained within any of the medicament containers shown herein can be epinephrine. In other embodiments, the medicament contained within any of the medicament containers shown herein can be naloxone, including any of the naloxone formulations described in U.S. patent application Ser. No. 13/036,720, entitled “Medicament Delivery Device for Administration of Opioid Antagonists Including Formulation for Naloxone,” filed on Feb. 28, 2011.

In other embodiments, the medicament contained within any of the medicament containers shown herein can include insulin, glucagon, human growth hormone (HGH), erythropoiesis-stimulating agents (ESA), DeMab, Interferon and other chronic therapies, or the like. Such formulations can be produced using a general lyophilization process with glucagon (of recombinant origin) using bulking agents, stabilizers, buffers, acidifying agents or other excipients comprising of, but not limited to, one or more of the following combinations: lactose, hydrochloric acid; glucose, histidine, hydrochloric acid; trehalose, mannitol, citrate; trehalose, mannitol, hydrochloric acid; trehalose, glycine, hydrochloric acid; Mannitol, ascorbic acid; and Glycine, hydrochloric acid.

In other embodiments any of the injectors described herein can be filled with and/or used to inject medicament formulations, including lyophilized biologics and/or biopharmaceuticals, such as, for example, canakinumab, certolizumab, golimumab, and/or interleukins, for the treatment of crypyrin associated periodic syndromes, hereditary andioedema, and other auto-immune diseases. In yet other embodiments any of the injectors described herein can be filled with and/or used to inject intranasal biologics, such as glucagon or human growth hormone, formulated for use in an auto injector, for the treatment of musculoskeletal diseases, growth disorders, diabetes & treatment related disorders.

In other embodiments, any of the injectors described herein can be filled with and/or used to inject an anti-thrombotics, such as LMWH, ULMWH, Xa Inhibitors, biotinylated idraparinux, etc., for either the acute management and/or surgical prophylaxis of deep vein thrombosis and/or pulmonary embolism or for the management of other conditions which may require anticoagulation to prevent thromboembolism, such as its use in cardiovascular diseases including atrial fibrillation and ischemic stroke. In another example, in some embodiments an injector according to an embodiment can be filled with and/or used to inject formulations for the treatment of asthma and/or chronic obstructive pulmonary disease.

In other embodiments, any of the injectors described herein can be filled with and/or used to inject recombinant hyaluronidase.

In other embodiments, any of the injectors described herein can be filled with and/or used to inject depot medroxyprogesterone acetate for the treatment of infertility.

In other embodiments, any of the injectors described herein can be filled with and/or used to inject environmental, food, and household allergen formulations for the treatment of allergic disease, specifically for use in immunotherapy.

In still other embodiments, the medicament contained within any of the medicament containers shown herein can be a placebo substance (i.e., a substance with no active ingredients), such as water.

The medicament containers and/or medicament delivery devices disclosed herein can contain any suitable amount of any medicament. For example, in some embodiments, a medicament delivery device as shown herein can be a single-dose device containing an amount medicament to be delivered of approximately 0.4 mg, 0.8 mg, 1 mg, 1.6 mg or 2 mg. As described above, the fill volume can be such that the ratio of the delivery volume to the fill volume is any suitable value (e.g., 0.4, 0.6 or the like). In some embodiments, an electronic circuit system can include a “configuration switch” (similar to any of the switches shown and described above, such as the switch 6972) that, when actuated during the assembly of the delivery device, can select an electronic output corresponding to the dose contained within the medicament container.

Although the electronic circuit system 6900 is shown and described above as having two irreversible switches (e.g., switch 6972 and switch 6973), in other embodiments, an electronic circuit system can have any number of switches. Such switches can be either reversible or irreversible.

What is claimed is:

1. An apparatus, comprising:
a housing;
a medicament container assembly at least partially disposed within the housing, the medicament container assembly including a first elastomeric member and a second elastomeric member, the first elastomeric member defining, at least in part, a first volume, the second elastomeric member defining, at least in part, a second volume; and
a movable assembly including a first movable member and a second movable member, the second movable member configured to move within the medicament container assembly in a distal direction relative to the first movable member to change the movable assembly from a first configuration to a second configuration, a distal end portion of the second movable member configured to move at least the second elastomeric member in the distal direction within the medicament container assembly when the movable assembly changes from the first configuration to the second configuration, the first movable member configured to move the first elastomeric member in the distal direction within the medicament container assembly to expel at least a portion of the contents of the first volume and the second volume.

2. An apparatus, comprising:
a housing;
a medicament container at least partially disposed within the housing, the medicament container including a first elastomeric member and a second elastomeric member, the first elastomeric member defining, at least in part, a first volume, the second elastomeric member defining, at least in part, a second volume; and
a movable assembly configured to move at least the second elastomeric member in a distal direction a first distance within the medicament container when the movable assembly moves from a first configuration to a second configuration such that a first substance within the first volume is mixed with a second substance within the second volume, a length of the movable assembly increasing when the movable assembly moves from the first configuration to the second configuration, the movable assembly configured to move at least the first elastomeric member a second distance within the medicament container when at least a portion of the movable assembly moves within the housing after the movable assembly has been moved to the second configuration to expel at least a portion of the contents of the first volume and the second volume, the movable assembly includes a first movable member and a second movable member, the second movable member configured to move relative to the first movable member to change the movable assembly from the first configuration to the second configuration, the movable assembly includes a retention portion configured to limit movement of the second movable member when the movable assembly is in the second configuration.
